(19) United States
(12) Patent Application Publication
(10) Pub. No.: US 2024/0208898 A1
(43) Pub. Date: Jun. 27, 2024

(54) ENAMINE N-OXIDES: SYNTHESIS AND APPLICATION TO HYPOXIA-RESPONSIVE PRODRUGS AND IMAGING AGENTS

(71) Applicant: DANA-FARBER CANCER INSTITUTE, INC., Boston, MA (US)

(72) Inventors: Justin Kim, Jamaica Plain, MA (US); Dahye Kang, Brookline, MA (US); Sheldon T. Cheung, Spring House, PA (US)

(73) Assignee: DANA-FARBER CANCER INSTITUTE, INC., Boston, MA (US)

(21) Appl. No.: 18/283,264

(22) PCT Filed: Mar. 24, 2022
(86) PCT No.: PCT/US2022/021639

§ 371 Date: Sep. 21, 2023

Related U.S. Application Data
(60) Provisional application No. 63/165,992, filed on Mar. 25, 2021.

(51) Int. Cl. C07C 291/04; A61K 31/04; A61K 31/5375; A61K 31/553; A61K 31/695; C07C 215/24; C07D 498/22; C07F 7/08

(52) U.S. Cl. CPC C07C 291/04; A61K 31/04; A61K 31/5375; A61K 31/553; A61K 31/695; C07C 215/24; C07D 498/22; C07F 7/0816

(57) ABSTRACT

Disclosed are compounds and pharmaceutically acceptable salts and stereoisomers thereof that are suitable for diagnosis and the treatment of diseases and disorders characterized by, associate with or which exhibit tissue hypoxia, such as, for example, solid tumors. Also disclosed are pharmaceutical compositions containing same, and methods of making and using the compounds.

### RELATED APPLICATIONS

This application claims the benefit of priority under 35 U.S.C. § 119(e) to U.S. Provisional Application No. 63/165,992, filed Mar. 25, 2021, which is incorporated herein by reference in its entirety.

### GOVERNMENT SUPPORT

This invention was made with government support under grant number DP2 OD025643 awarded by The National Institutes of Health. The government has certain rights in the invention.

### BACKGROUND OF THE INVENTION

Tumor hypoxia refers to a state of oxygen deficiency in tumor tissue arising from the inadequate and irregular vascularization of rapidly proliferating cancer cells (Hockel et al., J. Natl. Cancer Inst. 93(4):266-276 (2001); Harris, A. L., Rev. Cancer 2(1):38-47 (2002); Eales et al., Oncogenesis 5(1):e190 (2016); Carreau et al., J. Cell. Mol. Med. 15(6):1239-1253 (2011)). Diffusion and perfusion limitations in these regions can lead to both persistent and fleeting levels of hypoxia that feature oxygen tensions less than 2%, and in the most severe cases of radiobiological hypoxia, below 0.1% (Mistry et al., Int. J. Radiat. Oncol, Biol., Phys. 98(5):1183-1196 (2017); McKeown, S. R., Br. J. Radiol. 87(1035):20130676 (2014)). Malnourishment and insufficient oxygenation of hypoxic tissue lead to large-scale adaptive reprogramming of cancer cells, transforming them into highly invasive and metastatic species with vastly altered metabolism and enhanced potential for proliferation and survival (Schito et al., Trends Cancer 2(12):758-770 (2016); Terry, et al., Int. J. Mol. Sci. 19(10):3044 (2018); Shah et al., Cancer Lett. 492:63-70 (2020)).

The onset of hypoxia in tumors is highly correlated with low survival rates and negative prognoses for cancer patients with advanced solid tumors. Current therapies have shortcomings. For example, radiotherapy is ineffective against hypoxic tissues given the essential role that oxygen plays as a radiosensitizer; chemotherapies, which target actively proliferating cells, are ineffective against cells in hypoxia-induced quiescence; and surgical options are often curtailed by the enhanced metastatic spread of cancers exhibiting hypoxia (Terry, et al., Int. J. Mol. Sci. 19(10):3044 (2018); Teicher, B. A., Cancer Metastasis Rev. 13(2):139-168 (1994); Rohwer et al., Drug Resist. Updates 14(3):191-201 (2011); Eckert et al., Front. Immunol. 10:407 (2019); Rankin et al., Science 352(6282):175-180 (2016)).

In addition, over the past six decades, dozens of hypoxia-activated prodrugs (HAPs) have undergone development; 11 have entered the clinic (Brown et al., Nat. Rev. Cancer 4(6):437-447 (2004); Wilson et al., Nat. Rev. Cancer 11(6):393-410 (2011); Hunter et al., Br. J. Cancer 114(10):1071-1077 (2016); Phillips, R. M., Cancer Chemother. Pharmacol. 77(3):441-457 (2016); Sharma et al., Chem. Soc. Rev. 48(3):771-813 (2019)). Clinical success, however, has been elusive (Spiegelberg et al., Clin. Transl. Radiat. Oncol. 15:62-69 (2019)). Most recently, HAPs tirapazamine (Brown, J. M., Br. J. Cancer 67(6):1163-1170 (1993)) and TH-302 (Duan et al., J. Med. Chem. 51(8):2412-2420 (2008)) each failed to meet their primary endpoints in phase 3 clinical trials (Rischin et al., J. Clin. Oncol. 28(18):2989-2995 (2010); Tap et al., Lancet Oncol. 18(8):1089-1103 (2017)), sparking a reassessment of the approach to prodrug development (Hunter et al., Br. J. Cancer 114(10):1071-1077 (2016); Spiegelberg et al., Clin. Transl. Radiat. Oncol. 15:62-69 (2019)). In particular, these studies highlighted the imperative of patient stratification (Wilson et al., Nat. Rev. Cancer 11(6):393-410 (2011); Spiegelberg et al., Clin. Transl. Radiat. Oncol. 15:62-69 (2019)).

In view of the foregoing, therapeutic agents targeting tumor hypoxia are urgently needed. The presence, extent, and severity of hypoxia vary greatly among patients, yet no clinical factors, such as size or stage, or genomic markers sufficiently predictive of hypoxia have been identified Hunter et al., Br. J. Cancer 114(10):1071-1077 (2016); Spiegelberg et al., Clin. Transl. Radiat. Oncol. 15:62-69 (2019)). This heterogeneity in hypoxia that can develop between tumors of the same type as well as across patient subpopulations exacerbates development of not only effective therapies but diagnostic agents as well. Retrospective studies from the tirapazamine trial revealed that while efficacy was not established in the general population, among the subgroup of patients in whom tumor hypoxia was detected by [¹⁸F]-misonidazole (MISO)-based positron emission tomography (PET) imaging, significant reduction in locoregional failure was observed in the treatment versus control cohorts (Rischin et al., J. Clin. Oncol. 24(13):2098-2104 (2006)). The results of this study underscore the need to exploit hypoxia to co-develop therapeutics and companion, non-invasive, diagnostic agents.

### SUMMARY OF THE INVENTION

The present invention provides novel branched enamine N-oxide compounds, methods of making them, and their uses in the diagnosis and treatment of diseases and disorders characterized by or that exhibit tissue hypoxia, particularly solid tumors. Their dual functionality imparts clinical versatility; hence they are referred to herein as theranostic agents. The inventive compounds undergo hypoxia-selective and hemeprotein-dependent reduction to induce the concomitant activation of an active agent such as a drug and/or diagnostic imagining agent once the compound reaches a site of hypoxic tissue. Therefore, inventive compounds that contain a therapeutic agent such as an anti-cancer drug may be viewed as a hypoxia-responsive prodrug. As shown in various working examples, an enamine N-oxide-caged cytotoxin staurosporine displayed hypoxic to normoxic cytotoxicity ratios that compare favorably with and are complementary to those of AQ₄N, a well-investigated aliphatic amine N-oxide hypoxia-activated prodrug. Other working examples confirm the dual function of the inventive enamine N-oxides, and demonstrate, both in cells and in in vivo tumor xenograft mouse models, that inventive compounds containing a near-infrared probe selectively labeled hypoxic tumor tissue.

The enamine N-oxide group acts as a cage for the active moiety. Sensitivity of inventive compounds to hypoxic environments facilitates cleavage of the therapeutic moiety, and its release from the enamine N-oxide into the hypoxic tissue. In so doing, the inventive compounds provide a mode of localized delivery to sites of tissue hypoxia such as tumor microenvironments that might be otherwise difficult to target.

Accordingly, aspects of the present invention are directed to compounds represented by formulas I, II, III, and IV:

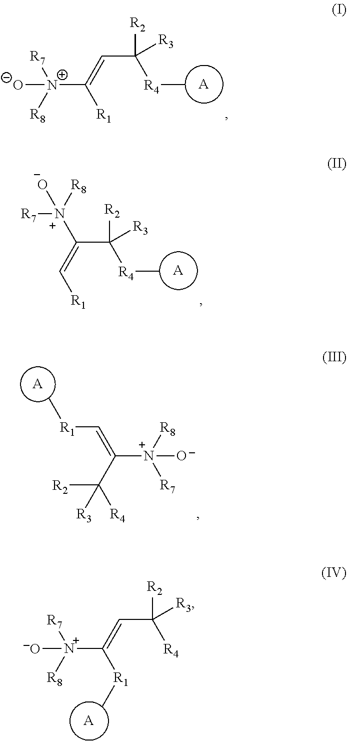

and pharmaceutically acceptable salts and stereoisomers thereof,
wherein R₁, R₂, R₃, R₄, R₇, R₈, and A are as defined herein.

Other aspects of the present invention are directed processes or methods for preparing compounds of formulas I, II, III, and IV. Compounds of formula (I) and compounds of formula (II) are regioisomers. Processes for making compounds of formulas (I and II) entail reacting a compound of formula (V),

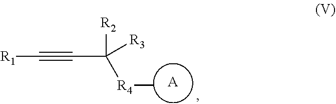

with a compound of formula (VII),

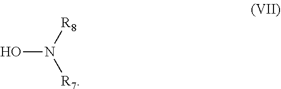

Compounds of formula (III) and compounds of formula (IV) are regioisomers. Processes for making compounds of formula (III) and (IV) entail reacting a compound of formula (VI)

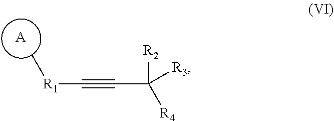

with a compound of formula (VII).

Yet other aspects of the present invention are directed to pharmaceutical compositions that include therapeutically and/or diagnostically effective amounts of compounds of formula (I-IV) and their pharmaceutically acceptable salts and stereoisomers, and a pharmaceutically acceptable carrier.

Further aspects of the present invention are directed to methods of treating diseases or disorders characterized by, associated with or exhibiting tissue hypoxia, that entail administration to a subject in need thereof a therapeutically effective amount of a compound of any one of formulas (I-IV) which contain a therapeutic moiety, e.g., an anti-cancer agent. In some embodiments, the methods are directed to methods of treating solid tumors characterized by a hypoxic tumor microenvironment.

Yet further aspects of the present invention are directed to methods of diagnosing diseases or disorders characterized by, associated with or exhibiting tissue hypoxia, that entail administration to a subject in need thereof a diagnostically effective amount of a compound of any one of formulas (I-IV) which contain at least one diagnostic moiety, e.g., a diagnostic imaging agent.

### BRIEF DESCRIPTION OF THE DRAWINGS

FIG. 1A-FIG. 1D is a series of schematics of mechanisms and designs of hypoxia-activated prodrugs. FIG. 1A shows hypoxia-activated prodrugs commonly exploit a futile redox cycle to achieve selectivity. Oxygen continually reverses the reduction of the prodrug by 1e⁻ reductases. FIG. 1B shows two sequential 2e⁻ reductions by hemeproteins convert aliphatic N-oxide prodrug AQ₄N into the cytotoxic agent AQ₄. FIG. 1C shows the design of new hypoxia-activated prodrugs termed enamine N-oxides. Enamine N-oxides can release small molecules upon 2e⁻ bioreduction selectively under hypoxic conditions. The resulting unsaturated iminium ion can readily react with biological nucleophiles. FIG. 1D shows a retro-Cope elimination between alkynes and dialkylhydroxylamine provide access to novel enamine N-oxides under mild reaction conditions. LG=leaving group.

FIG. 2A-FIG. 2B depicts the hydroamination reaction between alkynes and N,N-dialkylhydroxylamines. FIG. 2A shows the alkyne substrate scope of the reaction. FIG. 2B shows the hydroxylamine substrate scope. The major regioisomer of the product is depicted and yields are reported as the average isolated yield from two experiments. Regioisomeric ratios (r.r.) represent the ratio of major to minor products as determined by ¹H NMR analysis. When no r.r. is presented, only the depicted regioisomer is observed. Reactions were monitored by thin layer chromatography for disappearance of limiting reagent, and the reaction times are provided. ^{a}alkyne (1 equiv), N,N-diethylhydroxylamine (5.0 equiv). ^{b}N,N-dialkylhydroxylamine (1 equiv), alkyne (2.0 equiv).

FIG. 3A-FIG. 3F illustrate that enamine N-oxides are bioreduced in an oxygen-dependent manner in vitro and in cells in tissue culture. FIG. 3A is a panel of chromogenic enamine N-oxide probes that release 2-nitroaniline upon reduction. These probes were incubated with human liver microsomes under hypoxic conditions (0.1% pO₂) and their initial rates of reduction were measured and reported as relative rates of reduction (kᵣₑₗ) normalized to probe 32a. FIG. 3B shows the time dependent reduction of enamine N-oxide probe 32c by human liver microsomes under hypoxic and normoxic conditions. The data represent the concentration of 2-nitroaniline released based on its absorbance measurement at λ=412 nm, and show that there was a 21-fold enhancement in the initial rate of reduction under hypoxic conditions. FIG. 3C is a bar graph for oxygen, NADPH depletion, microsomal heat-inactivation, as well as a panel of CYP450 inhibitors that were evaluated for their ability to inhibit the reduction of enamine N-oxide probe 32c in the A412 microsomal assay using human liver microsomes. FIG. 3D depicts the chemical synthesis of enamine N-oxide caged staurosporine 37 and non-reducible control compound 36. FIG. 3E shows dose response curves of prodrug 37, staurosporine, and the non-reducible alkyne derivative 36 under both normoxic and hypoxic conditions in A431 cells (epidermoid carcinoma cells). pO₂ of each condition is denoted in parenthesis. FIG. 3F is a comparison table of the HCR values of compound 37 and AQ₄N in H460 and A431 cells. TFA=trifluoroacetic acid; HCR=hypoxic-to-normoxic cytotoxicity ratio; NADPH=nicotinamide adenine dinucleotide phosphate; H.I.=heat-inactivated; DPI=diphenyleneiodonium chloride; TAO=troleandomycin; DDC=diethyldithiocarbamate.

FIG. 4A-FIG. 4F illustrate that hypoxia-specific bioreduction of enamine N-oxides leads to intracellular protein labeling in cells and in vivo. FIG. 4A is a workflow diagram for visualizing hypoxia-dependent cellular or tumor tissue slice labeling by alkyne-containing enamine N-oxide probes. UnderUnder hypoxic conditions, these probes are reduced and covalently modify proteins with an alkyne handle. Cell lysates or tumor tissue slices from probe-treated samples are labeled with a TAMRA-azide fluorophore via copper-catalyzed azide-alkyne cycloaddition (CuAAC) click chemistry. FIG. 4B depicts the structure of alkyne-containing enamine N-oxide imaging probes and pimonidazole. FIG. 4C depicts A431 cells treated with probes 38-40 for 48 h and visualized by in-gel fluorescence after CuAAC with a TAMRA-azide fluorophore. FIG. 4D shows activation of enamine N-oxide probe 39 at ca. 1% pO₂ over 48 h and oxygen-dependent labeling in cell culture using a BxPC-3 pancreatic cancer cell line. FIG. 4E depicts the labeling profile of probe 39 and shows hypoxia selectivity in a series of cancer cell lines. Scale bar represents 100 μm. H/N=hypoxic-to-normoxic ratio; Nec=region of necrosis; TAMRA=tetramethylrhodamine; DAPI=4′,6-diamidino-2-phenylindole. FIG. 4F is a immunofluorescence image of tumor tissue slices obtained from BxPC-3 xenografts in mice that were intraperitoneally inoculated with enamine N-oxide 39 and pimonidazole. Localization of compound 39 shows strong correlation with the staining patterns from immunofluorescent labeling of known hypoxia markers GLUT1, HIF1α, CAIX, and pimonidazole.

FIG. 5A-FIG. 5B illustrates that hypoxia-responsive bioreduction of enamine N-oxides enabled near-infrared (NIR) fluorescence imaging of tumors in vivo. FIG. 5A depicts the structure of alkyne-containing enamine N-oxide NIR probe 42. FIG. 5B is a series of near-infrared images of a BxPC-3 xenograft mouse model showed preferential accumulation of enamine N-oxide probe 42 in the tumor. hpi=hours post-injection.

FIG. 6 shows that enamine N-oxides undergo thermal decomposition via Cope elimination. When model substrate S1 was heated to 60° C. in CDCl₃ for 1 h, two new products were isolated in addition to the starting material. The presence of α,β-unsaturated nitrone S2 and alkene S3 suggested that the Cope elimination is the degradation pathway for these enamine N-oxides.

FIG. 7A-FIG. 7B depicts that enamine N-oxide reduction leads to the release of a leaving group. FIG. 7A shows that incubation of enamine N-oxide 6 with reductants B₂(OH)₄ or Fe²⁺ in 100 mM HEPES, pH 7.4 led to the release of p-cresol as analyzed by HPLC. Due to the aerobic oxidation of Fe²⁺, sodium dithionite (Na₂S₂O₄) was used to reduce Fe³⁺ to Fe²⁺. FIG. 7B shows that incubation of alkyl N-oxide S4 did not lead to the release of p-cresol. Reduction lead to the generation of tertiary amine S5, confirming the essentiality of the internal olefin in translating an N-oxide reduction event to the release of a leaving group. Values represent mean±SD (n=2).

FIG. 8A-FIG. 8B illustrates the nucleophilic trapping experiment of an α,β-unsaturated iminium ion generated in situ. FIG. 8A shows that enamine N-oxide S6 is not reduced in the absence of diboron. FIG. 8B shows that enamine N-oxide S6 was reduced with B₂(OH)₄ in the presence of benzyl mercaptan (10 equiv) at room temperature. Michael adduct S7 was obtained in 94% yield. The aldehyde-containing product S7 is consistent with the nucleophilic addition of benzyl mercaptan into an α,β-unsaturated iminium ion intermediate that is generated upon leaving group extrusion. Subsequent hydrolysis of the iminium ion would yield the isolated product.

FIG. 9A-FIG. 9C is a series of graphs that show enamine N-oxide 32c in the presence of different reducing agents. FIG. 9A shows probe 32c (200 μM) incubated with tetrahydroxydiboron (B₂(OH)₄, 1 equiv, 200 μM) at room temperature. FIG. 9B shows probe 32c (200 μM) incubated with 5 mM cysteine in phosphate buffer, pH 7.4, at 37° C. FIG. 9C shows probe 32c (200 μM) incubated with 5 mM glutathione (GSH) in phosphate buffer, pH 7.4, at 37° C. The probe is fully reduced by diboron within 10 minutes but is unreactive toward cysteine or glutathione over 2 h.

FIG. 10 depicts the enamine N-oxide structure-dependent effects on microsomal reduction rate. Enamine N-oxide probes (32a-f) were incubated with human liver microsomes under both anaerobic (line graph) and aerobic conditions and measured for the release of 2-nitroaniline by UV spectroscopy. Relative reduction rates (kᵣₑₗ) are normalized to the reduction rate of the dimethyl-containing probe 32a. Microsomal reduction rates of both anaerobic and aerobic conditions are quantified in the table. Values represent mean±SD (n=3).

FIG. 11 is a bar graph that shows the stability of enamine N-oxide 6 in the presence of different metal cations. An enamine N-oxide (200 μM) containing a p-cresol leaving group was incubated with 2 mM of the following cations for 1 h at room temperature in 100 mM HEPES. pH 7.4. Only Fe²⁺ was capable of reducing enamine N-oxides. Fe²⁺ incubation included 10 mM sodium dithionite (see FIG. 7 for effect of sodium dithionite). Values represent mean±SD (n=2).

FIG. 12 shows dose response cell viability curves for prodrug 37 in A431 and H460 cell lines. Cells were treated with a derivative for 48 h under normoxic (20% pO₂) or hypoxic (0.1% pO₂) conditions and then tested for cell viability by the MTT assay. Values represent mean±SEM of data from biological replicates (n=3).

FIG. 13 shows dose response cell viability curves for AQ₄N in H460 and A431 cell lines. Cells were treated with AQ₄N for 24 h under normoxic (20% pO₂) or hypoxic (0.1% pO₂) conditions and then allowed to propagate under normoxic conditions for an additional 72 h. Cell viability was determined by the MTT assay. Values represent mean±SEM of data from biological replicates (n=3).

FIG. 14 is a graph depicting compound 37 metabolism in the presence of A431 cells. 7.5×10⁶ cells were incubated with 100 μM of compound 37 in serum free RPMI media and the presence of metabolic products was analyzed at the indicated time points by HPLC. A431 cells specifically converted enamine N-oxide 37 to staurosporine under hypoxic conditions at a ˜4-fold greater rate. Values represent mean±SEM (n=3).

FIG. 15A-FIG. 15B shows a dose response study for the activation of pro-apoptotic caspases in the A431 cell line by enamine N-oxides. FIG. 15A is a graph of a Caspase-Glo® assay that was used to measure the activities of caspases 3 and 7. Cells were treated with each compound at various concentrations for 24 h under normoxic (20% pO₂) or hypoxic (0.1% pO₂) conditions then the degree of apoptosis was measured using the Caspase-Glo® assay. Values represent mean±SEM of data from biological replicates (n=4). FIG. 15B is a Western blot of procaspase 3, cleaved caspase 3, cleaved PARP, and actin. Cells were treated with each compound 24 h at indicated concentrations under normoxic (20% pO₂) or hypoxic (0.1% pO₂) conditions then analyzed by Western blot using an apoptosis Western blot cocktail.

FIG. 16A-FIG. 16B are images of gels that show additional oxygen tensions used in the study of probe activation provided finer resolution of oxygen dependence. FIG. 16A shows Bx-PC3 cells were treated with enamine N-oxide probe 39 at the indicated oxygen tensions, lysed, labeled with TAMRA-azide fluorophore using CuAAC, then visualized by in-gel fluorescence. The hypoxia to normoxia (H/N) ratios are displayed. FIG. 16B is a Coosmassie stain of the gel that is provided as loading control.

FIG. 17 is Western Blot showing pimonidazole activation in BxPC-3 cells at different oxygen tensions. Cells were treated with pimonidazole for 48 h at 10 μM, lysed, and analyzed by Western blot. Pimonidazole does not reach its apparent half maximal labeling capacity (0.1% pO₂) until after 1% pO₂. H/N=Hypoxic to normoxic labeling ratio.

FIG. 18 is a series of images showing the comparison of probe 39, pimonidazole, and Hoechst 33342 localization. Tumor tissue slices were obtained from BxPC-3 xenografts in nude mice that were intraperitoneally inoculated with enamine N-oxide 39, pimonidazole, and Hoechst 33342. Probe 39 is visualized by copper-catalyzed azide-alkyne cycloaddition with a TAMRA-azide fluorophore. Pimonidazole and GLUT1 are visualized by immunofluorescence. Enamine N-oxide 39 co-localized with the GLUT1 and pimonidazole hypoxia markers. These hypoxia markers localize away from the perfusion marker Hoechst 33342. This pattern is consistent with the hypoxia-specific labeling of probe 39 in regions distal to well-perfused regions. TAMRA=tetramethylrhodamine; GLUT1=glucose transporter 1. Scale bar=100 μm.

FIG. 19 is a series of images showing the comparison of probe 39, CD31, and DAPI localization. Tumor tissue slices were obtained from BxPC-3 xenografts in nude mice that were intraperitoneally inoculated with enamine N-oxide 39. Probe 39 is visualized by copper-catalyzed azide-alkyne cycloaddition with a TAMRA-azide fluorophore. Immunofluorescent imaging of the CD31 endothelial cell marker shows that the vasculature is primarily localized in regions distal to regions that are labeled well by enamine N-oxide 39 staining and even further out from the region of necrosis. This pattern supports hypoxia-specific activation of enamine N-oxides in poorly vascularized regions of the tumor. TAMRA=tetramethylrhodamine; DAPI=4′,6-diamidino-2-phenylindole. Scale bar=100 μm.

FIG. 20A-FIG. 20C is a series of images of hematoxylin and eosin (H&E) stains and negative staining controls of BxPC-3 tumor xenograft tissue. FIG. 20A is a series of H&E and immunofluorescence images were generated from slices adjacent to the slices of images presented in FIG. 4D. FIG. 20B is a series of H&E and immunofluorescence images were generated from slices adjacent to the slices of images presented in FIG. 16. FIG. 20C is a series of H&E and immunofluorescence images were generated from slices adjacent to the slices of images presented in FIG. 17. Control images for the images visualized were generated via a copper-mediated azide-alkyne click reaction in the absence of copper sulfate. Secondary only control images for the immunofluorescence images are also presented. H&E=hematoxylin and eosin; TAMRA=tetramethylrhodamine; DAPI=4′,6-diamidino-2-phenylindole. Scale bar=100 μm.

FIG. 21 is a series of H&E stains and immunofluorescence images of BxPC-3 tumor xenograft tissue from mice injected with saline control. Images for each hypoxia marker were generated from adjacent tissue slices. Secondary only controls for CAIX and GLUT1 are from the same image but were processed with the same image settings as those used in the primary-containing sample within the same row. H&E=hematoxylin and eosin; DAPI=4′,6-diamidino-2-phenylindole. Scale bar=100 μm. *Antigen retrieval was performed by incubating slides in 10 mM citrate, pH 6.0 at 90° C. for 15 minutes.

FIG. 22A is an image of a gel showing that probe 42 labeled Bx-PC3 cells in a hypoxia-dependent manner in cell culture. Bx-PC3 cells were treated with probe 42 (10 μM) for 12 h under normoxia and hypoxia, then lysed and visualized by in-gel fluorescence after CuAAC with a TAMRA-azide fluorophore. FIG. 22B is a series of images confirming the presence of hypoxia in the tumor of a mouse imaged by near-infrared (NIR) probe 42 in FIG. 5. Copper-catalyzed azide-alkyne cycloaddition-mediated fluorescent labeling of the enamine N-oxide NIR probe 42 and immunofluorescent labeling of pimonidazole in tumor tissue slices exhibit robust co-localization and confirm the presence of hypoxia. TAMRA=tetramethylrhodamine; DAPI=4′,6-diamidino-2-phenylindole. Scale bar=100 μm.

FIG. 23 is a bar graph showing the relative average radiant efficiency of the tumoral region on the right flank compared to that of the no tumor region of the left flank over time (n=3). The 0 hours post-injection (hpi) time point represents a measurement prior to probe injection.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O— alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁2). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF3), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

As used herein, the term “π-electron withdrawing group” refers to functional group containing π-electrons which has a formal +ve or δ+ve charge, such as a carbonyl or nitro group, that attracts electron density.

As used herein, the term “inductive electron withdrawing group” refers to an atom or functional group containing an electronegative atom that attracts more electron density from the atoms to which they are attached, such as a fluoro or alkoxy group.

As used herein, the term “small molecule” refers to a molecule, whether naturally-occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.).

As used herein, the term active moiety refers to a distinct, definable portion or unit of an inventive compound that performs some function or activity or that is reactive with other molecules. Representative types of active moieties include therapeutic moieties and diagnostic moieties.

As used herein, the term “therapeutic moiety” refers to a portion of a portion of an inventive compound that provides a therapeutic effect with respect to a disease or disorder when it reaches its intended site of action, which in this case is hypoxic tissue.

As used herein, the term “diagnostic moiety” and “detectable moiety” are used interchangeably and refer to a portion of an inventive compound that provides a diagnostic effect in connection with a disease or disorder and permits visualization of cells or tissues in which inventive compounds accumulate, which in this case is hypoxic tissue.

In one aspect, compounds of the invention are represented by formulas I and II.

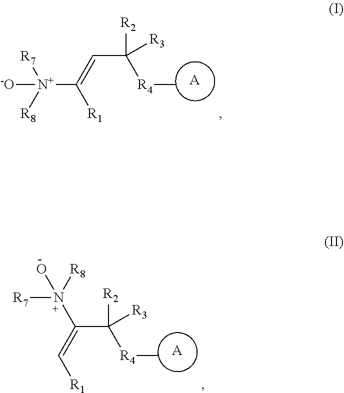

or a pharmaceutically acceptable salt or stereoisomer thereof,
wherein:
R₁ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a leaving group, or -[L]-diagnostic moiety, wherein R₁ may be optionally substituted;
   [L] is absent or a linking group that is capable of carrying a plurality of diagnostic moieties, which may be the same or different;
R₂ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a r-electron withdrawing group, or -[L]-diagnostic moiety, wherein R₂ may be optionally substituted;
   [L] is absent or a linking group that is capable of carrying a plurality of diagnostic moieties, which may be the same or different;
R₃ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a r-electron withdrawing group, or -[L]-diagnostic moiety, wherein R₃ may be optionally substituted;
   [L] is absent or a linking group that is capable of carrying a plurality of diagnostic moieties, which may be the same or different;
R₄ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a leaving group, a cleavable linking group, or -[L]-diagnostic moiety, wherein R₄ may be optionally substituted;
   [L] is absent or a linking group that is capable of carrying a plurality of diagnostic moieties, which may be the same or different;
R₇ is (C₁-C₅) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted, or
R₇ and R₈ together with the nitrogen atom to which they are attached, form a 4- to 7-membered heterocyclyl comprising 1 to 3 heteroatoms selected from O, N, and S;
R₈ is (C₁-C₅) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted; and
A is absent or a therapeutic moiety;
provided that the compound of formula (I or II) contains at least one -[L]-diagnostic moiety or therapeutic moiety,
and when A is a therapeutic moiety, R₄ is a cleavable linking group;
and when the compound contains at least one -[L]-diagnostic moiety and A is absent, R₁ and/or R₄ is a leaving group.

In some embodiments, R₁ is hydrogen.

In some embodiments, R₁ is C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, or 5- to 6-membered heterocyclyl.

In some embodiments, R₁ is an inductive electron withdrawing group. In some embodiments, the inductive electron withdrawing group is halogen, OR₅, SR₅, NR₅R₅, or a cyclic or acyclic amide, wherein each R₅ is independently hydrogen, (C₁-C₆) alkyl, (C₃-C₁₀) carbocyclyl, or 4- to 7-membered heterocyclyl, wherein said alkyl, carbocyclyl, or heterocyclyl is optionally substituted.

In some embodiments, R₁ is a leaving group, which as known in the art refers to an atom or group of atoms which breaks away from the rest of the molecule, taking with it the electron pair which used to be the bond between the leaving group and the rest of the molecule. In the context of the present disclosure, the leaving group breaks away from the compound upon contact with hypoxic tissue. Representative examples of leaving groups include iodo, bromo, chloro, OR₉, SR₉, —OC(O)R₉, —OC(O)OR₉, —OC(O)NR₉R₉, —OC(S)R₉, —OC(S)OR₉, —OC(S)NR₉R₉, —OS(O)₂R₉, —OS(O)₂OR₉, —OP(O)OR₉OR₉, —OP(O)R₉R₉, —SC(O)R₉, —SC(O)SR₉, or —SC(S)SR₉, wherein each R₉ is independently hydrogen, (C₁-C₆) alkyl, (C₃-C₁₀) carbocyclyl, or 4- to 7-membered heterocyclyl, wherein said alkyl, carbocyclyl, or heterocyclyl is optionally substituted.

In some embodiments, R₁ is a -[L]-diagnostic moiety. Diagnostic moieties typically contain a detectable moiety such as a label. Representative examples of diagnostic moieties include dyes, chromogenic agents, positron emission tomography (PET) tracers, and magnetic resonance imaging (MRI) contrast agents. The term “label” includes any moiety that allows the compound to which it is attached to be captured, detected, or visualized. A label may be directly detectable (i.e., it does not require any further reaction or manipulation to be detectable, e.g., a fluorophore or chromophore is directly detectable) or it may be indirectly detectable (i.e., it is made detectable through reaction with or binding to another entity that is detectable, e.g., a hapten is detectable by immunostaining after reaction with an appropriate antibody comprising a reporter such as a fluorophore). Representative examples of labels include affinity tags, radiometric labels (e.g., radionuclides (such as, for example, ³²P, ³⁵S, ³H, ¹⁴C, ¹²⁵I, ¹³¹I, and the like)), fluorescent dyes, phosphorescent dyes, chemiluminescent agents (such as, for example, acridinium esters, stabilized dioxetanes, and the like), spectrally resolvable inorganic fluorescent semiconductor nanocrystals (i.e., quantum dots), metal nanoparticles (e.g., gold, silver, copper, and platinum) or nanoclusters, enzymes (such as, for example, those used in an ELISA, i.e., horseradish peroxidase, beta-galactosidase, luciferase, alkaline phosphatase), colorimetric labels (such as, for example, dyes, colloidal gold, and the like), magnetic labels (such as, for example, Dynabeads™), and haptens.

In certain embodiments, the label comprises a fluorescent dye. Representative examples of fluorescent dyes include fluorescein and fluorescein dyes (e.g., fluorescein isothiocyanine (FITC), naphthofluorescein, 4′,5′-dichloro-2′,7′-dimethoxy-fluorescein, 6-carboxyfluorescein or FAM), carbocyanine, merocyanine, styryl dyes, oxonol dyes, phycoerythrin, erythrosin, eosin, rhodamine dyes (e.g., 5-carboxytetramethylrhodamine (TAMRA), carboxyrhodamine 6G, carboxy-X-rhodamine (ROX), lissamine rhodamine B, rhodamine 6G, rhodamine Green, rhodamine Red, or tetramethylrhodamine (TMR)), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g. Cy_3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R₆G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for coupling fluorescent dyes to other chemical entities see, for example, The Handbook of Fluorescent Probes and Research Products, 9th Ed., Molecular Probes, Inc., Eugene, Oregon and Molecular Probes Handbook, A Guide to Fluorescent Probes and Labeling Technologies, 11^{th} Ed., Life Technologies.

In some embodiments, the diagnostic moiety includes a rhodamine dye. In some embodiments, the diagnostic moiety includes tetramethylrhodamine (TAMRA) or a derivative thereof.

In some embodiments, the diagnostic moiety is an affinity tag, which as known in the art refers to agents that take part in an interaction (e.g., antigen and antibody, enzyme and substrate, receptor and ligand) that facilitates capture and/or purification of the molecule. Representative examples include small chemical compounds (such as biotin and derivatives thereof), short amino acid sequences (e.g., 2 to 20 amino acids in length, 4 to 12 amino acids in length, such as the (His)₆ tag, (His)₄ tag, (His)₃ tag, (His)₂ tag, (Leu)₄ tag, (Leu)₃ tag, (Leu)₂ tag, HA tag, FLAG tag, VSV-G tag, HSV tag, and V5 tag), chitin binding protein (CBP), maltose binding protein (MBP), Strep-tag, and glutathione-S-transferase (GST).

In some embodiments, the diagnostic moiety is a chromogenic agent, which as known in the art refers to a chemical compound that induces a color reaction. Representative examples include azo reagents such as methyl orange and methyl red, nitrophenols, phthaleins such as phenolphthalein or thymolphthalein, sulfonephthaleins such as bromophenol blue or bromocresol green, indophenols such as 2,6-dichlorophenolindophenol, azine reagents such as thiazine dye methylene blue, indigo carmine, derivatives of diphenylamine such as diphenylamine-4-sulfonic acid and variamine blue, arsenazo III, catechol violet, dithizone, 1-(2′-pyridylazo)-2-naphthol, 4-(2′-pyridylazo)resorcinol, chrome azurol S, eriochrome black T, eriochrome blue-black B, pyrogallol red, alizarin complexone, methylthymol blue, and xylenol orange.

In some embodiments, the diagnostic moiety is a PET tracer, which as known in the art refers to a radioligand used for imaging purposes. Representative examples include acetate (C-11), chline (C-11), fludeoxyglucose (F-18), sodium fluoride (F-18), fluoro-ethyl-spirpersone (F-18), methionine (C-11), prostate-specific membrane antigen (PSMA) (Ga-68), DOTATOC/DOTANOC/DOTATATE (Ga-68), florbetaben/florbetapir (F-18), rubidium (Rb-82), and FDDNP (F-18).

In some embodiments, the diagnostic moiety is a MRI contrast agent, which as known in the art refers to an agent that is used to improve the visibility of internal body structures. Representative examples include gadoterate, gadodiamide, gadobenate, gadopentetate, gadoteridol, gadofosveset, gadoveresetamide, gadoxetate, and gadobutrol.

In some embodiments, the diagnostic moiety is an intercalating agent, which as known in the art refers to an agent that inserted between the stacked base pairs of DNA. Intercalating agents are hydrophobic heterocyclic ring molecules that resemble the ring structure of base pairs. Representative examples include ethidium bromide, acridine orange, and actinomycin D.

In some embodiments, R₁ is a -[L]-diagnostic moiety, wherein [L] is a linking group that is optionally substituted by the same or a different -[L]-diagnostic moiety. In some embodiments, [L] is an alkylene chain, that may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different. In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —O—, —S—, —N(R′)—, —C(O)—, —C(O)O—, —OC(O)—, —C(O)N(R′)—, —N(R′)C(O)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —O—. In some embodiments, the alkylene chain is interrupted by, and/or terminates (at either or both termini) with —S—.

In some embodiments, [L] is a polyethylene glycol chain that may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different. In some embodiments, the polyethylene glycol chain has 1 to 20 —(CH₂CH₂—O)— units. In some embodiments, the polyethylene glycol chain has 1 to 15 —(CH₂CH₂—O)— units. In some embodiments, the polyethylene glycol chain has 1 to 10 —(CH₂CH₂—O)— units. In some embodiments, the polyethylene glycol chain has 1 to 5 —(CH₂CH₂—O)— units. In some embodiments, the polyethylene glycol chain has 1 to 2 —(CH₂CH₂—O)— units. In some embodiments, the polyethylene glycol is interrupted by, and/or terminates (at either or both termini) in at least one of —S—, —N(R′)—, —C(O)—, —C(O)O—, —OC(O)—, —C(O)N(R′)—, —N(R′)C(O)—, or a combination thereof. In some embodiments, the polyethylene glycol chain is interrupted by, and/or terminates (at either or both termini) with —C(O)—. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —S—.

Labels suitable for use in the present invention may be detectable by any of a variety of means including spectroscopic, photochemical, biochemical, immunochemical, electrical, optical, and chemical means.

In some embodiments, R₂ is hydrogen.

In some embodiments, R₂ is C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, or 5- to 6-membered heterocyclyl.

In some embodiments, R₂ is an inductive electron withdrawing group. In some embodiments, the inductive electron withdrawing group is halogen, OR₆, SR₆, or NR₆R₆, wherein each R₆ is independently hydrogen, C₁-C₆ alkyl, C₆-C₁₂ aryl, 5- to 10-membered heteroaryl, carbonyl, sulfonyl, sulfinyl, or phosphoryl. In some embodiments, the inductive electron withdrawing group is halogen. In some embodiments, the halogen is fluoro or chloro.

In some embodiments, R₂ is a π-electron withdrawing group. In some embodiments, the π-electron withdrawing group is —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, or —P(O)NR₆R₆NR₆R₆, wherein each R₆ is independently hydrogen, C₁-C₆ alkyl, C₆-C₁₂ aryl, or 5- to 10-membered heteroaryl.

In some embodiments, R₂ is a -[L]-diagnostic moiety. In some embodiments, R₂ is a -[L]-diagnostic moiety as described above for R¹. In some embodiments, R₂ is a -[L]-diagnostic moiety, wherein [L] is a linking group that is optionally substituted by the same or a different -[L]-diagnostic moiety.

In some embodiments, R₃ is hydrogen.

In some embodiments, R₃ is C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, or 5- to 6-membered heterocyclyl.

In some embodiments, R₃ is an inductive electron withdrawing group. In some embodiments, the inductive electron withdrawing group is halogen, OR₆, SR₆, or NR₆R₆, wherein each R₆ is independently hydrogen, C₁-C₆ alkyl, C₆-C₁₂ aryl, 5- to 10-membered heteroaryl, carbonyl, sulfonyl, sulfinyl, or phosphoryl. In some embodiments, the inductive electron withdrawing group is halogen. In some embodiments, the halogen is fluoro or chloro.

In some embodiments, R₃ is a π-electron withdrawing group. In some embodiments, the π-electron withdrawing group is —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, or —P(O)NR₆R₆NR₆R₆, wherein each R₆ is independently hydrogen, C₁-C₆ alkyl, C₆-C₁₂ aryl, or 5- to 10-membered heteroaryl.

In some embodiments, R₃ is a -[L]-diagnostic moiety. In some embodiments, R₃ is a diagnostic moiety as described above for R¹. In some embodiments, R₃ is a -[L]-diagnostic moiety, wherein [L] is a linking group that is optionally substituted by the same or a different -[L]-diagnostic moiety.

In some embodiments, R₁ and R₃ are each a -[L]-diagnostic moiety which may be the same or different. In some embodiments, R₁, R₂ and R₃ are each a -[L]-diagnostic moiety which may be the same or different from each other.

In some embodiments, R₄ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, or a 5- to 6-membered heterocyclyl.

In some embodiments, R₄ is a leaving group.

In some embodiments, R₄ is a -[L]-diagnostic moiety. In some embodiments, R₄ is a diagnostic moiety as described above for R¹. In some embodiments, R₄ is a -[L]-diagnostic moiety, wherein [L] is a linking group that is optionally substituted by the same or a different -[L]-diagnostic moiety.

In some embodiments, R₄ is a cleavable linking group.

In some embodiments, R₄ is an alkylene chain, that is interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different. In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, —OP(O)O(R′)O—, —N(R′)P(O)N(R′R′)N(R′)—, or a combination thereof. In some embodiments, the alkylene chain is interrupted by, and/or terminates (at either or both termini) with —OC(O)—. In some embodiments, the alkylene chain is interrupted by, and/or terminates (at either or both termini) with —OC(O)O—. In some embodiments, the alkylene chain is interrupted by, and/or terminates (at either or both termini) with —OC(O)N(R′)—.

In some embodiments, R₄ is a polyethylene glycol chain, that is interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different. In some embodiments, the polyethylene glycol chain has 1 to 20 —(CH₂CH₂—O)— units. In some embodiments, the polyethylene glycol chain has 1 to 15 —(CH₂CH₂—O)— units. In some embodiments, the polyethylene glycol chain has 1 to 10 —(CH₂CH₂—O)— units. In some embodiments, the polyethylene glycol chain has 1 to 5 —(CH₂CH₂—O)— units. In some embodiments, the polyethylene glycol chain has 1 to 2 —(CH₂CH₂—O)— units. In some embodiments, the polyethylene glycol is interrupted by, and/or terminates (at either or both termini) in at least one of —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, —OP(O)O(R′)O—, —N(R′)P(O)N(R′R′)N(R′)—, or a combination thereof. In some embodiments, the polyethylene glycol chain is interrupted by, and/or terminates (at either or both termini) with —OC(O)—. In some embodiments, the polyethylene glycol chain is interrupted by, and/or terminates (at either or both termini) with —OC(O)O—. In some embodiments, the polyethylene glycol chain is interrupted by, and/or terminates (at either or both termini) with —OC(O)N(R′)—.

In some embodiments, R₇ is Me, Et, ⁿBu, iPr, Cy,

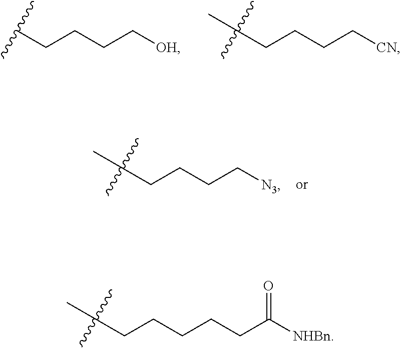

In some embodiments, R₇ and R₈ together with the nitrogen atom to which they are attached, form a 6-membered heterocyclyl comprising 2 heteroatoms selected from 0 and N. In some embodiments, R₇ and R₈ together with the nitrogen atom to which they are attached, form a piperidinyl, piperazinyl, or morphinyl group.

In some embodiments, R₈ is Me, Et, ⁿBu, iPr, Cy,

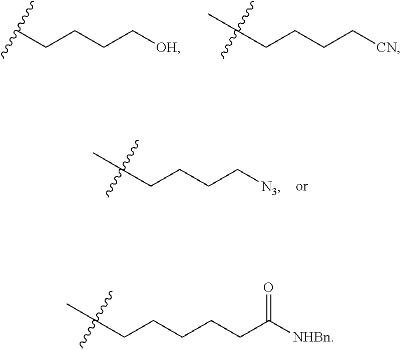

In some embodiments, the optionally substituent for R₁, R₂, R₃, R₄, R₇, and R₈, is a substituent selected from the group comprising of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, and a -[L]-diagnostic moiety. In some embodiments, the optional substituents may be one or more additional -[L]-diagnostic moieties, which may be the same or different.

In some embodiments, A is a therapeutic moiety. Broadly, the therapeutic moiety may be any agent that is effective in the treatment of a disease or disorder characterized by, associated with or that exhibits tissue hypoxia. In some embodiments, the therapeutic moiety is a small molecule. In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. In certain embodiments, the small molecule is a therapeutically active agent such as a drug (e.g., a molecule approved by the U.S. Food and Drug Administration as provided in the Code of Federal Regulations (C.F.R.)).

In some embodiments, the therapeutic moiety is an anti-cancer agent. Representative types of anti-cancer agents include anti-angiogenic agents, alkylating agents, antimetabolites, microtubulin polymerization perturbers, platinum coordination complexes, anthracenediones, substituted ureas, methylhydrazine derivatives, adrenocortical suppressants, hormones and antagonists, anti-cancer polysaccharides and anthracycline (e.g., an aclarubicin, daunorubicin, doxorubicin, epirubicin, idarubicin, mitoxantrone, pirarubicin, valrubicine and derivatives and analogs thereof), and kinase inhibitors (e.g., pan-Her inhibitors (e.g., HKI-272, BIBW-2992, PF299, SN29926 and PR-509E).

In some embodiments, the anti-cancer agent is a non-targeted agent, which as known in the art refers to agents with relatively broad modes of action that do not involve one or more specific molecular targets. Representative examples of non-targeted anti-cancer agents include alkylating agents (e.g., busulfan, chlorambucil, cyclophosphamide, ifosfamide, mechlorethamine, melphalan, carmustine, streptozocin, dacarbazine, temozolomide, altretamine, and thioTEPA), antimetabolites (e.g., capecitabine, cytarabine, 5′-fluorouracil, gemcitabine, cladribine, fludarabine, 6-mercaptopurine, and pentostatin), folate antagonists (e.g., methotrexate and pemetrexed), mitotic inhibitors (e.g., ocetaxel, paclitaxel, vinblastine, vincristine, vindesine, and vinorelbine), DNA inhibitors (e.g., hydroxyurea, carboplatin, cisplatin, oxaliplatin, mitomycin C, and pyrrolobenzodiazepine), topoisomerase inhibitors (e.g., topotecan, irinotecan, daunorubican, doxorubicin, etoposide, teniposide, and mitoxantrone), inducers of DNA breaks (e.g., bleomycin), ozogamicin, vedotin, emtansine, pasudotox, deruxtecan, govitecan, and mafodotin, or derivatives thereof.

In some embodiments, the therapeutic moiety is a targeted anti-cancer agent, which as known in the art, refers to agents with specific modes of action that involve one or more specific molecular targets. Representative examples of targeted anti-cancer agents include afatinib (EGFR, HER2), axitinib (KIT, PDGFRβ, VEGFR1/2/3), bosutinib (ABL), cabozantinib (FLT3, KIT, MET, RET, VEGFR2), ceritinib (ALK), crizotinib (ALK, MET), dabrafenib (ABL), erlotinib (EGFR), ibrutinib (BTK), idelalisib (PI3Kδ), imatinib (KIT, PDGFR, ABL), lapatinib (HER2, EGFR), lenvatinib (VEGFR2), nilotinib (ABL), olaparib (PARP), palbociclib (CDK4, CDK6), panobinostat (HDAC), pazopanib (VEGFR, PDGFR, KIT), ponatinib (ABL, FGFR1-3, FLT3, VEGFR2), regorafenib (KIT, PDGFRβ, RAF, RET, VEGFR1/2/3), romidepsin (HDAC), ruxolitinib (JAK1/2), sorafenib (VEGFR, PDGFR, KIT, RAT), temsirolimus (mTOR), trametinib (MEK), vandetanib (EGFR, RET, VEGFR2), vemurafenib (BRAF), vismodegib (PTCH), and vorinostat (HDAC). In some embodiments, the targeted anti-cancer agent is a kinase inhibitor. Representative examples of kinase inhibitors include abemaciclib, acalabrutinib, afatinib, alectinib, avapritinib, axitinib, baricitinib, benimetinib, bosutinib, brigatinib, cabozantinib, ceritinib, capmatinib, cobimetinib, crizotinib, dabrafenib, dacomitinib, dasatinib, encorafenib, entrectinib, erdafitinib, erlotinib, everolimus, fedratinib, fostamatinib, gefitinib, gilteritinib, ibrutinib, icotinib, imatinib, lapatinib, larotrectinib, lenvatinib, lorlatinib, midostaurin, neratinib, netarsudil, nilotinib, nintedanib, osimertinib, palbociclib, pazopanib, pemigatinib, pexidartinib, ponatinib, pralsetinib, regorafenib, ribociclib, ripretinib, ruxolitinib, selpercatinib, selumetinib, sirolimus, sorafenib, sunitinib, temsirolimus, tofacitinib, tremetinib, tucatinib, upadacitinib, vandetanib, vemurafenib, and zanubrutinib.

In some embodiments, the therapeutic moiety is a hypoxia-inducible factor inhibitor (HIF). Representative types of HIFs include daprodustat, desidustat, molidustat, roxadustat, vadadustat, wortmannin, LY94002, GDC-0941, PI-103, rapamycin, PP242, aminoflavone, glyceollins (e.g. glyceollin I, glyceollin II, glyceollin III, and glyceollin IV), topotecan, EZN-2968, ENMD-1198, geldanamycin, vorinostat, YC-1, PX-478, pleurotin, cardiac glycosides (e.g., convallotoxin, antarin, ouabin, digoxin, digitoxin, oleandrin, adonitoxin, daigremontianin, scillarenin, and proscillaridine A), FM19G11, acriflavine, PT2385, echinomycin, chetomin, bortezomib, amphotericin B, triptolide, and AJM290. Yet other HIF inhibitors which may be suitable for use in the present invention are disclosed in U.S. Patent Application Publications 2017/0157112 and 2017/0157111.

In some embodiments, the therapeutic moiety is an apoptotic agent, which as known in the art refers to agents that work to stop cells from cycling and cause apoptosis activation by blocking growth and survival, also known as programmed cell death. Representative examples include staurosporine, raptinal, anthracyclines (e.g., doxorubicin, daunorubicin, epirubicin, and idarubicin), prodigiosins (e.g., prodigiosin, nonylprodigiosin, undecylprodigiosin, metacycloprodigiosin, streptorubin B, and obatoclax), bortezomib, HGS-ETR1, HGS-ETR2, HGS-TR2J, PRO1762, TRA-8, CD95-Fc, adalimumab, etanercept, remicade, CDP571, IDN-6556, IDN-6734, VX-799, MX1013, VX-740, VX-756, M-920, M-826, immunocaps-3, immunocaps-6, Ad-G/iCasp3, TWX024, embelin, LY2181308, chelerythrine, antimycin A, HA14-1, genasense, chalcones, ONYX-015, CDB3, amifostine, and pifithrin-α.

In some embodiments, the therapeutic moiety is a non-steroidal anti-inflammatory drug (NSAID). Representative examples of NSAIDs agents include celecoxib, diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, sulindac, and tolmetin.

In some embodiments, the therapeutic moiety is a disease-modifying antirheumatic drug (DMARD). Representative examples of DMARDs include hydroxychloroquine, leflunomide, methotrexate, sulfasalazine, minocycline, penicillamine, cyclophosphamide, azathiopurine, cyclosporine, apremilast, and mycophenolate mofetil.

In some embodiments, the compound of formula (I) is represented by any one of the following structures:

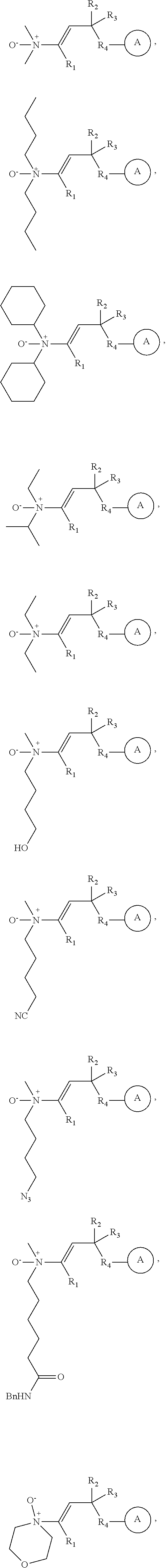

or a pharmaceutically acceptable salt or stereoisomer thereof. In some embodiments, R₁ is hydrogen, C₁-C₆ alkyl, C₁-C₆ haloalkyl, C₁-C₆ haloalkoxy, CN, NO₂, NH₂, (C₁-C₆ alkyl)NH, halogen, OR₅, SR₅, NR₅R₅, or a -[L]-diagnostic moiety, wherein each R₅ is independently hydrogen, (C₁-C₆) alkyl, (C₃-C₁₀) carbocyclyl, 4- to 7-membered heterocyclyl; and/or R₂ is halogen, OR₆, SR₆, NR₆R₆, —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, —P(O)NR₆R₆NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₃ is halogen, OR₆, SR₆, NR₆R₆, —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, —P(O)NR₆R₆NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₄ is a leaving group, a cleavable linking group, or a -[L]-diagnostic moiety; and/or A is absent or a therapeutic moiety. In some embodiments, halogen is fluoro or chloro. In some embodiments, C₆-C₁₂ aryl is phenyl. In some embodiments, 5- to 10-membered heteroaryl is pyrrole, furan, thiophene, pyridine, or pyrimidine. In some embodiments, C₁-C₆ alkyl is methyl, ethyl or propyl or isopropyl. In some embodiments, A is an anti-cancer agent. In some embodiments, the diagnostic moiety is a fluorescent dye.

In some embodiments for the compound of formula (I), R₁ is hydrogen, C₁-C₆ alkyl, CN, NH₂, (C₁-C₆ alkyl)NH, halogen, OR₅, SR₅, or NR₅R₅, wherein each R₅ is independently hydrogen, (C₁-C₆) alkyl, (C₃-C₁₀) carbocyclyl, or 4- to 7-membered heterocyclyl; and/or R₂ is halogen, OR₆, SR₆, NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₃ is halogen, OR₆, SR₆, or NR₆R₆, wherein each R₆ is independently hydrogen, C₁-C₆ alkyl, C₆-C₁₂ aryl, or 5- to 10-membered heteroaryl; and/or R₄ is a cleavable linking group; and/or A is a therapeutic moiety. In some embodiments, halogen is fluoro or chloro. In some embodiments, C₆-C₁₂ aryl is phenyl. In some embodiments, 5- to 10-membered heteroaryl is pyrrole, furan, thiophene, pyridine, or pyrimidine. In some embodiments, C₁-C₆ alkyl is methyl, ethyl or propyl or isopropyl. In some embodiments, A is an anti-cancer agent.

In some embodiments, the compound of formula (I) is represented by any one of formulas Ia-If:

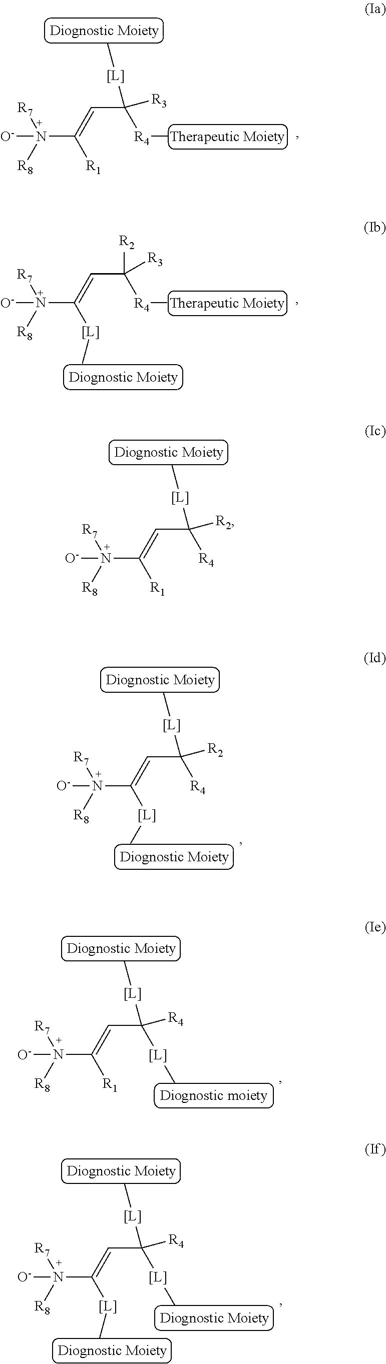

or a pharmaceutically acceptable salt or stereoisomer thereof.

In some embodiments, the compound of formula (Ia) is represented by any one of the following structures:

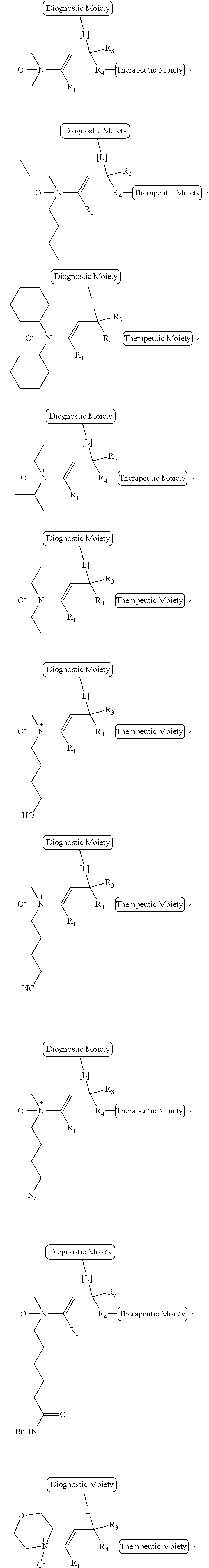

or a pharmaceutically acceptable salt or stereoisomer thereof.

In some embodiments, the compound of formula (Ib) is represented by any one of the following structures:

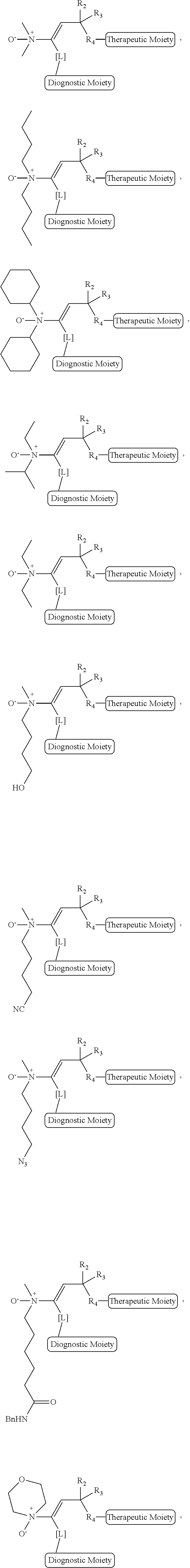

or a pharmaceutically acceptable salt or stereoisomer thereof.

In some embodiments, the compound of formula (Ic) is represented by any one of the

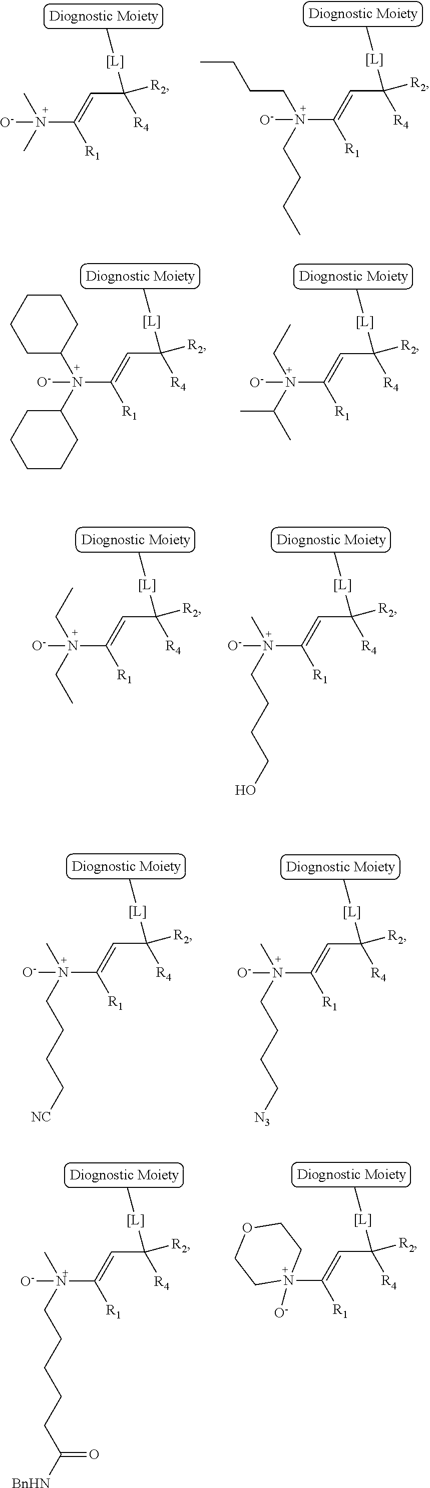

or a pharmaceutically acceptable salt or stereoisomer thereof.

In some embodiments, the compound of formula (Id) is represented by any one of the following structures:

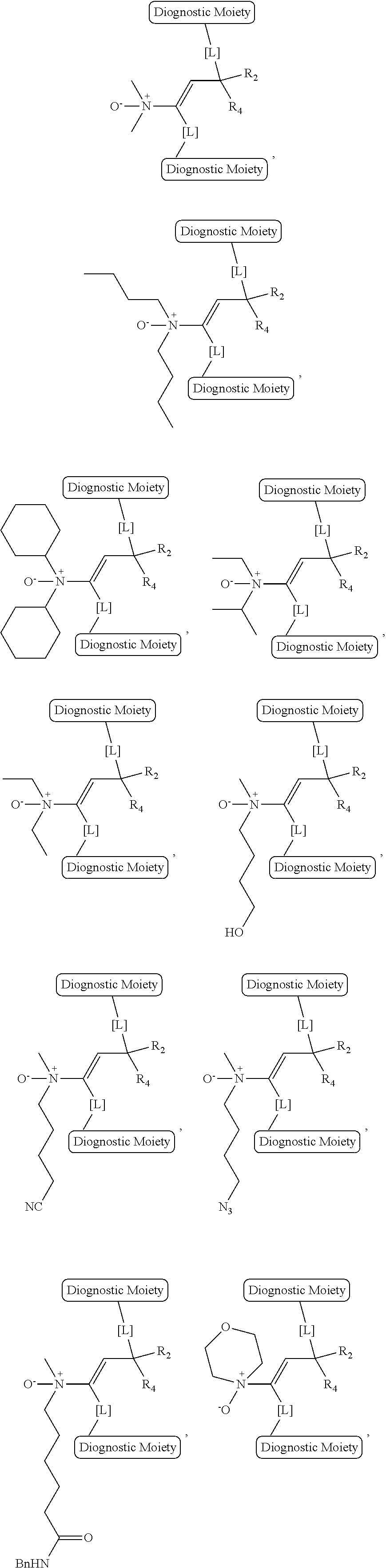

or a pharmaceutically acceptable salt or stereoisomer thereof.

In some embodiments, the compound of formula (Ie) is represented by any one of the following structures:

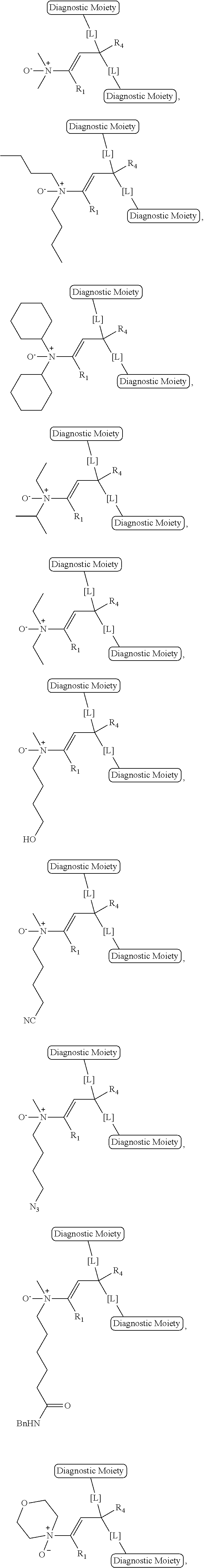

or a pharmaceutically acceptable salt or stereoisomer thereof.

In some embodiments, the compound of formula (If) is represented by any one of the following structures:

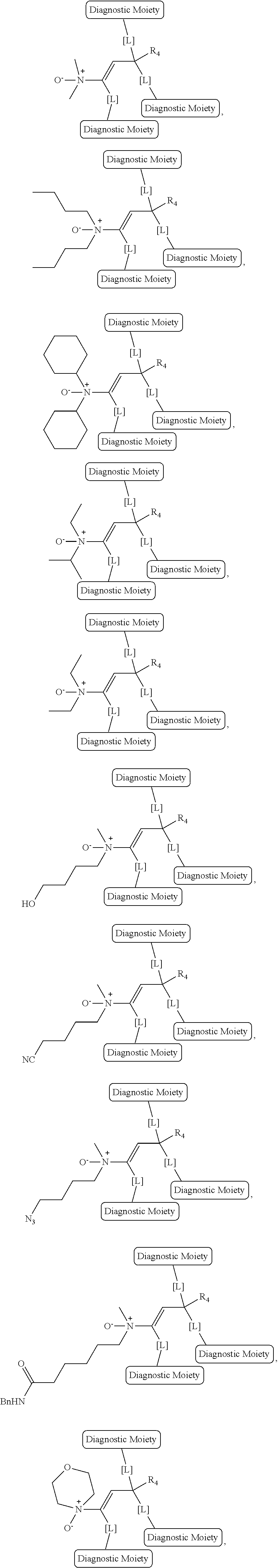

or a pharmaceutically acceptable salt or stereoisomer thereof.

In some embodiments, the compound of formula (II) is represented by any one of the following structures.

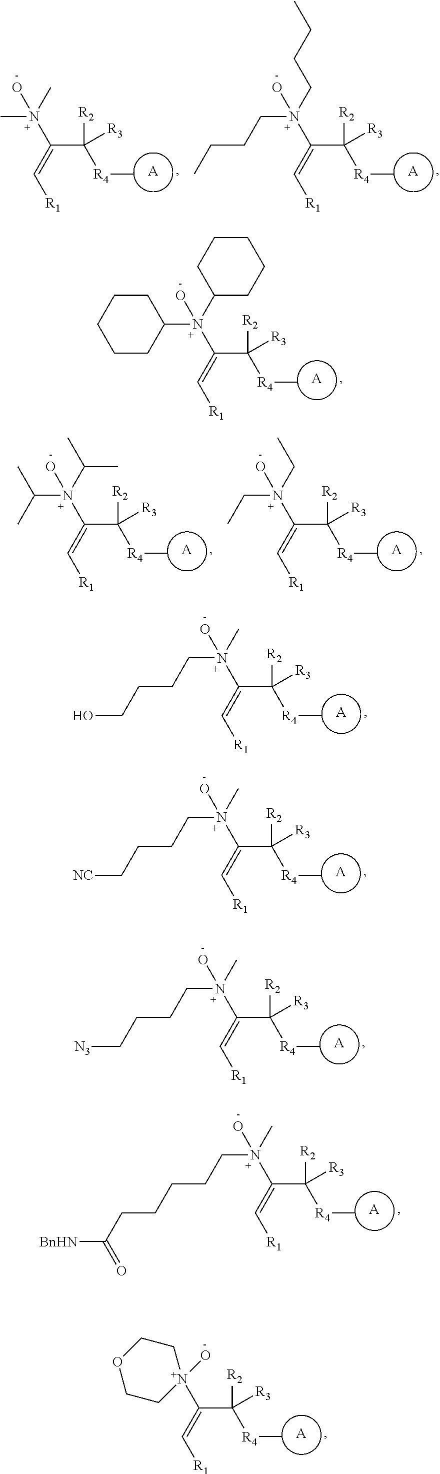

or a pharmaceutically acceptable salt or stereoisomer thereof. In some embodiments, R₁ is hydrogen, C₁-C₆ alkyl, C₁-C₆ haloalkyl, C₁-C₆ haloalkoxy, CN, NO₂, NH₂, (C₁-C₆ alkyl)NH, halogen, OR₅, SR₅, NR₅R₅, or a -[L]-diagnostic moiety, wherein each R₅ is independently hydrogen, (C₁-C₆) alkyl, (C₃-C₁₀) carbocyclyl, or 4- to 7-membered heterocyclyl; and/or R₂ is halogen, OR₆, SR₆, NR₆R₆, —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, —P(O)NR₆R₆NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₃ is halogen, OR₆, SR₆, NR₆R₆, —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, —P(O)NR₆R₆NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₄ is a leaving group, a cleavable linking group, or a -[L]-diagnostic moiety; and/or A is absent or a therapeutic moiety. In some embodiments, halogen is fluor or chloro. In some embodiments, C₆-C₁₂ aryl is phenyl. In some embodiments, 5- to 10-membered heteroaryl is pyrrole, furan, thiophene, pyridine, or pyrimidine. In some embodiments, C₁-C₆ alkyl is methyl, ethyl or propyl or isopropyl. In some embodiments, A is an anti-cancer agent. In some embodiments, the diagnostic moiety is a fluorescent dye.

In some embodiments for a compound of formula (II), R₁ is hydrogen, C₁-C₆ alkyl, CN, NH₂, (C₁-C₆ alkyl)NH, halogen, OR₅, SR₅, or NR₅R₅, wherein each R₅ is independently hydrogen, (C₁-C₆) alkyl, (C₃-C₁₀) carbocyclyl, or 4- to 7-membered heterocyclyl; and/or R₂ is halogen, OR₆, SR₆, NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₃ is halogen, OR₆, SR₆, or NR₆R₆, wherein each R₆ is independently hydrogen, C₁-C₆ alkyl, C₆-C₁₂ aryl, or 5- to 10-membered heteroaryl; and/or R₄ is a cleavable linking group and/or A is a therapeutic moiety. In some embodiments, halogen is fluor or chloro. In some embodiments, C₆-C₁₂ aryl is phenyl. In some embodiments, 5- to 10-membered heteroaryl is pyrrole, furan, thiophene, pyridine, or pyrimidine. In some embodiments, C₁-C₆ alkyl is methyl, ethyl or propyl or isopropyl. In some embodiments, A is an anti-cancer agent.

In some embodiments, the compound of formula (II) is represented by formula IIa-IIf:

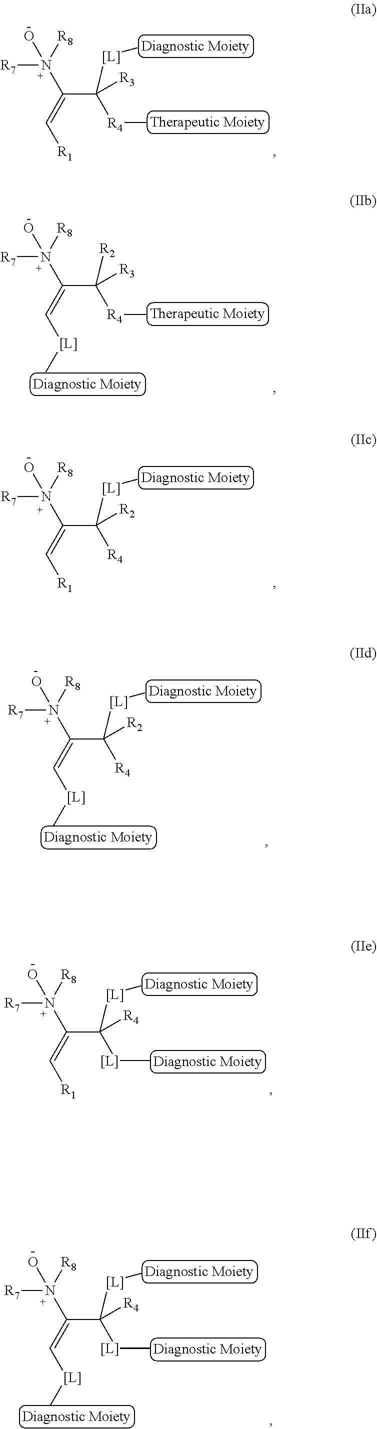

or a pharmaceutically acceptable salt or stereoisomer thereof.

In some embodiments, the compound of formula (IIa) is represented by any one of the following structures:

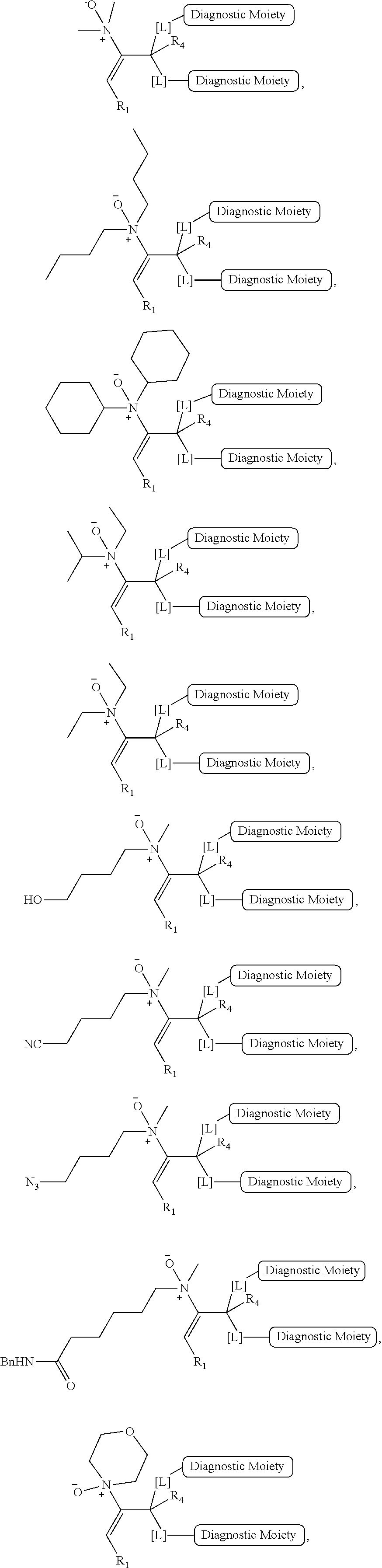

or a pharmaceutically acceptable salt or stereoisomer thereof.

In some embodiments, the compound of formula (IIb) is represented by any one of the following structures:

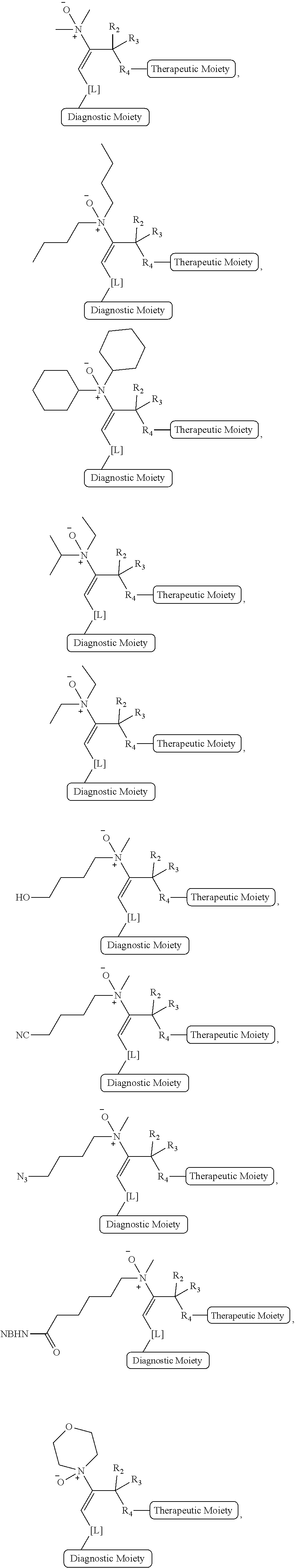

or a pharmaceutically acceptable salt or stereoisomer thereof.

In some embodiments, the compound of formula (IIc) is represented by any one of the following structures:

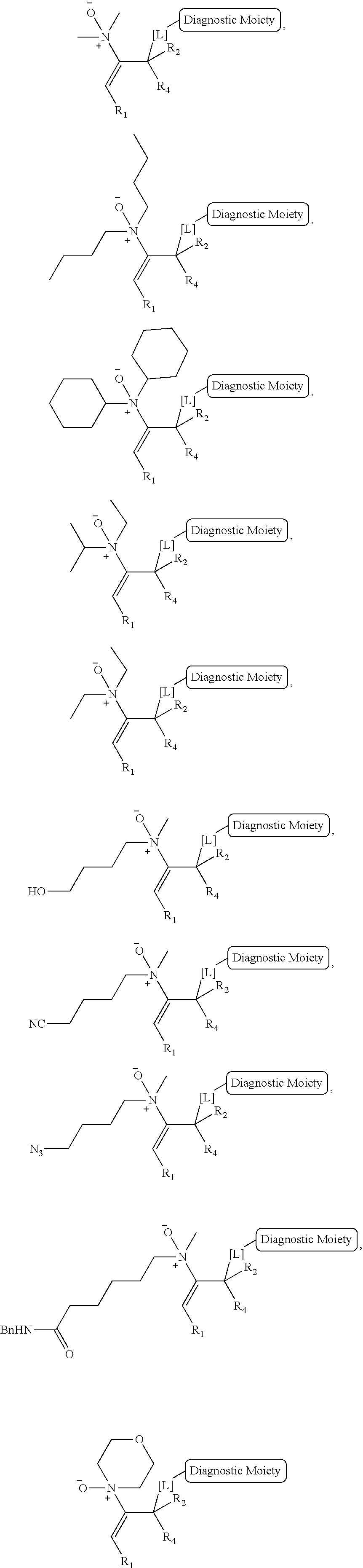

or a pharmaceutically acceptable salt or stereoisomer thereof.

In some embodiments, the compound of formula (IId) is represented by any one of the following structures:

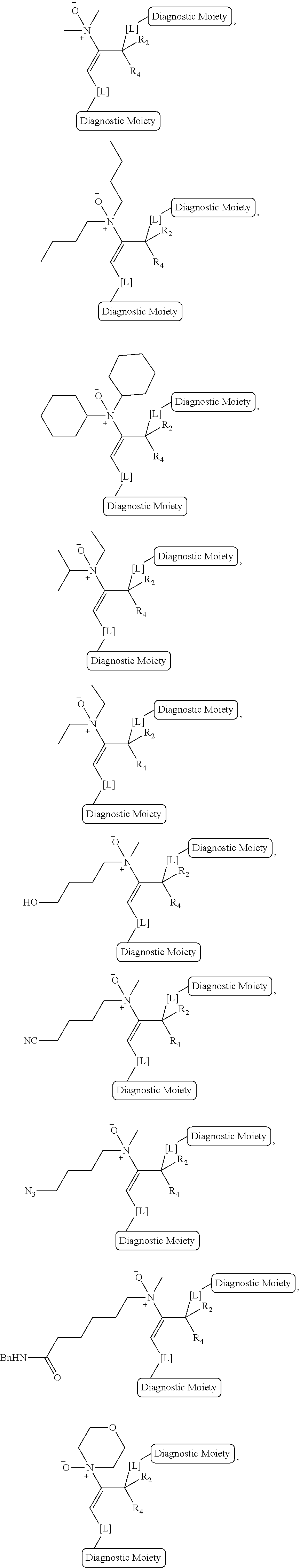

or a pharmaceutically acceptable salt or stereoisomer thereof.

In some embodiments, the compound of formula (IIe) is represented by any one of the following structures:

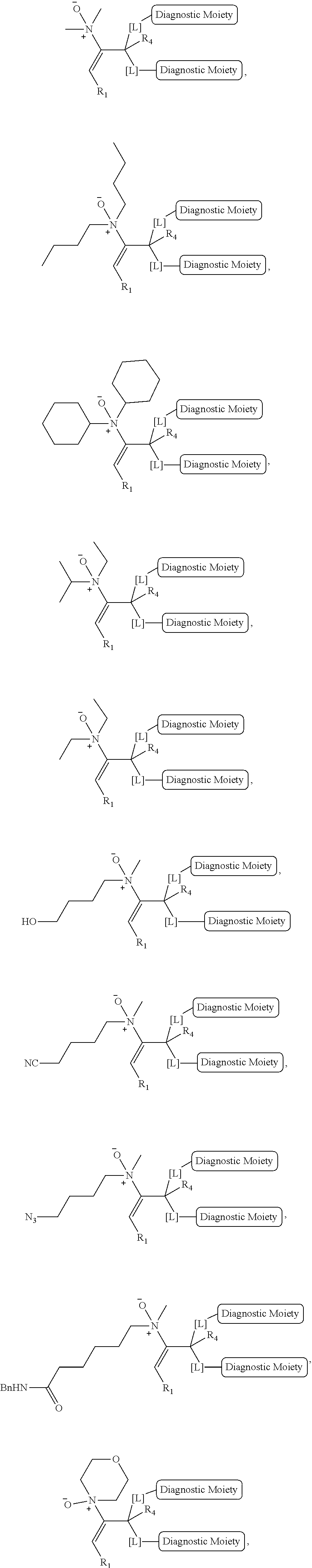

or a pharmaceutically acceptable salt or stereoisomer thereof.

In some embodiments, the compound of formula (IIf) is represented by any one of the following structures:

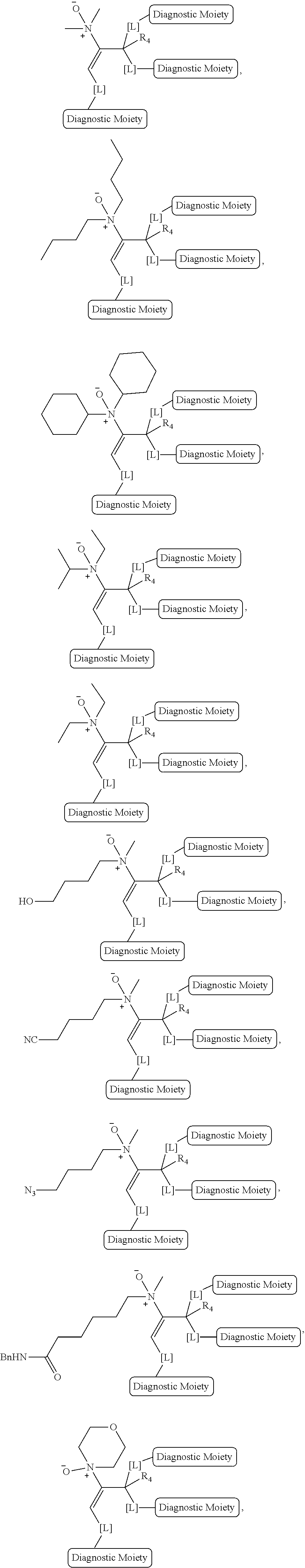

or a pharmaceutically acceptable salt or stereoisomer thereof.

In another aspect, compounds of the invention are represented by formulas III, and IV:

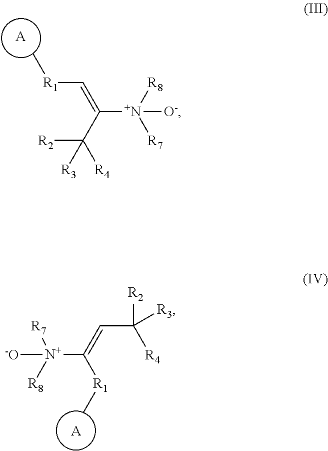

or a pharmaceutically acceptable salt or stereoisomer thereof,
wherein:
R₁ is hydrogen, CH₂, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a cleavable linking group, or -[L]-diagnostic moiety, wherein R₁ may be optionally substituted;
   [L] is absent or a linking group that is capable of carrying a plurality of diagnostic moieties, which may be the same or different;
R₂ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a π-electron withdrawing group, a leaving group, or -[L]-diagnostic moiety, wherein R₂ may be optionally substituted;
   [L] is absent or a linking group that is capable of carrying a plurality of diagnostic moieties, which may be the same or different;
R₃ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a π-electron withdrawing group, a leaving group, or -[L]-diagnostic moiety, wherein R₃ may be optionally substituted;
   [L] is absent or a linking group that is capable of carrying a plurality of diagnostic moieties, which may be the same or different;
R₄ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a π-electron withdrawing group, a leaving group, or -[L]-diagnostic moiety, wherein R₄ may be optionally substituted;
   [L] is absent or a linking group that is capable of carrying a plurality of diagnostic moieties, which may be the same or different;
R₇ is (C₁-C₅) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted, or
R₇ and R₈ together with the nitrogen atom to which they are attached, form a 4- to 7-membered heterocyclyl comprising 1 to 3 heteroatoms selected from O, N, and S;
R₈ is (C₁-C₅) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted; and
A is absent, a leaving group or a therapeutic moiety,
provided that the compound of formula (III or IV) contains at least one -[L]-diagnostic moiety or therapeutic moiety,
and when A is a therapeutic moiety, R₁ is a cleavable linking group;
and when at least one of R₂, R₃, or R₄ is a -[L]-diagnostic moiety for a compound of formula (III), R₁ is CH₂ and A is a leaving group;
and when at least one of R₁, R₂, or R₃ is a -[L]-diagnostic moiety and A is absent for a compound of formula (IV), R₄ is a leaving group.

In some embodiments, R₁ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, or a 5- to 6-membered heterocyclyl.

In some embodiments, R₁ is CH₂.

In some embodiments, R₁ is a -[L]-diagnostic moiety. Diagnostic moieties typically contain a detectable moiety such as a label. In certain embodiments, the label comprises a fluorescent dye. In some embodiments, the diagnostic moiety includes a rhodamine dye. In some embodiments, the diagnostic moiety includes tetramethylrhodamine (TAMRA) or a derivative thereof.

In some embodiments, the diagnostic moiety is an affinity tag.

In some embodiments, the diagnostic moiety is a chromogenic.

In some embodiments, the diagnostic moiety is a PET tracer.

In some embodiments, the diagnostic moiety is a MRI contrast agent.

In some embodiments, the diagnostic moiety is an intercalating agent.

In some embodiments, R₁ is a -[L]-diagnostic moiety, wherein [L] is a linking group that is optionally substituted by the same or a different -[L]-diagnostic moiety. In some embodiments, [L] is an alkylene chain, that may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different. In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —O—, —S—, —N(R′)—, —C(O)—, —C(O)O—, —OC(O)—, —C(O)N(R′)—, —N(R′)C(O)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —O—. In some embodiments, the alkylene chain is interrupted by, and/or terminates (at either or both termini) with —S—.

In some embodiments, [L] is a polyethylene glycol chain, that may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different. In some embodiments, the polyethylene glycol chain has 1 to 20 —(CH₂CH₂—O)— units. In some embodiments, the polyethylene glycol chain has 1 to 15 —(CH₂CH₂—O)— units. In some embodiments, the polyethylene glycol chain has 1 to 10 —(CH₂CH₂—O)— units. In some embodiments, the polyethylene glycol chain has 1 to 5 —(CH₂CH₂—O)— units. In some embodiments, the polyethylene glycol chain has 1 to 2 —(CH₂CH₂—O)— units. In some embodiments, the polyethylene glycol is interrupted by, and/or terminates (at either or both termini) in at least one of —S—, —N(R′)—, —C(O)—, —C(O)O—, —OC(O)—, —C(O)N(R′)—, —N(R′)C(O)—, or a combination thereof. In some embodiments, the polyethylene glycol chain is interrupted by, and/or terminates (at either or both termini) with —C(O)—. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —S—.

In some embodiments, R₁ is a cleavable linking group.

In some embodiments, R₁ is an alkylene chain, that is interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different. In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, —OP(O)O(R′)O—, —N(R′)P(O)N(R′R′)N(R′)—, or a combination thereof. In some embodiments, the alkylene chain is interrupted by, and/or terminates (at either or both termini) with —OC(O)—. In some embodiments, the alkylene chain is interrupted by, and/or terminates (at either or both termini) with —OC(O)O—. In some embodiments, the alkylene chain is interrupted by, and/or terminates (at either or both termini) with —OC(O)N(R′)—.

In some embodiments, R₁ is a polyethylene glycol chain, that is interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different. In some embodiments, the polyethylene glycol chain has 1 to 20 —(CH₂CH₂—O)— units. In some embodiments, the polyethylene glycol chain has 1 to 15 —(CH₂CH₂—O)— units. In some embodiments, the polyethylene glycol chain has 1 to 10 —(CH₂CH₂—O)— units. In some embodiments, the polyethylene glycol chain has 1 to 5 —(CH₂CH₂—O)— units. In some embodiments, the polyethylene glycol chain has 1 to 2 —(CH₂CH₂—O)— units. In some embodiments, the polyethylene glycol is interrupted by, and/or terminates (at either or both termini) in at least one of —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, —OP(O)O(R′)O—, —N(R′)P(O)N(R′R′)N(R′)—, or a combination thereof. In some embodiments, the polyethylene glycol chain is interrupted by, and/or terminates (at either or both termini) with —OC(O)—. In some embodiments, the polyethylene glycol chain is interrupted by, and/or terminates (at either or both termini) with —OC(O)O—. In some embodiments, the polyethylene glycol chain is interrupted by, and/or terminates (at either or both termini) with —OC(O)N(R′)—.

In some embodiments, R₂ is hydrogen.

In some embodiments, R₂ is C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, or 5- to 6-membered heterocyclyl.

In some embodiments, R₂ is a leaving group.

In some embodiments, R₂ is an inductive electron withdrawing group. In some embodiments, the inductive electron withdrawing group is halogen, OR₆, SR₆, or NR₆R₆, wherein each R₆ is independently hydrogen, C₁-C₆ alkyl, C₆-C₁₂ aryl, 5- to 10-membered heteroaryl, carbonyl, sulfonyl, sulfinyl, or phosphoryl. In some embodiments, the inductive electron withdrawing group is halogen. In some embodiments, the halogen is fluoro or chloro.

In some embodiments, R₂ is a 7-electron withdrawing group. In some embodiments, the π-electron withdrawing group is —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, or —P(O)NR₆R₆NR₆R₆, wherein each R₆ is independently hydrogen, C₁-C₆ alkyl, C₆-C₁₂ aryl, or 5- to 10-membered heteroaryl.

In some embodiments, R₂ is a -[L]-diagnostic moiety. In some embodiments, R₂ is a diagnostic moiety as described above for R¹. In some embodiments, R₂ is a -[L]-diagnostic moiety, wherein [L] is a linking group that is optionally substituted by the same or a different -[L]-diagnostic moiety.

In some embodiments, R₃ is hydrogen.

In some embodiments, R₃ is C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, or 5- to 6-membered heterocyclyl.

In some embodiments, R₃ is a leaving group.

In some embodiments, R₃ is an inductive electron withdrawing group. In some embodiments, the inductive electron withdrawing group is halogen, OR₆, SR₆, or NR₆R₆, wherein each R₆ is independently hydrogen, C₁-C₆ alkyl, C₆-C₁₂ aryl, 5- to 10-membered heteroaryl, carbonyl, sulfonyl, sulfinyl, or phosphoryl. In some embodiments, the inductive electron withdrawing group is halogen. In some embodiments, the halogen is fluoro or chloro.

In some embodiments, R₃ is a 7-electron withdrawing group. In some embodiments, the π-electron withdrawing group is —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, or —P(O)NR₆R₆NR₆R₆, wherein each R₆ is independently hydrogen, C₁-C₆ alkyl, C₆-C₁₂ aryl, or 5- to 10-membered heteroaryl.

In some embodiments, R₃ is a -[L]-diagnostic moiety. In some embodiments, R₃ is a diagnostic moiety as described above for R¹. In some embodiments, R₃ is a -[L]-diagnostic moiety, wherein [L] is a linking group that is optionally substituted by the same or a different -[L]-diagnostic moiety.

In some embodiments, R₄ is hydrogen.

In some embodiments, R₄ is C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl.

In some embodiments, R₄ is a leaving group.

In some embodiments, R₄ is an inductive electron withdrawing group. In some embodiments, the inductive electron withdrawing group is halogen, OR₆, SR₆, or NR₆R₆, wherein each R₆ is independently hydrogen, C₁-C₆ alkyl, C₆-C₁₂ aryl, 5- to 10-membered heteroaryl, carbonyl, sulfonyl, sulfinyl, or phosphoryl. In some embodiments, the inductive electron withdrawing group is halogen. In some embodiments, the halogen is fluoro or chloro.

In some embodiments, R₄ is a -[L]-diagnostic moiety. In some embodiments, R₄ is a diagnostic moiety as described above for R¹. In some embodiments, R₄ is a -[L]-diagnostic moiety, wherein [L] is a linking group that is optionally substituted by the same or a different -[L]-diagnostic moiety.

In some embodiments, R₄ is hydrogen.

In some embodiments, R₇ is Me, Et, ⁿBu, iPr, Cy,

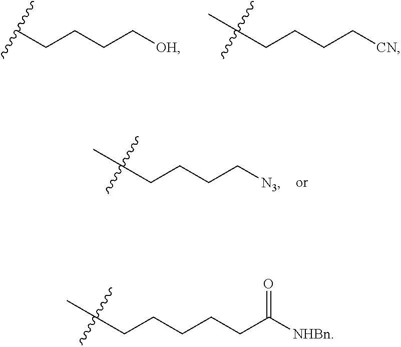

In some embodiments, R₇ and R₈ together with the nitrogen atom to which they are attached, form a 6-membered heterocyclyl comprising 2 heteroatoms selected from O and N. In some embodiments, R₇ and R₈ together with the nitrogen atom to which they are attached, form a piperidinyl, piperazinyl, or morphinyl group.

In some embodiments, R₈ is Me, Et, ⁿBu, iPr, Cy,

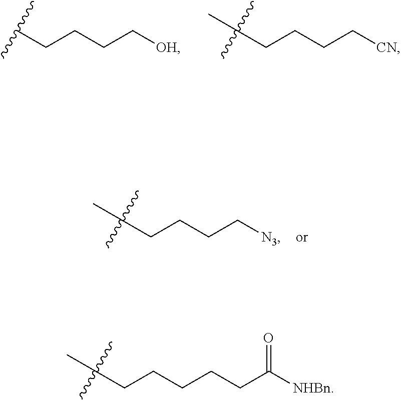

In some embodiments, the optionally substituent for R₁, R₂, R₃, R₄, R₇, and R₈, is a substituent selected from the group comprising of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, and a -[L]-diagnostic moiety. In some embodiments, the optional substituents may be one or more additional -[L]-diagnostic moieties, which may be the same or different.

In some embodiments, A is a therapeutic moiety. In some embodiments, the therapeutic moiety is a small molecule. In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. In certain embodiments, the small molecule is a therapeutically active agent such as a drug (e.g., a molecule approved by the U.S. Food and Drug Administration as provided in the Code of Federal Regulations (C.F.R.)).

In some embodiments, the therapeutic moiety is an anti-cancer agent.

In some embodiments, the anti-cancer agent is a non-targeted agent.

In some embodiments, the therapeutic moiety is a targeted anti-cancer agent. In some embodiments, the targeted anti-cancer agent is a kinase inhibitor.

In some embodiments, the therapeutic moiety is a hypoxia-inducible factor inhibitor (HIF).

In some embodiments, the therapeutic moiety is an apoptotic agent.

In some embodiments, the therapeutic moiety is a non-steroidal anti-inflammatory drug (NSAID).

In some embodiments, the therapeutic moiety is a disease-modifying anti-rheumatic drug (DMARD).

In some embodiments, A is absent.

In some embodiments, A is a leaving group.

In some embodiments, the compound of formula (III) is represented by any one of the following structures:

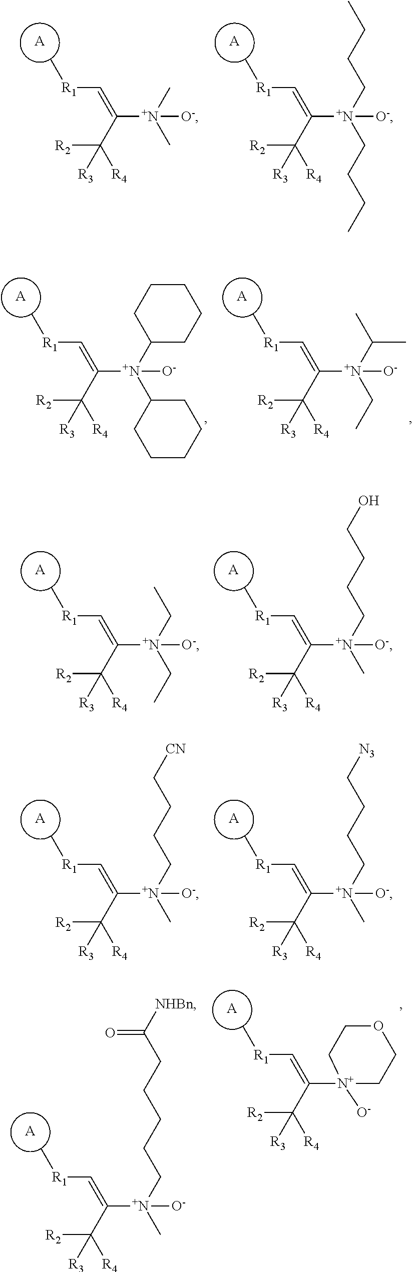

or a pharmaceutically acceptable salt or stereoisomer thereof. In some embodiments, R₁ is CH₂, a cleavable linking group, or -[L]-diagnostic moiety; and/or R₂ is halogen, OR₆, SR₆, NR₆R₆, —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, —P(O)NR₆R₆NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₃ is halogen, OR₆, SR₆, NR₆R₆, —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, —P(O)NR₆R₆NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₄ is hydrogen, C₁-C₆ alkyl, C₁-C₆ haloalkyl, C₁-C₆ haloalkoxy, CN, NO₂, NH₂, (C₁-C₆ alkyl)NH, halogen, OR₅, SR₅, NR₅R₅, or a -[L]-diagnostic moiety, wherein each R₅ is independently hydrogen, (C₁-C₆) alkyl, (C₃-C₁₀) carbocyclyl, 4- to 7-membered heterocyclyl; and/or A is absent, a leaving group, or a therapeutic moiety. In some embodiments, halogen is fluor or chloro. In some embodiments, C₆-C₁₂ aryl is phenyl. In some embodiments, 5- to 10-membered heteroaryl is pyrrole, furan, thiophene, pyridine, or pyrimidine. In some embodiments, C₁-C₆ alkyl is methyl, ethyl or propyl or isopropyl. In some embodiments, A is an anti-cancer agent. In some embodiments, the diagnostic moiety is a fluorescent dye.

In some embodiments for a compound of formula (III), R₁ is a cleavable linking group; and/or R₂ is halogen, OR₆, SR₆, NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₃ is halogen, OR₆, SR₆, NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₄ hydrogen, C₁-C₆ alkyl, CN, NH₂, (C₁-C₆ alkyl)NH, halogen, OR₅, SR₅, or NR₅R₅; and/or A is a therapeutic moiety. In some embodiments, halogen is fluor or chloro. In some embodiments, C₆-C₁₂ aryl is phenyl. In some embodiments, 5- to 10-membered heteroaryl is pyrrole, furan, thiophene, pyridine, or pyrimidine. In some embodiments, C₁-C₆ alkyl is methyl, ethyl or propyl or isopropyl. In some embodiments, A is an anti-cancer agent.

In some embodiments, the compound of formula (III) is represented by any one of formulas IIIa-IIId:

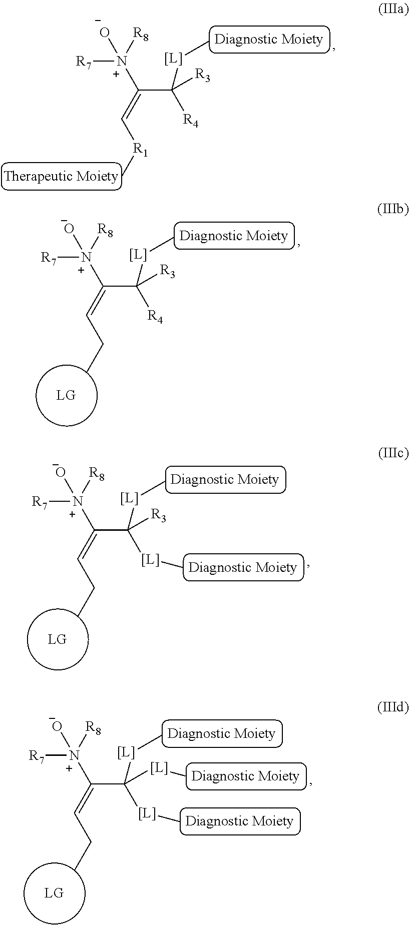

or a pharmaceutically acceptable salt or stereoisomer thereof, wherein LG=leaving group.

In some embodiments, the compound of formula (IIIa) is represented by any one of the following structures

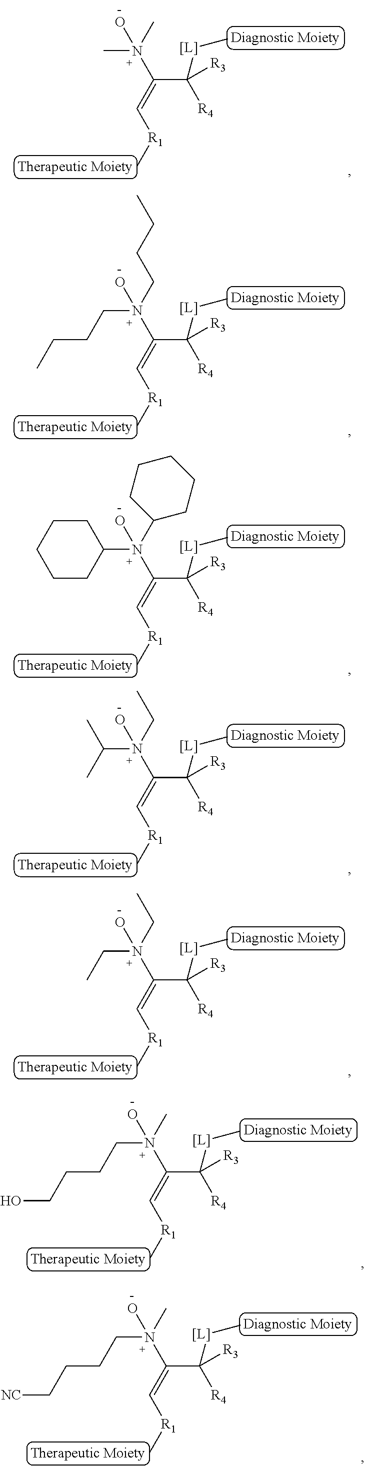

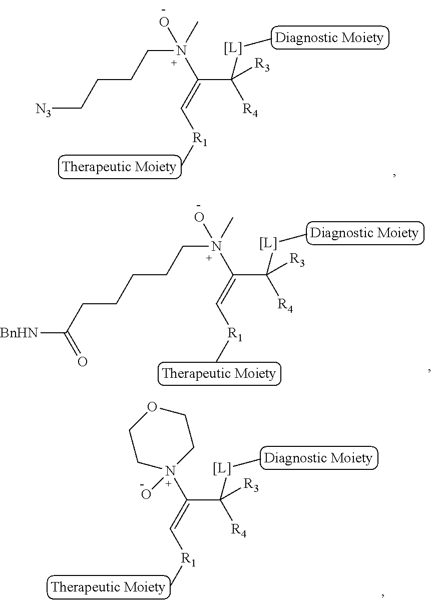

or a pharmaceutically acceptable salt or stereoisomer thereof.

In some embodiments, the compound of formula (IIIb) is represented by any one of the following structures:

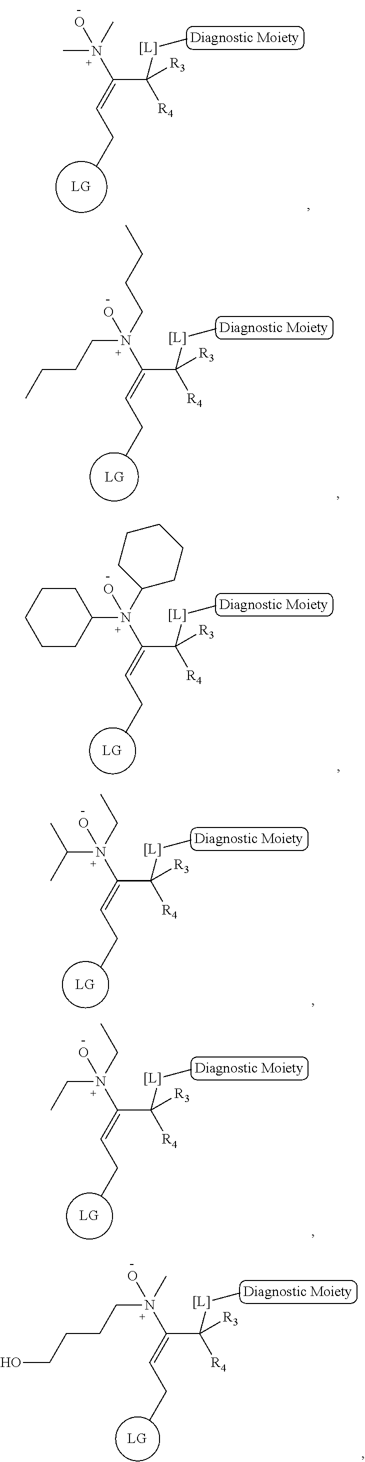

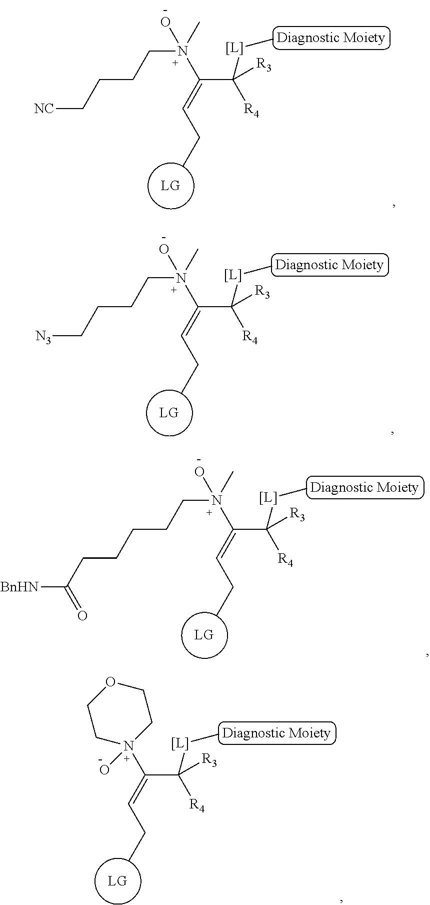

or a pharmaceutically acceptable salt or stereoisomer thereof.

In some embodiments, the compound of formula (IIIc) is represented by any one of the following structures:

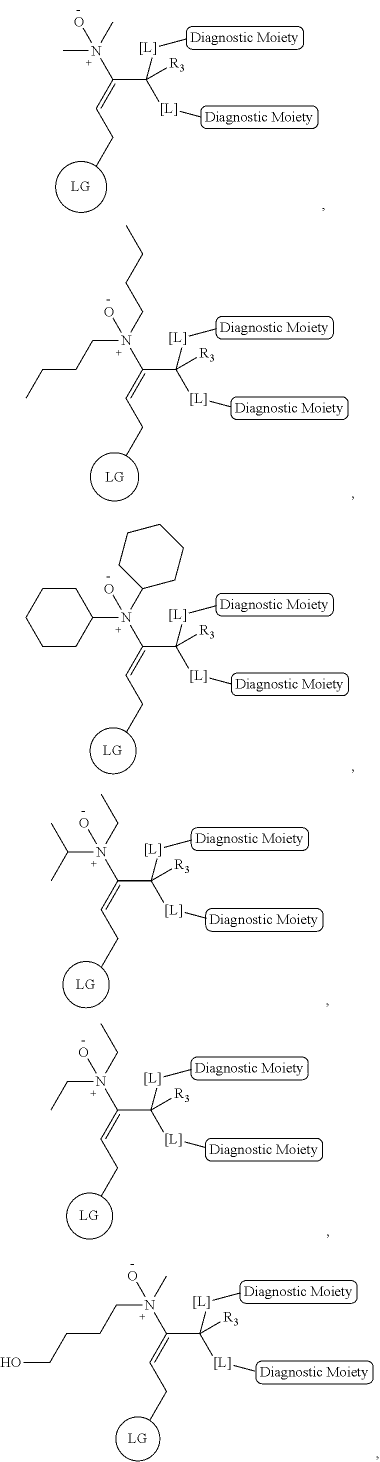

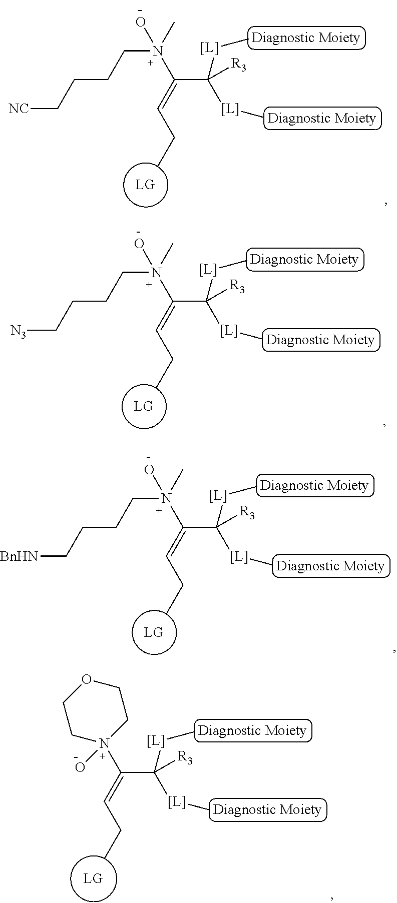

or a pharmaceutically acceptable salt or stereoisomer thereof.

In some embodiments, the compound of formula (IIId) is represented by any one of the following structures:

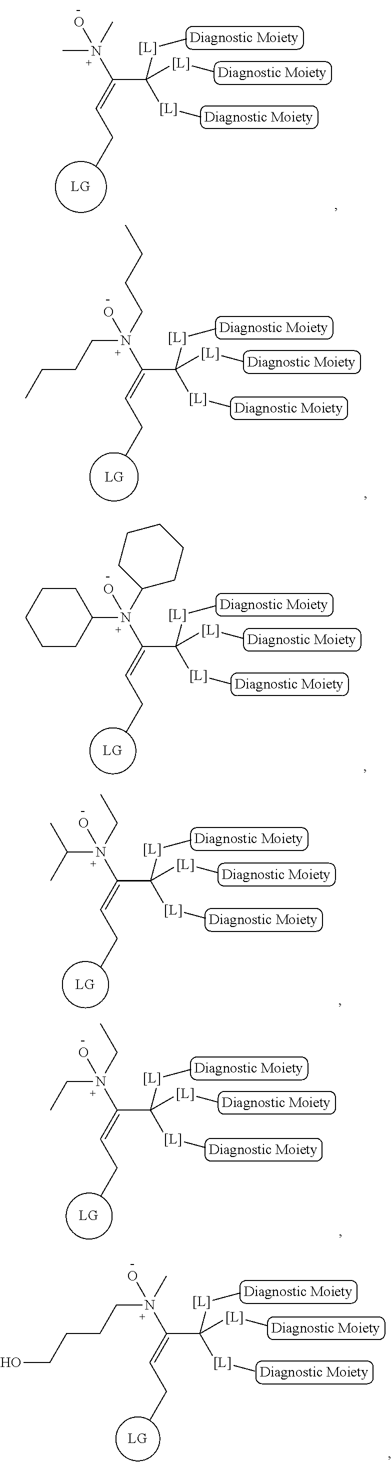

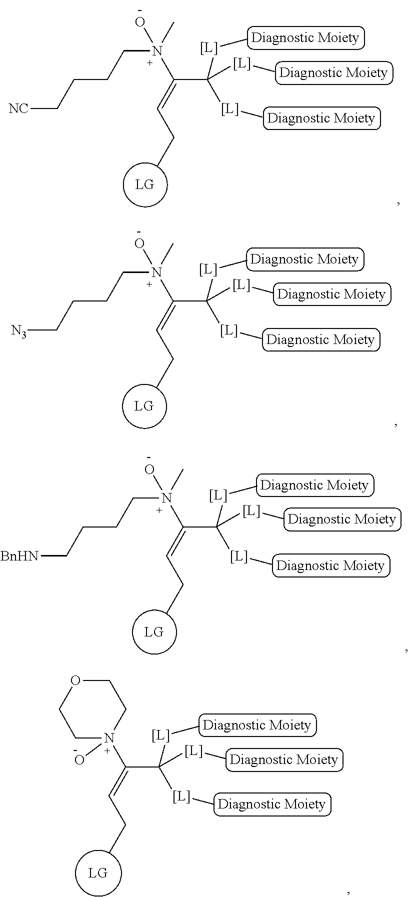

or a pharmaceutically acceptable salt or stereoisomer thereof.

In some embodiments, the compound of formula (IV) is represented by any one of the following structures:

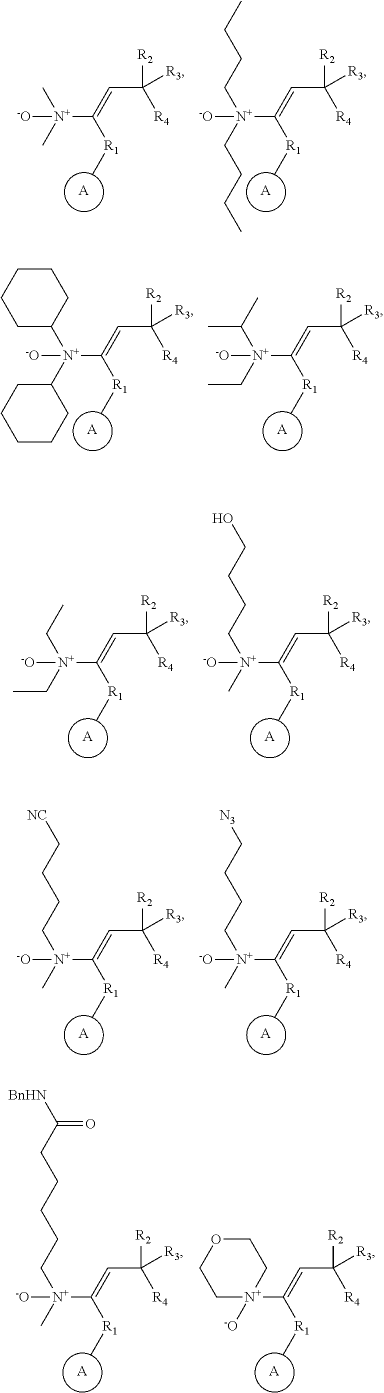

or a pharmaceutically acceptable salt or stereoisomer thereof. In some embodiments, R₁ is CH₂, a cleavable linking group, or -[L]-diagnostic moiety; and/or R₂ is halogen, OR₆, SR₆, NR₆R₆, —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, —P(O)NR₆R₆NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₃ is halogen, OR₆, SR₆, NR₆R₆, —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, —P(O)NR₆R₆NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₄ is hydrogen, C₁-C₆ alkyl, C₁-C₆ haloalkyl, C₁-C₆ haloalkoxy, CN, NO₂, NH₂, (C₁-C₆ alkyl)NH, halogen, OR₅, SR₅, NR₅R₅, or a -[L]-diagnostic moiety, wherein each R₅ is independently hydrogen, (C₁-C₆) alkyl, (C₃-C₁₀) carbocyclyl, 4- to 7-membered heterocyclyl; and/or A is absent, a leaving group, or a therapeutic moiety. In some embodiments, halogen is fluor or chloro. In some embodiments, C₆-C₁₂ aryl is phenyl. In some embodiments, 5- to 10-membered heteroaryl is pyrrole, furan, thiophene, pyridine, or pyrimidine. In some embodiments, C₁-C₆ alkyl is methyl, ethyl or propyl or isopropyl. In some embodiments, A is an anti-cancer agent. In some embodiments, the diagnostic moiety is a fluorescent dye.

In some embodiments for a compound of formula (IV), R₁ is a cleavable linking group; and/or R₂ is halogen, OR₆, SR₆, NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₃ is halogen, OR₆, SR₆, NR₆R₆, or a -[L]-diagnostic moiety, wherein each R₆ is independently hydrogen, C₁-C₆ alkyl, C₆-C₁₂ aryl, or 5- to 10-membered heteroaryl; and/or R₄ is hydrogen, C₁-C₆ alkyl, CN, NH₂, (C₁-C₆ alkyl)NH, halogen, OR₅, SR₅, or NR₅R₅; and/or A is a therapeutic moiety. In some embodiments, halogen is fluor or chloro. In some embodiments, C₆-C₁₂ aryl is phenyl. In some embodiments, 5- to 10-membered heteroaryl is pyrrole, furan, thiophene, pyridine, or pyrimidine. In some embodiments, C₁-C₆ alkyl is methyl, ethyl or propyl or isopropyl. In some embodiments, A is an anti-cancer agent.

In some embodiments, the compound of formula (IV) is represented by formula IVa-IVd:

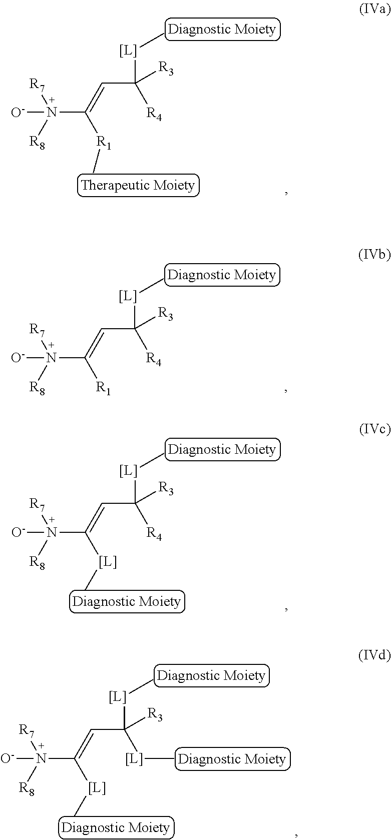

or a pharmaceutically acceptable salt or stereoisomer thereof.

In some embodiments, the compound of formula (IVa) is represented by any one of the

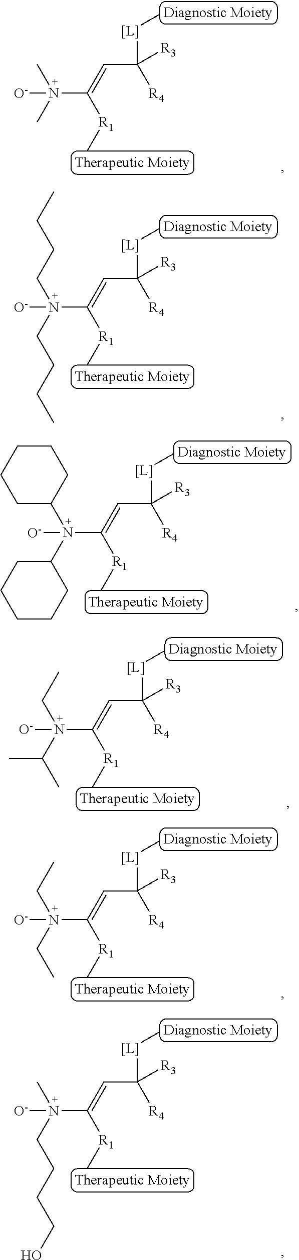

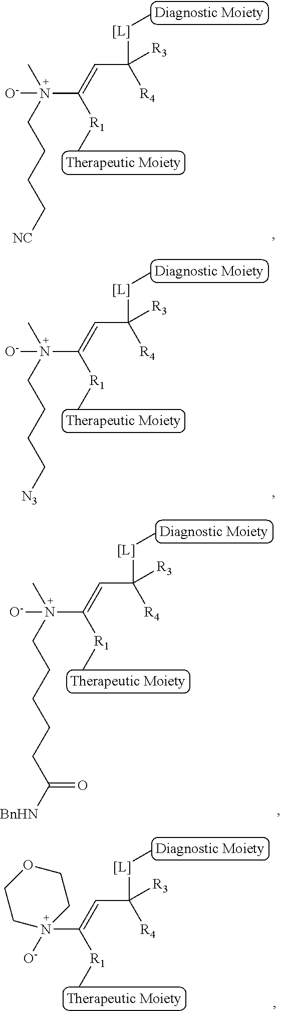

or a pharmaceutically acceptable salt or stereoisomer thereof.

In some embodiments, the compound of formula (IVb) is represented by any one of the following structures:

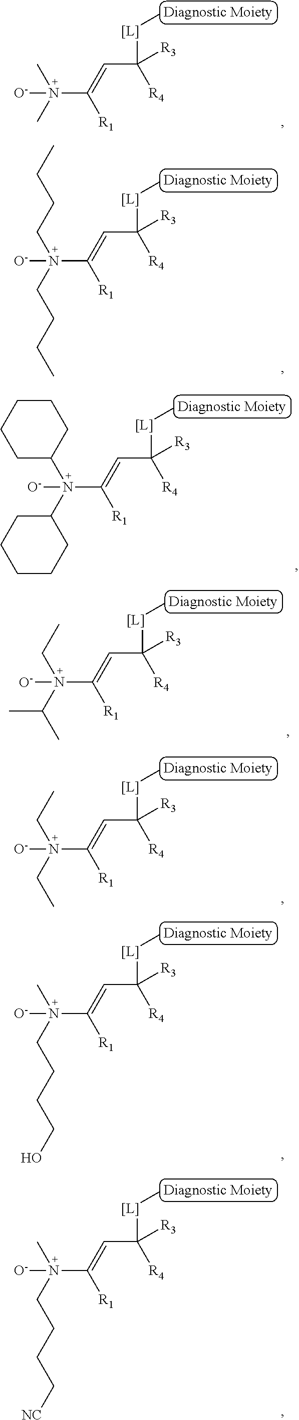

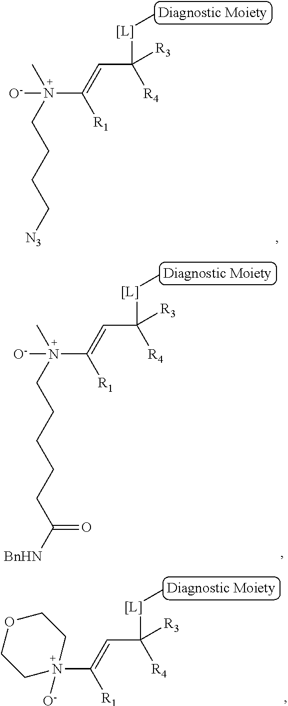

or a pharmaceutically acceptable salt or stereoisomer thereof.

In some embodiments, the compound of formula (IVc) is represented by any one of the following structures:

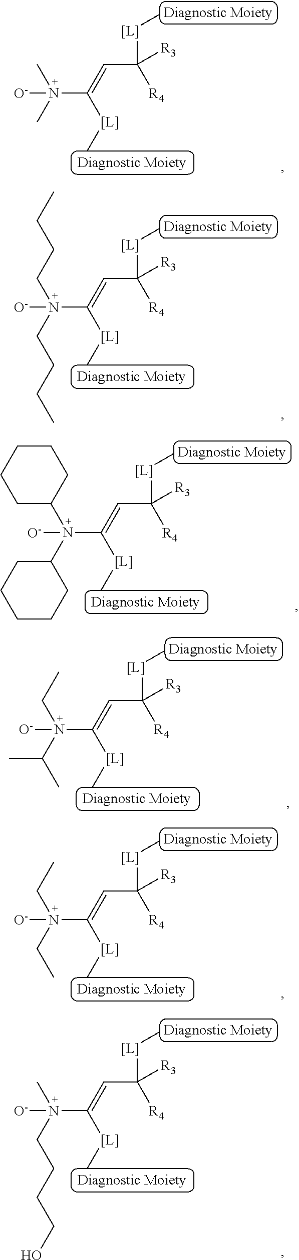

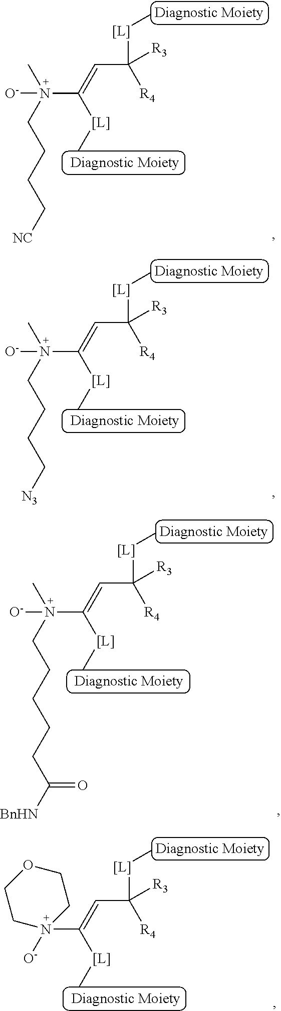

or a pharmaceutically acceptable salt or stereoisomer thereof.

In some embodiments, the compound of formula (IVd) is represented by any one of the following structures:

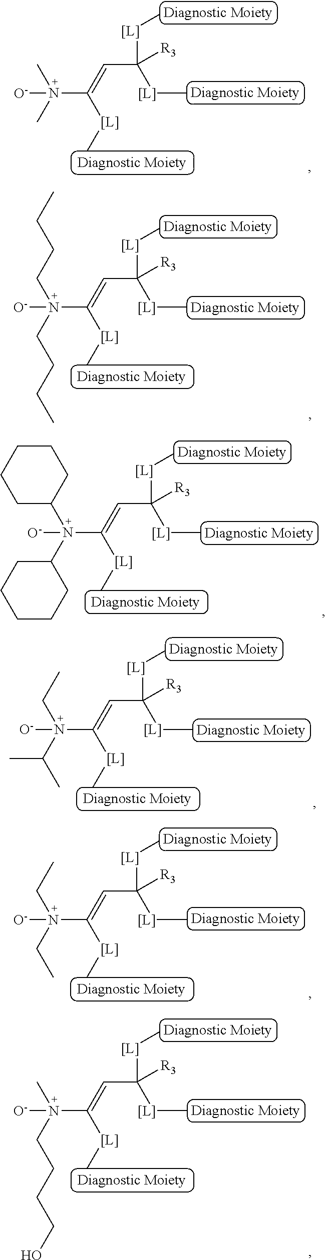

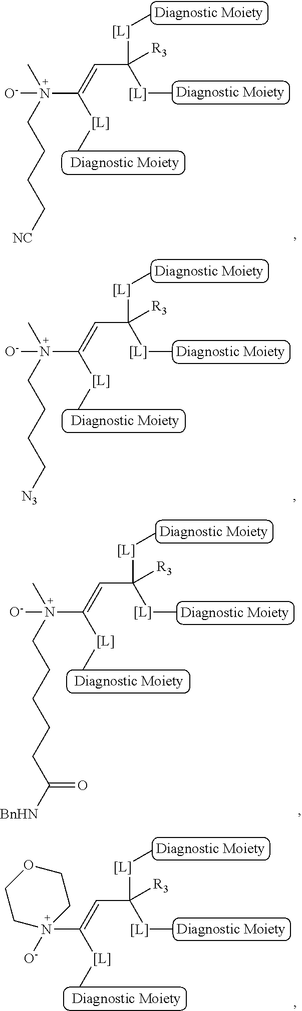

or a pharmaceutically acceptable salt or stereoisomer thereof.

In some embodiments, inventive compounds include

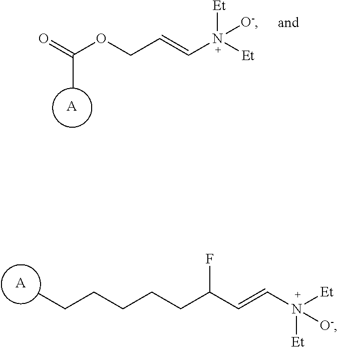

or a pharmaceutically acceptable salt or stereoisomer thereof.

In some embodiments, inventive compounds include

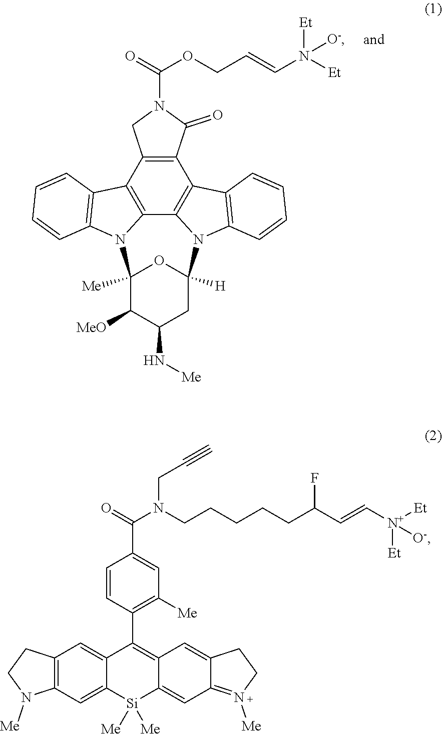

or a pharmaceutically acceptable salt or stereoisomer thereof.

Compounds of the present invention may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin. Suitable base salts include aluminum, calcium, lithium, magnesium, potassium, sodium, or zinc salts.

Compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

In some embodiments, an inventive compound may be in the form of an isotopic derivative in that it has at least one desired isotopic substitution of an atom, at an amount above the natural abundance of the isotope, i.e., enriched. In one embodiment, the compound includes deuterium or multiple deuterium atoms. In one embodiment, the compound includes ¹¹C or multiple ¹¹C atoms. In one embodiment, the compound includes ¹³N or multiple ¹³N atoms. In one embodiment, the compound includes ¹⁵O or multiple ¹⁵O atoms. In one embodiment, the compound includes ¹⁸F or multiple ¹⁸F atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

The compounds of the present invention may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

In some embodiments, the pharmaceutical composition comprises a co-crystal of an inventive compound. The term “co-crystal”, as used herein, refers to a stoichiometric multi-component system comprising a compound of the invention and a co-crystal former wherein the compound of the invention and the co-crystal former are connected by non-covalent interactions. The term “co-crystal former”, as used herein, refers to compounds which can form intermolecular interactions with a compound of the invention and co-crystallize with it. Representative examples of co-crystal formers include benzoic acid, succinic acid, fumaric acid, glutaric acid, trans-cinnamic acid, 2,5-dihydroxybenzoic acid, glycolic acid, trans-2-hexanoic acid, 2-hydroxycaproic acid, lactic acid, sorbic acid, tartaric acid, ferulic acid, suberic acid, picolinic acid, salicyclic acid, maleic acid, saccharin, 4,4′-bipyridine p-aminosalicyclic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present invention is directed to a method for making an inventive compound, or a pharmaceutically acceptable salt or stereoisomer thereof. Broadly, the inventive compounds or a pharmaceutically acceptable salt or stereoisomer thereof may be prepared by any process known to be applicable to the preparation of chemically related compounds. The compounds of the present invention will be better understood in connection with the synthetic schemes that described in various working examples and which illustrate non-limiting methods by which the compounds of the invention may be prepared.

In another aspect, the present invention is directed to a process of preparing compounds of formulas I and II:

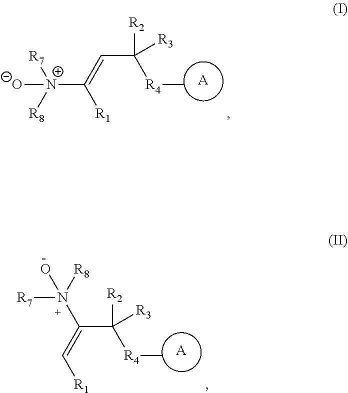

comprising reacting a compound of formula V:

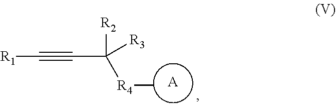

with a compound of formula VII:

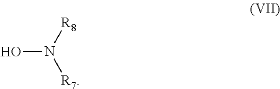

In another aspect, the present invention is directed to a process of preparing a compound of formula III or IV:

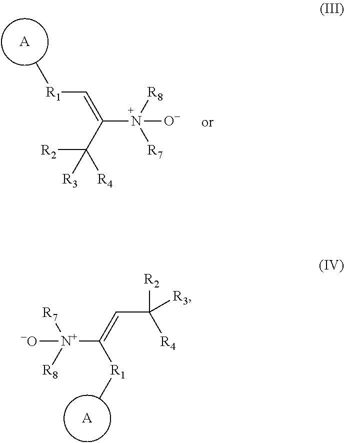

comprising reacting a compound of formula VI:

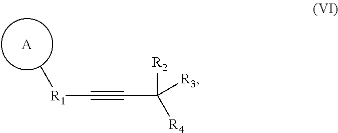

with a compound of formula VII:

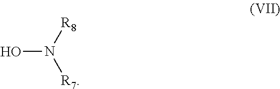

Synthetic schemes for attaching active moieties to chemical compounds (e.g., alkynyl-group containing compounds) are known in the art. See, e.g., Wang et al., J. Am. Chem. Soc. 141(43):17133-17141 (2019), and Guerrant et al., Bioorg. Med. Chem. Lett. 23(11):3283-3287 (2013).

In some embodiments, the reacting is carried out in the presence of a solvent.

In some embodiments, the solvent is an aprotic solvent. In some embodiments, the aprotic solvent is DCM, CHCl₃, CCl₄, DCE, toluene, MeCN, or THF.

In some embodiments, the solvent is a protic solvent. In some embodiments, the protic solvent is MeOH, EtOH, iPrOH, nBuOH, TFE, or HFIP.

In some embodiments, the solvent is a solvent mixture. In some embodiments, the solvent mixture is a mixture of an aprotic solvent and a protic solvent. In some embodiments, the solvent mixture is 0-100% protic to aprotic. In some embodiments, the solvent mixture is 0-100% TFE in CHCl₃. In some embodiments, the solvent mixture is about 20% TFE in CHCl₃.

In some embodiments, the reacting is carried out in the presence of an aqueous buffer. In some embodiments, the aqueous buffer is an acidic buffer. In some embodiments, the aqueous buffer is an alkaline buffer.

In some embodiments, the reacting is carried out in the presence of a biological fluid. In some embodiments, the biological fluid is blood, synovial fluid, lymph, or vitrious fluid.

In some embodiments, the reacting is carried out in the presence of an aqueous solution with biological components such as cell lysate, proteins, nucleic acids, or lipids.

In some embodiments, the reaction is carried out with the addition of a buffering reagent. Representative examples of buffered reagents include ascorbic acid, glutathione, citric acid, acetic acid, monopotassium phosphate, N-cyclohexyl-2-aminoethanesulfonic acid (CHES), and borate. In some embodiments, the buffering reagent is ascorbic acid or glutathione.

In some embodiments, the reaction is carried out at a temperature from about −40° C. to 80° C. In some embodiments, the reaction is carried out at a temperature from about 0° C. to 60° C. In some embodiments, the reaction is carried out at a temperature from about 20° C. to 60° C. In some embodiments, the reaction is carried out at a temperature of about 60° C. In some embodiments, the reaction is carried out at a temperature from about 20° C. to 25° C.

In some embodiments, the compound of formula (VII) is in excess of the compound of formula (V) or (VI). In some embodiments, the excess is about 10 equivalents. In some embodiments, the excess is about 5 equivalents.

In some embodiments, the reaction is carried out over a week. In some embodiments, the reaction is carried out over five days. In some embodiments, the reaction is carried out over three days. In some embodiments, the reaction is carried out over a period of 24 hours. In some embodiments, the reaction is carried out over a period of 18 hours. In some embodiments, the reaction is carried out over a period of 12 hours. In some embodiments, the reaction is carried out over a period of 6 hours. In some embodiments, the reaction is carried out over a period of 3 hours. In some embodiments, the reaction is carried out over a period of 2 hours. In some embodiments, the reaction is carried out over a period of 1 hour. In some embodiments, the reaction is carried out over a period of 45 minutes. In some embodiments, the reaction is carried out over a period of 30 minutes. In some embodiments, the reaction is carried out over a period of 15 minutes. In some embodiments, the reaction is carried out over a period of 5 minutes. In some embodiments, the reaction is carried out over a period of 1 minute.

In some embodiments, the reaction is carried out at a temperature of about 60° C.; and/or the solvent mixture is about 20% TFE in CHCl₃; and/or the reaction is carried out over a period of 18 hours.

### Pharmaceutical Compositions

Another aspect of the present invention is directed to a pharmaceutical composition that includes a therapeutically effective amount of an inventive compound or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of the invention and their pharmaceutically acceptable salts, or stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds are formulated for oral or intravenous administration (e.g., systemic intravenous injection).

Accordingly, compounds of the invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of the invention may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of the invention may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of the invention may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of the invention may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of an inventive compound (that contains a therapeutic moiety), or a pharmaceutically acceptable salt or stereoisomer thereof that is effective in producing the desired therapeutic response in a patient suffering from a disease or disorder characterized by, or associated with or exhibiting tissue hypoxia. The term “therapeutically effective amount” thus includes the amount of the inventive compound or a pharmaceutically acceptable salt or stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or inhibits the growth of diseased cells.

As used herein, the term, “diagnostically effective amount” refers to an amount of an inventive compound (that contains a diagnostic moiety), or a pharmaceutically acceptable salt or stereoisomer thereof that is effective in producing the desired detectable response in a patient suffering from a disease or disorder characterized by, or associated with or exhibiting tissue hypoxia. The term “diagnostically effective amount” thus includes the amount of the inventive compound or a pharmaceutically acceptable salt or stereoisomer thereof, that when administered, induces a signal that may be detected or visualized by any of a variety of means including spectroscopic, photochemical, biochemical, immunochemical, electrical, optical, or chemical means.

The total daily dosage of the compounds and usage thereof may be decided in accordance with standard medical practice, e.g., by the attending physician using sound medical judgment. The specific therapeutically effective dose for any particular subject will depend upon a variety of factors, including the following: the disease or disorder being treated and the severity thereof (e.g., its present status); the activity of the compound employed; the specific composition employed; the age, body weight, general health, sex and diet of the subject; the time of administration, route of administration, and rate of excretion of the compound employed; the duration of the treatment; drugs used in combination or coincidental with the specific compound employed; and like factors well known in the medical arts (see, for example, Hardman et al., eds., Goodman and Gilman's The Pharmacological Basis of Therapeutics, 10th Edition, McGraw-Hill Press, 155-173, 2001).

Compounds of the invention may be effective over a wide dosage range. In some embodiments, the total daily dosage (e.g., for adult humans) may range from about 0.001 to about 1600 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the compound may be administered at a dose in range from about 0.01 mg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

### Methods of Use

In some aspects, the present invention is directed to methods of labeling hypoxic tissue, e.g., a solid tumor, that entails administration of a compound of formula (I, II, III, or IV), or a pharmaceutically acceptable salt or stereoisomer thereof, to a subject in need thereof. The active moiety contained in the inventive compounds is selected accordingly.

In some aspects, the present invention is directed to methods of treating a disease or disorder characterized by, or associated with or exhibiting tissue hypoxia, that entails administration of a therapeutically effective amount of a compound of formula (I, II, III, or IV), or a pharmaceutically acceptable salt or stereoisomer thereof, to a subject in need thereof. The active moiety contained in the inventive compounds is selected accordingly.

The term “subject” (or “patient”) as used herein includes all members of the animal kingdom prone to or suffering from a solid tumor. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a disease or disorder that may have been positively diagnosed or otherwise presents with a sufficient number of risk factors or a sufficient number or combination of signs or symptoms such that a medical professional could diagnose or suspect that the subject was suffering from a disease or disorder. Thus, subjects suffering from, and suspected of suffering from, a disease or disorder are not necessarily two distinct groups.

Diseases and disorders characterized by, associated with or that exhibit tissue hypoxia are known in the art. See, e.g., Wigerup et al., Pharm. Thera. 164:152-169 (2016); Sharma et al., Chem. Soc. Rev. 48(3):771-813 (2019); Bernauer et al., Brit. J. Cancer 124:539-551 (2021); Jing et al., Mol. Cancer 18:157 (2019). Tissue hypoxia may be an inherent property of the disease or disorder. In some embodiments, localized tissue hypoxia may be induced by administration of an hypoxia activating agent.

Exemplary types of non-cancerous (e.g., cell proliferative) diseases or disorders characterized by, associated with or exhibit tissue hypoxia and that may be amenable to treatment with the compounds of the present invention include inflammatory diseases and conditions, anemia, renal failure, cardiovascular disease, reperfusion injury, and metabolic diseases.

Representative examples of specific non-cancerous diseases and disorders include coronary heart disease, stroke, peripheral arterial disease, aortic disease, cerebrovascular disease, rheumatic heart disease, congenital heart disease, deep vein thrombosis, pulmonary embolism, arrhythmia, hyperlactatemia, metabolic brain disease, DNA repair-deficiency disorder, porphyrias, metabolic skin disease, and proteostatis deficiency.

In some embodiments, the methods are directed to treating subjects having cancer. Generally, the compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), polycythemia vera, lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), germ cell tumor, ovarian germ cell tumor, head and neck cancer, Hodgkin's lymphoma, leukemia, lymphoma, multiple myeloma, hepatocellular carcinoma, hypopharyngeal cancer, intraocular melanoma, ocular cancer, islet cell tumors (endocrine pancreas), renal cancer (e.g., Wilm's Tumor, clear cell renal cell carcinoma), liver cancer, lung cancer (e.g., non-small cell lung cancer and small cell lung cancer), Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, essential thrombocythemia, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, pituitary tumor, plasma cell neoplasm, pleuropulmonary blastoma, prostate cancer, retinoblastoma rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, transitional cell cancer of the renal pelvis and ureter and other urinary organs, urethral cancer, gestational trophoblastic tumor, vaginal cancer and vulvar cancer.

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types).

In some embodiments, methods of the present invention entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, compounds of the present invention may be used to treat non-metastatic or metastatic lung cancer (e.g., NSCLC, ALK-positive NSCLC, NSCLC harboring ROS1 Rearrangement, Lung Adenocarcinoma, and Squamous Cell Lung Carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, the methods of labeling are directed to labeling a solid tumor in a hypoxic tumor microenvironment, that entail administration of a compound of formula (I, II, III, or IV), or a pharmaceutically acceptable salt or stereoisomer thereof, to a subject in need thereof. In some embodiments, the solid tumor is in the brain, breast, cervix, kidney, liver, lungs, pancreas, or rectum. In some embodiments, the solid tumor is epidermoid carcinoma, lung carcinoma, glioblastoma, or pancreatic adenocarcinoma.

In some embodiments, the methods of treating are directed to methods of treating a solid tumor in a hypoxic tumor microenvironment, that entail administration of a therapeutically effective amount of a compound of formula (I, II, III, or IV), or a pharmaceutically acceptable salt or stereoisomer thereof, to a subject in need thereof. In some embodiments, the solid tumor is in the brain, breast, cervix, kidney, liver, lungs, pancreas, or rectum. In some embodiments, the solid tumor is epidermoid carcinoma, lung carcinoma, glioblastoma, or pancreatic adenocarcinoma. Hypoxic tumor microenvironments are known in the art. See, e.g., Petrova et al., Oncogenesis 7:10 (2018); Muz et al., Hypoxia (Auckl). 3:83-92 (2015); Hockel et al., J. Natl. Cancer Inst. 93(4):266-276 (2001).

The compounds of the present invention and their pharmaceutically acceptable salts and stereoisomers may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been unsuccessful, or partially successful but who became non-responsive or intolerant to the particular treatment. Therapy may also be given as an adjuvant treatment, i.e., to prevent reoccurrence of cancer in patients with no currently detectable disease or after surgical removal of a tumor. Thus, in some embodiments, the compound may be administered to a patient who has received prior therapy, such as chemotherapy, radioimmunotherapy, surgical therapy, immunotherapy, radiation therapy, targeted therapy or any combination thereof.

The methods of the present invention may entail administration of an inventive compound or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day off period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of five days.

### Combination Therapy

The compounds of the present invention and their pharmaceutically acceptable salts or stereoisomers may be used in combination or concurrently with at least one other active agent e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second agent, the first of the two agents is in some cases still detectable at effective concentrations at the site of treatment. The sequence and time interval may be determined such that they can act together (e.g., synergistically to provide an increased benefit than if they were administered otherwise). For example, the agents may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of the present invention or a pharmaceutically acceptable salt or stereoisomer thereof in combination with one or more additional therapeutic agents known for use in treating the disease or disorder (e.g., cancer). The dosage of the additional anticancer therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference 60th ed., 2006. For example, anti-cancer agents that may be used in combination with the inventive compounds are known in the art. See, e.g., U.S. Pat. No. 9,101,622 (Section 5.2 thereof) and U.S. Pat. No. 9,345,705 B2 (Columns 12-18 thereof). Representative examples of additional anti-cancer agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immune-modulators, therapeutic antibodies (e.g., mono-specific and bispecific antibodies) and CAR-T therapy.

In some embodiments, the compound of the invention and the additional anticancer therapeutic agent may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more anticancer therapeutics may be administered within the same patient visit.

In some embodiments, the compound of the present invention and the additional therapeutic agent (e.g., an anti-cancer therapeutic) are cyclically administered. By way of example in the context of cancer treatment, cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, an inventive compound may be administered with an agent that locally forms, within a tumor or a defined area containing one or more tumors, a region of hypoxia, e.g., 10% or lower oxygen. Representative types of such agents include anti-angiogenic agents and vascular disruptive agents. See, e.g., U.S. Patent Application Publication 2017/0224693 A1.

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a compound of the present invention or a pharmaceutical composition which contains the compound and a pharmaceutically acceptable carrier wherein the compound and the carrier may be disposed in the same or separate containers. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

### Example 1: General Information, Materials, and Instrumentations

### General Information

All reactions were conducted in flame-dried round-bottom flasks under a positive pressure of nitrogen unless otherwise stated. Gas-tight syringes with stainless steel needles or cannulae were used to transfer air- and moisture-sensitive liquids. Flash column chromatography was performed using granular silica gel (60-A pore size, 40-63 μm, Silicycle). Analytical thin layer chromatography (TLC) was performed using glass plates pre-coated with 0.25 mm silica gel impregnated with a fluorescent indicator (254 nm, Silicycle). TLC plates were visualized by exposure to short wave ultraviolet light (254 nm) and/or an aqueous solution of potassium permanganate (KMnO₄). Organic solutions were concentrated at 20° C. on rotary evaporators capable of achieving a minimum pressure of ˜2 torr unless otherwise stated. Room temperature is defined as 22.5±2.5° C. Reaction heating was performed using a UCON™ fluid heating bath.

### Materials

All solvents were purchased from Fisher Scientific or Sigma-Aldrich. Unless otherwise stated chemical reagents were purchased from Fisher Scientific, Sigma-Aldrich, Alfa Aesar, Oakwood Chemical, Acros Organics, Combi-Blocks, or TCI America. CMA refers to a solution of 80:18:2 v/v/v chloroform:methanol:ammonium hydroxide (28-30% ammonia solution). Chloroform used in CMA solutions and as co-eluents in silica gel column chromatography were stabilized with 0.75% v/v ethanol. Chloroform used in all hydroamination reactions were stabilized with pentene.

### General Chemical Instrumentation

Proton nuclear magnetic resonance (H NMR) spectra, recorded with a 500 MHz Avance III Spectrometer with multi-nuclear Smart probe, are reported in parts per million on the 6 scale, and are referenced from the residual protium in the NMR solvent (CDCl₃: δ 7.24 (chloroform), CD₃OD: δ 3.31 (CHD₂OD)). Data are reported as follows: chemical shift [multiplicity (s=singlet, d=doublet, t=triplet, q=quartet, p=pentet, sx=sextet, sp=septet, dd=doublet of doublets, dt=doublet of triplets, ddd=doublet of doublets of doublets, dtd=doublet of triplets of doublets, td=triplet of doublets, tdd=triplet of doublets of doublets, qd=quartet of doublets, m=multiplet), coupling constant(s) in Hertz, integration, assignment]. Carbon-13 nuclear magnetic resonance (¹³C NMR) spectra are referenced from the carbon resonances of the solvent (CDCl₃: δ 77.23, CD₃OD: δ 49.15). Fluorine-19 nuclear magnetic resonance (¹⁹F NMR) is calibrated from the fluorine resonances of benzotrifluoride (CDCl₃: δ −62.76, CD₃OD: δ −64.24). Data are reported as follows: chemical shift (assignment). Infrared data (IR) were obtained with a Cary 630 Fourier transform infrared spectrometer equipped with a diamond ATR objective and are reported as follows: frequency of absorption (cm⁻¹), intensity of absorption (s=strong,m=medium, w=weak, br=broad). High resolution mass spectra (HRMS) were recorded on a Q Exactive™ Plus Hybrid Quadrupole-Orbitrap™ Mass Spectrometer using an electrospray ionization (ESI), atmospheric pressure ionization (API), or electron ionization (EI) source. Automated C₁₈ reverse phase chromatography was performed using an Isolera One (Biotage) purification system. High performance liquid chromatography (HPLC) purification was performed using an Agilent 1260 Infinity system.

### General Biological Instrumentation

UV/vis absorbance measurements were acquired on an Agilent Technologies Cary 60 UV-Vis spectrophotometer. In-gel fluorescence imaging was performed on a GE Healthcare Life Sciences Typhoon™ FLA 9500. Slides were imaged at the Confocal and Light Microscopy Core at Dana-Farber Cancer Institute using a Nikon Ti Eclipse. Images were acquired with a 10× objective at 0.64 micron/pixel using a Hamamatsu camera. Hoechst 33342 and DAPI were imaged with a 405/20 filter (excitation) and a 460/50 filter (emission) and false-colored blue; FITC and Alexa Fluor 488 were imaged with a 482/35 filter (excitation) and a 536/75 filter (emission) and false-colored green; and TAMRA was imaged with a 560/40 filter (excitation) and a 630/75 filter (emission) and false-colored red. In vivo imaging was obtained by a Xenogen IVIS 100 using Living Image (Version 4.2). Images were processed with Fiji ImageJ software.

### Example 2: Design of a Hypoxia-Responsive Chemical Motif with Drug Release and Labeling Properties

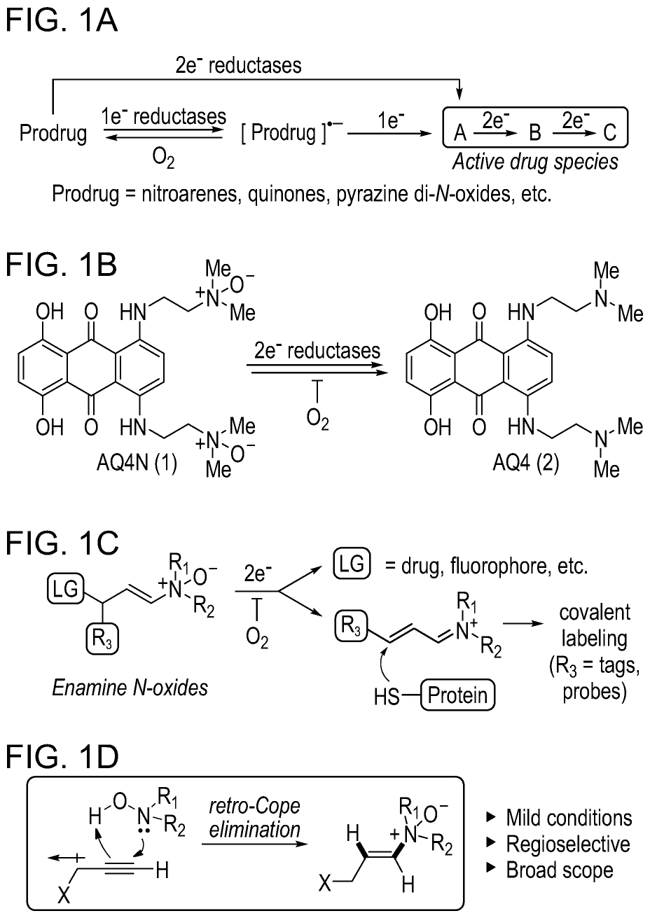

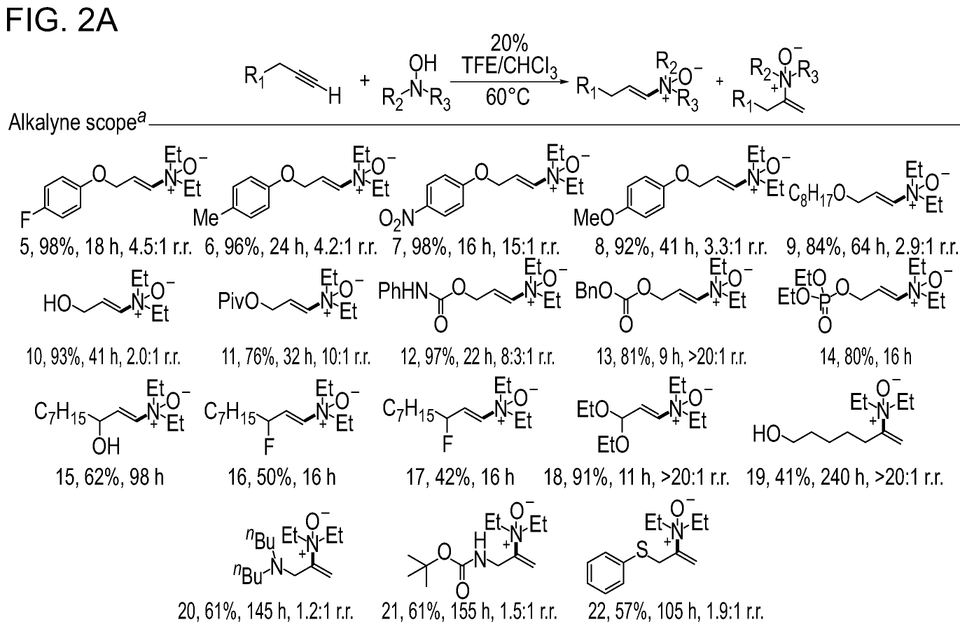

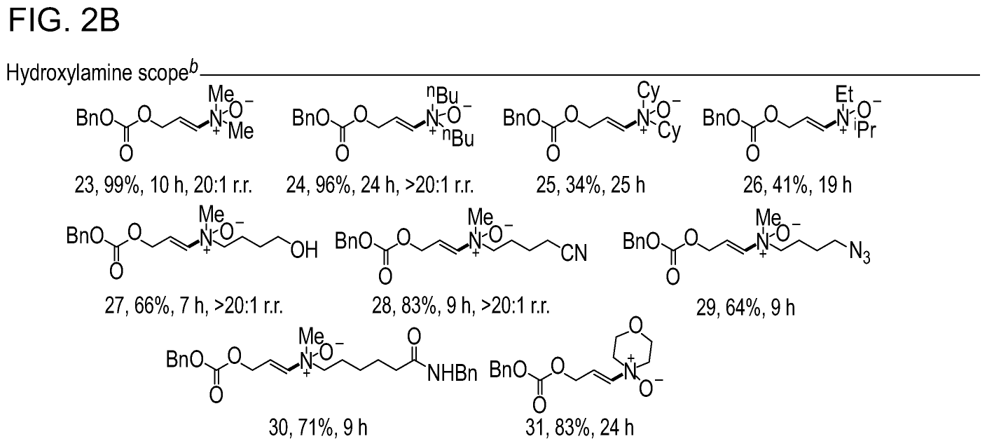

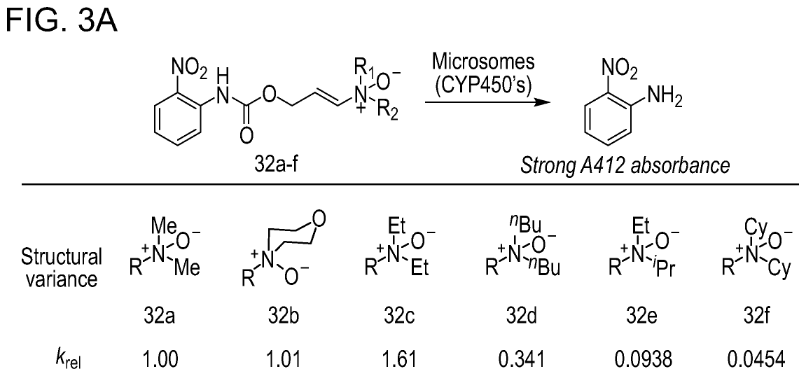

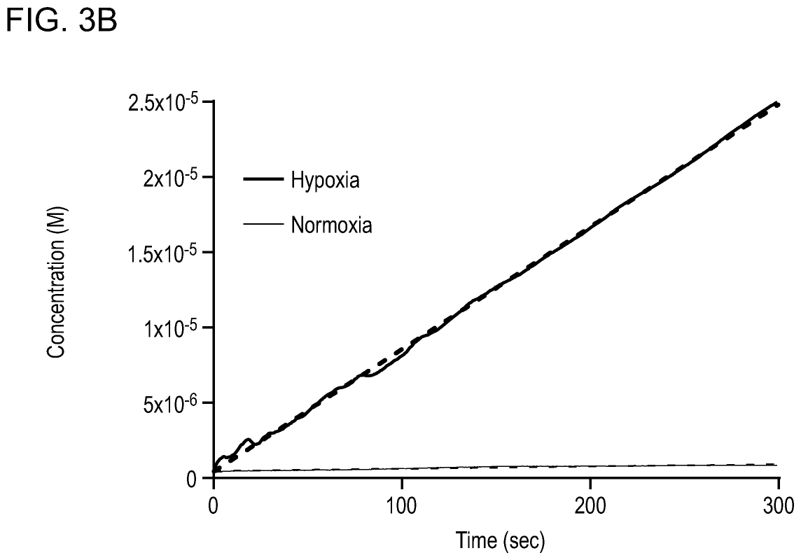

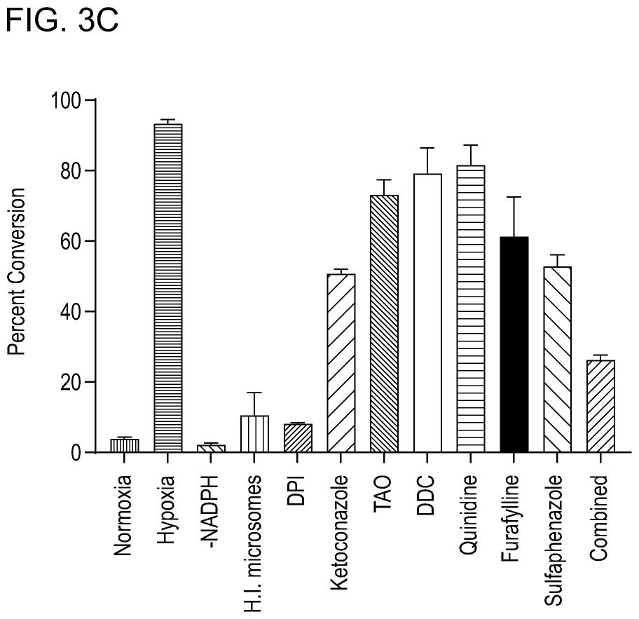

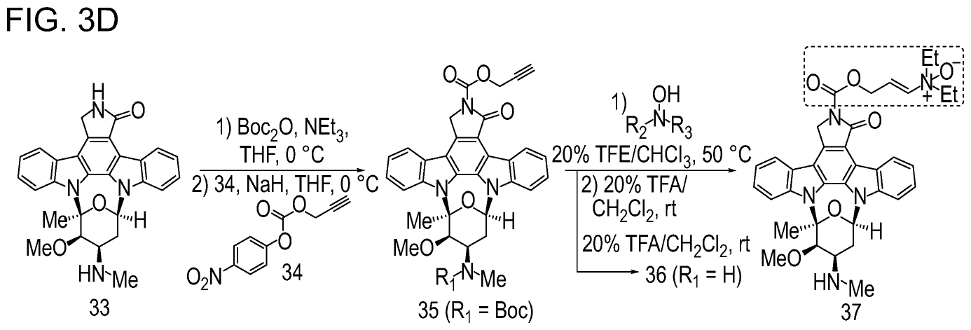

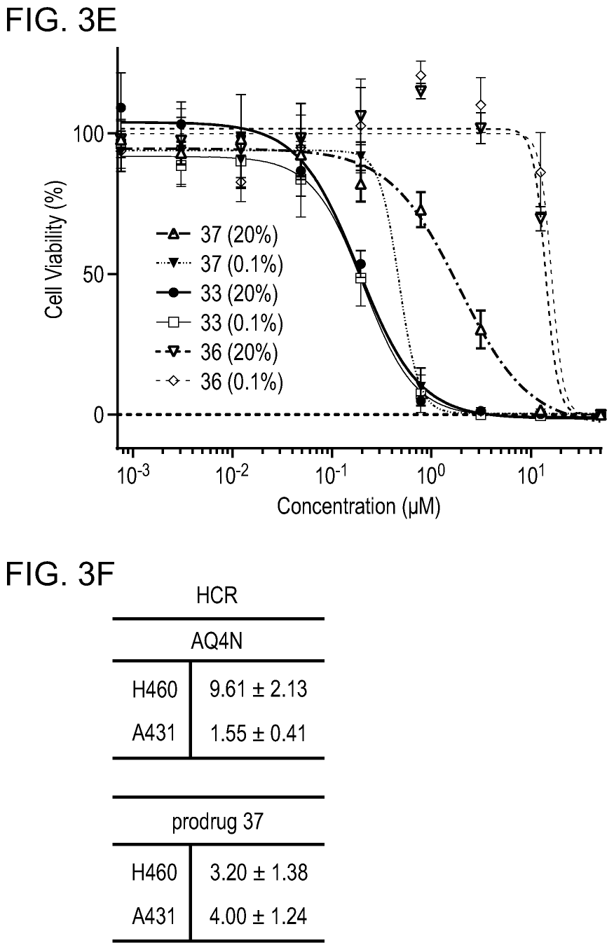

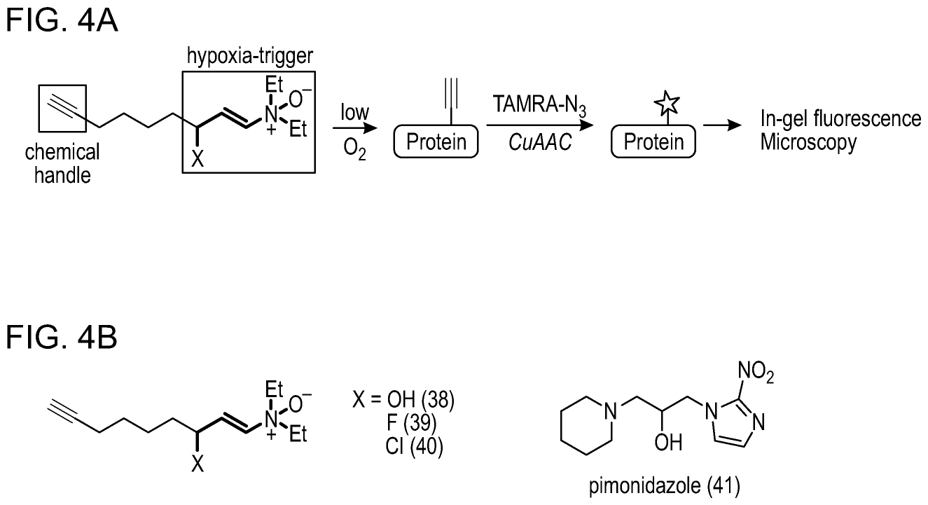

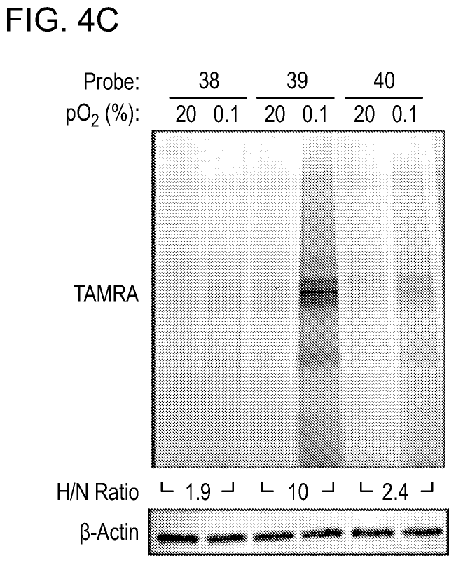

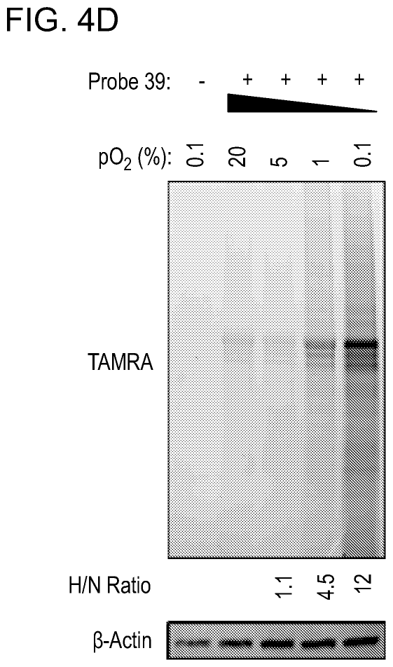

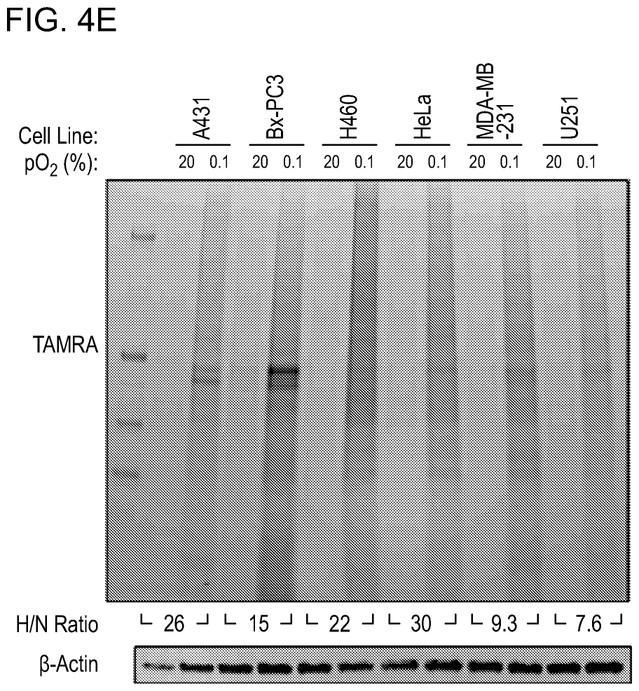

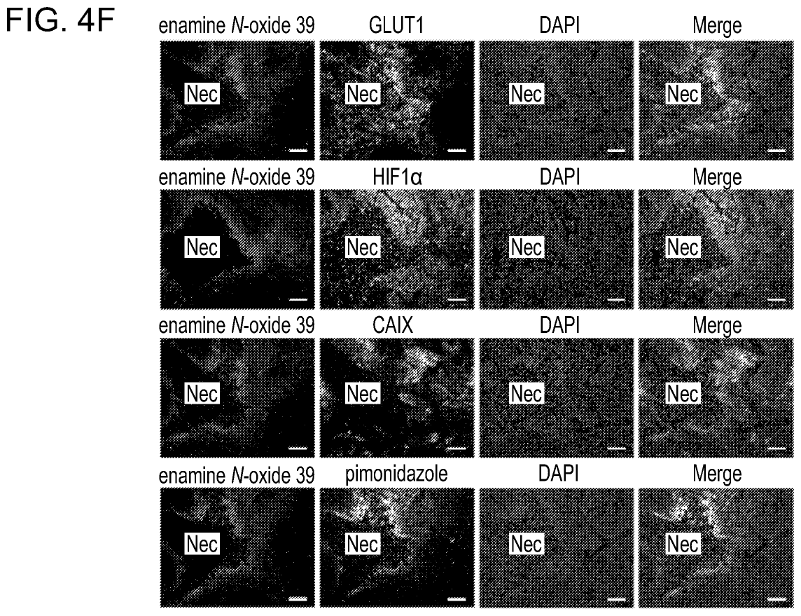

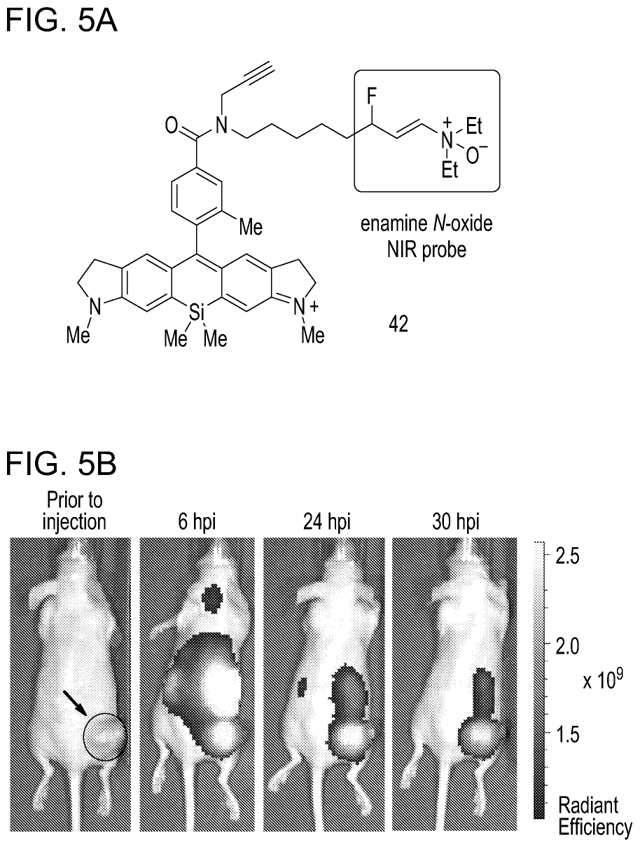

Introduction of α,β-unsaturation on the amine N-oxide provided the enamine N-oxide structure (FIG. 1C). A signal output mechanism was designed into the structure by embedding a leaving group at the allylic position. Enamine N-oxide reduction produced an enamine from which β-elimination would generate two functionally relevant species: 1) a leaving group and 2) an electrophilic α,β-unsaturated iminium ion. If a prodrug is desired, the allylic leaving group (LG) could be a caged drug, if a probe, a fluorophore, and if nothing, an inert halogen or chalcogen. The function of the electrophilic component would likewise be defined by the payload appended at the allylic position (R₃). Affinity tags such as biotin or an alkyne, probes such as a fluorophore, or PET tracers such as an [¹⁸F] fluorine atom are installed to suit the application. Both labeling and release potential are captured in the design of the enamine N-oxide.

### Example 3: Synthesis of Enamine N-Oxides

Intermolecular retro-Cope elimination of unactivated alkynes is complicated by the propensity of enamine N-oxides to undergo Cope elimination (Bourgeois et al., J. Am. Chem. Soc. 131(3):874-875 (2009); Beauchemin, A. M., Org. Biomol. Chem. 11(41):7039-7050 (2013)) and [1,2]-Meissenheimer rearrangement (Bernier et al., J. Org. Chem. 73(11):4229-4232 (2008); Castagnoli et al., Tetrahedron 26(18):4319-4327 (1970); Bottle et al., J. Chem. Soc., Perkin Trans. 2(7):1001-1007 (1991)) at mildly elevated temperatures. Activation through inductive effects was investigated (FIG. 1D). p-fluorophenyl propargyl ether (3) was used as a model substrate, an initial temperature screen from room temperature to 80° C. in chloroform (CHCl₃) demonstrated that the desired enamine N-oxide can be obtained, but there was a significant tradeoff between conversion and degradation with a steep drop off in yield within a tight ±10° C. window. Still, the maximum yield was 50% (Table 1).

TABLE 1
Temperature effects on hydroamination in CHCl₃.^{a}

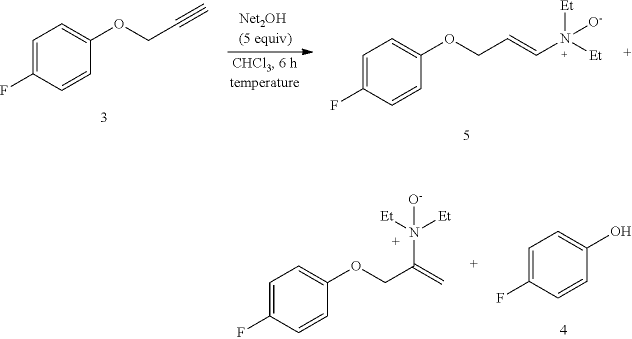

| Temperature | Yield (%)^{b} | | | | |
|---|---|---|---|---|---|
| (° C.) | 3 | 5 + 5′ | 4 | Total | r.r. (5:5′) |
| rt | 93 | 5 | 0 | 98 | 7.8:1 |
| 40 | 72 | 26 | <1 | 98 | 6.8:1 |
| 50 | 35 | 50 | 5 | 90 | 15:1 |
| 60 | 16 | 48 | 23 | 87 | 77:1 |
| 70 | 0 | 31 | 49 | 80 | 90:1 |
| 80 | 0 | 0 | 54 | 54 | N/A |

ªConditions: alkyne 3 (0.2 mmol, 1 equiv, 0.2 M), N,N-diethylhydroxylamine (1 mmol, 5.0 equiv, 1 M). ^{b}Yields were determined by NMR using benzotrifluoride as an internal standard. r.r. = regioisomeric ratio.

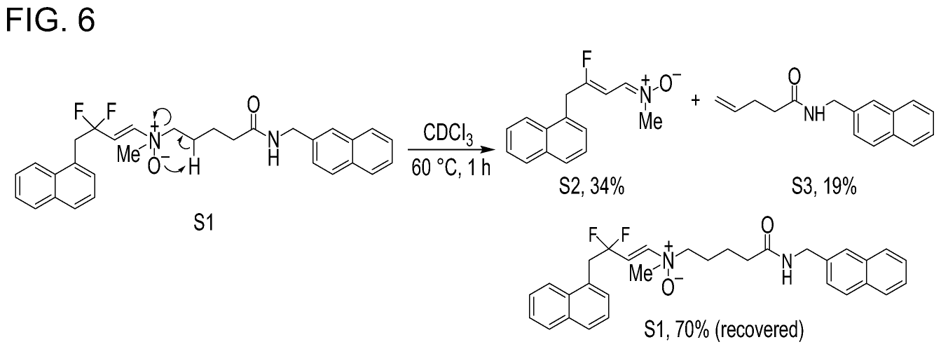

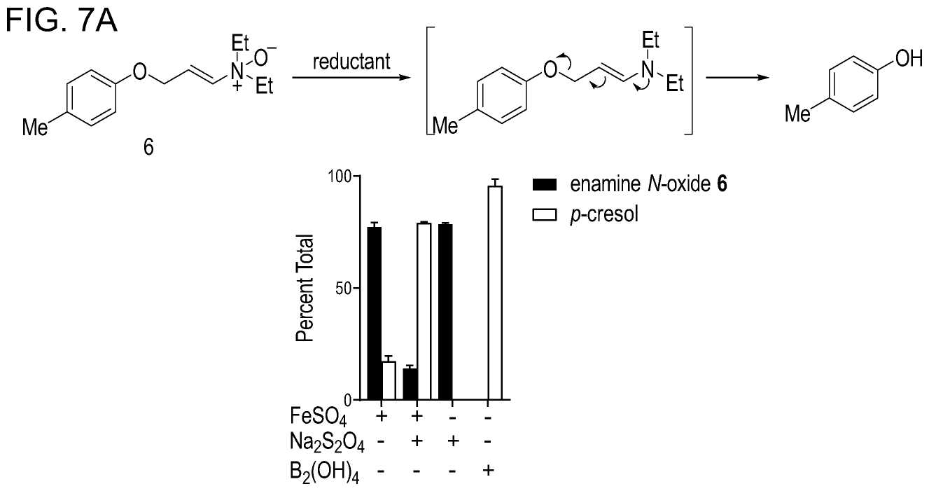

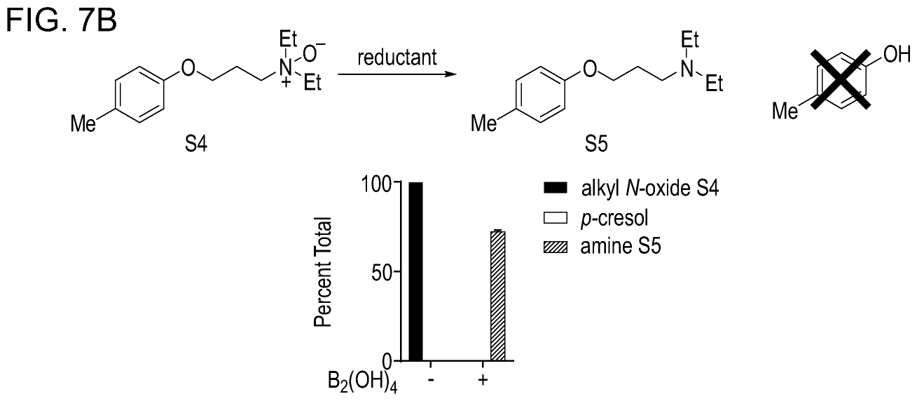

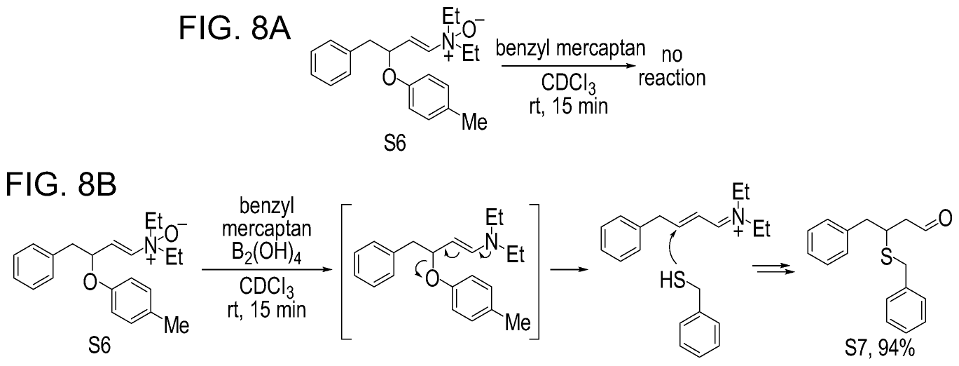

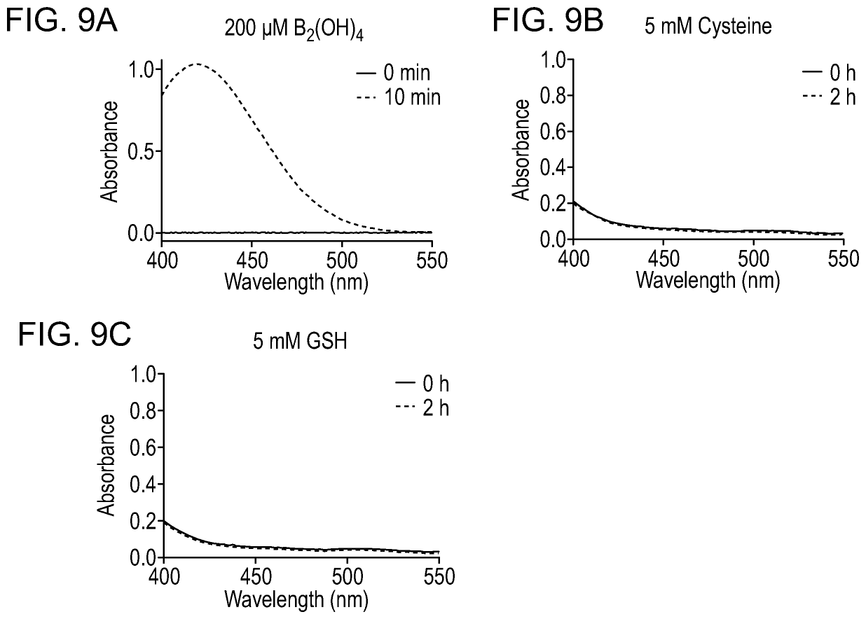

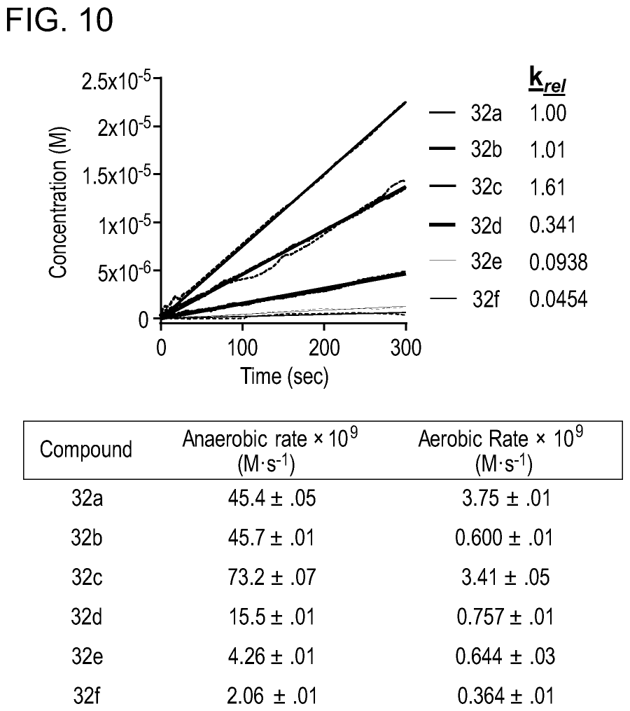

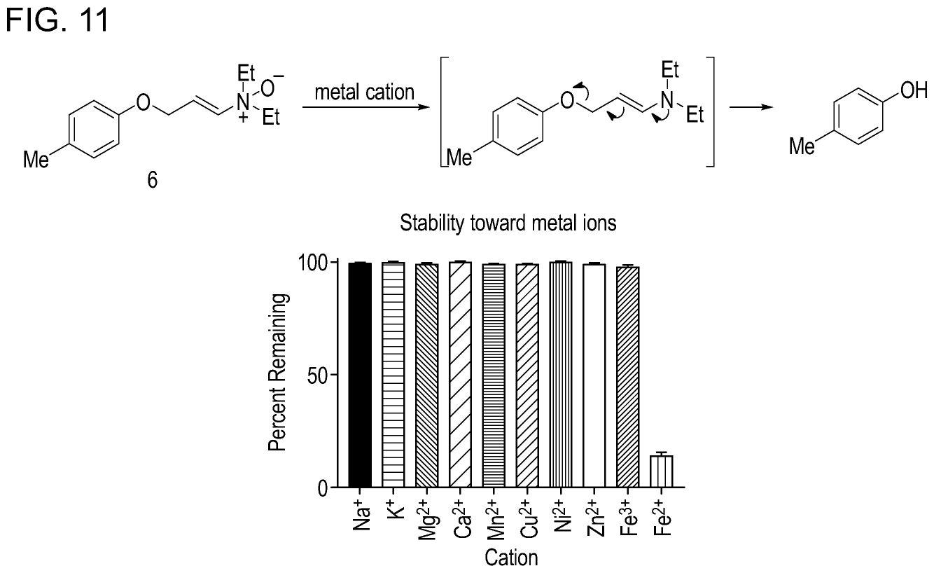

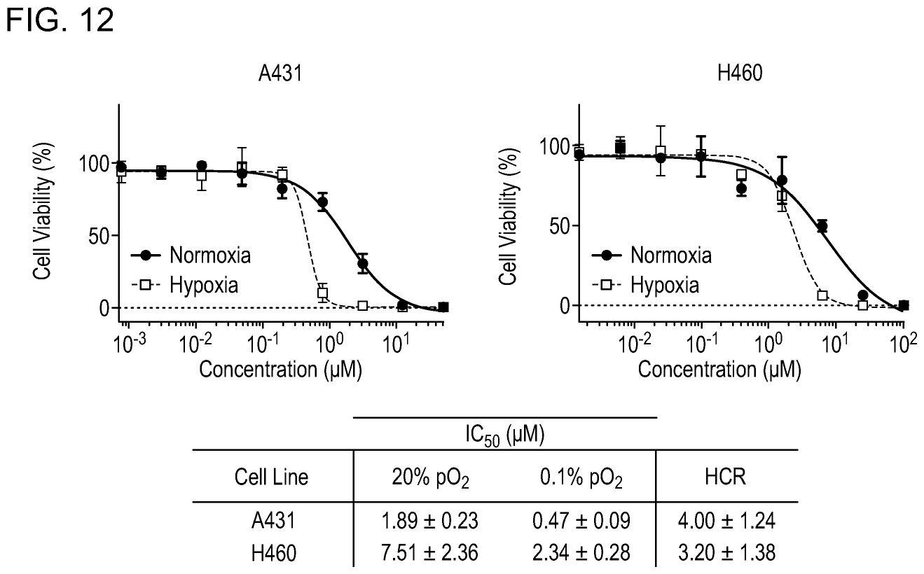

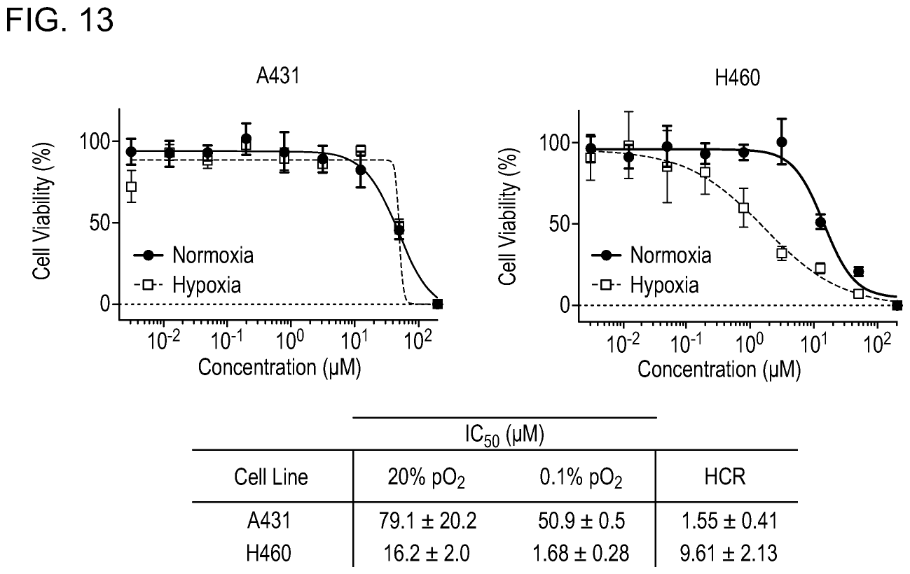

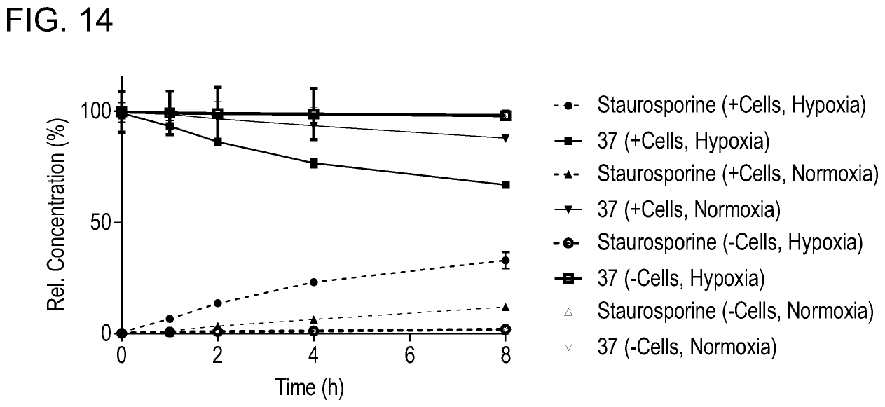

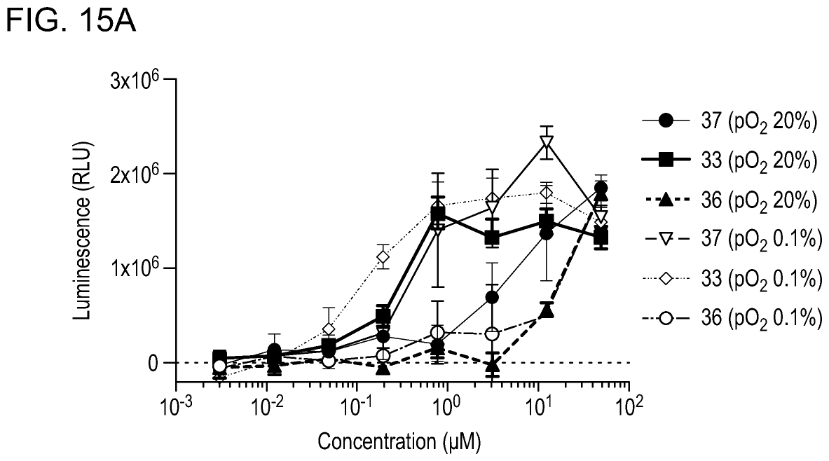

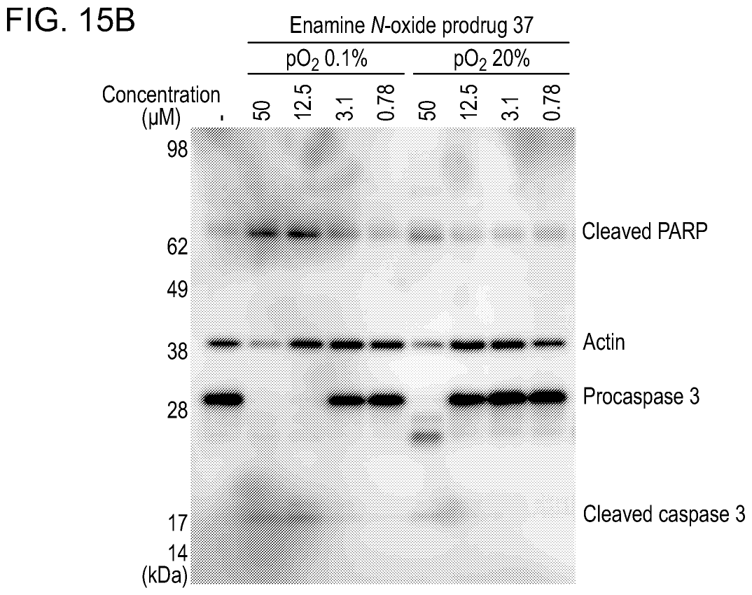

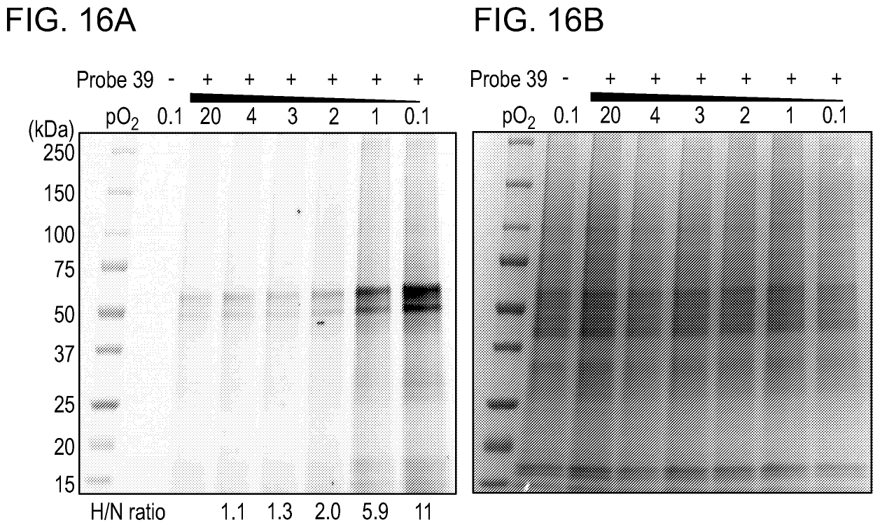

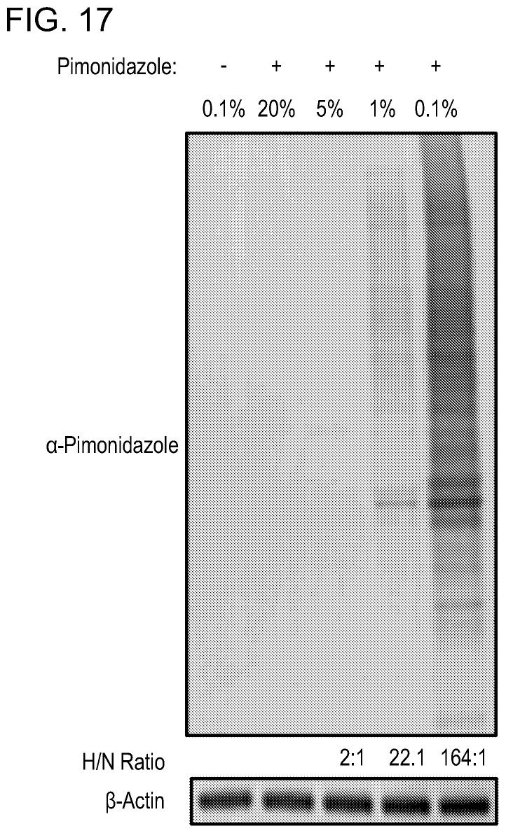

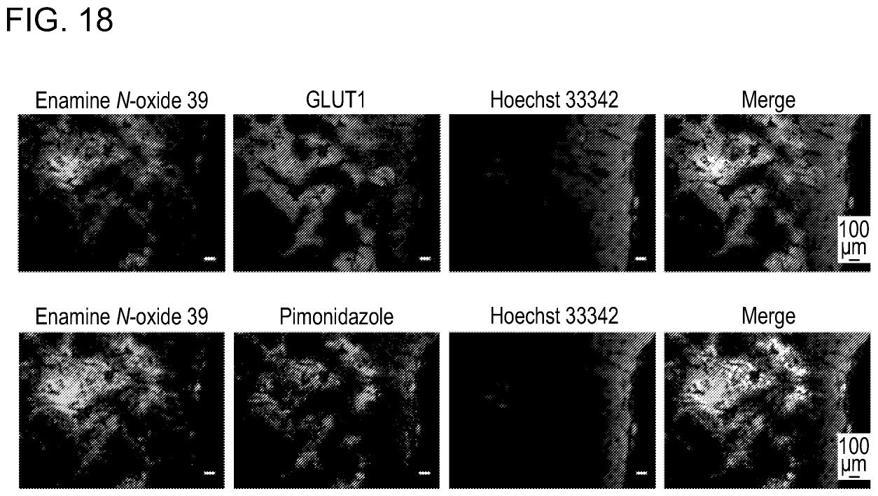

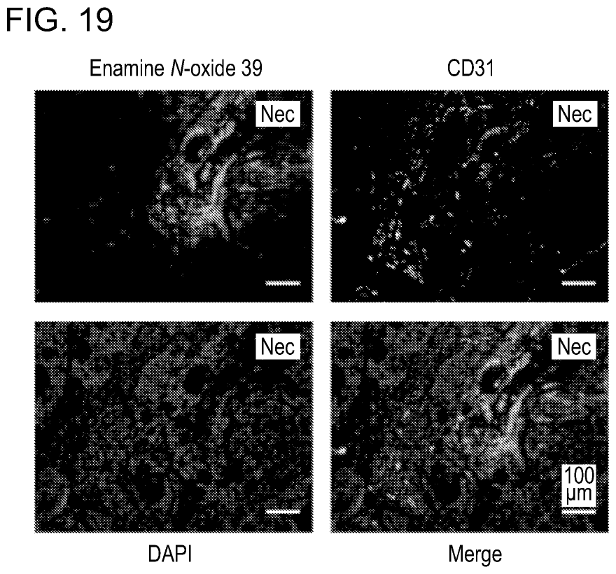

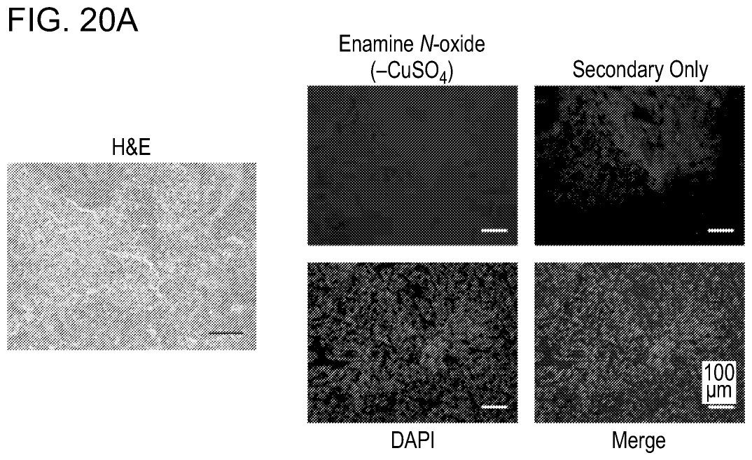

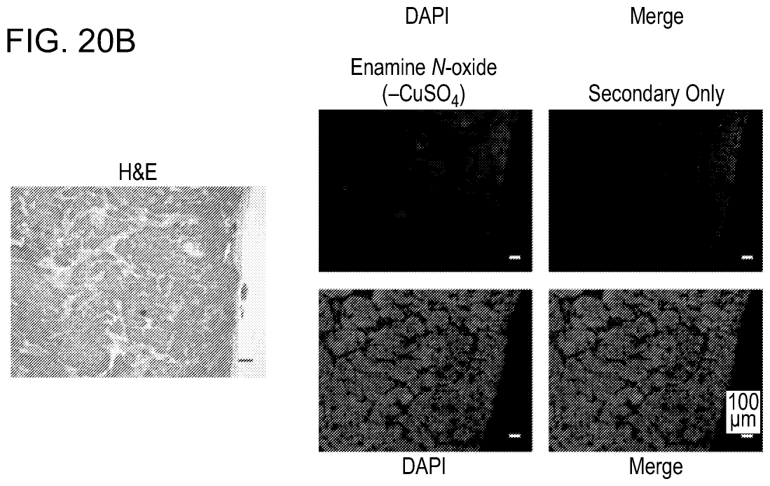

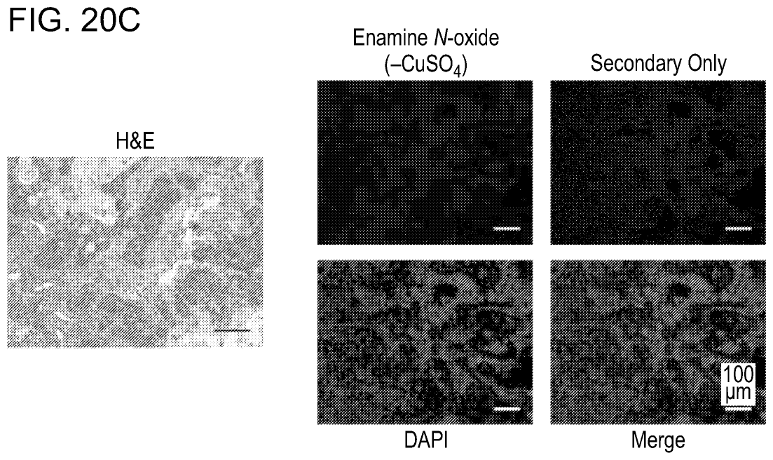

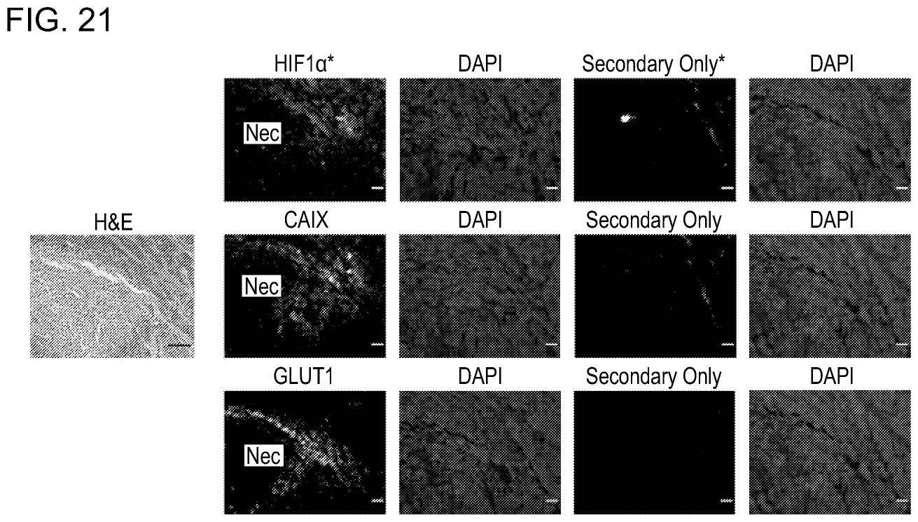

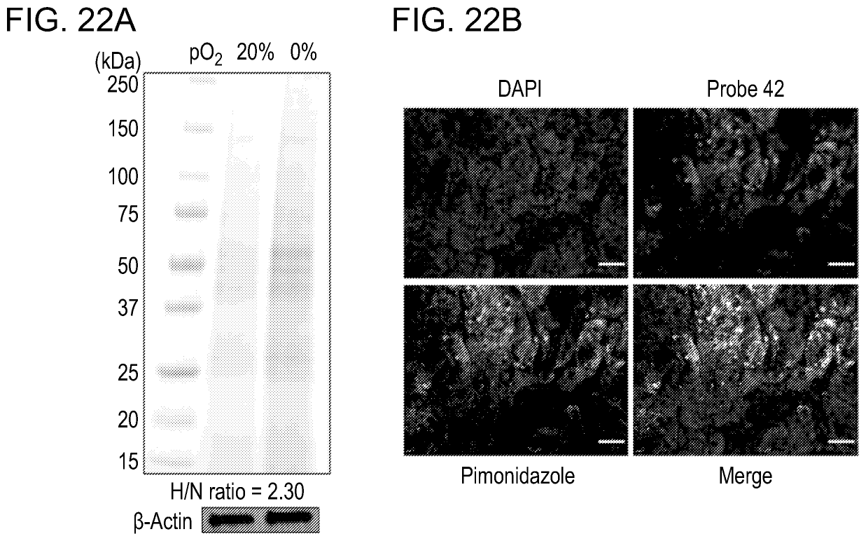

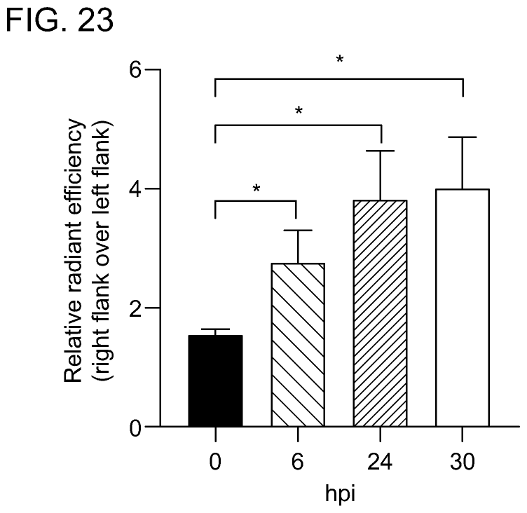

A solvent screen indicated that hydroamination rates were fastest but the products most prone to degradation in low polarity aprotic solvents (CH₂Cl₂, CHCl₃, CCl₄, DCE, PhMe) while reaction conversions were lower in polar protic ones (MeOH, EtOH, iPrOH, nBuOH) where fewer degradation products were observed (Tables 2-4). Given the centrality of the N-oxide oxygen atom in both Cope (FIG. 6) and Meissenheimer degradation processes, the role of solvent pKa was explored and it was discovered that 2,2,2-trifluoroethanol (TFE) mitigates degradation better than the less acidic alcohols isopropanol and n-butanol, likely through increased enamine N-oxide stabilization. Lower pKa solvents, however, adversely affected the reaction rate presumably by inhibition of the hydroxylamine reagent through protonation which was observed for 1,1,1,3,3,3-hexafluoroisopropanol (HFIP).

TABLE 2
Solvent effects on hydroamination at 60° C.^{a}

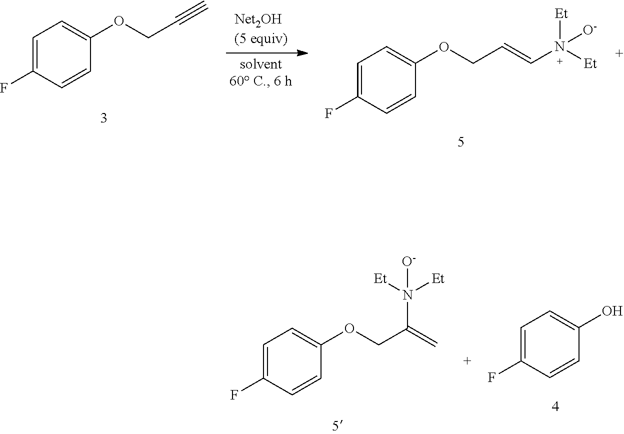

| | Yield (%)^{b} | | | | |
|---|---|---|---|---|---|
| Solvent | 3 | 5 + 5′ | 4 | Total | r.r. (5:5′) |
| CH₂Cl₂ | 0 | 43 | 46 | 89 | 7.0:1 |
| CHC₃ | 16 | 48 | 23 | 87 | 77:1 |
| CC₄ | 14 | 17 | 42 | 73 | 31:1 |
| DCE | 11 | 20 | 45 | 76 | 16:1 |
| PhMe | 19 | 30 | 38 | 87 | 41:1 |
| MeCN | 17 | 14 | 42 | 73 | 20:1 |
| THF | 45 | 5 | 24 | 74 | 12:1 |
| MeOH | 69 | 17 | 4 | 90 | 3.8:1 |
| EtOH | 59 | 37 | <1 | 96 | 3.9:1 |
| ⁱPrOH | 41 | 47 | 3 | 91 | 5.5:1 |
| ⁿBuOH | 46 | 53 | 1 | 100 | 4.0:1 |
| TFE | 51 | 47 | 2 | 100 | 3.7:1 |
| HFIP | 81 | 7 | 2 | 90 | 8.3:1 |

^{a}Conditions: alkyne 3 (0.2 mmol, 1 equiv, 0.2 M), N,N-diethylhydroxylamine (1 mmol, 5.0 equiv, 1 M). ^{b}Yields were determined by NMR using benzotrifluoride as an internal standard. r.r. = regioisomeric ratio.

TABLE 3
Effects of protic solvents on hydroamination at 60° C.^{a}

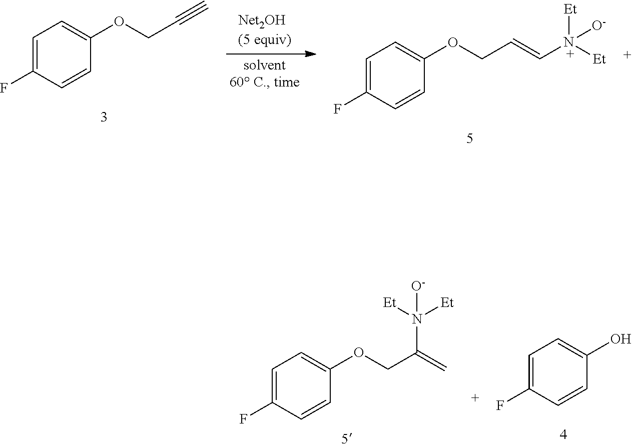

| | | Yield (%)^{b} | | | | r.r. |
|---|---|---|---|---|---|---|
| Solvent | Time (h) | 3 | 5 + 5′ | 4 | Total | (5:5′) |
| ⁱPrOH | 12 | 23 | 51 | 26 | 100 | 5.9:1 |
| ⁿBuOH | 12 | 21 | 75 | 4 | 100 | 4.6:1 |
| ⁿBuOH | 24 | 6.8 | 77 | 15 | 99 | 7.4:1 |
| TFE | 12 | 19 | 76 | 4 | 99 | 3.7:1 |
| TFE | 24 | 0 | 95 | 5 | 100 | 4.1:1 |

^{a}Conditions: alkyne 3 (0.2 mmol, 1 equiv, 0.2 M), N,N-diethylhydroxylamine (1 mmol, 5.0 equiv, 1 M). ^{b}Yields were determined by NMR using benzotrifluoride as an internal standard. r.r. = regioisomeric ratio.

TABLE 4
Enamine N-oxide 5 decomposes into p-fluorophenol (4) under different conditions.ª

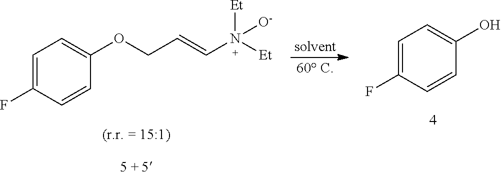

| | Yield (%)^{b} | | | |
|---|---|---|---|---|
| Time (h) | 5 + 5′ | 4 | Total | r.r. (5:5′) |
| CHCl₃ | 45 | 29 | 78 | N/A |
| TFE | 96 | <1 | 97 | 18:1 |

ªConditions: enamine N-oxide (0.1 mmol, 0.1 M). ^{b}Yields were determined by NMR using benzotrifluoride as an internal standard. r.r. = regioisomeric ratio.

Ultimately, the best balance between reaction rate and product stability was achieved by employing a low polarity solvent supplemented with a minimal quantity of a strong hydrogen bond donating solvent additive (entries 10 and 11, Table 5). The hydroamination of alkyne 3 with N,N-diethylhydroxylamine in 20% TFE/CHCl₃ (v/v) at 60° C. for 18 h provided the corresponding enamine N-oxides in 96% yield (entry 14, Table 5 and Table 6).

TABLE 5
Reaction optimization for the hydroamination reaction between alkynes and N,N-dialkylhydroxylaminesª

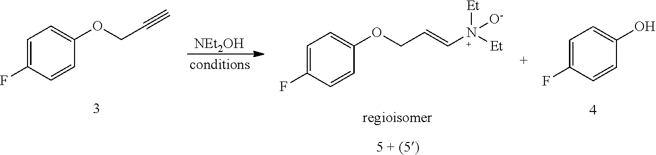

| | | Temp. | Time | Conv. | Yield (%)^{b} | |
|---|---|---|---|---|---|---|
| Entry | Solvent | (C) | (h) | (%) | 4 | 5 + 5′ |
| 1 | CHCl₃ | 50 | 6 | 65 | 5 | 50 |
| 2 | CHCl₃ | 60 | 6 | 84 | 23 | 48 |
| 3 | CHCl₃ | 70 | 6 | 100 | 49 | 31 |
| 4 | CH₂Cl₂ | 60 | 6 | 100 | 46 | 43 |
| 5 | DCE | 60 | 6 | 89 | 45 | 20 |
| 6 | ⁱPrOH | 60 | 6 | 59 | 3 | 47 |
| 7 | TFE | 60 | 6 | 49 | 2 | 47 |
| 8 | ⁱPrOH | 60 | 12 | 77 | 26 | 51 |
| 9 | TFE | 60 | 12 | 81 | 4 | 76 |
| 10 | 50% TFE/CHCl₃ | 60 | 6 | 55 | <1 | 52 |
| 11 | 20% TFE/CHCl₃ | 60 | 6 | 62 | <1 | 61 |
| 12^{c} | 20% TFE/CHCl₃ | 60 | 6 | 33 | <1 | 28 |
| 13^{d} | 20% TFE/CHCl₃ | 60 | 6 | 35 | <1 | 26 |
| 14 | 20% TFE/CHCl₃ | 60 | 18 | 95 | <1 | 96 |

ªConditions: alkyne 3 (0.2 mmol, 1 equiv, 0.2 M), N,N-diethylhydroxylamine (1 mmol, 5.0 equiv, 1 M). ^{b}Yields were determined by NMR using benzotrifluoride as an internal standard. ^{c}2 equivalents of N,N-diethylhydroxylamine was used. 0.1 M concentration. Temp. = temperature; Conv. = conversion.

TABLE 6
Reaction progression of hydroamination in 20% TFE/CHCl₃ at 60° C.^{a}

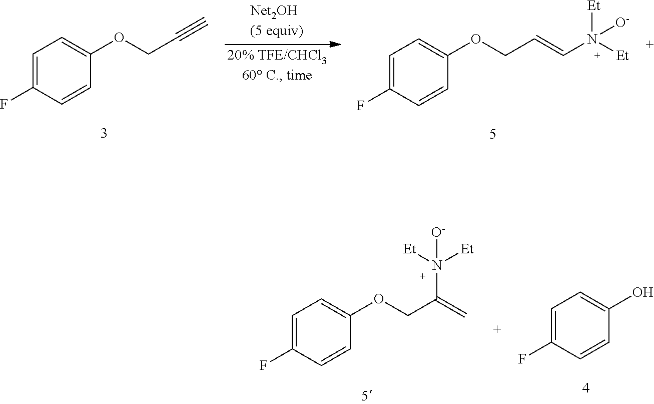

| | Yield (%)^{b} | | | | r.r. |
|---|---|---|---|---|---|
| Time (h) | 3 | 5 + 5′ | 4 | Total | (5:5′) |
| 6 | 38 | 61 | <1 | 99 | 4.2:1 |
| 12 | 14 | 84 | <1 | 99 | 4.4:1 |
| 15 | 10 | 89 | <1 | 100 | 4.3:1 |
| 18 | 5 | 96 | <1 | 102 | 4.6:1 |
| 21 | 5 | 95 | 1 | 101 | 4.5:1 |
| 24 | 4 | 95 | 1 | 100 | 4.5:1 |

^{a}Conditions: alkyne 3 (0.2 mmol, 1 equiv, 0.2 M), N,N-diethylhydroxylamine (1 mmol, 5.0 equiv, 1 M). ^{b}Yields were determined by NMR using benzotrifluoride as an internal standard. r.r. = regioisomeric ratio.

### Example 4: Reaction Optimization

Hydroamination reactions performed under the indicated conditions were carried out in 1-dram vials, flushed with nitrogen, sealed with Parafilm, and heated using a UCON™ oil bath. A vial was charged with alkyne 3 (30.0 mg, 0.200 mmol) as a 0.4 M solution in the reaction solvent at room temperature. N,N-diethylhydroxylamine was then added via syringe as a 2 M solution in the reaction solvent at room temperature. The vial was sealed and heated to the indicated temperature for the indicated amount of time. After heating, the reaction mixture was concentrated under reduced pressure and dissolved in CDCl₃. The amounts of enamine N-oxides (5 and 5′) and p-fluorophenol (4) were determined by ¹H and ¹⁹F-NMR integrations using benzotrifluoride as an internal standard. Regioisomeric ratios in different solvents were determined by ¹H-NMR integrations of vicinal versus geminal olefinic protons.

Degradation studies were performed using isolated enamine N-oxides (5:5′, 15:1). An LC-MS vial was charged with a 0.1 M solution of enamine N-oxides (23.9 mg, 100 μmol) in the reaction solvent of interest and heated to 60° C. for 6 hours. After heating, the reaction mixture was concentrated under reduced pressure and dissolved in CDCl₃. The amounts of enamine N-oxides (5 and 5′) and p-fluorophenol (4) were determined by ¹H and ¹⁹F-NMR integrations using benzotrifluoride as an internal standard. Regioisomer ratios in different solvents were determined by ¹H-NMR integrations of vicinal versus geminal olefinic protons.

### Example 5: General Procedure A: Hydroxylamine-Alkyne Hydroamination (Alkyne Scope)

A 1-dram glass vial was charged with alkyne (50.0 mg, 1) at room temperature. A solution of N,N-diethylhydroxylamine (1.00 M, 5.00 equiv) in 20% v/v trifluoroethanol in chloroform was then added via syringe. The vial was then flushed with nitrogen, capped, and sealed with Parafilm. The reaction mixture was heated to 60° C. in a UCON™ fluid heating bath until completion as determined by TLC. Upon completion, the oil bath was removed, and the reaction was cooled to room temperature. The reaction mixture was purified directly by flash chromatography on silica gel (eluent: CMA in chloroform). Fractions containing the desired compound were combined, and the solvent was removed under reduced pressure at 0° C. with a rotary evaporator.

### Example 6: General Procedure B: Hydroxylamine-Alkyne Hydroamination (Hydroxylamine Scope)

A 1-dram glass vial was charged with benzyl prop-2-yn-1-yl carbonate (Achard et al., Angew. Chem., Int. Ed. 50:3552-3556 (2011)) (2.00 equiv) at room temperature. A solution of the hydroxylamine (1.00 M, 20.0 mg, 1 equiv) in 20% v/v trifluoroethanol in chloroform was then added via syringe. The vial was flushed with nitrogen, capped, and sealed with Parafilm. The reaction mixture was heated to 60° C. in a UCON™ fluid heating bath until completion as determined by thin layer chromatography. Upon completion, the oil bath was removed, and the reaction was cooled to room temperature. The reaction mixture was then concentrated under reduced pressure. The resulting residue was purified by automated C₁₈ reverse phase column chromatography (30 g C₁₈ silica gel, 25 μm spherical particles, eluent: H₂O+0.1% TFA (2 CV), gradient 0→100% MeCN/H₂O+0.1% TFA (10 to 15 CV)). Fractions containing the desired compound were combined, and the solvent was removed under reduced pressure at 0° C. with a rotary evaporator.

### Example 7: Synthesis of (E)-N,N-diethyl-3-(4-fluorophenoxy)prop-1-en-1-amine oxide (5) and N,N-diethyl-3-(4-fluorophenoxy)prop-1-en-2-amine oxide (5′)

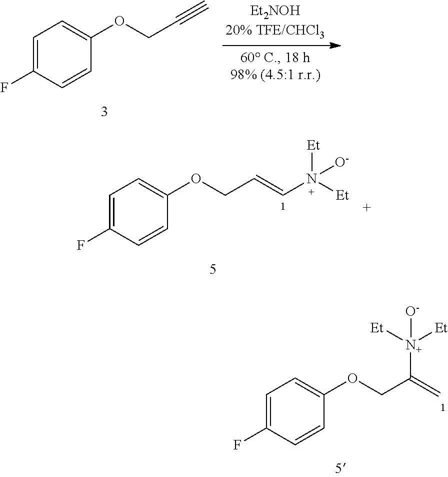

Enamine N-oxide 5 was synthesized following the general procedure A using 1-fluoro-4-(prop-2-yn-1-yloxy)benzene (3) (Tsuzuki et al., Bioorg. Med. Chem. Lett. 20:7269-7273, (2010)). The reaction mixture stirred at 60° C. for 18 hours and purified by flash column chromatography on silica gel (eluent: 30% CMA in chloroform) to provide regioisomeric enamine N-oxides 5 and 5′ (Trial 1: 77.2 mg, 97%; Trial 2: 78.4 mg, 98%) as a white solid. The regioisomeric ratio (5:5′, Trial 1: 4.4:1, Trial 2: 4.6:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): [5] δ 7.06-6.92 (m, 4H), 6.60 (dt, J=13.3, 4.9 Hz, 1H), 6.35 (dt, J=13.1, 1.8 Hz, 1H), 4.70 (dd, J=5.0, 1.9 Hz, 2H), 3.42-3.32 (m, 4H), 1.24 (t, J=7.1 Hz, 6H). [5′] δ 7.08-6.92 (m, 4H), 5.98 (d, J=1.7 Hz, 1H), 5.76 (d, J=1.5 Hz, 1H), 4.78 (d, J=1.1 Hz, 2H), 3.64 (dq, J=12.7, 7.2 Hz, 2H), 3.49-3.43 (m, 2H), 1.28 (t, J=7.1 Hz, 6H). ¹³C NMR (126 MHz, CD₃OD, 25° C.): [5] δ 159.1 (d, J=237.5 Hz), 155.9 (d, J=2.4 Hz), 140.1, 126.5, 117.4 (d, J=8.1 Hz), 116.9 (d, J=23.4 Hz), 66.4, 65.3, 8.7. [5′] δ 159.3 (d, J=238.0 Hz), 155.8 (d, J=2.9 Hz), 151.1, 117.4 (d, J=8.1 Hz), 117.1 (d, J=23.5 Hz), 115.8, 67.1, 64.7, 8.9. ¹⁹F NMR (471 MHz, CD₃OD, 25° C.) [5] δ −125.3. [5′] δ −124.8. FTIR (thin film) cm⁻¹: 3232 (w), 1506 (s), 1461 (w), 1200 (s), 962 (w), 828 (m) HRMS (ESI) (m/z): calc'd for C₁₃H₁₉FNO₂ [M+H]⁺: 240.1400, found: 240.1395. TLC (30% CMA in chloroform), Rf: 0.057 (UV, KMnO₄).

### Example 8: Synthesis of (E)-3-(((benzyloxy)carbonyl)oxy)-N,N-dimethylprop-1-en-1-amine oxide (6) and N,N-diethyl-3-(p-tolyloxy)prop-1-en-2-amine oxide (6′)

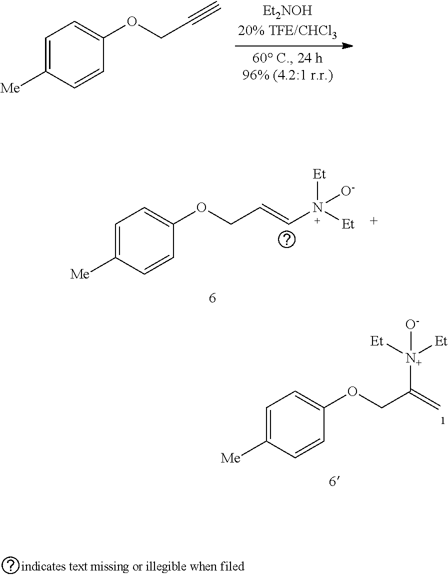

Enamine N-oxide 6 was synthesized following the general procedure A using 1-methyl-4-(prop-2-yn-1-yloxy)benzene (Efe et al., Chem. Commun. 47:803-805 (2011)). The reaction mixture stirred at 60° C. for 24 hours and purified by flash column chromatography on silica gel (eluent: 30% CMA in chloroform) to provide regioisomeric enamine N-oxides 6 and 6′ (Trial 1: 77.0 mg, 96%; Trial 2: 76.4 mg, 95%) as a white solid. The regioisomeric ratio (6:6′, Trial 1: 3.8:1, Trial 2: 4.6:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): [6] δ 7.04 (d, J=8.0 Hz, 2H), 6.80 (d, J=8.6 Hz, 2H), 6.55 (dt, J=13.2, 4.8 Hz, 1H), 6.27 (dt, J=13.2, 1.9 Hz, 1H), 4.64 (dd, J=4.8, 1.9 Hz, 2H), 3.37-3.32 (m, 2H), 3.30-3.25 (m, 2H), 2.22 (s, 3H), 1.19 (t, J=7.2 Hz, 6H). [6′] δ 7.06 (d, J=8.3 Hz, 2H), 6.84 (d, J=8.6 Hz, 2H), 5.93 (d, J=1.6 Hz, 1H), 5.70 (d, J=1.4 Hz, 1H), 4.72 (d, J=1.0 Hz, 2H), 3.63-3.54 (m, 2H), 3.45-3.37 (m, 2H), 2.23 (s, 3H), 1.23 (t, J=7.1 Hz, 6H). ¹³C NMR (126 MHz, CD₃OD, 25° C.): [6] δ 157.5, 151.3, 139.8, 131.1, 126.8, 115.9, 65.8, 65.3, 20.7, 8.7. [6′] δ 157.4, 132.2, 131.8, 131.2, 115.9, 115.7, 66.6, 64.7, 20.7, 8.9. FTIR (thin film) cm⁻¹: 2982 (w), 2941 (w), 1513 (s), 1238 (m), 1014 (w), 961 (w), 816 (w). HRMS (ESI) (m/z): calc'd for C₁₄H₂₂NO₂ [M+H]⁺: 236.1645, found: 236.1641. TLC (30% CMA in chloroform), Rf: 0.20 (UV, KMnO₄).

### Example 9: Synthesis of (E)-N,N-diethyl-3-(4-nitrophenoxy)prop-1-en-1-amine oxide (7) and N,N-diethyl-3-(4-nitrophenoxy)prop-1-en-2-amine oxide (7′)

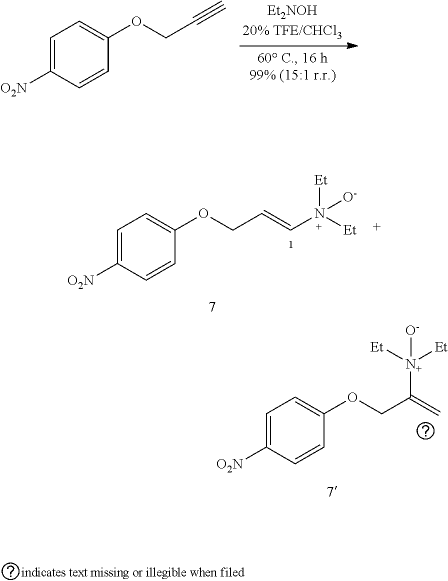

Enamine N-oxide 7 was synthesized following the general procedure A using 1-nitro-4-(prop-2-yn-1-yloxy)benzene (Tsuzuki et al., Bioorg. Med. Chem. Lett. 20:7269-7273, (2010)). The reaction mixture stirred at 60° C. for 16 hours and purified by flash column chromatography on silica gel (eluent: 30% CMA in chloroform) to provide regioisomeric enamine N-oxides 7 and 7′ (Trial 1: 74.5 mg, 99%; Trial 2: 73.4 mg, 97%) as a yellow oil. The regioisomeric ratio (7:7′, Trial 1: 15:1, Trial 2: 15:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): [7] δ 8.21 (d, J=9.3 Hz, 2H), 7.13 (d, J=9.3 Hz, 2H), 6.65 (dt, J=13.3, 5.0 Hz, 1H), 6.43 (dt, J=13.1, 1.7 Hz, 1H), 4.88 (dd, J=5.0, 1.8 Hz, 2H), 3.45-3.34 (m, 4H), 1.26 (t, J=7.2 Hz, 6H). [7′] δ 8.21 (d, J=9.3 Hz, 2H), 7.17 (d, J=9.3 Hz, 2H), 6.01 (d, J=2.1 Hz, 1H), 5.86-5.79 (m, 1H), 4.96 (d, J=0.9 Hz, 2H), 3.70-3.46 (m, 4H), 1.31 (t, J=7.1 Hz, 6H). ¹³C NMR (126 MHz, CD₃OD, 25° C.): [7] δ 163.2, 141.8, 139.2, 125.5, 124.1, 114.7, 65.0, 63.8, 7.1. [7′] δ 162.9, 149.1, 142.0, 125.8, 115.6, 114.7, 65.5, 63.3, 7.4. FTIR (thin film) cm⁻¹: 3213 (br), 2944 (w), 1591 (s), 1509 (s), 1334 (s), 1260 (s), 1110 (m). HRMS (ESI) (m/z): calc'd for C₁₃H₁₉N₂O₄ [M+H]⁺: 267.1345, found: 267.1337. TLC (30% CMA in chloroform), Rf: 0.097 (UV, KMnO₄).

### Example 10: Synthesis of (E)-N,N-diethyl-3-(4-methoxyphenoxy)prop-1-en-1-amine oxide (8) and N,N-diethyl-3-(4-methoxyphenoxy)prop-1-en-2-amine oxide (8′)

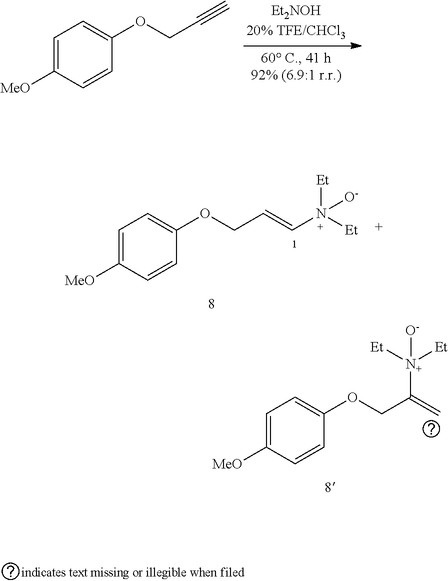

Enamine N-oxide 8 was synthesized following the general procedure A using 1-methoxy-4-(prop-2-yn-1-yloxy)benzene (Achard et al., Angew. Chem., Int. Ed. 50:3552-3556 (2011)). The reaction mixture was stirred at 60° C. for 41 hours and purified by flash column chromatography on silica gel (eluent: 30% CMA in chloroform) to provide regioisomeric enamine N-oxides 8 and 8′ (Trial 1: 70.4 mg, 91%; Trial 2: 71.7 mg, 93%) as a white solid. The regioisomeric ratio (8:8′, Trial 1: 9.3:1, Trial 2: 4.5:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): [8] δ 6.97-6.82 (m, 4H), 6.60 (dt, J=13.1, 4.9 Hz, 1H), 6.34 (dt, J=13.1, 1.9 Hz, 1H), 4.69 (dd, J=4.9, 1.8 Hz, 2H), 3.76 (s, 3H), 3.44-3.32 (m, 4H), 1.25 (t, J=7.2 Hz, 6H). [8′] δ 6.97-6.86 (m, 4H), 5.97 (d, J=1.7 Hz, 1H), 5.75 (d, J=1.4 Hz, 1H), 4.74 (s, 2H), 3.75 (s, 3H), 3.69-3.57 (m, 2H), 3.50-3.40 (m, 2H), 1.27 (t, J=7.1 Hz, 6H). ¹³C NMR (126 MHz, CD₃OD, 25° C.): [8] δ 155.9, 153.7, 139.9, 126.8, 117.1, 115.9, 66.4, 65.3, 56.3 8.7. [8′] δ 156.1, 153.6, 151.3, 117.1, 115.9, 115.7, 67.2, 64.6, 56.3, 8.9. FTIR (thin film) cm⁻¹: 3232 (br), 2945 (w), 1506 (d), 1461 (m), 1223 (s), 1033 (m), 828 (m). HRMS (ESI) (m/z): calc'd for C₁₄H₂₂NO₃ [M+H]⁺: 252.1600, found: 252.1593. TLC (30% CMA in chloroform), Rf: 0.13 (UV, KMnO₄).

### Example 11: Synthesis of (E)-N,N-diethyl-3-(octyloxy)prop-1-en-1-amine oxide (9) and N,N-diethyl-3-(octyloxy)prop-1-en-2-amine oxide (9′)

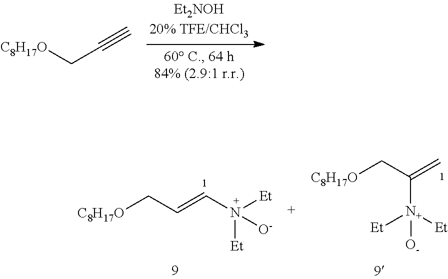

Enamine N-oxide 9 was synthesized following the general procedure A using 1-(prop-2-yn-1-yloxy)octane (Sahoo et al., Org. Biomol. Chem. 12:2615-2625, (2014)). The reaction mixture was heated for 64 hours and purified by flash column chromatography on silica gel (eluent: 30% CMA in chloroform) to provide regioisomeric enamine N-oxides 9 and 9′ (Trial 1: 65.1 mg, 85%; Trial 2: 63.0 mg, 82%) as a clear, colorless oil. The regioisomeric ratio (9:9′, Trial 1: 3.5:1, Trial 2: 2.3:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): [9] δ 6.45 (dt, J=13.1, 5.1 Hz, 1H), 6.21 (dt, J=13.1, 1.8 Hz, 1H), 4.12 (dd, J=5.0, 1.8 Hz, 2H), 3.48 (t, J=6.6 Hz, 2H), 3.45-3.31 (m, 4H), 1.39-1.28 (m, 12H), 1.26 (t, J=7.2 Hz, 6H), 0.90 (m, 3H). [9′] δ 5.95 (d, J=1.0 Hz, 1H), 5.59 (d, J=1.1 Hz, 1H), 4.18 (d, J=0.9 Hz, 2H), 3.64-3.55 (m, 2H), 3.50 (t, J=6.5 Hz, 2H), 3.45-3.31 (m, 2H), 1.63-1.55 (m, 6H), 1.39-1.28 (m, 6H), 1.23 (t, J=7.0 Hz, 6H), 0.90 (m, 3H). ¹³C NMR (126 MHz, CD₃OD, 25° C.): [9] δ 139.2, 127.8, 72.1, 68.3, 65.2, 33.2, 30.9, 30.7, 30.6, 27.4, 23.9, 14.6, 8.7. [9′] δ 151.8, 115.5, 72.4, 69.3, 64.4, 33.1, 30.8, 30.6, 30.6, 27.5, 23.9, 14.6, 8.8. FTIR (thin film) cm⁻¹: 2929 (s), 2855 (m), 1464 (w), 1367 (w), 1107 (m), 961 (w). HRMS (ESI) (m/z): calc'd for C₁₅H₃₂NO₂ [M+H]⁺: 258.2428, found: 258.2422. TLC (30% CMA in chloroform), Rf: 0.10 (KMnO₄).

### Example 12: Synthesis of (E)-N,N-diethyl-3-hydroxyprop-1-en-1-amine oxide (10) and N,N-diethyl-3-hydroxyprop-1-en-2-amine oxide (10′)

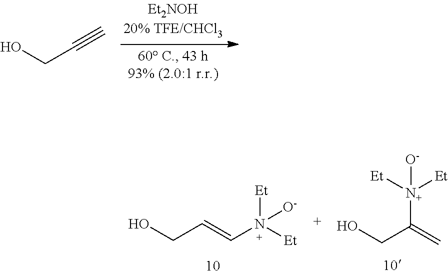

Enamine N-oxide 10 was synthesized following the general procedure A using prop-2-yn-1-ol (Sigma Aldrich). The reaction mixture was stirred at 60° C. for 43 hours and purified by flash column chromatography on silica gel (eluent: 40% CMA in chloroform) to provide enamine N-oxides 10 (Trial 1: 82.9 mg, 64%; Trial 2: 77.2 mg, 60%) as a brown oil and 10′ (Trial 1: 39.5 mg, 30%; Trial 2: 40.9 mg, 32%) as a brown oil. The regioisomeric ratio (10:10′, Trial 1: 2.1:1, Trial 2: 1.9:1) was determined by taking the ratio of the isolated amount of regioisomers. ¹H NMR (500 MHz, CD₃OD, 25° C.): [10] δ 6.48 (dt, J=13.1, 4.7 Hz, 1H), 6.18 (dt, J=13.3, 2.1 Hz, 1H), 4.23 (dd, J=4.7, 2.0 Hz, 2H), 3.50-3.33 (m, 4H), 1.27 (t, J=7.2 Hz, 6H). [10′] δ 5.78 (d, J=1.5 Hz, 1H), 5.58 (d, J=1.2 Hz, 1H), 3.60 (dq, J=12.4, 7.0 Hz, 2H), 3.45-3.34 (m, 2H), 1.25 (t, J=7.1 Hz, 4H). ¹³C NMR (126 MHz, CD₃OD, 25° C.): [10] δ 138.1, 130.6, 65.2, 60.0, 8.7. [10′] δ 155.1, 112.7, 64.9, 61.1, 8.8. FTIR (thin film) cm⁻¹: [10] 3213 (br), 2989 (m), 1659 (m), 1450 (m), 1103 (m), 954 (s). [10′] 3198 (br), 2989 (m), 1662 (m), 1450 (m), 1379 (m), 954 (s). HRMS (ESI) (m/z): [10] calc'd for C₇H₁₆NO₂ [M+H]⁺: 146.1181, found: 146.1176. [10′] calc'd for C₇H₁₆NO₂ [M+H]⁺: 146.1181, found: 146.1176. TLC (60% CMA in chloroform), Rf: [10] 0.14 (KMnO₄). [10′] 0.11 (KMnO₄).

### Example 13: Synthesis of (E)-N,N-diethyl-3-(pivalovloxy)prop-1-en-1-amine oxide (11) and N,N-diethyl-3-(pivaloyloxy)prop-1-en-2-amine oxide (11′)

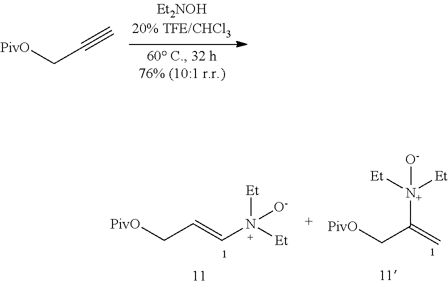

Enamine N-oxide 11 was synthesized following the general procedure A using prop-2-yn-1-yl pivalate (Achard et al., Angew. Chem., Int. Ed. 50:3552-3556 (2011)). The reaction mixture was heated for 32 hours and purified by flash column chromatography on silica gel (eluent: 30% CMA in chloroform) to provide regioisomeric enamine N-oxides 11 and 11′ (Trial 1: 60.5 mg, 74%; Trial 2: 63.6 mg, 78%) as a clear, yellow oil. The regioisomeric ratio (11:11′, Trial 1: 9.1:1, Trial 2: 11.1:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): [11] δ 6.50 (dt, J=13.2, 6.1 Hz, 1H), 6.29 (dt, J=13.2, 1.5 Hz, 1H), 4.72 (dd, J=6.1, 1.5 Hz, 2H), 3.43-3.32 (m, 4H), 1.29-1.22 (m, 6H), 1.22 (s, 9H). [11′] δ 5.92 (d, J=2.0 Hz, 1H), 5.61-5.56 (m, 1H), 4.85 (d, J=2.0 Hz, 2H), 3.64-3.52 (m, 2H), 3.49-3.41 (m, 2H), 1.29-1.22 (m, 6H), 1.22 (s, 9H). ¹³C NMR (126 MHz, CD₃OD, 25° C.): [11] δ 179.4, 141.3, 125.5, 65.3, 62.0, 27.6, 8.6. [11′] δ 179.0, 150.8, 115.3, 64.5, 40.0, 28.2, 8.8. FTIR (thin film) cm⁻¹: 2974 (m), 1729 (s), 1461 (w), 1367 (w), 1282 (m), 1155 (s), 957 (w). HRMS (ESI) (m/z): calc'd for C₁₂H₂₄NO₃ [M+H]⁺: 230.1751, found: 230.1746. TLC (30% CMA in chloroform), Rf: 0.10 (KMnO₄).

### Example 14: Synthesis of (E)-N,N-diethyl-3-((phenylcarbamoyl)oxy)prop-1-en-1-amine oxide (12) and N,N-diethyl-3-((phenylcarbamoyl)oxy)prop-1-en-2-amine oxide (12′)

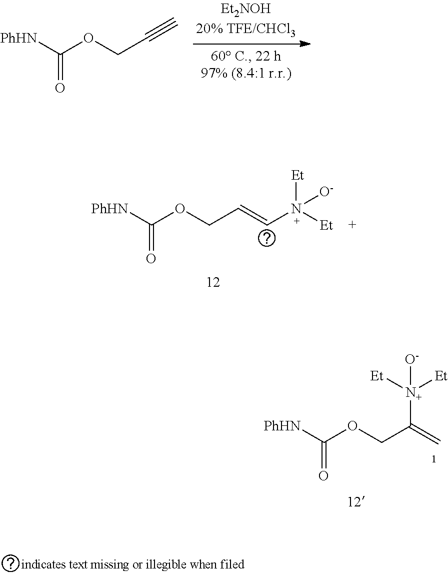

Enamine N-oxide 12 was synthesized following the general procedure A using prop-2-yn-1-yl phenylcarbamate (Newton et al., Aust. J. Chem. 61:432-437 (2008)). The reaction mixture was heated for 22 hours and purified by flash column chromatography on silica gel (eluent: 30% CMA in chloroform) to provide regioisomeric enamine N-oxides 12 and 12′ (Trial 1: 74.4 mg, 99%; Trial 2: 71.2 mg, 94%) as a clear, colorless oil. The regioisomeric ratio (12:12′, Trial 1: 8.5:1, Trial 2: 8.3:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): [12] δ 7.43 (d, J=8.0 Hz, 2H), 7.27 (t, J=8.0 Hz, 2H), 7.02 (t, J=7.4 Hz, 1H), 6.55 (dt, J=13.2, 5.7 Hz, 1H), 6.32 (dt, J=13.2, 1.7 Hz, 1H), 4.79 (dd, J=5.7, 1.6 Hz, 2H), 3.44-3.31 (m, 4H), 1.26 (t, J=7.2 Hz, 6H). [12′] δ 7.45 (d, 2H), 7.29 (t, 2H), 7.03 (t, 1H), 5.91 (d, J=1.9, 1H), 5.67 (s, 1H), 4.92 (s, 2H), 3.66-3.43 (m, 4H), 1.26 (t, J=7.2, 6H). ¹³C NMR (126 MHz, CD₃OD, 25° C.): [12] δ 153.2, 140.7, 140.2, 130.0, 125.9, 124.3, 120.0, 65.3, 62.2, 8.6. [12′]δ 153.2, 151.3, 140.2, 130.0, 124.4, 120.0, 114.8, 64.6, 62.0, 8.8. FTIR (thin film) cm⁻¹: 2944 (w), 1714 (m), 1599 (m), 1546 (m), 1446 (m), 1315 (m), 1218 (s), 1054 (m), 756 (s). HRMS (ESI) (m/z): calc'd for C₁₄H₂₁N₂O₃ [M+H]⁺: 265.1547, found: 265.1541. TLC (30% CMA in chloroform), Rf: 0.20 (UV, KMnO₄).

### Example 15: Synthesis of (E)-3-(((benzyloxy)carbonyl)oxy)-N,N-diethylprop-1-en-1-amine oxide (13

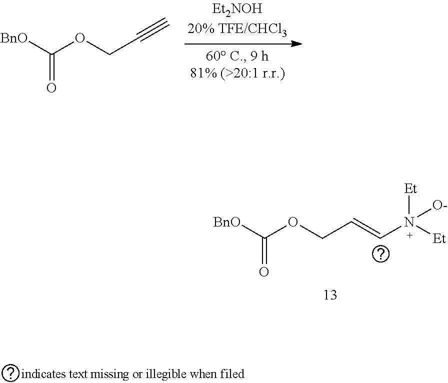

Enamine N-oxide 13 was synthesized following the general procedure A using benzyl prop-2-yn-1-yl carbonate (Achard et al., Angew. Chem., Int. Ed. 50:3552-3556 (2011)). The reaction mixture was heated for 9 hours and purified by flash column chromatography on silica gel (eluent: 30% CMA in chloroform) to provide enamine N-oxide 13 (Trial 1: 61.0 mg, 83%; Trial 2: 58.2 mg, 79%) as a clear, colorless oil. The regioisomeric ratio (>20:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): δ 7.41-7.32 (m, 5H), 6.51 (dtd, J=13.2, 5.1, 1.3 Hz, 1H), 6.30 (dt, J=13.5, 2.1 Hz, 1H), 5.17 (s, 2H), 4.80 (d, J=5.7 Hz, 2H), 3.41-3.31 (m, 4H), 1.22 (t, J=7.2 Hz, 6H). ¹³C NMR (126 MHz, CD₃OD, 25° C.): δ 156.3, 141.1, 137.1, 129.8, 129.7, 129.5, 125.1, 71.0, 65.4, 65.3, 8.6. FTIR (thin film) cm⁻¹: 1748 (m), 1666 (w), 1394 (w), 1244 (m), 1177 (s), 1129 (s), 946 (m). HRMS (ESI) (m/z): calc'd for C₁₅H₂₂NO₄ [M+H]⁺: 280.1543, found: 280.1538. TLC (30% CMA in chloroform), Rf: 0.30 (UV, KMnO₄).

### Example 16: Synthesis of (E)-3-((diethoxyphosphoryl)oxy)-N,N-diethylprop-1-en-1-amine oxide (14)

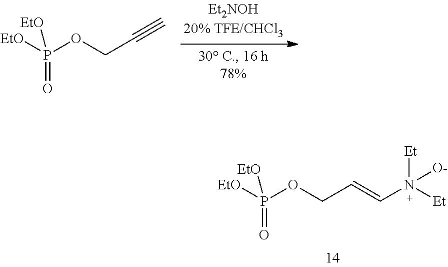

Enamine N-oxide 14 was synthesized following the general procedure A using diethyl prop-2-yn-1-yl phosphate (Jones et al., Org. Lett. 7:3271-3274, (2005)). The reaction mixture stirred at 60° C. for 16 hours and purified by flash column chromatography on silica gel (eluent: 30% CMA in chloroform) to provide enamine N-oxide 14 (Trial 1: 58.1 mg, 77%; Trial 2: 59.4 mg, 78%) as a yellow oil. ¹H NMR (500 MHz, CD₃OD, 25° C.): δ 6.55 (dtd, J=13.1, 5.4, 1.1 Hz, 1H), 6.37 (dt, J=13.1, 1.7 Hz, 1H), 4.71 (ddd, J=8.4, 5.4, 1.7 Hz, 2H), 4.15 (dq, J=8.2, 7.0 Hz, 4H), 3.49-3.32 (m, 4H), 1.35 (td, J=7.1, 1.1 Hz, 6H), 1.27 (t, J=7.2 Hz, 6H). ¹³C NMR (126 MHz, CD₃OD, 25° C.): δ 141.0, 125.8 (d, J=6.7 Hz), 65.8 (d, J=6.2 Hz), 65.4, 65.3, 16.6 (d, J=6.7 Hz), 8.6. ³¹P NMR (202 MHz, CD₃OD, 25° C.) δ −1.5. FTIR (thin film) cm⁻¹: 3399 (br), 2989 (m), 1599 (m), 1260 (m), 1029 (s), 805 (w). HRMS (ESI) (m/z): calc'd for C₁₁H₂₅NO₅P [M+H]⁺: 282.1470, found: 282.1463. TLC (30% CMA in chloroform), Rf: 0.079 (KMnO₄).

### Example 17: Synthesis of (E)-N,N-diethyl-3-hydroxydec-1-en-1-amine oxide (15)

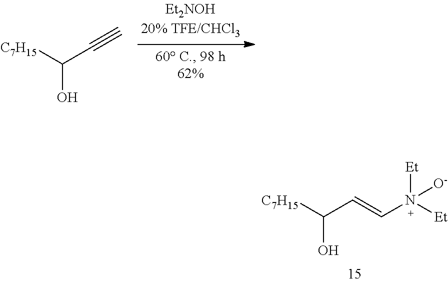

Enamine N-oxide 15 was synthesized following the general procedure A using dec-1-yn-3-ol (Ye et al., J. Am. Chem. Soc. 132:8550-8551 (2010)). The reaction mixture stirred at 60° C. for 98 hours and purified by flash column chromatography on silica gel (eluent: 40% CMA in chloroform) to provide enamine N-oxide 15 (Trial 1: 49.0 mg, 62%, Trial 2: 48.0 mg, 61%) as a yellow oil. ¹H NMR (500 MHz, CD₃OD, 25° C.): δ 6.39 (dd, J=13.1, 5.6 Hz, 1H), 6.13 (dd, J=13.1, 1.7 Hz, 1H), 4.25 (qd, J=6.4, 1.6 Hz, 1H), 3.42-3.31 (m, 4H), 1.61-1.53 (m, 2H), 1.47-1.29 (m, 10H), 1.27 (td, J=7.2, 5.0 Hz, 6H), 0.95-0.85 (m, 3H). ¹³C NMR (126 MHz, CD₃OD, 25° C.): δ 138.1, 134.0, 70.1, 65.3, 38.6, 33.1, 30.7, 30.5, 26.6, 23.9, 14.6, 8.7. FTIR (thin film) cm⁻¹: 3220 (br), 2926 (s), 2855 (s), 1595 (w), 1461 (m), 1379 (m), 965 (m). HRMS (ESI) (m/z): calc'd for C₈H₁₇NO₂ [M+H]⁺: 244.2271, found: 244.2271. TLC (50% CMA in chloroform), Rf: 0.11 (KMnO₄).

### Example 18: Synthesis of (3-fluorodec-1-yne (S9)

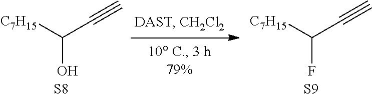

A round-bottom flask was charged with dec-1-yn-3-ol (Ye et al., J. Am. Chem. Soc. 132:8550-8551 (2010)) (300 mg, 1.94 mmol, 1 equiv), dissolved in dichloromethane (19 mL), and cooled to −10° C. using a calcium chloride-ice bath. Diethylaminosulfur trifluoride (308 μL, 2.33 mmol, 1.20 equiv) was added dropwise via syringe. After 3 hours, water (10 mL) was added to the reaction mixture. The organic layer was then washed with water (2×10 mL), dried with anhydrous sodium sulfate, filtered, and concentrated under reduced pressure. The crude residue was purified by flash column chromatography on silica gel (eluent: 100% hexanes) to provide fluoroalkyne S9 (240 mg, 79%) as a clear, colorless oil. ¹H NMR (500 MHz, CDCl₃, 25° C.): δ 5.06 (dtd, J=48.2, 6.4, 2.0 Hz, 1H), 2.64 (dd, J=5.6, 2.1 Hz, 1H), 1.92-1.70 (m, 2H), 1.52-1.39 (m, 2H), 1.34-1.22 (m, 8H), 0.86 (t, J=7.0 Hz, 3H). ¹³C NMR (126 MHz, CDCl₃, 25° C.): δ 82.65 (d, J=167.2 Hz), 80.73 (d, J=25.9 Hz), 76.28 (d, J=10.5 Hz), 36.01 (d, J=22.2 Hz), 31.9, 29.3, 29.3, 24.57 (d, J=4.2 Hz), 22.8, 14.3. ¹⁹F NMR (471 MHz, CDCl₃, 25° C.): δ 175.0. FTIR (thin film) cm⁻¹: 3306 (m), 2926 (s), 2858 (s), 1461 (m). HRMS (GC-MS) (m/z): calc'd for C₁₀H₁₇F [M]⁺: 156.1309, found: 156.1309. TLC (100% hexanes), Rf: 0.70 (KMnO₄).

### Example 19: Synthesis of (E)-N,N-diethyl-3-fluorodec-1-en-1-amine oxide (16)

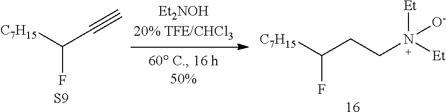

Enamine N-oxide 16 was synthesized following the general procedure A using 3-fluorodec-1-yne (S9). The reaction mixture was heated for 16 hours and purified by flash column chromatography on silica gel (eluent: 30% CMA in chloroform) to provide enamine N-oxide 16 (Trial 1: 43.3 mg, 55%; Trial 2: 35.1, 45%) as a clear, colorless oil. ¹H NMR (500 MHz, CD₃OD, 25° C.): δ 6.49 (ddd, J=18.3, 13.1, 5.3 Hz, 1H), 6.30 (dt, J=13.1, 1.8 Hz, 1H), 5.24-5.07 (m, 1H), 3.46-3.33 (m, 4H), 1.80-1.67 (m, 2H), 1.49-1.41 (m, 2H), 1.39-1.29 (m, 8H), 0.95-0.87 (m, 3H). 13C NMR (126 MHz, CD₃OD, 25° C.): δ 139.61 (d, J=12.4 Hz), 129.89 (d, J=18.2 Hz), 91.52 (d, J=170.7 Hz), 65.36 (d, J=9.0 Hz), 36.58 (d, J=21.2 Hz), 33.06, 30.53, 30.45, 25.80 (d, J=4.2 Hz), 23.84, 14.56, 8.67. ¹⁹F NMR (471 MHz, CDCl₃, 25° C.): δ 181.2. FTIR (thin film) cm⁻¹: 2929 (s), 2858 (s), 1684 (w), 1464 (m), 1375 (m), 1129 (w), 965 (s). HRMS (ESI) (m/z): calc'd for C₁₄H₂₉FNO [M+H]⁺: 246.2228, found: 246.2222. TLC (30% CMA in chloroform), Rf: 0.30 (KMnO₄).

### Example 20: Synthesis of 3-chlorodec-1-yne (S10)

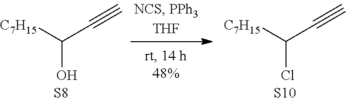

A round-bottom flask was charged with dec-1-yn-3-ol (Ye et al., J. Am. Chem. Soc. 132:8550-8551 (2010)) (250 mg, 1.62 mmol, 1 equiv) and dissolved into tetrahydrofuran (16 mL) at room temperature. Solid N-chlorosuccinimide (303 mg, 2.27 mmol, 1.40 equiv) and triphenylphosphine (638 mg, 2.43 mmol, 1.50 equiv) were then added to the reaction mixture. After 14 hours, the reaction was concentrated under reduced pressure. The crude product was purified by flash column chromatography on silica gel (eluent: 10% dichloromethane in hexanes) to provide chloroalkyne S10 (134 mg, 48%) as a clear, colorless oil. ¹H NMR (500 MHz, CDCl₃, 25° C.): δ 4.48 (td, J=6.7, 2.3 Hz, 1H), 2.57 (d, J=2.3 Hz, 1H), 1.97-1.89 (m, 2H), 1.54-1.47 (m, 2H), 1.35-1.23 (m, 8H), 0.87 (t, J=7.1 Hz, 3H). ¹³C NMR (126 MHz, CDCl₃, 25° C.): δ 82.3, 74.3, 48.1, 39.2, 31.9, 29.3, 29.0, 26.3, 22.8, 14.3. FTIR (thin film) cm⁻¹: 3302 (m), 2926 (s), 2855 (s), 1461 (m). HRMS (GC-MS) (m/z): calc'd for C₁₀H₁₇Cl [M]: 172.1013, found: 172.1014. TLC (10% dichloromethane in hexanes), Rf: 0.80 (KMnO₄).

### Example 21: Synthesis of (E)-3-chloro-N,N-diethyldec-1-en-1-amine oxide (17)

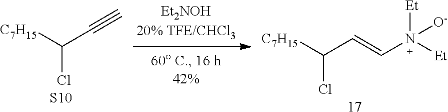

Enamine N-oxide 17 was synthesized following the general procedure A using 3-chlorodec-1-yne (S10). The reaction mixture was heated for 16 hours and purified by flash column chromatography on silica gel (eluent: 30% CMA in chloroform) to provide enamine N-oxide 17 (Trial 1: 27.2 mg, 36%; Trial 2: 36.2 mg, 48%) as a clear, colorless oil. ¹H NMR (500 MHz, CD₃OD, 25° C.): δ 6.49 (dd, J=12.9, 8.7 Hz, 1H), 6.36 (d, J=12.9 Hz, 1H), 4.63 (dt, J=8.9, 6.9 Hz, 1H), 3.44-3.31 (m, 4H), 1.92-1.83 (m, 2H), 1.52-1.40 (m, 2H), 1.37-1.29 (m, 8H), 1.26 (q, J=7.2 Hz, 6H), 0.90 (m, 3H). ¹³C NMR (126 MHz, CD₃OD, 25° C.): δ 140.3, 131.8, 65.5, 65.5, 59.2, 39.8, 33.0, 30.4, 30.2, 27.6, 23.8, 14.6, 8.7, 8.6. FTIR (thin film) cm⁻¹: 2929 (s), 2855 (s), 1681 (w), 1461 (w), 1375 (m), 969 (s). HRMS (ESI) (m/z): calc'd for C₁₄H₂₉ClNO [M+H]⁺: 262.1932, found: 262.1927. TLC (30% CMA in chloroform), Rf: 0.20 (KMnO₄).

### Example 22: Synthesis of (E)-3,3-diethoxy-N,N-diethylprop-1-en-1-amine oxide (18)

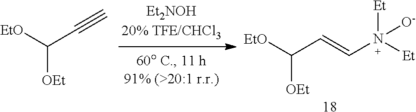

Enamine N-oxide 18 was synthesized following the general procedure A using 3,3-diethoxyprop-1-yne. The reaction mixture was stirred at 60° C. for 11 hours and purified by flash column chromatography on silica gel (eluent: 20% CMA in chloroform) to provide enamine N-oxide 18 (Trial 1: 76.5 mg, 90%; Trial 2: 77.2 mg, 91%) as a white solid. The regioisomeric ratio (>20:1 r.r.) was determined by taking the ratio of the ¹H-NMR integrations between the C₁ vinyl proton of the major isomer and the C₁ vinyl proton of the minor isomer. ¹H NMR (500 MHz, CD₃OD, 25° C.): δ 6.43-6.32 (m, 2H), 5.16 (d, J=3.7 Hz, 1H), 3.67 (dq, J=9.5, 7.0 Hz, 2H), 3.55 (dq, J=9.5, 7.0 Hz, 2H), 3.44-3.35 (m, 4H), 1.26 (t, J=7.2 Hz, 6H), 1.21 (t, J=7.0 Hz, 6H). ¹³C NMR (126 MHz, CD₃OD, 25° C.): δ 141.8, 128.6, 99.6, 65.2, 62.5, 15.7, 8.7. FTIR (thin film) cm⁻¹: 3362 (br), 2978 (m), 1684 (w), 1375 (w), 1126 (m), 1051 (s), 969 (m). HRMS (ESI) (m/z): calc'd for C₁₁H₂₄NO₃ [M+H]⁺: 218.1756, found: 218.1750. TLC (30% CMA in chloroform), Rf: 0.13 (KMnO₄).

### Example 23: Synthesis of N,N-diethyl-7-hydroxyhept-1-en-2-amine oxide (19)

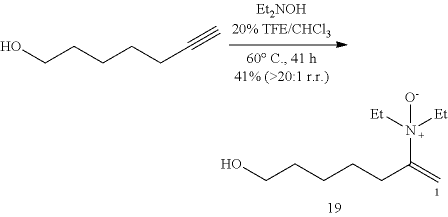

Enamine N-oxide 19 was synthesized following the general procedure A using hept-6-yn-1-ol. The reaction mixture stirred at 60° C. for 240 hours and purified by flash column chromatography on silica gel (eluent: 30% CMA in chloroform) to provide enamine N-oxide 19 (Trial 1: 37.8 mg, 42%; Trial 2: 35.7 mg, 40%) as a white solid. The regioisomeric ratio (>20:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): δ 5.93 (d, J=1.2 Hz, 1H), 5.30 (d, J=1.5 Hz, 1H), 3.56 (dt, J=12.7, 6.7 Hz, 4H), 3.37-3.30 (m, 2H), 2.24 (t, J=7.8 Hz, 2H), 1.69-1.56 (m, 4H), 1.53-1.44 (m, 2H), 1.19 (t, J=7.1 Hz, 6H). ¹³C NMR (126 MHz, CD₃OD, 25° C.): δ 155.3, 111.3, 63.4, 62.8, 33.5, 30.3, 29.0, 26.8, 8.7. FTIR (thin film) cm⁻¹: 3258 (br), 2937 (s), 2863 (s), 1670 (m), 1461 (m), 1375 (m), 958 (s). HRMS (ESI) (m/z): calc'd for C₁₁H₂₄NO₂ [M+H]⁺: 202.1807, found: 202.1802. TLC (30% CMA in chloroform), Rf: 0.060 (KMnO₄).

### Example 24: Synthesis of 3-(dibutylamino)-N,N-diethylprop-1-en-2-amine oxide (20) and (E)-3-(dibutylamino)-N,N-diethylprop-1-en-1-amine oxide (20′)

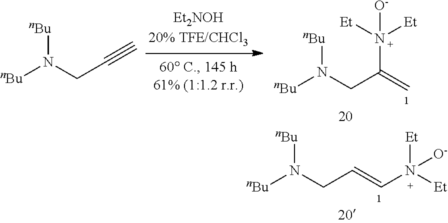

Enamine N-oxide 20 was synthesized following the general procedure A using N-butyl-N-(prop-2-yn-1-yl)butan-1-amine (Acquaah-Harrison et al., J. Comb. Chem. 12:491-496 (2010)). The reaction mixture was heated for 145 hours and purified by flash column chromatography on silica gel (eluent: 50% CMA in chloroform) to provide regioisomeric enamine N-oxides 20 and 20′ (Trial 1: 43.6 mg, 57%, Trial 2: 49.7 mg, 65%) as a clear, colorless oil. The regioisomeric ratio (20:20′, Trial 1: 1:1.2, Trial 2: 1:1.2) was determined by taking the ratio of the ¹H-NMR integrations between the C₁ vinyl proton of the major isomer and the C₁ vinyl proton of the minor isomer. Further purification by flash chromatography on silica gel (eluent: 30% CMA in chloroform) afforded the regioisomers separately for analytical characterization. ¹H NMR (500 MHz, CD₃OD, 25° C.): [20] δ 5.92 (s, 1H), 5.67 (s, 1H), 3.59 (dq, J=12.5, 7.0 Hz, 2H), 3.39 (dq, J=12.5, 7.1 Hz, 2H), 3.22 (s, 2H), 2.51-2.44 (m, 2H), 1.51-1.41 (m, 4H), 1.34 (dq, J=14.2, 7.3 Hz, 4H), 1.22 (t, J=7.0 Hz, 6H), 0.93 (t, J=7.3 Hz, 6H). [20′] δ 6.45 (dt, J=13.6, 6.9 Hz, 1H), 6.19 (dt, J=13.1, 1.4 Hz, 1H), 3.43-3.31 (m, 4H), 3.28 (dd, J=6.9, 1.4 Hz, 2H), 2.52-2.45 (m, 4H), 1.53-1.43 (m, 4H), 1.34 (dq, J=14.6, 7.3 Hz, 4H), 1.26 (t, J=7.1 Hz, 6H), 0.94 (t, J=7.4 Hz, 6H). ¹³C NMR (126 MHz, CD₃OD, 25° C.): [20] δ 153.2, 114.4, 63.8, 55.7, 55.0, 30.5, 21.8, 14.6, 8.8. [20′] δ 140.38, 126.83, 65.12, 54.73, 52.36, 30.07, 21.75, 14.37, 8.64. FTIR (thin film) cm⁻¹: [20] 2959 (s), 2870 (m), 2803 (w), 1669 (w), 1461 (m), 1375 (m), 954 (w). [20′] 2955 (s), 2862 (m), 2803 (w), 1461 (m), 1375 (m), 976 (w). HRMS (ESI) (m/z): [20] calc'd for C₁₅H₃₃N₂O [M+H]⁺: 257.2587, found: 257.2582. [20′] calc'd for C₁₅H₃₃N₂O [M+H]⁺: 257.2587, found: 257.2581. TLC (50% CMA in chloroform), Rf: 0.40 (20, KMnO₄), 0.35 (20′, KMnO₄).

### Example 25: Synthesis of 3-((tert-butoxycarbonyl)amino)-N,N-diethylprop-1-en-2-amine oxide (21) and (E)-3-((tert-butoxycarbonyl)amino)-N,N-diethylprop-1-en-1-amine oxide (21′)

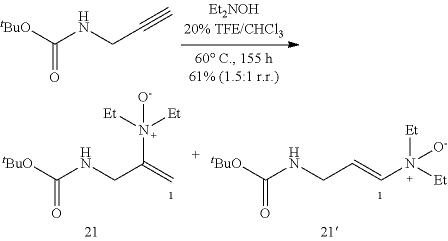

Enamine N-oxide 21 was synthesized following the general procedure A using tert-butyl prop-2-yn-1-ylcarbamate (Wipf et al., Org. Lett. 6:3593-3595 (2004)). The reaction mixture stirred at 60° C. for 155 hours and purified by flash column chromatography on silica gel (eluent: 30% CMA in chloroform) to provide regioisomeric enamine N-oxides 21 and 21′ (Trial 1: 48.8 mg, 62%; Trial 2: 47.6 mg, 61%) as a yellow oil. The regioisomeric ratio (21:21′, Trial 1: 1.5:1, Trial 2: 1.5:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): [21] δ 5.78 (s, 1H), 5.43 (s, 1H), 3.88 (s, 2H), 3.44-3.31 (m, 4H), 1.45 (s, 9H), 1.25 (t, J=7.1 Hz, 6H). [21′] δ 6.33 (dt, J=12.4, 6.0 Hz, 1H), 6.09 (d, J=13.3 Hz, 1H), 3.77 (d, J=6.0 Hz, 2H), 3.58 (dq, J=14.0, 7.0 Hz, 4H), 1.44 (s, 9H), 1.26 (t, J=7.2 Hz, 6H). ¹³C NMR (126 MHz, CD₃OD, 25° C.): [21] δ 158.1, 153.4, 111.9, 80.7, 65.3, 41.0, 28.9, 8.7. [21′] δ 158.3, 139.2, 128.0, 80.4, 64.6, 39.6, 28.9, 8.6. FTIR (thin film) cm⁻¹: 3343 (br), 2978 (m), 1696 (s), 1521 (m), 1167 (s). HRMS (ESI) (m/z): calc'd for C₁₂H₂₅N₂O₃ [M+H]⁺: 245.1865, found: 245.1859. TLC (30% CMA in chloroform), Rf: 0.054 (KMnO₄).

### Example 26: Synthesis of N,N-diethyl-3-(phenylthio)prop-1-en-2-amine oxide (22) and (E)-N,N-diethyl-3-(phenylthio)prop-1-en-1-amine oxide (22′)

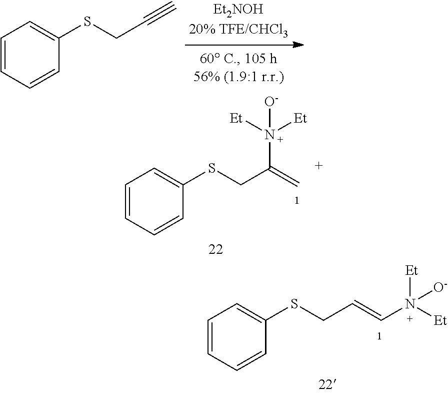

Enamine N-oxide 22 was synthesized following the general procedure A using phenyl(prop-2-yn-1-yl)sulfane. The reaction mixture stirred at 60° C. for 105 hours and purified by flash column chromatography on silica gel (eluent: 30% CMA in chloroform) to provide regioisomeric enamine N-oxides 22 and 22′ (Trial 1: 46.4 mg, 57%; Trial 2: 44.9 mg, 55%) as a yellow oil. The regioisomeric ratio (22:22′, Trial 1: 2.5:1, Trial 2: 1.3:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): [22] δ 7.53-7.13 (m, 5H), 5.84 (d, J=1.8 Hz, 1H), 5.60-5.53 (m, 1H), 3.91 (d, J=1.2 Hz, 2H), 3.58 (dq, J=12.5, 7.0 Hz, 2H), 3.39 (dq, J=12.5, 7.1 Hz, 2H), 1.17 (t, J=7.1 Hz, 6H). [22′] δ 7.57-7.13 (m, 5H), 6.39 (dt, J=13.0, 7.6 Hz, 1H), 6.07-5.97 (m, 1H), 3.68 (dd, J=7.6, 1.3 Hz, 2H), 3.25-3.13 (m, 4H), 1.05 (t, J=7.2 Hz, 6H). ¹³C NMR (126 MHz, CD₃OD, 25° C.): [22] δ 151.9, 136.4, 132.0, 130.4, 128.5, 114.8, 64.0, 34.4, 8.8. [22′] δ 140.1, 136.0, 132.0, 130.3, 128.1, 126.7, 65.3, 32.9, 8.5. FTIR (thin film) cm⁻¹: 3243 (br), 1655 (w), 1439 (m), 738 (s), 693 (s). HRMS (ESI) (m/z): calc'd for C₁₃H₂₀NOS [M+H]⁺: 238.1266, found: 238.1260. TLC (30% CMA in chloroform), Rf: 0.071 (UV, KMnO₄).

### Example 27: Synthesis of (E)-3-(((benzyloxy)carbonyl)oxy)-N,N-dimethylprop-1-en-1-amine oxide (23)

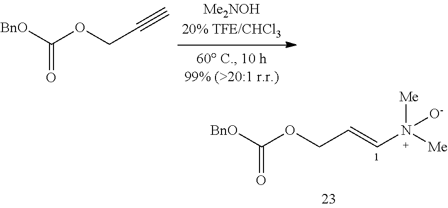

Enamine N-oxide 23 was synthesized following the general procedure A using N,N-dimethylhydroxylamine (Liu et al., Chinese Patent No. 2009/101503374 (2009)). The reaction mixture was heated for 10 hours, concentrated under reduced pressure, and purified by automated C₁₈ reverse phase column chromatography (30 g C₁₈ silica gel, 25 μm spherical particles, eluent: [symbol] H₂O+0.10% TFA (2 CV), gradient 0→100% MeCN/H₂O+0.1% TFA (15 CV), t_{R}=6.1 CV) to provide enamine N-oxide 23 (Trial 1: 82.2 mg, 100%; Trial 2: 80.5 mg, 98%) as a clear, colorless oil. The regioisomeric ratio (>20:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): δ 7.43-7.31 (m, 5H), 6.65 (dt, J=13.2, 1.6, 1H), 6.54 (dt, J=13.3, 5.5, 1H), 5.17 (s, 2H), 4.77 (dd, J=5.6, 1.5, 2H), 3.26 (s, 6H). ¹³C NMR (126 MHz, CD₃OD, 25° C.): δ 156.2, 144.7, 137.1, 129.8, 129.7, 129.5, 121.3, 71.0, 65.1, 60.5. FTIR (thin film) cm⁻¹: 1744 (s), 1453 (w), 1394 (w), 1267 (s), 957 (w). HRMS (ESI) (m/z): calc'd for C₁₃H₁₈NO₄ [M+H]⁺: 252.1230, found: 252.1227. TLC (30% CMA in chloroform), Rf: 0.30 (UV, KMnO₄).

### Example 28: Synthesis of N,N-dibutylhydroxylamine (S11)

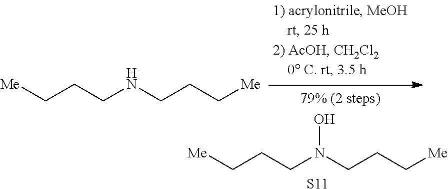 [SMILES: CCCCNCCCC ; CCCCN(O)CCCC ; "S11"]

Acrylonitrile (180 μL, 2.74 mmol, 1.00 equiv) was added via syringe to a solution of dibutylamine (354 mg, 2.74 mmol, 1 equiv) in methanol (135 mL) at room temperature. After 25 hours, the reaction mixture was concentrated and used without further purification. The crude product was dissolved in dichloromethane (27 mL) and solid sodium carbonate (581 mg, 5.48 mmol, 2.00 equiv) was added in one portion. After the resultant suspension was cooled to 0° C. in an ice-water bath, 39% peracetic acid/acetic acid (465 μL, 2.74 mmol, 1.00 equiv) was added via syringe, and the reaction mixture was allowed to warm to room temperature. After 3.5 hours, methanol (0.5 mL) was added and the reaction mixture was loaded directly onto a silica gel column. The reaction mixture was purified by flash column chromatography (eluent: 10% CMA in chloroform) to provide hydroxylamine S11 (316 mg, 79%) as a white solid. ¹H NMR (500 MHz, CD₃OD, 25° C.): δ 2.62 (t, J=7.4, 4H), 1.60-1.49 (m, 4H) 1.34 (sxt, J=7.8, 7.3, 4H), 0.90 (t, J=7.3, 6H). ¹³C NMR (126 MHz, CD₃OD, 25° C.): δ 60.8, 29.7, 20.8, 14.3. FTIR (thin film) cm⁻¹: 3165 (br), 2956 (s), 2870 (s), 1465 (s), 1372 (s), 1070 (s), 742 (s). HRMS (ESI) (m/z): calc'd for C₈H₂₀NO [M+H]⁺: 146.1539, found: 146.1537. TLC (10% CMA in chloroform), Rf: 0.33 (KMnO₄).

[symbol]

### Example 29: Synthesis of (E)-N-(3-(((benzyloxy)carbonyl)oxy)prop-1-en-1-yl)-N-butylbutan-1-amine oxide (24)

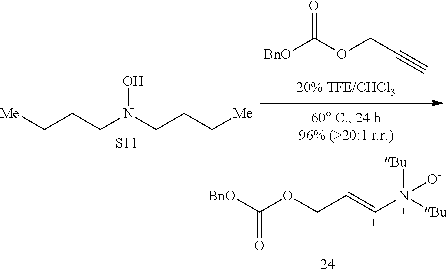

Enamine N-oxide 24 was synthesized following the general procedure B using N,N-dibutylhydroxylamine (S11). The reaction mixture was heated for 24 hours, concentrated under reduced pressure, and purified by automated C₁₈ reverse phase column chromatography (30 g C₁₈ silica gel, 25 μm spherical particles, eluent: H₂O+0.1% TFA (2 CV), gradient 0→100% MeCN/H₂O+0.1% TFA (15 CV), t_{R}=8.3 CV) to provide enamine N-oxide 24 (Trial 1:42.1 mg, 91%; 46.2 mg, 99%) as a clear, colorless oil. The regioisomeric ratio (>20:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): δ 7.42-7.31 (m, 5H), 6.51 (dt, J=13.2, 5.6, 1H), 6.36 (dt, J=13.0, 1.6, 1H), 5.17 (s, 2H), 4.79 (dd, J=5.7, 1.5, 2H), 3.35-3.21 (m, 4H), 1.86-1.54 (m, 4H), δ 1.40-1.29 (m, 4H), 0.95 (t, J=7.4, 6H). ¹³C NMR (126 MHz, CD₃OD, 25° C.): δ 156.3, 142.1, 137.1, 129.8, 129.7, 129.4, 124.3, 70.9, 70.7, 65.3, 26.0, 21.1, 14.3. FTIR (thin film) cm⁻¹: 2959 (m), 2873 (w), 1748 (s), 1457 (w), 1394 (w), 1267 (s), 950 (w). HRMS (ESI) (m/z): calc'd for C₁₉H₃₀NO₄ [M+H]⁺: 336.2169, found: 336.2167. TLC (30% CMA in chloroform), Rf: 30.40 (UV, KMnO₄).

### Example 30: Synthesis of (E)-N-(3-(((benzyloxy)carbonyl)oxy)prop-1-en-1-yl)-N-cyclohexylcyclohexanamine oxide (25)

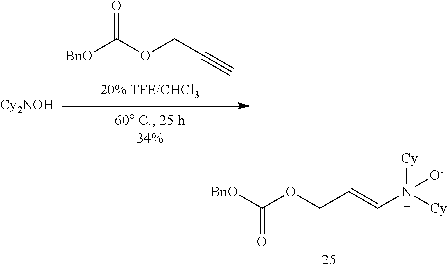

Enamine N-oxide 25 was synthesized following the general procedure B using N,N-dicyclohexylhydroxylamine (Murray et al., Synth. Commun. 19:3509-3522 (1989)). The reaction mixture was heated for 25 hours, concentrated under reduced pressure, and purified by automated [symbol] C₁₈ reverse phase column chromatography (30 g C₁₈ silica gel, 25 μm spherical particles, eluent: H₂O+0.1% TFA (2 CV), gradient 0→100% MeCN/H₂O+0.1% TFA (15 CV), t_{R}=7.0 CV) to provide enamine N-oxide 25 (Trial 1: 10.8 mg, 27%; Trial 2: 15.7 mg, 40%) as a clear, colorless oil. ¹H NMR (500 MHz, CD₃OD, 25° C.): δ 7.42-7.30 (m, 5H), 6.43 (dt, J=12.1, 5.9, 1H), 6.21 (d, J=13.2, 1H), 5.17 (s, 2H), 4.79 (d, J=5.8, 2H), 3.37 (tt, J=11.4, 2.9, 2H), 2.19 (d, J=11.9, 2H), 1.96-1.80 (m, 6H), 1.65 (d, J=13.0, 2H), 1.51-1.38 (m, 4H), 1.39-1.25 (m, 4H), 1.18-1.04 (m, 2H). ¹³C NMR (126 MHz, CD₃OD, 25° C.): δ 156.4, 139.4, 137.2, 129.8, 129.7, 129.4, 125.8, 74.5, 70.9, 65.5, 28.4, 27.0, 26.7, 26.6, 26.4. FTIR (thin film) cm⁻¹: 2929 (m), 2855 (m), 1744 (s), 1453 (m), 1394 (w), 1263 (s), 950 (m). HRMS (ESI) (m/z): calc'd for C₂₃H₃₄NO₄ [M+H]⁺: 388.2482, found: 388.2476. TLC (30% CMA in chloroform), Rf: 0.50 (UV, KMnO₄).

### Example 31: Synthesis of N-ethyl-N-isopropylhydroxylamine (S12)

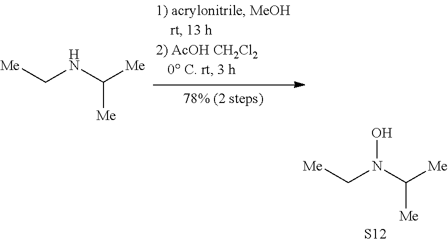 [SMILES: CCNC(C)C ; CCN(O)C(C)C ; "S12"]

Acrylonitrile (1.07 mL, 16.4 mmol, 1.00 equiv) was added via syringe to a solution of N-methylpropan-2-amine (1.00 g, 13.7 mmol, 1 equiv) in methanol (135 mL) at room temperature. After 13 hours, the reaction mixture was concentrated and used without further purification. The crude product was dissolved in dichloromethane (135 mL) and solid sodium carbonate (2.88 g, 27.3 mmol, 2.00 equiv) was added in one portion. After the resultant suspension was cooled to 0° C. in an ice-water bath, 39% peracetic acid/acetic acid (2.32 mL, 13.7 mmol, 1.00 equiv) was added via syringe, and the reaction mixture was allowed to warm to room temperature. After 3 hours, methanol (2.5 mL) was added and the reaction mixture was loaded directly onto a silica gel column. The reaction mixture was purified by flash column chromatography (eluent: 15% CMA in chloroform). The fractions containing the desired product were collected and concentrated under reduced pressure. The resulting liquid was then purified again on a short silica plug (1 in) to remove excess acetic acid (eluent: 50% CMA in chloroform) to provide hydroxylamine S12 (953 mg, 78%) as a yellow liquid. ¹H NMR (500 MHz, CD₃OD, 25° C.): δ 5.72 (bs, 1H), 2.91 (sx, J=6.5, 1H), 2.71 (q, J=7.1, 2H), 1.13 (t, J=7.1, 3H), 1.08 (d, J=6.5, 6H). ¹³C NMR (126 MHz, CD₃OD, [symbol] 25° C.): δ 56.7, 49.2, 18.3, 12.4. FTIR (thin film) cm⁻¹: 3191 (br), 2971 (s), 2851 (s), 1457 (s), 1379 (s), 1141 (s), 742 (s). HRMS (ESI) (m/z): calc'd for C₅H₁₄NO [M+H]⁺: 104.1070, found: 104.1068. TLC (20% CMA in chloroform), Rf: 0.20 (KMnO₄).

### Example 32: Synthesis of (E)-3-(((benzyloxy)carbonyl)oxy)-N-ethyl-N-isopropylprop-1-en-1-amine oxide (26)

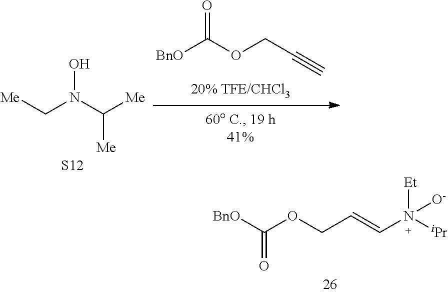

Enamine N-oxide 26 was synthesized following the general procedure B using N-ethyl-N-isopropylhydroxylamine (S12). The reaction mixture was heated for 19 hours, concentrated under reduced pressure, and purified by automated C₁₈ reverse phase column chromatography (30 g C₁₈ silica gel, 25 μm spherical particles, eluent: H₂O+0.1% TFA (2 CV), gradient 0→100% MeCN/H₂O+0.1% TFA (15 CV), t_{R}=7.5 CV) to provide enamine N-oxide 26 (Trial 1: 23.4 mg, 41%; Trial 2: 23.6 mg, 41%) as a clear, colorless oil. ¹H NMR (500 MHz, CD₃OD, 25° C.): δ 7.43-7.31 (m, 5H), 6.53 (dt, J=13.4, 5.2, 1H), 6.44 (dt, J=13.5, 1.5, 1H), 5.19 (s, 2H), 4.85 (d, 2H), 4.04 (sp, J=6.5, 1H), 3.81 (qd, J=7.1, 1.2, 2H), 1.40 (d, J=6.4, 3H), 1.37 (d, J=6.6, 3H), 1.32 (t, J=7.1, 3H). ¹³C NMR (126 MHz, CD₃OD, 25° C.): δ 156.2, 137.0, 133.5, 129.8, 129.8, 129.6, 128.8, 73.7, 71.2, 64.9, 62.5, 17.4, 16.2, 8.4. FTIR (thin film) cm⁻¹:1751 (m), 1684 (s), 1457 (w), 1397 (w), 1267 (s), 1200 (s), 1133 (s). HRMS (ESI) (m/z): calc'd for C₁₆H₂₄NO₄ [M+H]⁺: 294.1700, found: 294.1696. TLC (30% CMA in chloroform), Rf: 0.30 (UV, KMnO₄).

### Example 33: Synthesis of 4-(hydroxy(methyl)amino)butan-1-ol (S13)

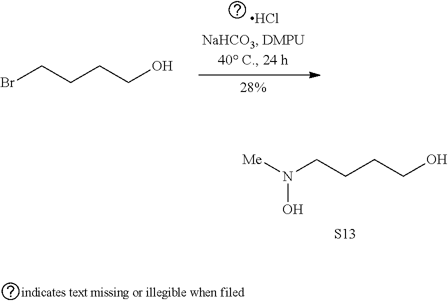 [SMILES: Cl ; OCCCCBr ; CN(O)CCCCO ; "S13"]

A round-bottom flask was charged sequentially with 4-bromobutan-1-ol (860 mg, 5.36 mmol, 1 equiv), N-methylhydroxylamine hydrochloride (540 mg, 6.45 mmol, 1.20 equiv), and [symbol] sodium carbonate (1.40 g, 16.1 mmol, 3.00 equiv). N,N′-dimethylpropyleneurea (0.5 mL) was added to the flask via syringe and the reaction mixture was stirred at 40° C. After 24 hours, the reaction mixture was purified directly by flash column chromatography on silica gel (eluent: 5% methanol in dichloromethane) to afford the product S13 (174 mg, 28%) as a white solid. ¹H NMR (500 MHz, CDCl₃, 25° C.): δ 3.65-3.57 (m, 2H), 2.81-2.50 (m, 5H), 1.80-1.61 (m, 4H). ¹³C NMR (126 MHz, CDCl₃, 25° C.): δ 63.0, 62.0, 48.6, 31.5, 25.3. FTIR (thin film) cm⁻¹: 3295 (br, m), 2944 (m), 2870 (m), 1476 (m), 1397 (m), 1036 (s), 805 (m). HRMS (ESI) (m/z): calc'd for C₅H₁₄NO₂ [M+H]⁺: 120.1019, found: 120.1017. TLC (5% methanol in dichloromethane), Rf: 0.25 (KMnO₄).

### Example 34: Synthesis of (E)-N-(3-(((benzyloxy)carbonyl)oxy)prop-1-en-1-yl)-4-hydroxy-N-methylbutan-1-amine oxide (27)

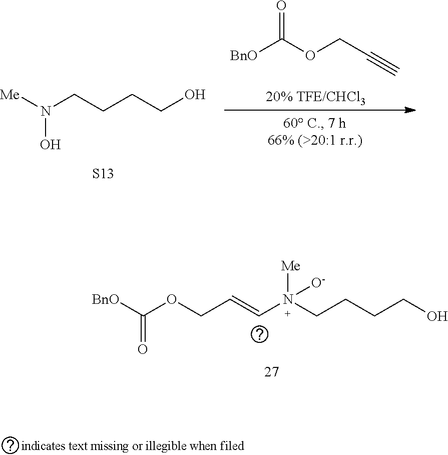

Enamine N-oxide 27 was synthesized following the general procedure B using 4-(hydroxy(methyl)amino)butan-1-ol (S13). The reaction mixture was heated for 7 hours, concentrated under reduced pressure, and purified by automated C₁₈ reverse phase column chromatography (30 g C₁₈ silica gel, 25 μm spherical particles, eluent: H₂O+0.1% TFA (2 CV), gradient 0→100% MeCN/H₂O+0.1% TFA (15 CV), t_{R}=6.5 CV) to provide enamine N-oxide 27 (Trial 1: 34.6 mg, 67%; Trial 2: 33.4 mg, 64%) as a clear, colorless oil. The regioisomeric ratio (>20:1) was determined by taking the ratio of the ¹H-NMR integrations between the C₁ vinyl proton of the major isomer and the C₁ vinyl proton of the minor isomer. ¹H NMR (500 MHz, CD₃OD, 20° C.): δ 7.44-7.28 (m, 5H), 6.59-6.47 (m, 2H), 5.17 (s, 2H), 4.78 (d, J=4.2, 2H), 3.57 (t, J=6.3, 2H), 3.43-3.31 (m, 2H), 3.21 (s, 3H), 1.92-1.65 (m, 2H), 1.54 (p, J=7.1, 6.9, 2H). ¹³C NMR (126 MHz, CD₃OD, 25° C.): δ 156.3, 143.4, 137.1, 129.8, 129.7, 129.5, 122.9, 71.9, 71.0, 65.2, 62.4, 58.8, 30.5, 21.2. FTIR (thin film) cm⁻¹: 2952 (w), 1748 (s), 1453 (w), 1267 (s), 943 (w). HRMS (ESI) (m/z): calc'd for C₁₆H₂₄NO₅ [M+H]⁺: 310.1649, found: 310.1644. TLC (30% CMA in chloroform), Rf: 0.10 (UV, KMnO₄).

[symbol]

### Example 35: Synthesis of 5-(hydroxy(methyl)amino)pentanenitrile (S14)

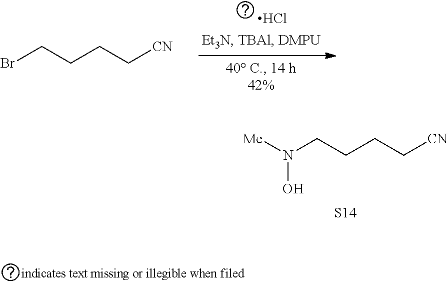 [SMILES: Cl ; N#CCCCCBr ; CN(O)CCCCC#N ; "S14"]

A round-bottom flask was charged sequentially with 5-bromovaleronitrile (720 μL, 6.17 mmol, 1 equiv) triethylamine (1.71 mL, 12.3 mmol, 2.00 equiv), N-methylhydroxylamine hydrochloride (1.03 g, 12.3 mmol, 2.00 equiv), and tetrabutylammonium iodide (342 mg, 926 μmol, 0.150 equiv). N,N-dimethylpropyleneurea (5 mL) was then added to the flask via syringe and the reaction mixture stirred at 40° C. After 14 h, diethyl ether (30 mL) and water (30 mL) were added to the reaction. The aqueous phase was then washed with diethyl ether (10×30 mL). The combined organic phases were dried over anhydrous sodium sulfate, filtered, and concentrated under reduced pressure. The resulting residue was purified by flash column chromatography on silica gel (eluent: 10% CMA in chloroform) to provide hydroxylamine S14 (336 mg, 42%) as a white solid. ¹H NMR (500 MHz, CDCl₃, 25° C.): δ 2.67-2.59 (m, 5H), 2.38 (t, J=6.6 Hz, 2H), 1.75-1.70 (m, 4H). ¹³C NMR (126 MHz, CDCl₃, 25° C.): δ 119.8, 61.1, 49.0, 26.4, 23.4, 17.4. FTIR (thin film) cm⁻¹: 3201 (br, m), 2952 (s), 2870 (s), 2247 (m), 1438 (s), 1382 (m), 1028 (m), 805 (m). HRMS (ESI) (m/z): calc'd for C₆H₁₃N₂O [M+H]⁺: 129.1022, found: 129.1020. TLC (30% CMA in chloroform), Rf: 0.30 (KMnO₄).

### Example 36: Synthesis of (E)-N-(3-(((benzyloxy)carbonyl)oxy)prop-1-en-1-yl)-4-cyano-N-methylbutan-1-amine oxide (28)

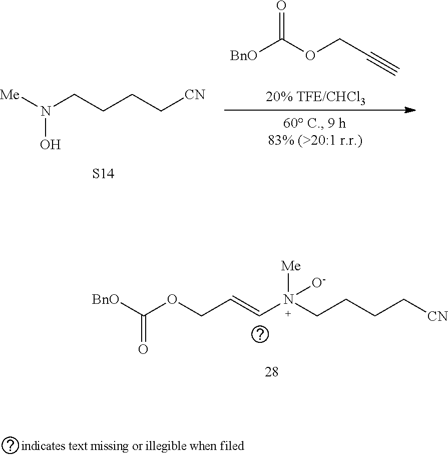

Enamine N-oxide 28 was synthesized following the general procedure B using 5-(hydroxy(methyl)amino)pentanenitrile (S14). The reaction mixture was heated for 9 hours, concentrated under reduced pressure, and purified by automated C₁₈ reverse phase column chromatography (30 g C₁₈ silica gel, 25 μm spherical particles, eluent: H₂O+0.1% TFA (2 CV), gradient 0→100% MeCN/H₂O+0.1% TFA (10 CV), t_{R}=5.6 CV) to provide enamine N-oxide 28 [symbol] (Trial 1: 39.2 mg, 79%; Trial 2: 43.2 mg, 87%) as a clear, colorless oil. The regioisomeric ratio (>20:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): δ 7.44-7.32 (m, 5H), 6.70-6.60 (m, 2H), 5.19 (s, 2H), 4.85 (d, J=3.4, 2H), 3.87-3.72 (m, 2H), 3.61 (s, 3H), 2.52 (t, J=7.1, 2H), 2.03-1.81 (m, 2H), 1.77-1.66 (m, 2H). ¹³C NMR (126 MHz, CD₃OD, 25° C.): δ 156.1, 137.8, 136.9, 129.8, 129.8, 129.6, 126.9, 120.6, 71.2, 70.2, 64.6, 57.4, 23.3, 23.2, 17.1. FTIR (thin film) cm⁻¹: 2952 (s), 2855 (m), 1748 (s), 1572 (m), 1457 (m), 1394 (w), 1267 (s), 946 (w). HRMS (ESI) (m/z): calc'd for C₁₇H₂₃N₂O₄ [M+H]⁺: 319.1652, found: 319.1650. TLC (30% CMA in chloroform), Rf: 0.30 (UV, KMnO₄).

### Example 37: Synthesis of N-(4-azidobutyl)-N-methylhydroxylamine (S15)

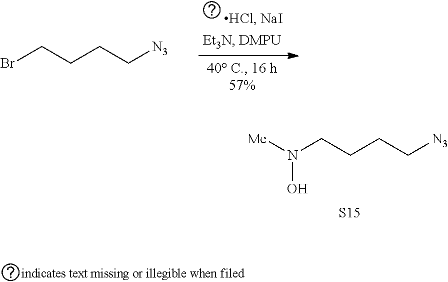

A round-bottom flask was charged with 1-azido-4-bromobutane (Satyanarayana et al., Chem. Commun. 48:1461-1463 (2012)) (220 mg, 1.23 mmol, 1 equiv), N-methylhydroxylamine hydrochloride (113 mg, 1.35 mmol, 1.10 equiv) and sodium iodide (27.8 mg, 0.185 mmol, 0.150 equiv). N,N′-dimethylpropyleneurea (500 μL) and triethylamine (190 μL, 1.35 mmol, 1.10 equiv) were then sequentially added via syringe and the reaction mixture stirred at 40° C. After 16 hours, the residue was purified directly by flash column chromatography on silica gel (eluent: 5% CMA in chloroform) to provide hydroxylamine S15 (100 mg, 57%) as a clear, colorless oil. ¹H NMR (500 MHz, CDCl₃, 25° C.): δ 3.25 (t, J=6.3, 2H), 2.67-2.57 (m, 5H), 1.67-1.61 (m, 4H). ¹³C NMR (126 MHz, CDCl₃, 25° C.): δ 61.5, 51.5, 48.8, 26.8, 24.6. FTIR (thin film) cm⁻¹: 3220 (br, w), 2929 (m), 2870 (m), 2094 (s), 1457 (w), 1256 (m). HRMS (ESI) (m/z): calc'd for C₅H₁₃N₄O [M+H]⁺: 145.1084, found: 145.1083. TLC (30% CMA in chloroform), Rf: 0.55 (KMnO₄).

[symbol]

### Example 38: Synthesis of (E)-4-azido-N-(3-(((benzyloxy)carbonyl)oxy)prop-1-en-1-yl)-N-methylbutan-1-amine oxide (29)

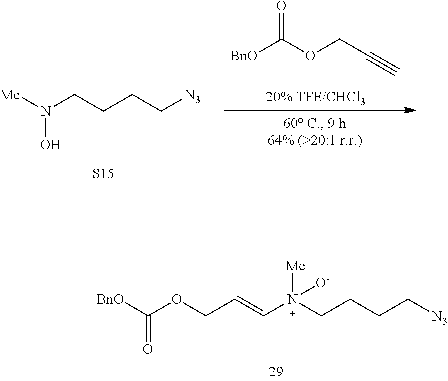

Enamine N-oxide 29 was synthesized following the general procedure B using N-(4-azidobutyl)-N-methylhydroxylamine (S15). The reaction mixture was heated for 9 hours, concentrated under reduced pressure, and purified by automated C₁₈ reverse phase column chromatography (30 g C₁₈ silica gel, 25 μm spherical particles, eluent: H₂O+0.1% TFA (2 CV), gradient 0→100% MeCN/H₂O+0.1% TFA (10 CV), t_{R}=6.0 CV) to provide enamine N-oxide 29 (Trial 1: 32.0 mg, 69%; Trial 2: 26.8, 58%) as a clear, colorless oil. The regioisomeric ratio (>20:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): δ 7.42-7.32 (m, 5H), 6.71-6.58 (m, 2H), 5.19 (s, 2H), 4.85 (d, J=3.5 Hz, 2H), 3.85-3.71 (m, 2H), 3.60 (s, 3H), 3.37 (t, J=6.6 Hz, 2H), 1.96-1.75 (m, 2H), 1.70-1.58 (m, 2H). ¹³C NMR (126 MHz, CD₃OD, 25° C.): δ 156.1, 137.9, 136.9, 129.8, 129.8, 129.5, 126.8, 71.2, 70.6, 64.6, 57.2, 51.9, 26.6, 21.5. FTIR (thin film) cm⁻¹: 2952 (w), 2098 (s), 1748 (s), 1453 (w), 1263 (s), 943 (w). HRMS (ESI) (m/z): calc'd for C₁₆H₂₃N₄O₄ [M+H]⁺: 335.1714, found: 335.1710. TLC (30% CMA in chloroform), Rf: 0.30 (UV, KMnO₄).

### Example 39: Synthesis of N-benzyl-5-bromopentanamide (S16)

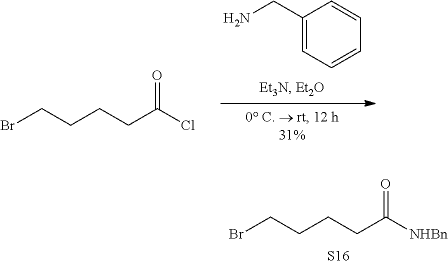

Benzylamine hydrochloride (1.80 g, 12.5 mmol, 1.00 equiv) was added as a solid to a solution of triethylamine (3.66 mL, 26.3 mmol, 2.10 equiv) in diethyl ether (125 mL) at room temperature. The solution was cooled to 0° C. in an ice-water bath. After 15 minutes, 5-bromovaleryl chloride (2.50 g, 12.5 mmol, 1 equiv) was subsequently added dropwise via syringe. [symbol] The ice-water bath was removed and the reaction mixture was allowed to warm to room temperature. After 3 hours, the reaction mixture was filtered over Celite® and concentrated under reduced pressure. The crude residue was purified by flash column chromatography on silica gel (eluent: 50% ethyl acetate in hexanes) to provide pentanamide S16 (1.05 g, 31%) as an off-white solid. ¹H NMR (500 MHz, CDCl₃, 25° C.): δ 7.38-7.20 (m, 5H), 5.88 (s, 1H), 4.41 (d, J=5.7, 2H), 3.39 (t, J=6.6, 2H), 2.22 (t, J=7.3, 2H), 1.94-1.74 (m, 4H). ¹³C NMR (126 MHz, CDCl₃, 25° C.): δ 172.3, 138.4, 128.9, 128.0, 127.7, 43.8, 35.7, 33.4, 32.3, 24.4. FTIR (thin film) cm⁻¹: 3265 (m), 3063 (w), 2929 (m), 1640 (s), 1543 (s), 1453 (s), 1230 (m), 1029 (m), 731 (m), 697 (s). HRMS (ESI) (m/z): calc'd for C₁₂H₁₇BrNO [M+H]⁺: 270.0488, found: 270.0485. TLC (30% ethyl acetate in hexanes), Rf: 0.25 (UV, KMnO₄).

### Example 40: Synthesis of N-benzyl-5-(hydroxy(methyl)amino)pentanamide (S17)

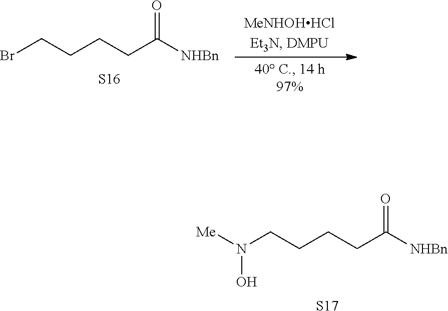

A round-bottom flask was charged with N-benzyl-5-bromopentanamide (S17, 1.05 g, 3.88 mmol, 1 equiv) and N-methylhydroxylamine hydrochloride (648 mg, 7.76 mmol, 2.00 equiv). N,N-dimethylpropyleneurea (5 mL) and triethylamine (1.08 ml, 7.76 mmol, 2.00 equiv) were then sequentially added via syringe and the reaction mixture stirred at 40° C. After 14 hours, the reaction mixture was purified directly by flash column chromatography on silica gel (eluent: 10% methanol in dichloromethane) to provide hydroxylamine S17 (889 mg, 97%) as a white solid. ¹H NMR (500 MHz, CDCl₃, 25° C.): δ 7.38-7.21 (m, 5H), 6.78 (t, J=5.9, 1H), 4.40 (d, J=5.6 Hz, 2H), 3.25 (t, J=7.3, 1H), 2.30 (td, J=7.2, 7.2, 2.3, 2H), 2.60 (s, 3H), 2.22 (t, J=7.1, 2H), 1.77 (dq, J=40.8, 7.4, 7.0, 4H). ¹³C NMR (126 MHz, CDCl₃, 25° C.): δ 173.1, 138.6, 128.9, 128.0, 127.6, 61.9, 48.8, 43.7, 36.5, 26.9, 23.5. FTIR (thin film) cm⁻¹: 3295 (m), 2926 (m), 1632 (s), 1539 (s), 1453 (m), 745 (m), 697 (s). HRMS (ESI) (m/z): calc'd for C₁₃H₂₁N₂O₂ [M+H]⁺: 237.1598, found: 237.1594. TLC (10% methanol in dichloromethane), Rf: 0.45 (UV, KMnO₄).

[symbol]

### Example 41: Synthesis of (E)-5-(benzylamino)-N-(3-(((benzyloxy)carbonyl)oxy)prop-1-en-1-yl)-N-methyl-5-oxopentan-1-amine oxide (30)

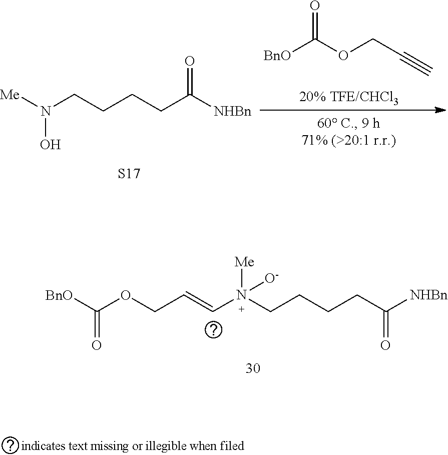

Enamine N-oxide 30 was synthesized following the general procedure B using N-benzyl-5-(hydroxy(methyl)amino)pentanamide (S17). The reaction mixture was heated for 9 hours, concentrated under reduced pressure, and purified by automated C₁₈ reverse phase column chromatography (30 g C₁₈ silica gel, 25 μm spherical particles, eluent: H₂O+0.1% TFA (2 CV), gradient 0→100% MeCN/H₂O+0.1% TFA (10 CV), t_{R}=6.4 CV) to provide enamine N-oxide 30 (Trial 1: 27.3 mg, 76%; Trial 2: 23.9 mg, 66%) as a clear, colorless oil. The regioisomeric ratio (>20:1) was determined by taking the ratio of the ¹H-NMR integrations between the C₁ vinyl proton of the major isomer and the C₁ vinyl proton of the minor isomer. ¹H NMR (500 MHz, CD₃OD, 25° C.): δ 7.48-7.18 (m, 10H), 6.64 (dt, J=13.4, 1.3, 1H), 6.59 (dt, J=13.5, 4.5, 1H), 5.19 (s, 2H), 4.83 (dd, J=4.5, 1.3, 2H), 4.36 (s, 2H), 3.84-3.68 (m, 2H), 3.56 (s, 3H), 2.30 (t, J=7.1, 2H), 1.87-1.72 (m, 2H), 1.73-1.64 (m, 2H). ¹³C NMR (126 MHz, CD₃OD, 25° C.): δ 175.0, 156.1, 140.2, 137.9, 136.9, 129.9, 129.8, 129.7, 129.6, 128.8, 128.4, 126.8, 71.3, 70.9, 64.6, 57.2, 44.3, 36.1, 23.6, 23.4. FTIR (thin film) cm⁻¹: 3067 (w), 1751 (s), 1654 (s), 1546 (m), 1263 (s), 1200 (s), 1133 (s), 719 (m). HRMS (ESI) (m/z): calc'd for C₂₄H₃₁N₂O₅ [M+H]⁺: 427.2227, found: 427.2221. TLC (40% CMA in chloroform), Rf: 0.25 (UV, KMnO₄).

### Example 42: Synthesis of (E)-4-(3-(((benzyloxy)carbonyl)oxy)prop-1-en-1-yl)morpholine 4-oxide (31)

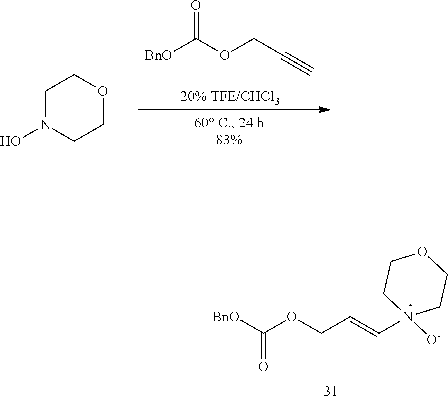

[symbol]

Enamine N-oxide 31 was synthesized following the general procedure B using morpholin-4-ol (O'Neil et al., Tetrahedron Lett. 42:8247-8249 (2001)). The reaction mixture was heated for 24 hours, concentrated under reduced pressure, and purified by automated C₁₈ reverse phase column chromatography (30 g C₁₈ silica gel, 25 μm spherical particles, eluent: H₂O+0.1% TFA (2 CV), gradient 0→100% MeCN/H₂O+0.1% TFA (15 CV), t_{R}=6.1 CV) to provide enamine N-oxide 31 (Trial 1: 48.6 mg, 85%; Trial 2: 45.4 mg, 80%) as a clear, colorless oil. ¹H NMR (500 MHz, CD₃OD, 25° C.): δ 7.41-7.30 (m, 5H), 6.62 (t, J=1.3 Hz, 2H), 5.17 (s, 2H), 4.80 (d, J=2.9, 2H), 4.26 (ddd, J=7.1, 2H), 3.81 (d, J=12.4, 2H), 3.64 (td, J=11.7, 3.7, 2H), 2.98 (d, J=11.5, 2H). ¹³C NMR (126 MHz, CD₃OD, 25° C.): δ 156.2, 144.7, 137.1, 129.8, 129.7, 129.5, 122.6, 71.0, 66.4, 65.2, 62.5. FTIR (thin film) cm⁻¹: 2944 (w), 1744 (s), 1457 (w), 1394 (w), 1263 (s), 1118 (m), 950 (m). HRMS (ESI) (m/z): calc'd for C₁₅H₂₀NO₅ [M+H]⁺: 294.1336, found: 294.1331. TLC (30% CMA in chloroform), Rf: 0.20 (UV, KMnO₄).

### Example 43: Synthesis of 1-(naphthalen-1-yl)but-3-yn-2-ol (S18)

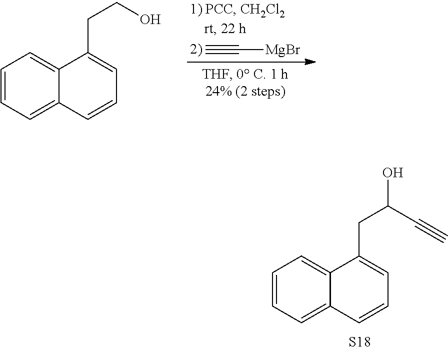

A round-bottom flask was charged with 2-(1-naphthyl)ethanol (300 mg, 1.74 mmol, 1 equiv) and dissolved in dichloromethane (17 mL) at room temperature. Pyridinium chlorochromate (413 mg, 1.92 mmol, 1.10 equiv) was added in one portion and the flask stirred at room temperature. After 22 hours, silica gel (1.00 g) was added and the reaction mixture stirred for 10 minutes. The slurry was then loaded directly onto a short silica gel plug and the crude product was eluted with diethyl ether (100 mL). The elution was concentrated under reduced pressure. The crude residue was then dissolved in tetrahydrofuran (17 mL) and cooled to 0° C. using an ice-water bath. A solution of ethynyl magnesium bromide in tetrahydrofuran (500 mM, 3.48 mL, 1.74 mmol, 1.00 equiv) was then added dropwise via syringe. After 1 hour, saturated aqueous ammonium chloride solution (10 mL) and ethyl acetate (50 mL) were added sequentially. The organic layer was washed with water (2×50 mL) and brine (50 mL), dried over anhydrous sodium sulfate, filtered, and concentrated under reduced pressure. The resulting residue was purified by [symbol] flash column chromatography on silica gel (eluent: 15% ethyl acetate/hexanes) to yield the product S18 (82.0 mg, 24%) as a clear, colorless oil. ¹H NMR (500 MHz, CDCl₃, 25° C.): δ 8.07 (dd, J=8.4, 1.2, 1H), 7.86 (dd, J=8.0, 1.5, 1H), 7.78 (dd, J=7.0, 2.4, 1H), 7.56-7.39 (m, 4H), 4.75 (ddd, J=7.8, 6.0, 2.1, 1H), 3.58-3.41 (m, 2H), 2.48 (d, J=2.1, 1H), 2.00 (s, 1H). ¹³C NMR (126 MHz, CDCl₃, 25° C.): δ 134.1, 132.6, 132.3, 129.1, 128.4, 128.1, 126.3, 125.9, 125.6, 123.8, 84.5, 74.1, 62.7, 41.2. FTIR (thin film) cm⁻¹: 3369 (br-w), 3287 (m), 3045 (w), 1025 (s), 775 (s). HRMS (ESI) (m/z): calc'd for C₁₄H₁₂NaO [M+Na]⁺: 219.0780, found: 219.0778. TLC (15% ethyl acetate in hexanes), Rf: 0.30 (UV, KMnO₄).

### Example 44: Synthesis of 1-(naphthalen-1-yl)but-3-yn-2-one (S19)

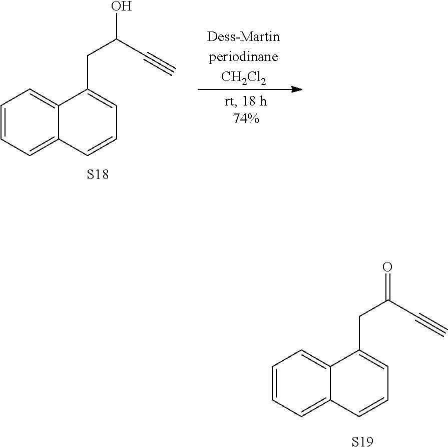

A round-bottom flask was charged with 1-(naphthalen-1-yl)but-3-yn-2-ol (S18, 15.0 mg, 76.4 μmol, 1 equiv) and dissolved into dichloromethane (1 mL) at room temperature. Dess-Martin periodinane (32.4 mg, 76.4 μmol, 1.00 equiv) was added in one portion to the flask. After 15 hours, a 1:1 aqueous solution of saturated sodium thiosulfate and sodium bicarbonate (1 mL) was added. The mixture was diluted with ethyl acetate (20 mL) and the organic layer was washed with water (20 mL), dried over anhydrous sodium sulfate, filtered, and concentrated under reduced pressure. The resulting residue was purified by flash column chromatography on silica gel chromatography (eluent: 5% ethyl acetate/hexanes) to yield the product S19 (11.0 mg, 74%) as a clear, colorless oil. ¹H NMR (500 MHz, CDCl₃, 25° C.): δ 7.91-7.79 (m, 3H), 7.56-7.37 (m, 4H), 4.28 (s, 2H), 3.14 (s, 1H). ¹³C NMR (126 MHz, CDCl₃, 25° C.): δ 184.6, 134.1, 132.4, 129.1, 129.0, 128.8, 128.8, 126.8, 126.2, 125.7, 123.9, 81.3, 80.4, 50.0. FTIR (thin film) cm⁻¹: 3272 (m), 3049 (w), 2094 (s), 1677 (s), 1088 (s), 786 (s). HRMS (ESI) (m/z): calc'd for C₁₄H₁₀NaO [M+Na]⁺: 217.0624, found: 217.0621. TLC (20% ethyl acetate in hexanes), Rf: 0.70 (UV).

[symbol]

### Example 45: Synthesis of 1-(2,2-difluorobut-3-yn-1-yl)naphthalene (S20)

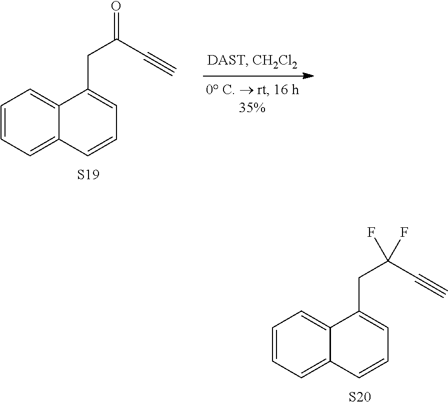 [SMILES: C#CC(=O)Cc1cccc2ccccc12 ; "S19" ; C#CC(F)(F)Cc1cccc2ccccc12 ; "S20"]

A round-bottom flask was charged with 1-(naphthalen-1-yl)but-3-yn-2-one (S19, 522 mg, 2.69 mmol, 1 equiv) and cooled to 0° C. with an ice-water bath. While stirring, neat diethylaminosulfur trifluoride (728 μL, 5.51 mmol, 2.05 equiv) was added dropwise via syringe. The ice-water bath was removed and the reaction mixture was stirred at room temperature. After 16 hours, the reaction was directly loaded onto silica gel and purified by flash column chromatography (eluent: 100% hexanes) to yield the product S20 (206 mg, 35%) as a white solid. ¹H NMR (500 MHz, CDCl₃, 25° C.): δ 8.13 (d, J=8.5, 1H), 7.95-7.85 (m, 2H), 7.64-7.45 (m, 4H), 3.88 (t, J=14.7, 2H), 2.70 (t, J=5.0, 1H). ¹³C NMR (126 MHz, CDCl₃, 25° C.): δ 134.1, 132.9, 130.0, 128.9, 128.9, 128.0 (t, J=3.5), 126.4, 125.9, 125.4, 124.4, 113.5 (t, J=235.5), 76.7 (t, J=6.7), 76.5 (t, J=40.5), 41.8 (t, J=26.8). ¹⁹F NMR (471 MHz, CDCl₃, 25° C.): δ 81.8. FTIR (thin film) cm⁻¹: 3295 (w), 2135 (w), 1271 (w), 1159 (m), 1054 (m), 1025 (m), 779 (s). HRMS (ESI) (m/z): calc'd for C₁₄H₁₁F2 [M+H]⁺: 217.0823, found: 217.0822. TLC (100% hexanes), Rf: 0.15 (UV, KMnO₄).

### Example 46: Synthesis of 5-bromo-N-(naphthalen-2-ylmethyl)pentanamide (S21)

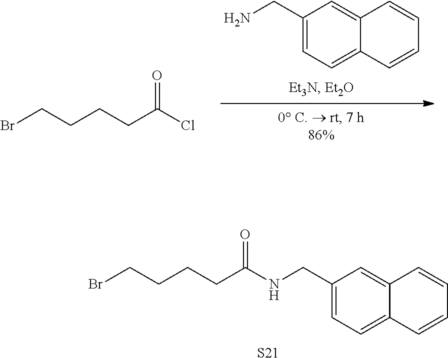 [SMILES: NCc1ccc2ccccc2c1 ; O=C(Cl)CCCCBr ; O=C(CCCCBr)NCc1ccc2ccccc2c1 ; "S21"]

5-Bromovaleryl chloride (1.01 mL, 7.52 mmol, 1 equiv) was added to a round-bottom flask via syringe containing diethyl ether (75 mL) cooled to 0° C. using an ice-water bath. Triethylamine (2.20 mL, 15.8 mmol, 2.10 equiv) and 1-naphthalenemethylamine (1.10 mL, 7.52 mmol, 1.00 equiv) were then sequentially added dropwise via syringe to the reaction mixture. The [symbol] ice-water bath was removed and stirring continued at room temperature. After 7 hours, the reaction mixture was concentrated under reduced pressure. The resulting residue was purified by flash column chromatography on silica gel (eluent: 50% ethyl acetate in hexanes) to provide pentanamide S21 (2.07 g, 86%) as a white solid. ¹H NMR (500 MHz, CDCl₃, 25° C.): δ 7.98 (d, J=8.2, 1H), 7.86 (dd, J=7.8, 1.6, 1H), 7.80 (dd, J=5.5, 4.0, 1H), 7.52 (dddd, J=17.5, 8.1, 6.8, 1.4, 2H), 7.57-7.46 (m, 2H), 5.68 (s, 1H), 4.87 (d, J=5.3, 2H), 3.36 (t, J=6.6, 2H), 2.19 (t, J=7.2, 2H), 1.94-1.73 (m, 4H). ¹³C NMR (126 MHz, CDCl₃, 25° C.): δ 172.0, 134.1, 133.7, 131.6, 129.0, 128.9, 127.1, 126.9, 126.3, 125.6, 123.7, 42.1, 35.7, 33.3, 32.3, 24.4. FTIR (thin film) cm⁻¹: 3280 (m), 3056 (w), 2933 (w), 1640 (s), 1543 (s), 1267 (m), 775 (s). HRMS (ESI) (m/z): calc'd for C₁₆H₁₉BrNO [M+H]⁺: 320.0645, found: 320.0641. TLC (40% ethyl acetate in hexanes), Rf: 0.30 (UV, KMnO₄).

### Example 47: Synthesis of 5-(hydroxy(methyl)amino)-N-(naphthalen-2-ylmethyl)pentanamide (S22)

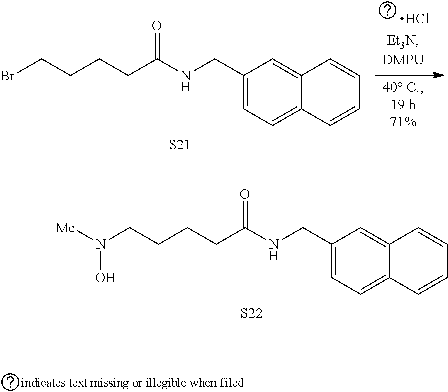 [SMILES: Cl ; O=C(CCCCBr)NCc1ccc2ccccc2c1 ; "S21" ; CN(O)CCCCC(=O)NCc1ccc2ccccc2c1 ; "S22"]

A round-bottom flask was charged with 5-bromo-N-(naphthalen-2-ylmethyl)pentanamide (S15, 1.00 g, 3.12 mmol, 1 equiv) and N-methylhydroxylamine hydrochloride (522 mg, 6.25 mmol, 2.00 equiv). N,N-dimethylpropyleneurea (3 mL) and triethylamine (871 μL, 6.25 mmol, 2.00 equiv) were then sequentially added via syringe and the reaction mixture stirred at 40° C. After 19 hours, the residue was purified directly by flash column chromatography on silica gel (eluent: 5% methanol in dichloromethane) to provide hydroxylamine S22 (638 mg, 71%) as a white solid. ¹H NMR (500 MHz, CD₃OD, 25° C.): δ 8.04 (d, J=8.4, 1H), 7.91-7.85 (m, 1H), 7.81 (d, J=7.8, 1H), 7.59-7.39 (m, 4H), 4.82 (s, 2H), 2.61-2.55 (m, 2H), 2.53 (s, 3H), 2.25 (t, J=7.4, 2H), δ 1.73-1.63 (m, 2H), 1.62-1.52 (m, 2H). ¹³C NMR (126 MHz, CD₃OD, 25° C.): δ 175.8, 135.5, 135.2, 132.9, 129.9, 129.4, 127.5, 127.4, 127.0, 126.5, 124.7, 63.0, 49.1, 42.4, 37.0, 27.9, 24.9. FTIR (thin film) cm⁻¹: 3283 (br, m) 3062 (w), 2952 (m), 1640 (s), 1543 (m), 779 (m). HRMS (ESI) (m/z): calc'd for C₁₇H₂₃N₂O₂ [M+H]⁺: 287.1754, found: 287.1751. TLC (5% methanol in dichloromethane), Rf: 0.30 (UV, KMnO₄).

### Example 48: Synthesis of (E)-N-(3,3-difluoro-4-(naphthalen-1-yl)but-1-en-1-yl)-N-methyl-5-((naphthalen-2-ylmethyl)amino)-5-oxopentan-1-amine oxide (S1)

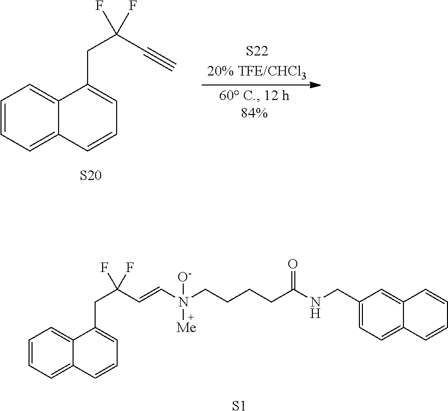

A glass 1-dram vial was charged with 1-(2,2-difluorobut-3-yn-1-yl)naphthalene (S20, 50.0 mg, 231 μmol, 1 equiv) and dissolved with 20% v/v trifluoroethanol in chloroform (116 μL) at room temperature. 5-(hydroxy(methyl)amino)-N-(naphthalen-2-ylmethyl)pentanamide (S22, 99.0 mg, 347 μmol, 1.50 equiv) was then added in one portion. The vial was flushed with nitrogen, sealed with a cap and Parafilm, and heated to 60° C. After 12 hours, the reaction mixture was removed from the oil bath and cooled to room temperature. The reaction mixture was directly purified by flash column chromatography on silica gel (eluent: 50% CMA in chloroform). Fractions containing the desired compound were combined and the solvent was removed under reduced pressure at 0° C. with a rotary evaporator to provide enamine N-oxide S1 (97.9 mg, 84%) as an off-white solid. ¹H NMR (500 MHz, CD₃OD, 25° C.): δ 8.10 (d, J=8.5, 1H), 8.04 (dd, J=8.4, 1.2, 1H), 7.91-7.77 (m, 4H), 7.57-7.38 (m, 8H), 6.63 (q, J=12.2, 1H), 6.41 (dt, J=13.1, 2.0, 1H), 4.85-4.77 (m, 2H), 3.84 (t, J=15.3, 2H), 3.05-2.97 (m, 2H), 2.88 (s, 3H), 2.15 (t, J=7.3, 2H), 1.53-1.36 (m, 3H), 1.28-1.16 (m, 1H). ¹³C NMR (126 MHz, CD₃OD, 25° C.): δ 175.0, 145.5 (t, J=9.4 Hz), 135.5, 135.5, 135.3, 134.1, 132.9, 131.2, 130.1 (t, J=4.6 Hz), 129.9, 129.9, 129.7, 129.5, 127.6, 127.5, 127.5, 127.0, 127.0, 127.0, 126.6, 126.5, 125.6, 124.7, 123.9 (t, J=26.8 Hz), 122.1 (t, J=241.9 Hz), 71.4, 58.5, 42.4, 40.9 (t, J=27.1 Hz), 36.4, 23.8, 23.6. ¹⁹F NMR (470.5 MHz, CDCl₃, 25° C.): δ −94.3 (q, J=244.2). FTIR (thin film) cm⁻¹: 3049 (m), 2937 (m), 1647 (m), 1550 (m), 1028 (m), 779 (s). HRMS (ESI) (m/z): calc'd for C₃₁H₃₃F2N₂O₂ [M+H]⁺: 503.2505, found: 503.2503. TLC (50% CMA in chloroform), Rf: 0.30 (UV, KMnO₄).

### Example 49: Synthesis of (1E,2Z)-3-fluoro-N-methyl-4-(naphthalen-1-yl)but-2-en-1-imine oxide (S2)

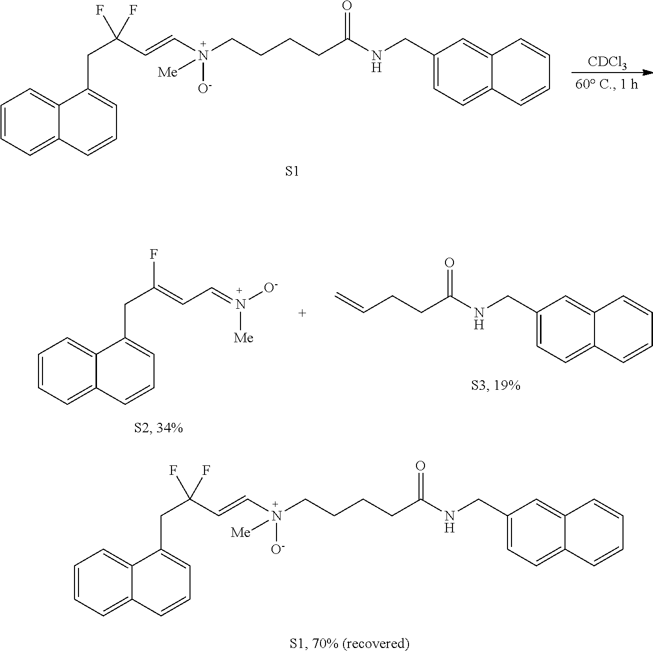

A 1-dram vial was charged with enamine N-oxide S1 (90.0 mg, 179 μmol, 1 equiv) and dissolved in chloroform-d (1.79 mL). The reaction vial was flushed with nitrogen, sealed with a cap and Parafilm, and heated to 60° C. After 1 hour, the oil bath was removed and the reaction was cooled to room temperature. The reaction mixture was purified by flash column chromatography on silica gel (eluent: 5% CMA in chloroform). Fractions containing the desired compound were combined and the solvent was removed under reduced pressure at 0° C. with a rotary evaporator to provide the product S2 (14.9 mg, 34%) as an off-white solid. ¹H NMR (500 MHz, CDCl₃, 25° C.): δ 7.94 (d, J=8.3, 1H), 7.86 (d, J=7.3, 1H), 7.79 (d, J=7.9, 1H), 7.55-7.35 (m, 4H), 7.31 (d, J=9.6, 1H), 6.16 (dd, J=36.1, 9.7, 1H), 4.08 (d, J=14.0, 2H), 3.65 (s, 3H). ¹³C NMR (126 MHz, CDCl₃, 25° C.): δ 165.7 (d, J=277.1), 134.2, 132.0, 130.7 (d, J=3.0), 130.4 (d, J=3.2), 129.1, 128.6, 128.1, 126.8, 126.1, 125.7, 123.6, 100.2 (d, J=8.1), 52.6, 36.5 (d, J=26.2). ¹⁹F NMR (471 MHz, CDCl₃, 25° C.): δ −89.5. FTIR (thin film) cm⁻¹: 2922 (m), 2855 (w), 1662 (m), 1569 (w), 1408 (m), 1140 (s), 779 (s). HRMS (ESI) (m/z): calc'd for C₁₅H₁₅FNO [M+H]⁺: 244.1132, found: 244.1129. TLC (5% CMA/chloroform), Rf: 0.30 (UV, KMnO₄).

### Example 50: Synthesis of N-(naphthalen-2-ylmethyl)pent-4-enamide (S3)

A 1-dram vial was charged with enamine N-oxide S1 (90.0 mg, 179 μmol, 1 equiv) and dissolved in chloroform-d (1.79 mL). The reaction vial was flushed with nitrogen, sealed with a cap and Parafilm, and heated to 60° C. After 1 hour, the oil bath was removed and the reaction was cooled to room temperature. The reaction mixture was purified by flash column chromatography on silica gel (eluent: 100% chloroform). Fractions containing the desired compound were combined and the solvent was removed under reduced pressure at 0° C. with a rotary evaporator to provide olefin S3 (8.10 mg, 19%) as a white solid. ¹H NMR (500 MHz, CDCl₃, 25° C.): δ 7.98 (d, J=8.1, 1H), 7.86 (d, J=9.1, 1H), 7.80 (dd, J=6.4, 3.0, 1H), 7.56-7.46 (m, 2H), 7.41 (q, J=3.7, 2H), 5.79 (ddt, J=16.8, 10.2, 6.5, 1H), 5.65 (s, 1H), 5.02 (dd, J=17.2, 1.6, 1H), 4.95 (dd, J=10.2, 1.5, 1H), 4.88 (d, J=5.3, 2H), 2.40 (q, J=7.3, 2H), 2.27 (dd, J=8.2, 6.6, 2H). ¹³C NMR (126 MHz, CDCl₃, 25° C.): δ 172.0, 137.1, 134.1, 133.8, 131.6, 129.0, 128.9, 127.1, 126.9, 126.2, 125.6, 123.8, 115.9, 42.0, 36.1, 29.8. FTIR (thin film) cm⁻¹: 3291 (s), 2926 (w), 1636 (s), 1535 (s), 775 (s). HRMS (ESI) (m/z): calc'd for C₁₆H₁₈NO [M+H]⁺: 240.1383, found: 240.1380. TLC (20% ethyl acetate in hexanes), Rf: 0.15 (UV).

### Example 51: Synthesis of Prop-2-yn-1-yl (2-nitrophenyl)carbamate (S23) triphosgene, Et₃N

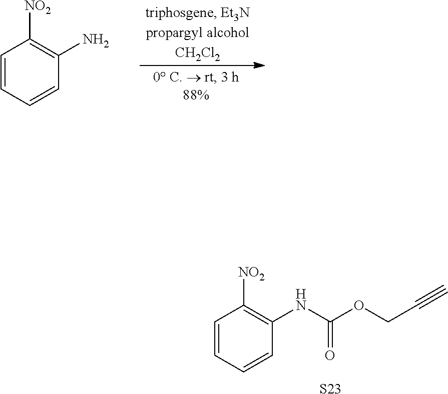 [SMILES: Nc1ccccc1[N+](=O)[O-] ; C#CCOC(=O)Nc1ccccc1[N+](=O)[O-] ; "S23"]

A round-bottom flask was charged with 2-nitroaniline (300 mg, 2.17 mmol, 1 equiv) and dissolved in dichloromethane (20 mL) at room temperature. Triethylamine (1.51 mL, 10.9 mmol, 5.00 equiv) was then added via syringe and the flask was cooled to 0° C. in an ice-water bath. Triphosgene (258 mg, 869 μmol, 0.400 equiv) was added as a solid in one portion and the ice-water bath was removed. After 1.5 hours at room temperature, the reaction was cooled back down to 0° C. and propargyl alcohol (192 μL, 3.26 mmol, 1.50 equiv) was added dropwise via syringe. The ice-water bath was removed and the reaction mixture was allowed to warm to room temperature. After 1.5 hours, the reaction mixture was diluted with dichloromethane (20 mL) and aqueous hydrogen chloride solution (1N, 10 mL) was added. The organic layer was washed with water (2×20 mL) and brine (20 mL) sequentially. The organic layer was then dried over anhydrous sodium sulfate, filtered, and concentrated under reduced pressure. The resulting crude product was purified by flash column chromatography on silica gel (eluent: 5% ethyl acetate in hexanes) to provide alkyne S23 (421 mg, 88%) as a yellow solid. ¹H NMR (500 MHz, CDCl₃, 25° C.): δ 9.94 (s, 1H), 8.54 (dd, J=8.6, 1.3, 1H), 8.20 (dd, J=8.5, 1.6, 1H), 7.63 (ddd, J=8.7, 7.2, 1.6, 1H), 7.14 (ddd, J=8.5, 7.2, 1.3, 1H), 4.80 (d, J=2.4, 2H), 2.53 (t, J=2.4, 7.2 1H). ¹³C NMR (126 MHz, CD₃OD, 25° C.): δ 152.1, 139.6, 136.4, 135.2, 126.9, 124.7, 123.2, 78.8, 76.8, 54.2. FTIR (thin film) cm⁻¹: 3339 (m), 3272 (m), 1744 (s), 1610 (m), 1498 (s), 1435 (m), 1334 (s), 1237 (s), 1054 (s), 976 (m), 738 (m). HRMS (ESI) (m/z): calc'd for C₁₀H₉N₂O₄ [M+H]⁺: 221.0557, found: 221.0555. TLC (10% ethyl acetate in hexanes), Rf: 0.30 (UV, KMnO₄).

### Example 52: Synthesis of (E)-N,N-dimethyl-3-(((2-nitrophenyl)carbamoyl)oxy)prop-1-en-1-amine oxide (32a)

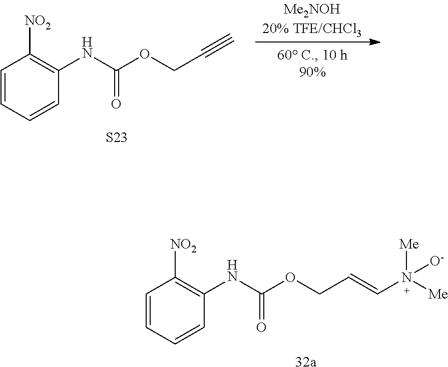

A glass 2 mL LC-MS vial was charged with prop-2-yn-1-yl (2-nitrophenyl)carbamate (S23, 20.0 mg, 90.8 μmol, 1 equiv) at room temperature. A solution of N,N-dimethylhydroxylamine (Liu et al., Chinese Patent No. 2009/101503374 A (2009)) (2.27 M, 200 μL, 454 μmol, 5.00 equiv) in 20% v/v trifluoroethanol in chloroform was then added via syringe. The vial was flushed with nitrogen, sealed with a septum cap and Parafilm, and heated to 60° C. After 19 hours, the reaction was removed from heat, allowed to cool to room temperature, and directly loaded onto a silica gel column. The reaction mixture was purified by flash column chromatography (eluent: 30% CMA in chloroform). Fractions containing the desired compound were combined, and the solvent was removed under reduced pressure at 0° C. with a rotary evaporator to provide enamine N-oxide 32a (23.0 mg, 90%) as a yellow solid. ¹H NMR (500 MHz, CD₃OD, 25° C.): δ=8.18 (dd, J=8.4, 1.3, 1H), 8.14 (dd, J=8.4, 1.6, 1H), 7.70 (ddd, J=8.6, 7.3, 1.6, 1H), 7.29 (ddd, J=8.5, 7.3, 1.3, 1H), 6.88 (dt, J=13.5, 1.8, 1H), 6.72 (dt, J=13.5, 5.0, 1H), 4.90 (dd, J=5.0, 1.9, 2H), 3.66 (s, 6H). ¹³C NMR (126 MHz, CD₃OD, 25° C.): δ 154.3, 140.4, 139.7, 136.3, 134.8, 126.9, 126.2, 125.2, 123.8, 62.4, 58.8. FTIR (thin film) cm⁻¹: 1744 (m), 1610 (m), 1505 (s), 1341 (m), 1192 (s), 1133 (m). HRMS (ESI) (m/z): calc'd for C₁₂H₁₆N₃O₅ [M+H]⁺: 282.1084, found: 282.1081. TLC (30% CMA in chloroform), Rf: 0.20 (UV, KMnO₄).

### Example 53: Synthesis of (E)-4-(3-(((2-nitrophenyl)carbamoyl)oxy)prop-1-en-1-yl)morpholine 4-oxide (32b)

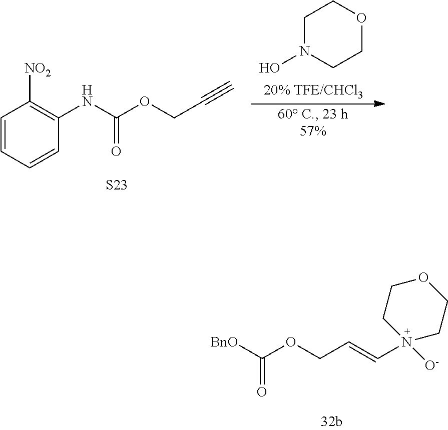

A glass 2 mL LC-MS vial was charged with prop-2-yn-1-yl (2-nitrophenyl)carbamate (S23, 20.0 mg, 90.8 μmol, 1 equiv) at room temperature. A solution of morpholin-4-ol (O'Neil et al., Tetrahedron Lett. 42:8247-8249 (2001)) (1.00 M, 454 μL, 454 μmol, 5.00 equiv) in 20% v/v trifluoroethanol in chloroform was then added via syringe. The vial was flushed with nitrogen, sealed with a septum cap and Parafilm, and heated to 60° C. After 23 hours, the reaction was removed from heat, allowed to cool to room temperature, and directly loaded onto a silica gel column. The reaction mixture was purified by flash column chromatography (eluent: 30% CMA in chloroform). Fractions containing the desired compound were combined, and the solvent was removed under reduced pressure at 0° C. with a rotary evaporator to provide enamine N-oxide 32b (16.6 mg, 57%) as a clear, yellow oil. ¹H NMR (500 MHz, CD₃OD, 25° C.): δ 8.16 (ddd, J=16.9, 8.4, 1.4, 2H), 7.70 (ddd, J=8.6, 7.3, 1.6, 1H), 7.30 (ddd, J=8.5, 7.3, 1.3, 1H), 6.89 (dt, J=13.6, 1.8, 1H), 6.78 (dt, J=13.6, 4.9, 1H), 4.93 (dd, J=4.9, 1.8, 2H), 4.20 (ddd, J=13.6, 10.7, 2.2, 2H), 4.07-3.96 (m, 4H), 3.73 (dt, J=10.9, 2.3, 2H). ¹³C NMR (126 MHz, CD₃OD, 25° C.): δ 154.3, 140.5, 139.1, 136.3, 134.8, 127.9, 126.9, 125.2, 123.8, 65.6, 62.6, 62.3. FTIR (thin film) cm⁻¹: 1744 (m) 1684 (m), 1610 (m), 1505 (s), 1341 (m), 1200 (s), 1129 (m). HRMS (ESI) (m/z): calc'd for C₁₄H₁₈N₃O₆ [M+H]⁺: 324.1190, found: 324.1187. TLC (30% CMA in chloroform), Rf: 0.10 (UV, KMnO₄).

### Example 54: Synthesis of (E)-N,N-diethyl-3-(((2-nitrophenyl)carbamoyl)oxy)prop-1-en-1-amine oxide (32c)

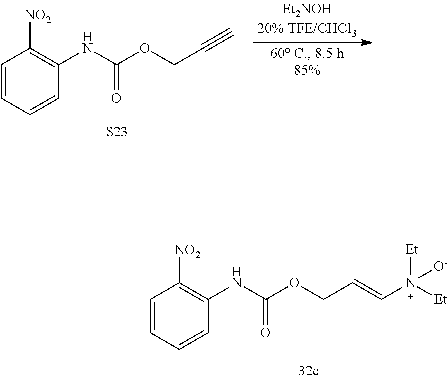

A glass 2 mL LC-MS vial was charged with prop-2-yn-1-yl (2-nitrophenyl)carbamate (S23, 300 mg, 1.36 mmol, 1 equiv) and dissolved in 20% v/v trifluoroethanol in chloroform (6.81 mL) at room temperature. N,N-diethylhydroxylamine (700 μL, 6.81 mmol, 5.00 equiv) was then added via syringe. The vial was flushed with nitrogen, sealed with a septum cap and Parafilm, and heated to 60° C. After 8.5 hours, the reaction was removed from heat, allowed to cool to room temperature, and directly loaded onto a silica gel column. The reaction mixture was purified by flash column chromatography (eluent: 30% CMA in chloroform). Fractions containing the desired compound were combined, and the solvent was removed under reduced pressure at 0° C. with a rotary evaporator to provide enamine N-oxide 32c (357 mg, 85%) as a yellow solid. ¹H NMR (500 MHz, CD₃OD, 25° C.): δ 8.15 (ddd, J=12.7, 8.4, 1.3, 2H), 7.70 (ddd, J=8.7, 7.3, 1.6, 1H), 7.29 (ddd, J=8.5, 7.3, 1.3, 1H), 6.62 (dt, J=13.5, 5.0, 1H), 6.47 (dt, J=13.4, 1.8, 1H), 4.92 (dd, J=5.0, 1.8, 2H), 3.83 (p, J=7.1, 4H), 1.39 (t, J=7.1, 6H). ¹³C NMR (126 MHz, CD₃OD, 25° C.): δ 154.5, 140.6, 136.2, 134.8, 128.9, 126.8, 125.2, 123.9, 120.0, 65.4, 62.8, 8.3. FTIR (thin film) cm⁻¹: 1725 (s), 1610 (m), 1528 (s), 1356 (m), 1237 (s). HRMS (ESI) (m/z): calc'd for C₁₄H₂₀N₃O₅ [M+H]⁺: 310.1397, found: 310.1393. TLC (30% CMA in chloroform), Rf: 0.20 (UV, KMnO₄).

### Example 55: Synthesis of (E)-N-butyl-N-(3-(((2-nitrophenyl)carbamoyl)oxy)prop-1-en-1-yl)butan-1-amine oxide (32d)

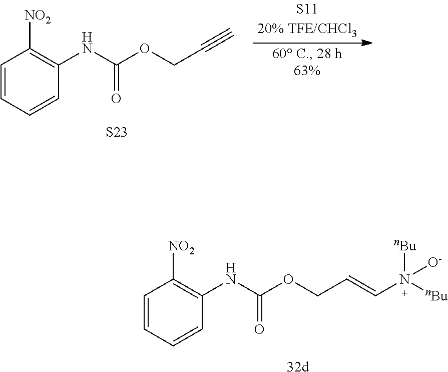

A glass 2 mL LC-MS vial was charged with prop-2-yn-1-yl (2-nitrophenyl)carbamate (S23, 40.0 mg, 182 μmol, 2.00 equiv) at room temperature. A solution of N,N-dibutylhydroxylamine (S11, 454 mM, 200 μL, 90.8 μmol, 1 equiv) in 20% v/v trifluoroethanol in chloroform was then added via syringe. The vial was flushed with nitrogen, sealed with a septum cap and Parafilm, and heated to 60° C. After 28 hours, the reaction was removed from heat, allowed to cool to room temperature, and directly loaded onto a silica gel column. The reaction mixture was purified by flash column chromatography (eluent: 30% CMA in chloroform). Fractions containing the desired compound were combined, and the solvent was removed under reduced pressure at 0° C. with a rotary evaporator to provide enamine N-oxide 32d (19.7 mg, 63%) as a clear, yellow oil. ¹H NMR (500 MHz, CD₃OD, 25° C.): δ 8.14 (ddd, J=8.2, 6.6, 1.4, 2H), 7.70 (ddd, J=8.6, 7.3, 1.5, 1H), 7.29 (ddd, J=8.5, 7.3, 1.3, 1H), 6.60 (dt, J=13.5, 4.9, 1H), 6.50 (dt, J=13.4, 1.7, 1H), 4.90 (dd, J=5.0, 1.7, 2H), 3.41-3.31 (m, 2H), 3.31-3.23 (m, 2H), 1.88-1.59 (m, 4H), 1.42 (sx, J=7.4, 4H), 0.98 (t, J=7.4, 6H). ¹³C NMR (126 MHz, CD₃OD, 25° C.): δ 154.5, 140.3, 136.2, 135.7, 134.8, 128.2, 126.9, 125.3, 123.9, 70.1, 62.7, 25.6, 20.5, 14.1. FTIR (thin film) cm⁻¹: 2907 (w), 1744 (m), 1610 (m), 1505 (s), 1341 (m), 1196 (s). HRMS (ESI) (m/z): calc'd for C₁₈H₂₈N₃O₅ [M+H]⁺: 366.2023, found: 366.2016. TLC (30% CMA in chloroform), Rf: 0.50 (UV, KMnO₄).

### Example 56: Synthesis of (E)-N-ethyl-N-isopropyl-3-(((2-nitrophenyl)carbamoyl)oxy)prop-1-en-1-amine oxide (32e)

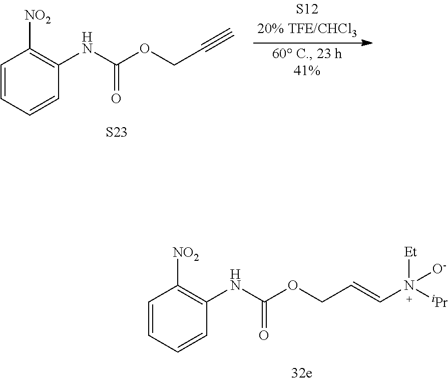

A glass 2 mL LC-MS vial was charged with prop-2-yn-1-yl (2-nitrophenyl)carbamate (S23, 20.0 mg, 90.8 μmol, 1 equiv) at room temperature. A solution of N-ethyl-N-isopropylhydroxylamine (S12, 2.27 M, 200 μL, 454 μmol, 5.00 equiv) in 20% v/v trifluoroethanol in chloroform was then added via syringe. The vial was flushed with nitrogen, sealed with a septum cap and Parafilm, and heated to 60° C. After 23 hours, the reaction was removed from heat, allowed to cool to room temperature, and directly loaded onto a silica gel column. The reaction mixture was purified by flash column chromatography (eluent: 30% CMA in chloroform). Fractions containing the desired compound were combined, and the solvent was removed under reduced pressure at 0° C. with a rotary evaporator to provide enamine N-oxide 32e (12.0 mg, 41%) as a clear, yellow oil. ¹H NMR (500 MHz, CD₃OD, 25° C.): δ 8.14 (ddd, J=8.4, 3.3, 1.4, 2H), 7.70 (ddd, J=8.7, 7.3, 1.6, 1H), 7.30 (ddd, J=8.5, 7.3, 1.3, 1H), 6.60 (dt, J=13.5, 5.0, 1H), 6.50 (dt, J=13.5, 1.6, 1H), 4.92 (dd, J=5.0, 1.6, 2H), 4.08 (sp, J=6.5, 1H), 3.85 (q, J=7.1, 2H), 1.45 (dd, J=9.8, 6.5, 6H), 1.38 (t, J=7.1, 3H). ¹³C NMR (126 MHz, CD₃OD, 25° C.): δ 154.6, 136.2, 134.7, 133.2, 129.5, 126.9, 125.3, 124.0, 73.7, 62.9, 62.6, 17.5, 16.3, 8.4. FTIR (thin film) cm⁻¹: 1744 (m), 1684 (m) 1613 (m), 1505 (s), 1341 (m), 1196 (s), 1133 (m). HRMS (ESI) (m/z): calc'd for C₁₅H₂₂N₃O₅ [M+H]⁺: 324.1554, found: 324.1550. TLC (30% CMA in chloroform), Rf: 0.20 (UV, KMnO₄).

### Example 57: Synthesis of (E)-N-cyclohexyl-N-(3-(((2-nitrophenyl)carbamoyl)oxy)prop-1-en-1-yl)cyclohexanamine oxide (32f)

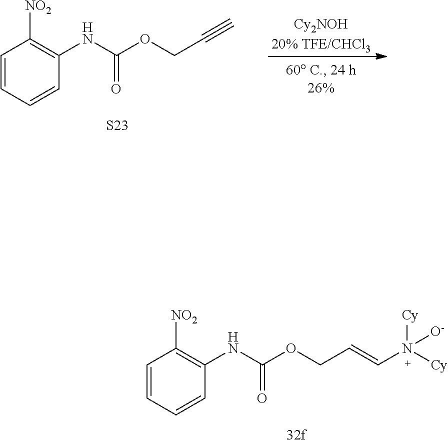

A glass 2 mL LC-MS vial was charged with prop-2-yn-1-yl (2-nitrophenyl)carbamate (S23, 40.0 mg, 0.182 mmol, 1 equiv) at room temperature. A solution of N,N-dicyclohexylhydroxylamine (Murray et al., Synth. Commun. 19:3509-3522 (1989)) (2.73 M, 200 μL, 546 μmol, 3.00 equiv) in 20% v/v trifluoroethanol in chloroform was then added via syringe. The vial was flushed with nitrogen, sealed with a septum cap and Parafilm, and heated to 60° C. After 24 hours, the reaction was removed from heat, allowed to cool to room temperature, and directly loaded onto a silica gel column. The reaction mixture was purified by flash column chromatography (eluent: 10% CMA in chloroform). Fractions containing the desired compound were combined, and the solvent was removed under reduced pressure at 0° C. with a rotary evaporator to provide enamine N-oxide 32f (20.0 mg, 26%) as a clear, yellow oil. ¹H NMR (500 MHz, CD₃OD, 25° C.): δ 8.12 (ddd, J=8.3, 6.8, 1.4, 2H), 7.70 (ddd, J=8.7, 7.3, 1.6, 1H), 7.30 (ddd, J=8.4, 7.3, 1.3, 1H), 6.51 (dt, J=13.4, 5.1, 1H), 6.41 (dt, J=13.4, 1.7, 1H), 4.90 (dd, J=5.2, 1.6, 2H), 3.93 (tt, J=11.7, 3.4, 2H), 2.22 (dt, J=11.9, 3.3, 2H), 2.12 (dt, J=11.8, 3.4, 2H), 1.96 (tdd, J=16.4, 6.3, 2.9, 4H), 1.70 (dt, J=13.5, 3.2, 2H), 1.56 (dddd, J=15.1, 11.9, 9.3, 3.3, 4H), 1.42 (qq, J=13.2, 3.5, 4H), 1.19 (qt, J=13.1, 3.8, 2H). ¹³C NMR (126 MHz, CD₃OD, 25° C.): δ 154.7, 141.0, 136.2, 134.7, 132.4, 129.4, 126.8, 125.4, 124.1, 75.9, 63.0, 28.1, 26.8, 26.3, 26.1, 26.0. FTIR (thin film) cm⁻¹: 2940 (m), 2862 (w), 1744 (m), 1677 (m), 1610 (m), 1505 (s), 1341 (m), 1192 (s), 1133 (s). HRMS (ESI) (m/z): calc'd for C₂₂H₃₂N₃O₅ [M+H]⁺: 418.2336, found: 418.2330. TLC (30% CMA in chloroform), Rf: 0.50 (UV, KMnO₄).

### Example 58: Synthesis of N,N-diethyl-3-(p-tolyloxy)propan-1-amine (S5)

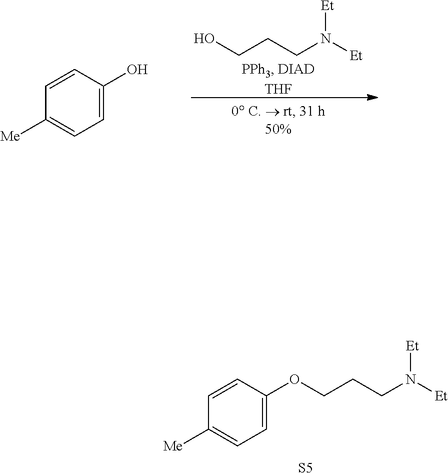

A round-bottom flask was charged sequentially with 3-diethylamino-1-propanol (0.300 g, 2.29 mmol, 1.40 equiv), triphenylphosphine (428 mg, 1.63 mmol, 1.00 equiv), and p-cresol (177 mg, 1.63 mmol, 1 equiv). Tetrahydrofuran (16 mL) was then introduced into the flask and the solution was cooled to 0° C. in an ice-water bath. After 5 minutes, diisopropylazodicarboxylate (330 mg, 1.63 mmol, 1.00 equiv) was added dropwise via syringe. The ice-water bath was removed and the reaction mixture was allowed to warm to room temperature. After 31 hours, the reaction was concentrated under reduced pressure. The resulting crude product was purified by flash column chromatography on silica gel (eluent: 20% CMA in chloroform) to provide amine S5 (181 mg, 50%) as an off-white solid. ¹H NMR (500 MHz, CDCl₃, 25° C.): δ 7.05 (d, J=8.5, 2H), 6.78 (d, J=8.5, 2H), 3.96 (t, J=6.3, 2H), 2.60 (t, J=7.4, 2H), 2.54 (q, J=7.2, 4H), 2.26 (s, 3H), 1.96-1.83 (m, 2H), 1.02 (t, J=7.2 6H). ¹³C NMR (126 MHz, CDCl₃, 25° C.): δ 156.9, 130.0, 129.9, 114.5, 66.3, 49.5, 47.1, 26.8, 20.6, 11.5. FTIR (thin film) cm⁻¹: 2967 (m), 1513 (s), 1472 (m), 1241 (s), 1058 (m), 816 (m). HRMS (ESI) (m/z): calc'd for C₁₄H₂₄NO [M+H]⁺: 222.1852, found: 222.1850. TLC (30% CMA in chloroform), Rf: 0.40 (UV, KMnO₄).

### Example 59: Synthesis of N,N-diethyl-3-(p-tolyloxy)propan-1-amine oxide (S4)

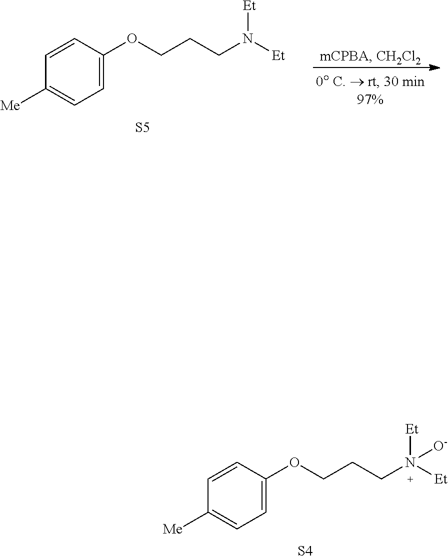

A round-bottom flask was charged with N,N-diethyl-3-(p-tolyloxy)propan-1-amine (S5, 100 mg, 824 μmmol, 1 equiv) at room temperature. Dichloromethane (8.24 mL) was then introduced into the flask and the solution was cooled to 0° C. in an ice-water bath. m-Chloroperoxybenzoic acid (142 mg, 824 μmmol, 1.00 equiv) was then added as a solid in one portion to the flask at 0° C. After 30 minutes, the reaction was concentrated under reduced pressure. The resulting crude product was purified by flash column chromatography on silica gel (eluent: 30% CMA in chloroform) to provide N-oxide S4 (105 mg, 97%) as a clear, colorless oil. ¹H NMR (500 MHz, CDCl₃, 25° C.): δ 6.99 (d, J=8.4, 2H), 6.70 (d, J=8.5, 2H), 3.97 (t, J=5.7, 2H), 3.28-3.20 (m, 2H), 3.16 (q, J=7.3, 4H), 2.24 (dq, J=11.5, 5.6 Hz, 2H), 2.20 (s, 3H), 1.25 (t, J=7.3, 6H). ¹³C NMR (126 MHz, CDCl₃, 25° C.): δ 156.3, 130.2, 129.9, 114.2, 65.8, 62.7, 60.2, 23.2, 20.4, 8.7. FTIR (thin film) cm⁻¹: 2948 (m) 1513 (s), 1461 (m), 1237 (s), 961 (m), 816 (m). HRMS (ESI) (m/z): calc'd for C₁₄H₂₄NO₂ [M+H]⁺: 238.1802, found: 238.1798. TLC (30% CMA in chloroform), Rf: 0.10 (UV, KMnO₄).

### Example 60: Synthesis of 1-Phenyl-4-(triisopropylsilyl)but-3-yn-2-ol (S24)

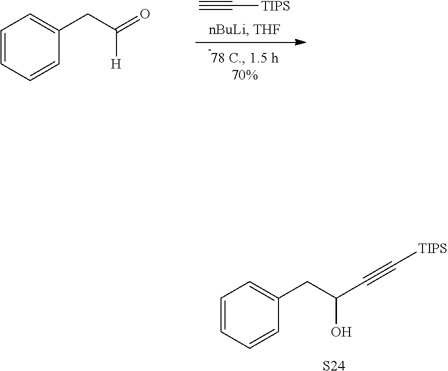

(Triisopropylsilyl)acetylene (2.24 mL, 9.99 mmol, 1.20 equiv) was added via syringe to a round-bottom flask containing dichloromethane (83 mL) and cooled to −78° C. in an acetone-dry ice bath. A solution of 2.5 M n-butyllithium (3.66 mL, 9.15 mmol, 1.10 equiv) in hexanes was added dropwise via syringe to the reaction mixture. The solution was then warmed to 0° C. in an ice-water bath. After 30 minutes, the solution was cooled to −78° C. and phenylacetaldehyde (973 μL, 8.32 mmol, 1 equiv) was added dropwise via syringe. After 1 hour, aqueous hydrogen chloride solution (1N, 50 mL) and ethyl acetate (100 mL) were added sequentially. The aqueous layer was extracted with ethyl acetate (2×30 mL). The combined organic layers were dried over anhydrous sodium sulfate, filtered, and concentrated under reduced pressure. The resulting residue was purified by flash column chromatography on silica gel (eluent: 2% ethyl acetate in hexanes) to provide alkyne S24 (1.76 g, 70%) as a clear, colorless oil. ¹H NMR (500 MHz, CDCl₃, 25° C.): δ 7.37-7.23 (m, 5H), 4.64 (q, J=6.0, 1H), 3.04 (d, J=6.4, 2H), 2.01 (d, J=5.4, 1H), 1.10-1.06 (m, 21H). ¹³C NMR (126 MHz, CDCl₃, 25° C.): δ 136.7, 130.1, 128.5, 127.0, 108.1, 86.8, 63.8, 44.3, 18.7, 11.3. FTIR (thin film) cm⁻¹: 3339 (br-w), 2944 (s), 2862 (s), 2173 (w), 1461 (m), 1032 (s), 883 (s). HRMS (ESI) (m/z): calc'd for C₁₉H₃₁OSi [M+H]⁺: 303.2139, found: 303.2137. TLC (5% ethyl acetate in hexanes), Rf: 0.27 (UV, KMnO₄).

### Example 61: Synthesis of 1-Methyl-4-((1-phenylbut-3-yn-2-yloxy)benzene (S25)

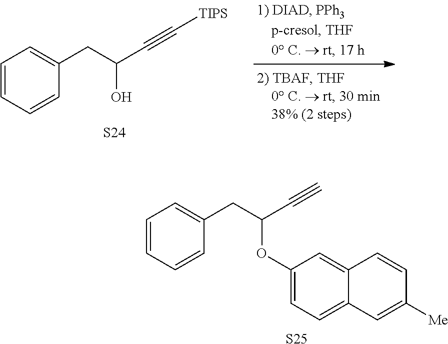

A round-bottom flask was charged sequentially with 1-phenyl-4-(triisopropylsilyl)but-3-yn-2-ol (S24, 1.00 g, 3.31 mmol, 1.40 equiv), triphenylphosphine (868 mg, 3.31 mmol, 1.40 equiv), and p-cresol (255 mg, 2.36 mmol, 1 equiv). Tetrahydrofuran (23 mL) was then introduced into the flask and the solution was cooled to 0° C. in an ice-water bath. After 5 minutes, diisopropylazodicarboxylate (652 μL, 3.31 mmol, 1.40 equiv) was added dropwise via syringe. The ice-water bath was removed and the reaction mixture was allowed to warm to room temperature. After 17 hours, the reaction was diluted with ethyl acetate (200 mL) and the organic layer was washed with water (50 mL) and brine (50 mL) successively. The organic layer was concentrated under reduced pressure. The crude product was then dissolved into tetrahydrofuran (23 mL) and cooled to 0° C. in an ice-water bath. Tetrabutylammonium fluoride (1.40 mL, 1.40 mmol, 0.600 equiv) was added dropwise via syringe and the reaction mixture was allowed to warm to room temperature after removal of the ice-water bath. After 30 minutes, the reaction was concentrated under reduced pressure. The resulting crude product was purified by flash column chromatography on silica gel (eluent: 100% hexanes) to provide alkyne S25 (213 mg, 38%) as a clear, colorless oil. ¹H NMR (500 MHz, CDCl₃, 25° C.): δ 7.39-7.23 (m, 5H), 7.06 (d, J=8.1, 2H), 6.87 (d, J=8.5, 2H), 4.84 (td, J=6.8, 2.0, 1H), 3.22 (ddd, J=49.0, 13.8, 6.7, 2H), 2.47 (d, J=1.9, 1H), 2.26 (s, 3H). ¹³C NMR (126 MHz, CDCl₃, 25° C.): δ 155.4, 136.7, 131.1, 130.0, 129.9, 128.5, 127.1, 116.0, 82.0, 75.4, 69.2, 42.3, 20.7. FTIR (thin film) cm⁻¹: 3287 (m), 3030 (w), 1509 (s), 1226 (s), 1021 (m), 700 (m). HRMS (ESI) (m/z): calc'd for C₁₇H₁₇O [M+H]⁺: 237.1274, found: 237.1271. TLC (100% hexanes), Rf: 0.20 (UV, KMnO₄).

[symbol]

### Example 62: Synthesis of (E)-N,N-diethyl-4-phenyl-3-(p-tolyloxy)but-1-en-1-amine oxide (S6)

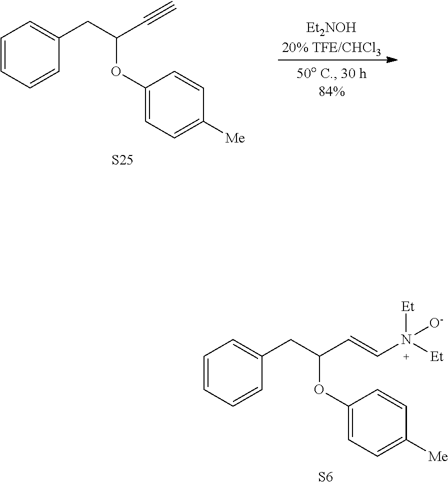

A glass 1-dram vial was charged with 1-methyl-4-((1-phenylbut-3-yn-2-yl)oxy)benzene (S25, 100 mg, 423 μmol, 1 equiv) and dissolved in 20% v/v trifluoroethanol in chloroform (2.12 mL) at room temperature. N,N-diethylhydroxylamine (218 μL, 2.12 mmol, 5.00 equiv) was then added via syringe. The vial was flushed with nitrogen, sealed with a septum cap and Parafilm, and heated to 60° C. After 30 hours, the reaction was removed from heat, allowed to cool to room temperature, and directly loaded onto a silica gel column. The reaction mixture was purified by flash column chromatography (eluent: 30% CMA in chloroform). Fractions containing the desired compound were combined, and the solvent was removed under reduced pressure at 0° C. with a rotary evaporator to provide enamine N-oxide S6 (116 mg, 84%) as an off-white solid. ¹H NMR (500 MHz, CD₃OD, 25° C.): δ 7.32-7.18 (m, 5H), 7.08 (d, J=8.2, 2H), 6.85 (d, J=8.6, 2H), 6.44 (dd, J=13.4, 6.0, 1H), 6.13 (dd, J=13.4, 1.3, 1H), 5.20 (qd, J=6.3, 1.3, 1H), 3.70-3.53 (m, 4H), 3.23-3.08 (m, 2H), 2.25 (s, 3H), 1.05 (td, J=7.2, 5.1, 6H). ¹³C NMR (126 MHz, CD₃OD, 25° C.): δ 156.5, 138.0, 134.7, 133.0, 132.6, 131.2, 131.0, 129.7, 128.0, 117.6, 77.3, 65.3, 65.4, 42.0, 20.7, 8.2, 8.1. FTIR (thin film) cm⁻¹: 3030 (w), 1666 (m), 1509 (s), 117 (s), 1136 (s). HRMS (ESI) (m/z): calc'd for C₂₁H₂₈NO₂ [M+H]⁺: 326.2115, found: 326.2111. TLC (30% CMA in chloroform), Rf: 0.15 (UV, KMnO₄).

### Example 63: Synthesis of 1-Methyl-4-((1-phenylbut-3-yn-2-yl)oxy)benzene (S7)

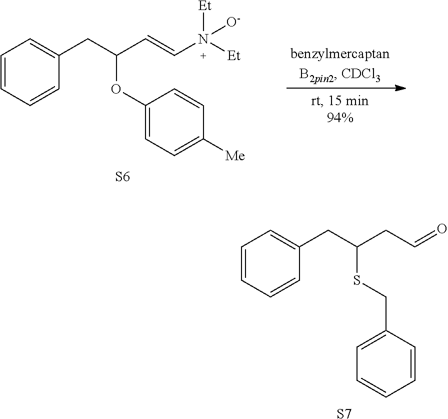

A round-bottom flask was charged with (E)-N,N-diethyl-4-phenyl-3-(p-tolyloxy)but-1-en-1-amine oxide (S6, 26.0 mg, 79.8 μmol, 1 equiv) and dissolved with chloroform-d (773 μL) at room temperature. Benzylmercaptan (93.7 μL, 798 μmol, 10.0 equiv) was then added via syringe. After 5 minutes, analysis by TLC indicated no reduction by benzylmercaptan. Solid bis(pinacolato)diboron (20.3 mg, 79.8 μmol, 1.00 equiv) was then added in one portion at room temperature. After 15 minutes, the reaction was directly loaded onto a silica gel column. The reaction mixture was purified by flash column chromatography (eluent: 100% chloroform) to provide aldehyde S7 (20.2 mg, 94%) as an off-white solid. ¹H NMR (500 MHz, CDCl₃, 25° C.): δ 9.51 (dd, J=2.5, 1.4 Hz, 1H), 7.33-7.18 (m, 8H), 7.14-7.08 (m, 2H), 3.68 (q, J=13.4, 10.4 Hz, 2H), 3.20 (tdd, J=8.1, 6.5, 5.6 Hz, 1H), 2.97 (dd, J=13.7, 6.4 Hz, 1H), 2.78 (dd, J=13.8, 8.2 Hz, 1H), 2.60-2.40 (m, 2H). ¹³C NMR (126 MHz, CDCl₃, 25° C.): δ 200.7, 138.6, 138.1, 129.6, 129.2, 128.8, 128.7, 127.4, 127.0, 47.8, 42.3, 40.5, 36.1. FTIR (thin film) cm⁻¹: 3026 (w), 2922 (w), 1722 (s), 1494 (m), 1453 (m), 700 (s). HRMS (ESI) (m/z): calc'd for C₁₇H₁₉OS [M+H]⁺: 271.1151, found: 271.1149. TLC (100% chloroform), Rf: 0.45 (UV, KMnO₄).

### Example 64: Synthesis of Boc-propargyl-staurosporine (35)

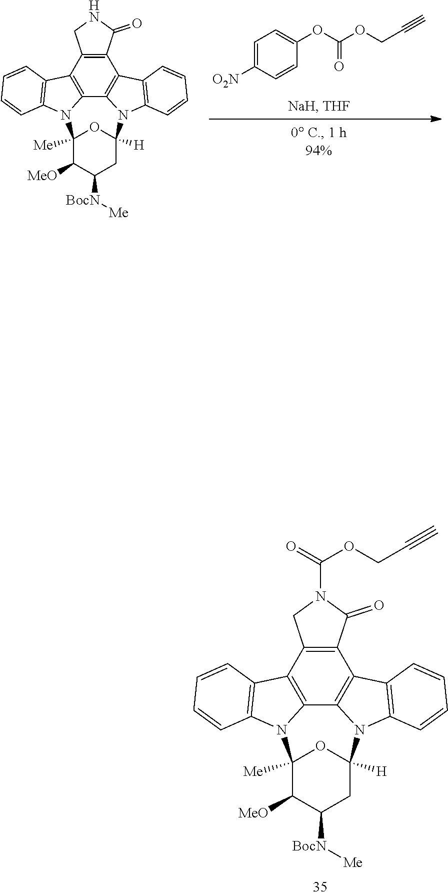 [SMILES: CO[C@@H]1[C@H](N(C)C(=O)OC(C)(C)C)C[C@H]2O[C@]1(C)n1c3ccccc3c3c4c(c5c6ccccc6n2c5c31)C(=O)NC4 ; C#CCOC(=O)Oc1ccc([N+](=O)[O-])cc1 ; C#CCOC(=O)N1Cc2c(c3c4ccccc4n4c3c3c2c2ccccc2n3[C@@]2(C)O[C@@H]4C[C@@H](N(C)C(=O)OC(C)(C)C)[C@H]2OC)C1=O]

A round-bottom flask was charged with Boc-staurosporine (Zimmermann et al., International Patent No. WO 1999/9902532) (85.0 mg, 150 μmol, 1 equiv) and 4-nitrophenyl prop-2-yn-1-yl carbonate (Egami et al., Org. Biomol. Chem. 9:7667-7670 (2011)) (66.3 mg, 300 μmol, 2.00 equiv) sequentially. Tetrahydrofuran (10 mL) was then introduced via syringe and the reaction mixture was cooled to 0° C. with an ice-water bath. After 5 minutes, solid sodium hydride (18.0 mg, 0.750 mmol, 5.00 equiv) was added in one portion at 0° C. After 2 hours, the reaction mixture was added dropwise to aqueous hydrogen chloride solution (1N, 50 mL) at 0° C. The mixture was then diluted with dichloromethane (100 mL). The aqueous layer was washed with dichloromethane (2×50 mL). The combined organic layers were dried with anhydrous sodium sulfate, filtered, and concentrated under reduced pressure. The crude solid was purified by flash chromatography on silica gel (eluent: 40% ethyl acetate in hexanes) to provide alkyne 35 (91.9 mg, 94%) as a white solid. ¹H NMR (500 MHz, CDCl₃, 25° C.): δ 9.37 (d, J=8.0 Hz, 1H), 7.94 (d, J=7.6 Hz, 1H), 7.74 (d, J=8.4 Hz, 1H), 7.51-7.42 (m, 2H), 7.37 (t, J=7.4 Hz, 1H), 7.32 (t, J=7.3 Hz, 1H), 7.18 (t, J=10.0 Hz, 1H), 6.58 (dd, J=8.5, 4.5 Hz, 1H), 5.33-5.23 (m, 1H), 5.21-5.07 (m, 1H), 5.09-4.96 (m, 2H), 4.84-4.56 (m, 1H), 3.91 (d, J=59.1 Hz, 1H), 2.66 (s, 3H), 2.62-2.53 (m, 2H), 2.51-2.39 (m, 7H), 1.59 (s, 3H), 1.48 (s, 6H). ¹³C NMR (126 MHz, CDCl₃, 25° C.): δ 167.3, 156.1, 151.8, 139.0, 136.8, 131.9, 130.3, 127.0, 126.1, 125.6, 124.7, 123.3, 121.7, 121.2, 120.4, 117.6, 116.6, 114.0, 112.9, 112.6, 108.0, 95.1, 85.2, 82.7, 80.5, 77.8, 75.8, 60.7, 54.0, 50.0, 49.5, 30.5, 29.9, 29.5, 28.9, 28.7, 28.4. FTIR (thin film) cm⁻¹: 2974 (w), 2933 (w), 1774 (m), 1722 (m), 1688 (m), 1632 (w), 1584 (w), 1341 (s), 1308 (s), 1282 (s), 1140 (m), 741 (m). HRMS (ESI) (m/z): calc'd for C₃₇H₃₇N₄O₇ [M+H]⁺: 649.2657, found: 649.2647. TLC (50% ethyl acetate in hexanes), Rf: 0.50 (UV).

### Example 65: Synthesis of Propargyl-staurosporine (36)

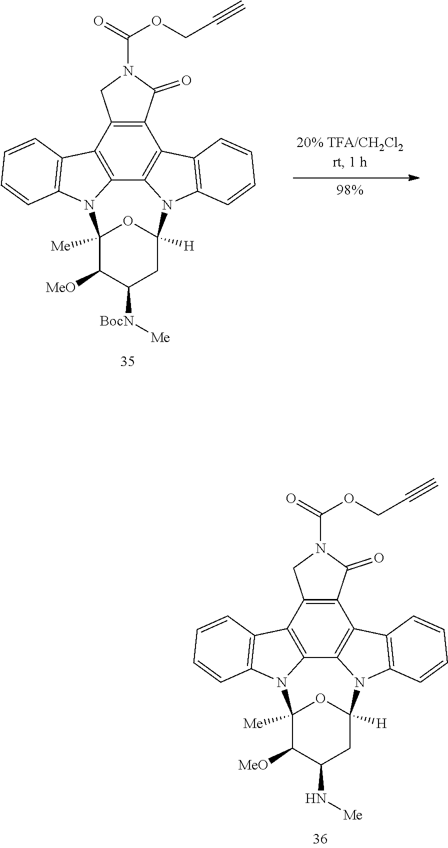 [SMILES: C#CCOC(=O)N1Cc2c(c3c4ccccc4n4c3c3c2c2ccccc2n3[C@@]2(C)O[C@@H]4C[C@@H](N(C)C(=O)OC(C)(C)C)[C@H]2OC)C1=O ; C#CCOC(=O)N1Cc2c(c3c4ccccc4n4c3c3c2c2ccccc2n3[C@@]2(C)O[C@@H]4C[C@@H](NC)[C@H]2OC)C1=O]

A glass 20 mL scintillation vial was charged with Boc-propargyl-staurosporine (35, 8.0 mg, 12.3 μmol, 1 equiv) at room temperature. A solution of 20% trifluoroacetic acid in dichloromethane (2.40 mL) was introduced via syringe. After 1 hour, the reaction mixture was concentrated under a stream of nitrogen. The crude residue was then purified by automated C₁₈ [symbol] reverse phase column chromatography (30 g C₁₈ silica gel, 25 μm spherical particles, eluent: H₂O+0.1% TFA (2 CV), gradient 0→100% MeCN/H₂O+0.1% TFA (15 CV), t_{R}=8.5 CV) to provide staurosporine-alkyne 36 (6.6 mg, 98%) as a white solid. ¹H NMR (500 MHz, CD₃OD, 25° C.): δ 9.13 (d, J=7.9 Hz, 1H), 7.99 (d, J=8.5 Hz, 1H), 7.82 (d, J=7.6 Hz, 1H), 7.59 (ddd, J=8.5, 7.2, 1.3 Hz, 1H), 7.47 (t, J=7.4 Hz, 1H), 7.40 (ddd, J=8.2, 7.0, 1.2 Hz, 1H), 7.25 (t, J=7.5 Hz, 1H), 7.04 (d, J=8.1 Hz, 1H), 6.19 (dd, J=9.5, 2.7 Hz, 1H), 5.03-4.95 (m, 3H), 4.41 (d, J=16.5 Hz, 1H), 4.14 (s, 1H), 3.98-3.89 (m, 1H), 3.22-3.08 (m, 2H), 2.76 (s, 3H), 2.57 (s, 3H), 2.09-1.94 (m, 4H). ¹³C NMR (126 MHz, CD₃OD, 25° C.): δ 169.3, 152.2, 139.5, 137.6, 132.8, 131.3, 127.7, 127.3, 127.0, 126.9, 125.6, 123.9, 122.8, 122.7, 121.0, 117.7, 116.5, 114.8, 113.8, 109.7, 94.4, 81.7, 81.5, 78.9, 77.5, 60.5, 55.8, 54.7, 49.9, 31.5, 28.8, 28.8. FTIR (thin film) cm⁻¹: 2929 (m), 1774 (s), 1718 (s), 1636 (w), 1580 (m), 1345 (s), 1312 (s), 1278 (s), 1095 (s), 741 (s). HRMS (ESI) (m/z): calc'd for C₃₂H₂₉N₄O₅ [M+H]⁺: 549.2132, found: 549.2127. TLC (5% methanol in dichloromethane), Rf: 0.40 (UV).

### Example 66: Synthesis of N,N-diethyl enamine N-oxide staurosporine (37)

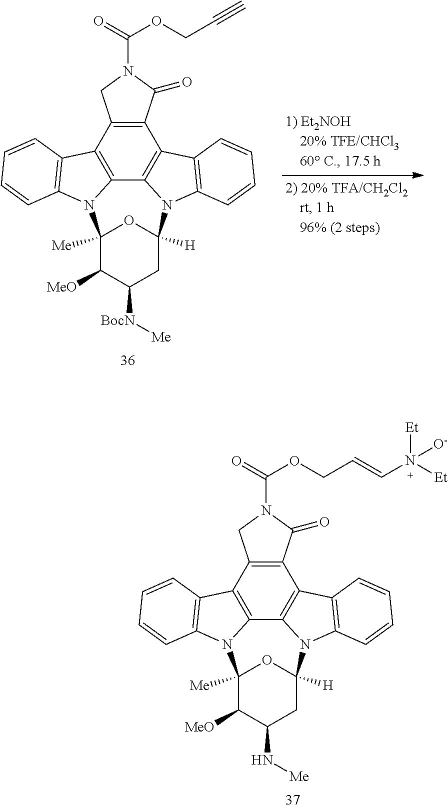

A glass 2 mL LC-MS vial was charged with N,N-diethylhydroxylamine (15.8 μL, 154 μmol, 10.0 equiv) at room temperature. A solution of Boc-propargyl staurosporine (36, 10.0 mg, 15.4 μmol, 1 equiv) in 20% v/v trifluoroethanol in chloroform (200 μL) was introduced via syringe. The mixture was transferred into a 4″ NMR tube, flushed with nitrogen, sealed with a cap and Parafilm, and heated to 50° C. After 20 hours, the reaction was removed from the oil bath and allowed to cool to room temperature. The reaction mixture was then concentrated under reduced pressure. A solution of 20% trifluoroacetic acid in dichloromethane (200 μL) was then added via syringe at room temperature. After 1 hour, the reaction was concentrated under reduced pressure. The resulting residue was purified by preparatory high-performance liquid chromatography (HPLC) using a C₁₈ reverse phase column (250×21.2 mm, 5 μm particle size, 20 mL/min flow rate, eluent: H₂O+0.1% TFA (2 min), gradient 0→100% MeCN/H₂O+0.1% TFA (18 min), t_{R}=14.0 min). Fractions containing the desired compound were combined, and the solvent was removed under reduced pressure at 0° C. with a rotary evaporator to provide enamine N-oxide 37 (9.40 mg, 96%) as a white solid. ¹H NMR (500 MHz, CD₃OD, 25° C.): δ 9.14 (d, J=7.9 Hz, 1H), 7.90 (d, J=8.6 Hz, 1H), 7.46 (td, J=7.5, 2.1 Hz, 2H), 7.29 (t, J=7.5 Hz, 1H), 7.18-7.00 (m, 4H), 6.80 (dt, J=13.5, 4.2 Hz, 1H), 5.98 (dd, J=9.5, 2.5 Hz, 1H), 5.27 (d, J=16.3 Hz, 1H), 5.10-4.90 (m, 3H), 4.20-4.10 (m, 2H), 4.09 (s, 1H), 4.04-3.91 (m, 2H), 3.94-3.85 (m, 1H), 3.14 (dt, J=13.1, 9.0 Hz, 1H), 2.73 (s, 3H), 2.61 (s, 3H), 1.88 (s, 4H), 1.62 (t, J=7.1 Hz, 3H), 1.52 (t, J=7.0 Hz, 3H). ¹³C NMR (125.8 MHz, CD₃OD, 25° C.): δ 169.8, 152.2, 139.4, 137.8, 134.5, 132.7, 131.8, 128.9, 128.0, 127.1, 126.8, 126.4, 125.1, 123.9, 122.5, 122.4, 121.2, 117.6, 116.1, 115.0, 113.5, 110.4, 94.5, 82.0, 81.6, 65.7, 63.3, 60.4, 55.7, 50.4, 31.4, 28.9, 28.8, 8.7, 8.6. FTIR (thin film) cm⁻¹: 1785 (m), 1681 (s), 1349 (m), 1282 (m), 1200 (s), 1133 (s), 801 (w). HRMS (ESI) (m/z): calc'd for C₃₆H₄₀N₅O₆ [M+H]⁺: 638.2973, found: 638.2964. TLC (50% CMA in chloroform), Rf: 0.20 (UV).

### Example 67: Synthesis of (E)-N,N-diethyl-3-hydroxynon-1-en-8-yn-1-amine oxide (38)

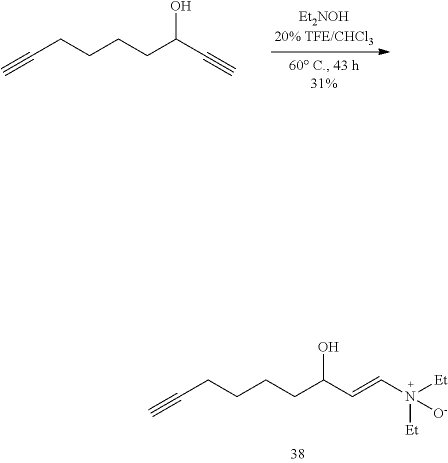

A glass 1-dram vial was charged with nona-1,8-diyn-3-ol (Feldman et al., Heterocycles 81:117-143 (2010)) (50.0 mg, 367 μmol, 1 equiv) and dissolved in 20% v/v trifluoroethanol in chloroform (1.84 mL) at room temperature. N,N-diethylhydroxylamine (189 μL, 1.84 mmol, 5.00 equiv) was then added via syringe. The vial was flushed with nitrogen, sealed with a septum cap and Parafilm, and heated to 60° C. After 43 hours, the reaction was removed from heat, allowed to cool to room temperature, and directly loaded onto a silica gel column. The reaction mixture was purified by flash column chromatography (eluent: 50% CMA in chloroform). Fractions containing the desired compound were combined, and the solvent was removed under reduced pressure at 0° C. with a rotary evaporator to provide enamine N-oxide 38 (25.5 mg, 31%) as a white solid. ¹H NMR (500 MHz, CD₃OD, 25° C.): δ 6.39 (dd, J=13.1, 5.7, 1H), 6.14 (dd, J=13.1, 1.5, 1H), 4.27 (q, J=5.8, 1H), 3.42-3.31 (m, 4H), 2.22-2.14 (m, 3H), 1.63-1.47 (m, 6H), 1.27 (td, J=7.1, 4.6, 6H). ¹³C NMR (126 MHz, CD₃OD, 25° C.): δ 138.3, 133.8, 70.0, 69.8, 65.3, 65.3, 38.0, 29.7, 25.7, 19.1, 8.8. FTIR (thin film) cm⁻¹: 3227 (br-s), 2940 (s), 2862 (s), 1591 (w), 1461 (m), 965 (m). HRMS (ESI) (m/z): calc'd for C₁₃H₂₄NO₂ [M+H]⁺: 226.1802, found: 226.1799. TLC (50% CMA in chloroform), Rf: 0.10 (KMnO₄).

### Example 67: Synthesis of 3-fluoronona-1,8-diyne (S26)

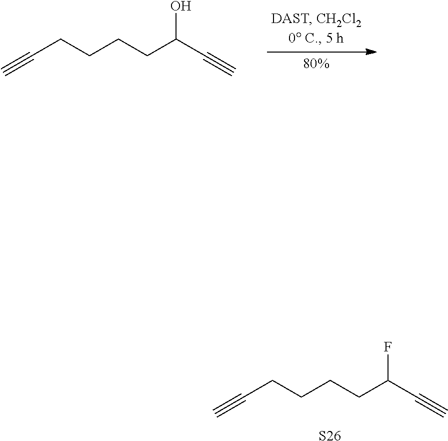

A round-bottom flask was charged with nona-1,8-diyn-3-ol (Feldman et al., Heterocycles 81: 117-143 (2010)) (100 mg, 734 μmol, 1 equiv) at room temperature. Dichloromethane (8 mL) was introduced into the flask and the solution was cooled to 0° C. in an ice-water bath. After 5 minutes, diethylaminosulfur trifluoride (107 μmol, 808 μmol, 1.10 equiv) was added slowly dropwise via syringe. After 5 hours, the reaction mixture was loaded directly onto a silica gel column. The crude mixture was purified by flash chromatography (eluent: 1% DCM/pentane) to provide fluoroalkyne S26 (81.2 mg, 80%) as a clear, colorless oil. ¹H NMR (500 MHz, CDCl₃, 25° C.): δ 5.06 (dtd, J=48.2, 6.3, 2.1, 1H), 2.65 (dd, J=5.6, 2.1, 1H), 2.19 (td, J=6.6, 2.6, 2H), 1.92 (t, J=2.7, 1H), 1.91-1.70 (m, 2H), 1.64-1.49 (m, 4H). ¹³C NMR (126 MHz, CDCl₃, 25° C.): δ 84.1, 82.4 (d, J=167.8 Hz), 80.4 (d, J=25.9 Hz), 76.5 (d, J=10.5 Hz), 68.8, 35.4 (d, J=22.2 Hz), 28.1, 23.71 (d, J=3.9 Hz), 18.4. ¹⁹F NMR (471 MHz, CDCl₃, 25° C.): δ 175.2. FTIR (thin film) cm⁻¹: 3298 (s), 2926 (s), 2855 (s), 1729 (m), 984 (m). HRMS (ACPI) (m/z): calc'd for C₉H₁₂F [M+H]⁺: 139.0918, found: 139.0918. TLC (100% hexanes), Rf: 0.10 (KMnO₄).

### Example 68: Synthesis of (E)-N,N-diethyl-3-fluoronon-1-en-8-yn-1-amine oxide (39)

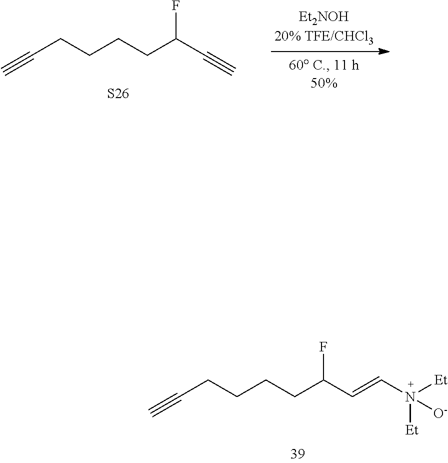

A glass 1-dram vial was charged with 3-fluoronona-1,8-diyne (S26, 46.7 mg, 338 μmol, 1 equiv) and dissolved in 20% v/v trifluoroethanol in chloroform (1.69 mL) at room temperature. N,N-diethylhydroxylamine (174 μL, 1.69 mmol, 5.00 equiv) was then added via syringe. The vial was flushed with nitrogen, sealed with a septum cap and Parafilm, and heated to 60° C. After 11 hours, the reaction was removed from heat, allowed to cool to room temperature, and directly loaded onto a silica gel column. The reaction mixture was purified by flash column chromatography (eluent: 40% CMA in chloroform). Fractions containing the desired compound were combined, and the solvent was removed under reduced pressure at 0° C. with a rotary evaporator to provide enamine N-oxide 39 (38.2 mg, 50%) as a white solid. ¹H NMR (500 MHz, CD₃OD, 25° C.): δ 6.50 (ddd, J=18.3, 13.1, 5.3, 1H), 6.31 (dt, J=13.1, 1.8, 1H), 5.26-5.10 (m, 1H), 3.45-3.33 (m, 4H), 2.26-2.16 (m, 3H), 1.84-1.70 (m, 2H), 1.63-1.51 (m, 4H), 1.27 (td, J=7.1, 5.7, 6H). ¹³C NMR (126 MHz, CD₃OD, 25° C.): δ 139.7 (d, J=12.5 Hz), 129.7 (d, J=19.0 Hz), 91.4 (d, J=170.7 Hz), 84.8, 69.9, 65.4 (d, J=9.3 Hz), 36.0 (d, J=21.8 Hz), 29.4, 24.9 (d, J=4.7 Hz), 19.1, 8.7. ¹⁹F NMR (471 MHz, CDCl₃, 25° C.): δ 181.3. FTIR (thin film) cm⁻¹: 3298 (s), 2944 (s), 2866 (m), 1684 (w), 1461 (m), 1375 (m), 965 (s). HRMS (ESI) (m/z): calc'd for C₁₃H₂₃FNO [M+H]⁺: 228.1758, found: 228.1754. TLC (50% CMA in chloroform), Rf: 0.20 (KMnO₄).

### Example 69: Synthesis of 3-chloronona-1,8-diyne (S27)

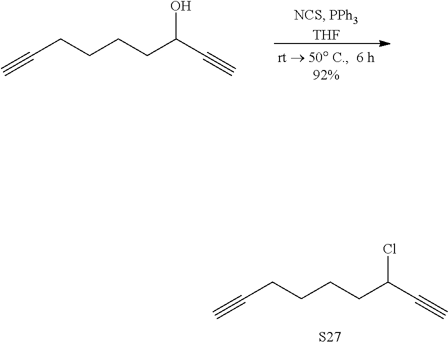

A round-bottom flask was charged with nona-1,8-diyn-3-ol ((Feldman et al., Heterocycles 81: 117-143 (2010))) (40.0 mg, 294 μmol, 1 equiv) and dissolved into tetrahydrofuran (3 mL) at room temperature. Solid N-chlorosuccinimide (59.0 mg, 441 μmol, 1.50 equiv) and triphenylphosphine (93.0 mg, 353 μmol, 1.20 equiv) were then added together in one portion. After 4 hours, the reaction mixture was heated to 50° C. for an additional 2 hours. The reaction was then removed from the oil bath and allowed to cool to room temperature. Ethyl acetate (10 mL) was added and the organic layer was washed with an aqueous solution of 1:1 saturated sodium thiosulfate and saturated sodium bicarbonate (2×5 mL). The organic layer was then dried with anhydrous sodium sulfate, filtered, and concentrated under reduced pressure. The crude mixture was purified by flash column chromatography on silica gel (eluent: 30% dichloromethane in hexanes) to provide alkyne S27 (41.8 mg, 92%) as a clear, colorless oil. ¹H NMR (500 MHz, CDCl₃, 25° C.): δ 4.49 (td, J=6.7, 2.3, 1H), 2.58 (d, J=2.3, 1H), 2.19 (td, J=6.9, 2.7, 2H), 1.99-1.90 (m, 3H), 1.70-1.48 (m, 4H). ¹³C NMR (126 MHz, CDCl₃, 25° C.): δ 84.1, 82.0, 74.6, 68.9, 47.9, 38.6, 27.9, 25.4, 18.5. FTIR (thin film) cm⁻¹: 3298 (s), 2922 (s), 2847 (m) 1438 (m) 1118 (m). HRMS (APCI) (m/z): calc'd for C₉H₁₂Cl [M+H]⁺: 155.0622, found: 155.0621. TLC (60% dichloromethane in hexanes), Rf: 0.85 (KMnO₄).

### Example 70: Synthesis of (E)-N,N-diethyl-3-fluoronon-1-en-8-yn-1-amine oxide (40)

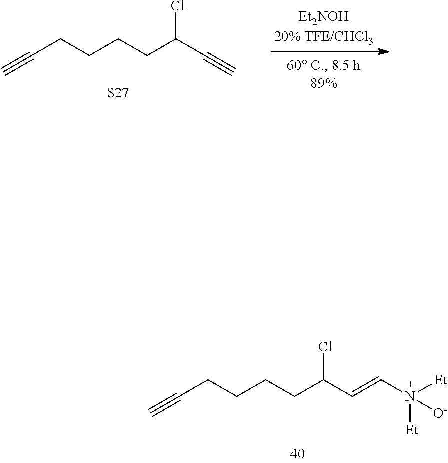

A glass 1-dram vial was charged with 3-chloronona-1,8-diyne (S27, 50.0 mg, 323 μmol, 1 equiv) and dissolved in 20% v/v trifluoroethanol in chloroform (3.23 mL) at room temperature. N,N-diethylhydroxylamine (166 μL, 1.62 mmol, 5.00 equiv) was then added via syringe. The vial was flushed with nitrogen, sealed with a septum cap and Parafilm, and heated to 60° C. After 8.5 hours, the reaction was removed from heat, allowed to cool to room temperature, and directly loaded onto a silica gel column. The reaction mixture was purified by flash column chromatography (eluent: 30% CMA in chloroform). Fractions containing the desired compound were combined, and the solvent was removed under reduced pressure at 0° C. with a rotary evaporator to provide enamine N-oxide 40 (70.0 mg, 89%) as a white solid. ¹H NMR (500 MHz, CD₃OD, 25° C.): δ 6.52 (dd, J=13.0, 8.7, 1H), 6.37 (dd, J=13.0, 0.8, 1H), 4.65 (dt, J=8.4, 6.7, 1H), 3.46-3.32 (m, 4H), 2.23-2.17 (m, 3H), 1.94-1.86 (m, 2H), 1.62-1.51 (m, 4H), 1.27 (td, J=7.1, 5.9, 6H). ¹³C NMR (126 MHz, CD₃OD, 25° C.): δ 140.4, 131.7, 84.8, 70.0, 65.5, 59.0, 39.2, 29.1, 26.7, 19.0, 8.8, 8.7. FTIR (thin film) cm⁻¹: 3298 (s), 2944 (s), 2866 (m), 1681 (w), 1461 (m), 1375 (m), 969 (s). HRMS (ESI) (m/z): calc'd for C₁₃H₂₃ClNO [M+H]⁺: 244.1463, found: 244.1459. TLC (30% CMA in chloroform), Rf: 0.25 (KMnO₄).

[symbol]

### Example 71: Synthesis of tert-Butyl (6-hydroxyhexyl)(prop-2-yn-1-yl)carbamate (S28)

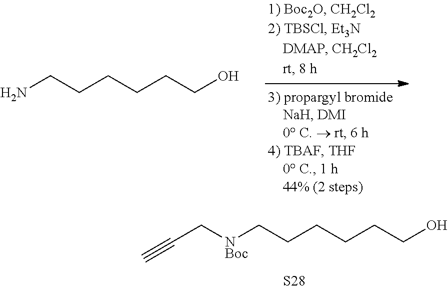 [SMILES: NCCCCCCO ; C#CCN(CCCCCCO)C(=O)OC(C)(C)C ; "S28"]

A round-bottom flask was charged with 6-amino-1-hexanol (2.00 g, 17.1 mmol, 1 equiv) and dissolved in dichloromethane (100 mL) at room temperature. Di-tert-butyl dicarbonate (4.31 mL, 18.8 mmol, 1.10 equiv) was then added via syringe. After 14 hours, the reaction mixture was concentrated under reduced pressure. The crude residue was used without further purification and dissolved in dichloromethane (100 mL) at room temperature. Pyridine (4.15 mL, 51.3 mmol, 3.00 equiv), tert-butyldimethylsilyl chloride (2.84 μg, 18.8 μmmol, 1.10 equiv), and 4-(dimethylamino)pyridine (0.209 g, 1.71 mmol, 0.100 equiv) were then sequentially added to the flask. After 8 hours, an aqueous solution of saturated ammonium chloride (30 mL) was added. The organic phase was further washed with water (30 mL) and brine (30 mL), dried over anhydrous sodium sulfate, filtered, and concentrated under reduced pressure. The crude residue was used without further purification and dissolved in dimethylformamide (100 mL) at room temperature. The reaction mixture was cooled to 0° C. with an ice-water bath, and sodium hydride (1.23 g, 51.3 mmol, 3.00 equiv) was added in one portion. After 30 min, propargyl bromide (2.86 mL, 25.7 mmol, 1.50 equiv) was added via syringe dropwise. The ice-water bath was removed, and the reaction was warmed to room temperature. After 6 h, an aqueous solution of saturated ammonium chloride (10 mL), ethyl acetate (200 mL), and water (200 mL) were added sequentially. The aqueous phase was washed with ethyl acetate (2×100 mL), dried over anhydrous sodium sulfate, filtered, concentrated under reduced pressure, and vacuum dried. The crude residue was used without further purification and dissolved in tetrahydrofuran (100 mL) at room temperature. The reaction mixture was cooled to 0° C. with an ice-water bath and tetrabutylammonium fluoride in tetrahydrofuran (1.00 M, 17.1 mL, 17.1 mmol, 1.00 equiv) was added dropwise. After 1 hour, the reaction mixture was concentrated under reduced pressure, and the crude mixture was purified by [symbol] flash column chromatography on silica gel (20% ethyl acetate in hexanes) to provide alkyne S28 (1.93 g, 44%) as a clear, colorless oil. ¹H NMR (500 MHz, DMSO-d6, 75° C.): δ 3.98 (t, J=3.3 Hz, 3H), 3.40 (d, J=6.7 Hz, 2H), 3.23 (t, J=7.3 Hz, 2H), 3.07 (s, 1H), 1.53 (q, J=6.9 Hz, 2H), 1.47-1.35 (m, 11H), 1.36-1.25 (m, 4H). ¹³C NMR (125.8 MHz DMSO-d6, 75° C.): δ 153.9, 80.0, 78.7, 72.8, 60.4, 45.9, 35.5, 32.1, 27.7, 27.2, 25.7, 24.8. FTIR (thin film) cm⁻¹: 3298 (s), 2944 (s), 2866 (m), 1681 (w), 1461 (m), 1375 (m), 969 (s). HRMS (ESI) (m/z): calc'd for C₁₄H₂₅NNaO₃ [M+Na]⁺: 278.1727, found: 278.1724. TLC (20% ethyl acetate in hexanes), Rf: 0.20 (KMnO₄).

### Example 73: Synthesis of tert-Butyl (6-hydroxyoct-7-yn-1-yl)(prop-2-yn-1-yl)carbamate (S29)

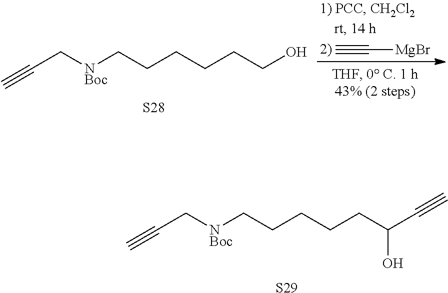

A round-bottom flask was charged with alcohol S28 (187 mg, 730 μmol, 1 equiv) and dissolved in dichloromethane (7 mL) at room temperature. Pyridinium chlorochromate (0.173 g, 803 μmol, 1.10 equiv) was added in one portion and the flask was stirred at room temperature. After 14 hours, silica gel (1.00 g) was added and the reaction mixture was stirred for 10 minutes. The slurry was then loaded directly onto a short silica gel plug and the crude product was eluted with diethyl ether (100 mL). The elution was concentrated under reduced pressure. The crude residue was then dissolved in tetrahydrofuran (7 mL) and cooled to 0° C. using an ice-water bath. A solution of ethynyl magnesium bromide in tetrahydrofuran (500 mM, 1.46 mL, 730 μmol, 1.00 equiv) was then added dropwise via syringe. After 1 hour, saturated aqueous ammonium chloride solution (10 mL) and ethyl acetate (50 mL) were added sequentially. The organic layer was washed with water (20 mL), dried over anhydrous sodium sulfate, filtered, and concentrated under reduced pressure. The resulting residue was purified by flash column chromatography on silica gel (eluent: 20% ethyl acetate in hexanes) to yield the product S29 (88.0 mg, 43%) as a clear, colorless oil. ¹H NMR (500 MHz, DMSO-d6, 75° C.): δ 5.30-5.24 (m, 1H), 4.16 (qd, J=6.6, 2.1 Hz, 1H), 3.97 (s, 2H), 3.23-2.99 (m, 3H), 1.63-1.44 (m, 4H), 1.42-1.33 (m, 11H), 1.28-1.16 (m, 2H). ¹³C NMR (126 MHz, MeOD, 45° C.): δ 155.5, 85.0, 80.2, 79.3, 72.0, 71.2, 61.2, 46.5, 37.5, 35.8, 27.6, 27.4, 26.1, 24.6. FTIR (thin film) cm⁻¹: 3298 (s), 2944 (s), 2866 (m), 1681 (w), 1461 (m), 1375 (m), 969 [symbol] (s). HRMS (ESI) (m/z): calc'd for C₁₆H₂₅NNaO₃ [M+H]⁺: 302.1727, found: 302.1723. TLC (20% ethyl acetate in hexanes), Rf: 0.20 (KMnO₄).

### Example 74: Synthesis of tert-Butyl (6-fluorooct-7-yn-1-yl)(prop-2-yn-1-yl)carbamate

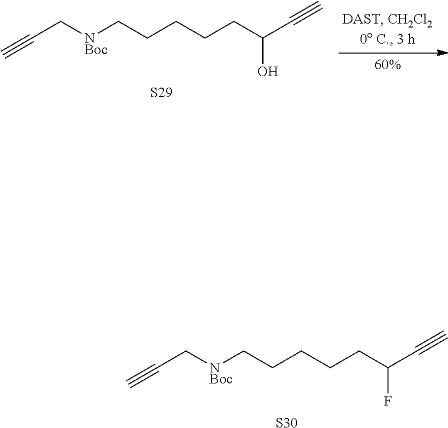

A round-bottom flask was charged with (510 mg, 1.83 mmol, 1 equiv), dissolved in dichloromethane (18 mL), and cooled to 0° C. using an ice-water bath. Diethylaminosulfur trifloride (253 μL, 1.92 mmol, 1.20 equiv) was added dropwise via syringe. After 3 hours, the reaction mixture was concentrated under reduced pressure. The resulting residue was purified by flash column chromatography on silica gel (eluent: 5% ethyl acetate in hexanes) to provide fluoroalkyne S30 (412 mg, 60%) as a clear, colorless oil. ¹H NMR (500 MHz, DMSO-d6, 75° C.): δ 5.40-5.04 (m, 1H), 3.99 (d, J=2.5 Hz, 2H), 3.74-3.45 (m, 1H), 3.30-3.13 (m, 2H), 2.99-2.78 (m, 1H), 1.86-1.72 (m, 2H), 1.59-1.30 (m, 15H). ¹³C NMR (126 MHz, DMSO-d6, 75° C.): δ 153.9, 81.8 (d, J=165.0 Hz), 80.2 (d, J=25.1 Hz), 80.0, 78.7, 78.4, 72.7, 45.7, 35.5, 34.8 (d, J=22.0 Hz), 27.6, 26.9, 25.2, 23.1 (d, J=4.1 Hz). ¹⁹F NMR (471 MHz, DMSO-d6, 75° C.): δ −173.6. FTIR (thin film) cm⁻¹: 3298 (s), 2944 (s), 2866 (m), 1681 (w), 1461 (m), 1375 (m), 969 (s). HRMS (ESI) (m/z): calc'd for C₁₆H₂₄FNNaO₂ [M+H]⁺: 304.1683, found: 304.1681. TLC (10% ethyl acetate in hexanes), Rf: 0.50 (KMnO₄).

[symbol]

### Example 75: Synthesis of Si700-fluoroalkyne (S31)

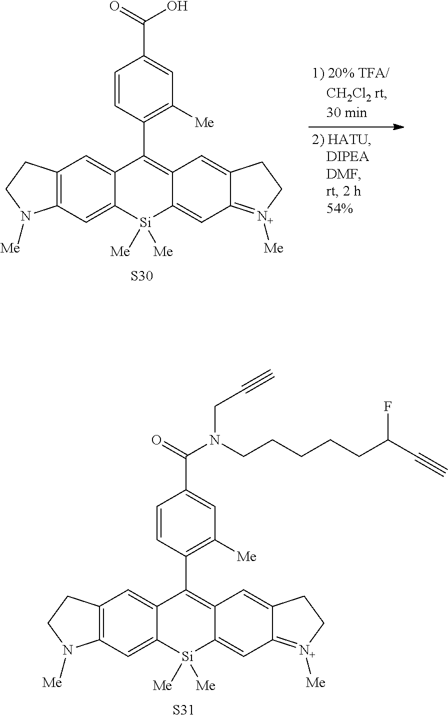 [SMILES: Cc1cc(C(=O)O)ccc1C1=c2cc3c(cc2[Si](C)(C)c2cc4c(cc21)CCN4C)=[N+](C)CC3 ; "S30" ; C#CCN(CCCCCC(F)C#C)C(=O)c1ccc(C2=c3cc4c(cc3[Si](C)(C)c3cc5c(cc32)CCN5C)=[N+](C)CC4)c(C)c1 ; "S31"]

A round-bottom flask was charged with tert-butyl (6-fluorooct-7-yn-1-yl)(prop-2-yn-1-yl)carbamate (S30, 14.5 mg, 51.5 μmol, 1 equiv) and dissolved in a mixture of 20% trifluoroacetic acid in dichloromethane (2 mL) at room temperature. After 30 minutes, the mixture was concentrated under reduced pressure. The crude mixture was then dissolved in dimethylformamide (2 mL) at room temperature and N,N-diisopropylethylamine (35.2 μL, 206 μmol, 4.00 equiv.) was added via syringe. After 5 minutes, Si700-acid (24.1 mg, 51.5 μmol, 1.00 equiv) and HATU (23.5 mg, 61.8 μmol, 1.20 equiv) were added successively and allowed to stir at room temperature in the dark. After 2 hours, the reaction mixture was diluted with dichloromethane (100 mL). The organic layer was washed with an aqueous solution of saturated sodium bicarbonate (2×20 mL), dried with anhydrous sodium sulfate, filtered, and concentrated under reduced pressure. The crude residue was purified by preparatory high-performance liquid chromatography (HPLC) using a C₁₈ reverse phase column (250×21.2 mm, 5 μm particle size, 20 mL/min flow rate, eluent: 40% MeCN/H₂O+0.1% TFA (2 min), gradient 40→100% MeCN/H₂O+0.1% TFA (18 min), t_{R}=15.0 min) to provide fluoroalkyne S31 (17.4 mg, 54%) as a blue solid. ¹H NMR (500 MHz, CD3CN, 65° C.): δ 7.49-7.38 (m, 2H), 7.16 (d, J=7.7 Hz, 1H), 7.07 (s, 2H), 6.70 (s, 2H), 5.14 (dd, J=47.6, 6.8 Hz, 1H), 4.27 (s, 2H), 3.81 (t, J=8.0 Hz, 4H), 3.54 (s, 2H), 3.19 (s, 6H), 3.04-2.84 (m, 5H), 2.62-2.53 (m, 1H), 2.08 (s, 3H), 1.89-1.63 (m, 4H), 1.58-1.27 (m, 4H), 0.57 (d, J=5.8 Hz, 6H). ¹³C NMR (126 MHz, CD3CN, 65° C.): δ 171.9, 165.8, 158.5, 151.8, 142.4, 138.1, 137.7, 134.8, 133.6, 130.3, 129.7, 129.5, 125.3, 116.1, 83.8 (d, J=165.2 Hz), 81.7 (d, J=25.6 Hz), 80.8, 78.0 (d, J=10.5 Hz), 73.5, 55.8, 36.7 (d, J=22.1 Hz), 34.3, 30.5, 28.6, 27.4, 27.2, 25.0 (d, J=4.2 Hz), 19.7, 12.9, −0.9, −1.0. ¹⁹F NMR (471 MHz, CD3CN, 55° C.): δ −76.6, −175.3. FTIR (thin film) cm⁻¹: 2937 (w), 1684 (m), 1602 (s), 1382 (s), 1274 (s), 1196 (s), 1140 (s), 980 (w), 842 (w), 801 (w), 723 (w). HRMS (ESI) (m/z): calc'd for C₄₀H₄₅FN₃OSi [M]⁺: 630.3310, found: 630.3299.

### Example 76: Synthesis of Si700-enamine N-oxide (42)

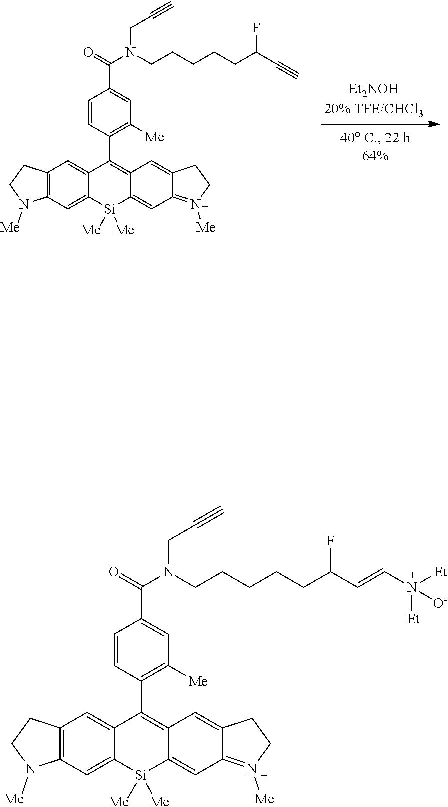

A 4″ NMR tube was charged with a solution of Si700-fluoroalkyne (S31, 2.00 mg, 3.10 μmol, 1 equiv) in 20% v/v trifluoroethanol in chloroform (250 μL) and a solution of N,N-diethylhydroxylamine (2 M, 250 μL, 500 μmol, 161 equiv) in 20% v/v trifluoroethanol in chloroform via syringe. The tube was sealed, flushed with nitrogen, and heated to 40° C. in the dark. After 22 hours, the reaction mixture was concentrated under reduced pressure. The crude residue was purified by preparatory high-performance liquid chromatography (HPLC) using a C₁₈ reverse phase column (250×21.2 mm, 5 μm particle size, 20 mL/min flow rate, eluent: 40% MeCN/H₂O+0.1% TFA (2 min), gradient 40→100% MeCN/H₂O+0.1% TFA (18 min), t_{R}=11.0 min). Fractions containing the desired compound were combined and the solvent was removed under reduced pressure at 0° C. with a rotary evaporator to enamine N-oxide 42 (1.48 mg, 64%) as a blue solid. Structural assignments were made using additional information from gCOSY, HSQC, and HMBC experiments. ¹H NMR (500 MHz, CD₃OD, 45° C.): δ 7.49 (s, 2H), 7.21 (d, J=7.7 Hz, 1H), 7.14 (s, 2H), 6.71-6.52 (m, 3H), 6.43 (d, J=13.4 Hz, 1H), 5.26 (d, J=47.7 Hz, 1H), 4.24 (s, 2H), 3.83 (t, J=7.9 Hz, 8H), 3.67 (s, 2H), 3.23 (s, 6H), 2.97-2.90 (m, 4H), 2.84 (s, 1H), 2.09 (s, 3H), 1.81 (s, 4H), 1.63-1.32 (m, 10H), 0.57 (d, J=11.8 Hz, 6H). ¹³C NMR (126 MHz, CD₃OD, 45° C.): δ 173.3, 165.7, 158.7, 152.2, 143.2, 138.1, 137.7, 135.0, 134.1 (d, J=14.6 Hz), 133.5, 132.9 (d, J=18.9 Hz), 130.7, 129.8, 129.6, 125.4, 116.1, 91.1 (d, J=173.4 Hz), 80.0, 74.8, 65.4 (d, J=6.8 Hz), 55.8, 47.2, 40.9, 36.0 (d, J=21.1 Hz), 33.9, 30.8, 27.7, 27.4, 25.5 (d, J=4.0 Hz), 19.5, 8.4, 8.3, −1.0, −1.4 ¹⁹F NMR (470.5 MHz, CD₃OD, 25° C.): δ −76.9, −184.5. FTIR (thin film) cm⁻¹: 2922 (w), 1673 (s), 1446 (w), 1192 (s), 1133 (s), 846 (m), 801 (m), 723 (m). HRMS (ESI) (m/z): calc'd for C₄₄H₅₆FN₄O₂Si [M+H]⁺: 719.4151, found: 719.4144.

### Example 77: Biological Procedures

### Cell Culture

Cells were cultured in RPMI (A431, Bx-PC3, H460, HeLa, MDA-MB-23) or DMEM (U251) containing 10% FBS (Sigma), 100 units/mL penicillin, and 0.1 mg/mL streptomycin (Sigma) in a humidified chamber at 37° C. under an ambient atmosphere with 5% CO₂ unless otherwise stated. Cells were passaged and dissociated with 0.25% trypsin, 0.1% EDTA in HBSS (Corning). The following cell lines were obtained from other laboratories: U251, HeLa, and A431 (Charles D. Stiles, Dana-Farber Cancer Institute); BxPC3 (Nathanael Gray, Dana-Farber Cancer Institute); and MDA-MB-231 (Jun Qi, Dana-Farber Cancer Institute). All cells tested negative for mycobacteria with the MycoAlert PLUS Mycoplasma Detection Kit (Lonza) following the manufacturer's protocol.

### Staurosporine Enamine N-Oxide Cell Viability Studies

A431 or H460 cells were seeded at a density of 12,000-13,000 cells per well in media [100 μL, RPMI supplemented with 5% heat-inactivated human serum (Sigma), penicillin (100 units/mL), and streptomycin (0.1 mg/mL)] in clear 96-well plates. PBS (100 μL) was added to the edge wells. The cells were incubated at 37° C. under ambient atmosphere with 5% CO₂. After 24 hours, the media was aspirated and replaced with media [50 μL, RPMI supplemented with 5% HS, penicillin (100 units/mL), and streptomycin (0.1 mg/mL)] containing the staurosporine derivative of interest. For A431 cells, staurosporine or staurosporine derivative treatment concentrations started at 50 μM and were serially diluted 4-fold across nine wells. For H460 cells, staurosporine or staurosporine derivative treatment concentrations started at 100 μM and were serially diluted 4-fold across nine wells. Treatment compounds were prepared from a stock solution containing ethanol (10 mM in ethanol; 0.5-1.0% ethanol final concentration), so vehicle control wells contained 1% ethanol. Plates were then incubated at 37° C. under an ambient (20% pO₂) or hypoxic (0.1% pO₂) atmosphere with 5% CO₂ for 48 h. Hypoxic conditions were maintained using a ProOx C21 chamber (Biospherix) in a cell culture incubator. After 48 hours, the media was aspirated, the wells were washed once with PBS (130 μL), and the cell viability was determined using the MTT assay. Thiazolyl blue tetrazolium bromide in FluoroBrite DMEM (100 μL, 0.5 mg/mL, Gibco) was added to each well, and the plates were incubated for 4 hours at 37° C. under ambient atmosphere with 5% CO₂. After 4 hours, the media was partially removed (75 μL) and replaced with DMSO (50 μL). Plates were incubated for 10 minutes on a shaker at room temperature. The absorbance at 570 nm was measured using a microplate reader (Clariostar Plus, BMG Labtech).

### AQ₄N Cell Viability Studies

These studies were performed as previously reported (Manley et al., J. Pharmacol. Exp. Ther. 344:368-377 (2013)). H460 or A431 cells were seeded at a density of 5000 cells per well in media [100 μL, RPMI supplemented with 5% FBS, penicillin (100 units/mL), and streptomycin (0.1 mg/mL)] in clear 96-well plates. PBS (100 μL) was added to the edge wells. The cells were incubated at 37° C. under ambient atmosphere with 5% CO₂. After 24 hours, the media was aspirated and replaced with media [50 μL, RPMI supplemented with 5% FBS, penicillin (100 units/mL), and streptomycin (0.1 mg/mL)] containing AQ₄N (Sigma). For both cell lines, AQ₄N treatment concentrations started at 200 μM and was serially diluted 4-fold across nine wells. AQ₄N solutions were prepared from a stock solution containing DMSO (20 mM in DMSO; 1% DMSO final concentration), so our vehicle control wells contained 1% DMSO. Plates were then incubated at 37° C. under an ambient (20% pO₂) or hypoxic (0.1% pO₂) atmosphere with 5% CO₂ for 24 hours. Hypoxic conditions were maintained using a hypoxia incubator chamber (StemCell Technologies) in a cell culture incubator. After 24 hours, the media was aspirated and replaced with fresh media [100 μL, RPMI supplemented with 5% FBS, penicillin (100 units/mL), and streptomycin (0.1 mg/mL)]. The plates were returned to 37° C. under an ambient atmosphere with 5% CO₂ for 72 hours. After 72 hours, cell viability was determined using the MTT assay. Thiazolyl blue tetrazolium bromide in FluoroBrite DMEM (100 μL, 0.5 mg/mL, Gibco) was added to each well, and the plates were incubated for 4 hours at 37° C. under an ambient atmosphere with 5% CO₂. After 4 hours, the media was partially removed (75 μL) and replaced with DMSO (50 μL). Plates were incubated for 10 minutes on a shaker at room temperature. The absorbance at 570 nm was then measured using a microplate reader (Clariostar Plus, BMG Labtech).

### Staurosporine Release by A431 Cells

Sterilized 2 mL HPLC vials were each charged with a suspension of 7.5×10⁶ cells in RPMI (1 mL) and sealed with a cap containing a septum. The vials were charged with only RPMI (1 mL) in the no cell controls. A stock solution of staurosporine enamine N-oxide 37 (10 μL, 10 mM in ethanol; 100 μM final concentration) was then added to each vial. Septa were pierced with an open 22-gauge needle for normoxic conditions. Hypoxic conditions were maintained using nitrogen lines connected to a vacuum gas manifold. The vials were maintained at 37° C. in a water bath and shaken every 15 minutes. At different time points (0, 1, 2, 4, and 8 hours), an aliquot of the cell suspension (100 μL) was removed from each vial and immediately added to acetonitrile (100 μL) containing p-nitrophenol (10 μM) as an internal standard. Quenched samples were then cooled to −20° C. for 1 hour and centrifuged at 10,000×g for 5 minutes at 4° C. The supernatant was collected and analyzed by HPLC (C₁₈ column, 4.6×250 mm, 5 μm particle size, 1 mL/minute flow rate, eluent: gradient 0→20% MeCN/H₂O+0.1% TFA (1 minute), gradient 20→40% MeCN/H₂O+0.1% TFA (7 minutes), gradient 40→100% MeCN/H₂O+0.1% TFA (3 minutes)). Staurosporine was quantified using its absorbance at 280 nm.

### In Vitro Microsomal Reduction Assay

All reactions were prepared and carried out in a semi-micro spectrophotometer quartz cell fitted with a septum screw cap (Starna Cells). For anaerobic conditions, all solutions were degassed for 30 minutes by sparging with nitrogen. Cells were maintained under anaerobic conditions using nitrogen lines connected to a nitrogen gas manifold or using a nitrogen-filled balloon. In experiments requiring normoxic conditions, the quartz cells were left open to ambient atmosphere. Unless otherwise stated, reactions were prepared as follows. A solution of human liver microsomes (10 μL, 20 mg/mL in phosphate buffer, pH 7.4, Corning; 200 μg/mL final concentration) followed by a solution of NADPH (16.7 μL, 60 mM in 10 mM NaOH solution; 1 mM final concentration) was added to a phosphate buffered solution (928.3 μL, 100 mM phosphate buffer, pH 7.4; 1 mL final volume) at room temperature. After 1 hour, reactions were initiated by the addition of a stock solution of the 2-nitroaniline enamine N-oxide probes 32a-f (50 μL, 4 mM in phosphate buffer, pH 7.4; 200 μM final concentration). The absorbance of the solution at 430 nm was immediately blanked then recorded at time points of 0 and 2 hours on the UV-vis spectrophotometer (Cary 60, Agilent).

Heat-inactivated microsomes were prepared for control experiments by heating a solution of human liver microsomes (20 mg/mL in phosphate buffer, pH 7.4, Corning) to 45° C. for 30 minutes.

For studies involving CYP450 inhibitors, a solution of a CYP450 inhibitor (10 μL, 20 mM in DMSO; 200 μM final concentration) was added to the solution of NADPH and human liver microsomes under anaerobic conditions 30 minutes prior to the addition of the 2-nitroaniline enamine N-oxide probes.

To determine the initial rates of microsomal reduction, the absorbance at 430 nm was measured every 0.1 or 0.5 seconds over the first 5 minutes of the reaction at room temperature.

Enamine N-Oxides with Reducing Agents

A stock solution of 2-nitroaniline enamine N-oxide probe 32c (50 μL, 4 mM in 100 mM phosphate buffer, pH 7.4; 200 μM final concentration) was added to a phosphate buffered solution (850 μL, 100 mM phosphate, pH 7.4; 1 mL final volume) in a semi-micro quartz cell open to ambient atmosphere. A solution of glutathione (100 μL, 50 mM in phosphate buffer, pH 7.4; 5 mM final concentration) or cysteine (100 μL, 50 mM in phosphate buffer, pH 7.4; 5 mM final concentration) or tetrahydroxydiboron (100 μL, 20 mM in methanol; 200 μM final concentration) were added to the solution. Reactions with glutathione or cysteine were incubated at 37° C. The reaction with tetrahydroxydiboron was carried out at room temperature. Wavelength scans (400-550 nm) were recorded at several time points (0, 1, and 2 hours).

### Assay Evaluating the Importance of Enamine N-Oxide Unsaturation

Enamine N-oxide 6 or alkyl N-oxide S4 (50 μL, 4 mM in water; 200 μM final concentration) were added to a HEPES buffered solution (750 μL, 100 mM HEPES, pH 7.4; 1 mL final volume) in an HPLC vial. N-oxide reduction was initiated by adding a solution of tetrahydroxydiboron (200 μL, 200 nmol, 1 equiv, 1 mM in ethanol) and the resulting solution was incubated at room temperature. After 1 hour, solutions were analyzed by HPLC (C₁₈ column, 4.6×50 mm, 2.4 μm particle size, 1 mL/minute flow rate, eluent: gradient H₂O+0.1% TFA (1 minute), gradient 0→100% MeCN/H₂O+0.1% TFA (4 minutes), MeCN+0.1% TFA (1 minute)). p-Cresol was quantified by its absorbance at 280 nm.

### Assay Evaluating the Stability of Enamine N-Oxides to Metal Cations

Enamine N-oxide 3a (50 μL, 4 mM in water; 200 μM final concentration) was added to a HEPES buffered solution (946 μL, 100 mM HEPES, pH 7.4; 1 mL final volume) in an HPLC vial. A solution of metal cation (4 μL, 500 mM in water, 2 mM final concentration) was then added and the solution was incubated at room temperature. After 1 hour, solutions were analyzed by HPLC (C₁₈ column, 4.6×50 mm, 2.4 μm particle size, 1 mL/minute flow rate, eluent: gradient H₂O+0.1% TFA (1 minute), gradient 0→100% MeCN/H₂O+0.1% TFA (4 minutes), MeCN+0.1% TFA (1 minute)). p-Cresol was quantified by its absorbance of 280 nm.

The following cations were assayed using this procedure: sodium chloride, potassium chloride, calcium chloride, magnesium chloride, manganese (II) chloride, cobalt (II) sulfate, zinc (II) chloride, nickel (II) acetate, and iron (III) chloride.

For iron (II) sulfate, a solution of iron (II) sulfate (2 μL, 1 M in 1 N HCl; 2 mM final concentration) was added followed by a solution of sodium dithionite (10 μL, 1 M in 1 N NaOH; 10 mM final concentration).

### Western Blot of Pimonidazole-Labeled Cells

Cells were seeded at a density of 200,000 cells per well in media [2 mL, RPMI supplemented with 5% heat-inactivated human serum (Sigma), penicillin (100 units/mL), and streptomycin (0.1 mg/mL)] in 6-well plates. Cells were incubated at 37° C. under ambient atmosphere with 5% CO₂. After 24 hours, the media was aspirated and replaced with media [1 mL, RPMI supplemented with 5% heat-inactivated HS, penicillin (100 units/mL), and streptomycin (0.1 mg/mL)] containing pimonidazole (1 μL, 10 mM in water; 10 μM final concentration). Plates were then incubated at 37° C. under ambient (20% pO₂) or oxygenated conditions (5%, 1%, or 0.1% pO₂) with 5% CO₂ for 48 hours. The oxygenated conditions were maintained using a hypoxia chamber (ProOx C21, Biospherix) in a cell culture incubator. After 48 hours, the media was aspirated, and the cells were washed with PBS (1 mL). PBS (1 mL) was then added to each well and the cells were scraped off with a cell scraper. The cell suspensions were placed into microcentrifuge tubes and pelleted by spinning at 10,000×g for 5 min at 4° C. The supernatant was then removed, and the pellet was dissolved with an SDS solution (100 μL, 1% SDS in 50 mM Tris buffer, pH 8.0). The protein concentration was then determined by BCA assay following the manufacturer's protocol (Pierce). The sample was then subject to chloroform/methanol extraction. Methanol (400 μL), chloroform (100 μL), and water (300 μL) were sequentially added to an aliquot of the cell lysate (100 μg) with vigorous mixing using a vortex after the addition of each solution. The mixture was centrifuged for 5 min at 10,000×g at 4° C. The top layer was removed, methanol (400 μL) was added, and the solution was vortexed then centrifuged. These steps were repeated twice. After the final removal of the supernatant, the pellet was left to air dry in the dark for 20 minutes. The dried pellet was then resuspended in an SDS solution (50 μL, 1% SDS in 50 mM Tris, pH 8.0), and the protein concentration was determined by BCA assay. Each sample (5 μg) was subjected to SDS PAGE, transferred to a 0.2 μm PVDF membrane, blocked with 5% milk in TBST, and Western blotted for pimonidazole (1:1000 in 5% milk in TBST, Hypoxyprobe clone 4.3.11.3) and -actin (1:1000 dilution in 5% milk in TBST, Cell Signaling Technologies, 4970). Detection was mediated by anti-rabbit IR680-dye (LI-COR Biosciences, 925-68071) and anti-mouse IR800-dye (LI-COR Biosciences, 925-32210) conjugated secondary antibodies. Blots were imaged using a fluorescence scanner (Odyssey CLx, LI-COR Biosciences) and quantified by ImageJ.

### In-Gel Fluorescence Imaging of Cells Labeled by Enamine N-Oxides

Cells were seeded at a density of 200,000 cells per well in media [2 mL, RPMI (A431, H460, HeLa, Bx-PC3, MDA-MB-231) or DMEM (U251) supplemented with 5% heat-inactivated human serum (Sigma), penicillin (100 units/mL), and streptomycin (0.1 mg/mL)] in 6-well plates. Cells were incubated at 37° C. under ambient atmosphere with 5% CO₂. After 24 h, the media was aspirated and replaced with fresh media [1 mL, RPMI (A431, H460, HeLa, Bx-PC3, MDA-MB-231) or DMEM (U251) supplemented with 5% heat-inactivated human serum, penicillin (100 units/mL), and streptomycin (0.1 mg/mL)] containing enamine N-oxide probe (200 mM in ethanol; 10 μM final concentration). Plates were then incubated at 37° C. under ambient (20% pO₂) or various oxygenated conditions (5%, 1%, or 0.1% pO₂) with 5% CO₂. Oxygenated conditions were maintained using a hypoxia chamber (ProOx C21, Biospherix) in a cell culture incubator. After 48 hours, the media was aspirated, and cells were washed with PBS (1 mL). PBS (1 mL) was then added to each well and cells were scraped off with a cell scraper. The cell suspensions were placed into microcentrifuge tubes and pelleted by spinning at 10,000×g for 5 minutes at 4° C. The supernatant was then removed, and the pellet was dissolved with an SDS solution (100 μL, 1% SDS in 50 mM Tris buffer, pH 8.0). The protein concentration was determined by BCA assay following the manufacturer's protocol (Pierce). The sample was then labeled using the copper-catalyzed azide-alkyne cycloaddition (CuAAC). Lysate (100 μg), TAMRA-azide (0.5 μL, 5 mM TAMRA-azide in DMSO; 25 μM final concentration), CuSO₄ (2 μL, 50 mM in water; 1 mM final concentration), tris(3-hydroxypropyltriazolylmethyl)amine (THPTA, 0.6 μL, 500 mM in DMSO; 3 mM final concentration), and sodium ascorbate (2 μL, 100 mM in water; 2 mM final concentration) were added to a Tris-buffered solution (50 mM Tris buffer, pH 8.0; final volume 100 μL). Reactions were incubated at room temperature in the dark for 1 hour. The sample was then subject to chloroform/methanol extraction. Methanol (400 μL), chloroform (100 μL), and water (300 μL) were sequentially added to an aliquot of the cell lysate (100 μg) with vigorous mixing using a vortex after the addition of each solution. The mixture was centrifuged for 5 minutes at 10,000×g at 4° C. The top layer was removed, methanol (400 μL) was added, and the solution was vortexed then centrifuged. These steps were repeated twice. After the final removal of the supernatant, the pellet was left to air dry in the dark for 20 minutes. The dried pellet was then resuspended in an SDS solution (50 μL, 1% SDS in 50 mM Tris, pH 8.0), and the protein concentration was determined by BCA assay. Each sample (5 μg) was subjected to SDS PAGE. In-gel fluorescence was visualized by a laser scanner (Typhoon™ FLA 9500, GE) and quantified using ImageJ software. Protein was then transferred to a 0.2 μm PVDF membrane, blocked with 5% milk in TBST, and Western blotted for j-actin (1:1000 dilution in 5% milk in TBST, Cell Signaling Technologies, 4970). Detection was mediated by an anti-rabbit IR680-dye conjugated secondary antibody (LI-COR Biosciences, 925-68071) and imaged using a fluorescence scanner (Odyssey CLx, LI-COR Biosciences).

### Bx-PC3 Tumor Xenograft Model

All animal experiments were performed according to procedures and protocols approved by the Dana-Farber Cancer Institute Institutional Animal Care and Use Committee. Bx-PC3 cells were grown to confluency in antibiotic-free RPMI supplemented with 10% FBS, trypsinized, and washed three times with ice cold PBS (50 mL). Cells were resuspended in PBS at a concentration of 1.0×10⁷ cells per 100 μL. 8-week old, female, homozygous NU/J mice (Jackson Laboratory) were subcutaneously injected with 1.0×10⁷ Bx-PC3 cells over the right hind flank under isoflurane anesthesia. Experiments were performed when tumors reached 1000 mm³ in size.

### Immunofluorescence Tissue Staining

Mice were intraperitoneally injected with a 200 μL bolus of 25.8 mM pimonidazole and enamine N-oxide probe in 0.9% saline. Two min prior to sacrifice, mice were intravenously injected with 15 mg/kg Hoechst 33342 in 100 μL of PBS. After 1 hour, mice were sacrificed by cervical dislocation under isoflurane anesthesia. Tumors were resected and fixed in 4% paraformaldehyde in PBS for 24 hours at 4° C. with shaking. Tumors were then stored in 70% ethanol/water and submitted to the Rodent Histopathology Core at the Dana-Farber/Harvard Cancer Center for paraffin sectioning and H&E staining on glass slides. Paraffin embedded slices on glass slides were first rehydrated by successively immersing in xylenes twice for 10 minutes, 100% ethanol twice for 10 minutes, 95% ethanol/water once for 5 minutes, 70% ethanol/water for 5 minutes, 50% ethanol/water for 5 minutes, water three times for 20 seconds, and ice cold PBS+0.3% Triton X-100 once for 10 minutes. For slices to be stained by HIF1α and CD31, slides were then incubated at 90° C. in 10 mM citrate, pH 6.0 for 15 minutes. After 15 minutes, the solution was removed from heat and allowed to cool to room temperature. All slides were then rinsed with deionized water and ice-cold PBS+0.025% Triton X-100 for 10 minutes. Areas on slides around the tissue were dried with a Kimwipe and a PAP pen was used to enclose the tissue. A solution (150-300 uL) with final concentrations of 100 mM Tris pH 7.6, 10 μM TAMRA-azide, 4 mM CuSO₄, 100 mM ascorbic acid, and 3 mM THPTA was applied to each tissue slice to label the alkyne-containing enamine N-oxide probe. Slides were incubated at room temperature in the dark for 2 hours. After CuAAC, slides were washed twice with ice cold PBS+0.025% Trition X-100 for 10 minutes. Slices were then blocked with 5% normal goat serum (NGS, Fisher) in PBS+0.025% Triton X-100 at room temperature for 1 hour in the dark. After blocking, slides were washed once with ice cold PBS+0.025% Triton X-100 for 10 minutes. Primary antibodies were then applied at 4° C. for 16 hours in 5% NGS in PBS+0.025% Triton X-100. Dilutions of anti-HIF1α (1:100, Novus Biological, NB100-134), anti-CD31 (1:250, Abcam, ab28364), anti-carbonic anhydrase IX (CAIX, 1:250, Abcam, ab15086), anti-glucose transporter 1 (GLUT1, 1:250, Abcam, ab652), or FITC-anti-pimonidazole (1:50, Hypoxyprobe) antibodies were used. After 16 hours, slides were washed twice with ice cold PBS+0.025% Triton X-100. Slides stained for HIF1α, CD31, CAIX, or GLUT1 were then probed with a solution containing goat anti-rabbit AF488 (1:2000, Abcam, ab150077) in 5% NGS in PBS+0.025% Triton X-100 for 1 hour at room temperature. When applicable, DAPI (10 mg/mL in water; 0.01 mg/mL final concentration) was included. Samples were then washed twice with ice cold PBS+0.025% Triton X-100 and twice with deionized water. Mounting media (20 mM Tris pH 8.0, 0.5% N-propyl gallate, 90% glycerol) was added and slides were fixed with glass coverslips. Slices were then imaged using a widefield fluorescence inverted microscope (Nikon Ti Eclipse) at the Dana-Farber Confocal and Light Microscopy Core according to the settings listed in the “General biological instrumentation” section.

### Near Infrared Imaging in Live Mice

Mice were intraperitoneally injected with a solution of Si700-enamine N-oxide probe 42 (20 mg/kg, 7.93 mM in 0.9% saline). Under isoflurane anesthesia, mice were then imaged with a Xenogen IVIS 100 fluorescence imager using an excitation of 675 nm and emission of 720 nm at various time points (0 hours (before treatment), 6 hours, 24 hours and 30 hours (after treatment)). Mice were then intraperitoneally injected with a solution of Si700-enamine N-oxide probe 42 (20 mg/kg, 7.93 mM in 0.9% saline) 30 hours post injection. After 16 hours, mice were intraperitoneally injected with a bolus of pimonidazole solution (200 μL, 25.8 mM pimonidazole in 0.9% saline). After 1 hour, mice were sacrificed by cervical dislocation under isoflurane anesthesia and tissue slices were stained according to the “Immunofluorescence tissue staining” protocol using anti-pimonidazole mouse IgG₁ monoclonal antibody (1:50, Hypoxyprobe, HP1-100Kit) and goat anti-rabbit AF488 (1:2000, Abcam, ab150077).

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound having a structure represented by formula I or II: 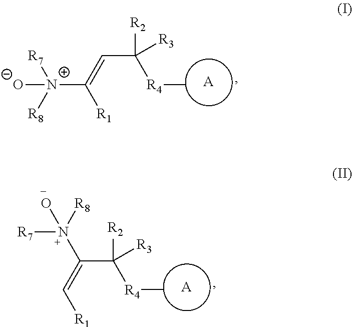
or a pharmaceutically acceptable salt or stereoisomer thereof,
wherein:
R₁ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a leaving group, or -[L]-diagnostic moiety, wherein R₁ may be optionally substituted;
[L] is absent or an optionally substituted linking group that is capable of carrying a plurality of diagnostic moieties, which may be the same or different;
R₂ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a n-electron withdrawing group, or -[L]-diagnostic moiety, wherein R₂ may be optionally substituted;
[L] is absent or a linking group that is capable of carrying a plurality of diagnostic moieties, which may be the same or different;
R₃ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a n-electron withdrawing group, or -[L]-diagnostic moiety, wherein R₃ may be optionally substituted;
[L] is absent or a linking group that is capable of carrying a plurality of diagnostic moieties, which may be the same or different;
R₄ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a leaving group, a cleavable linking group, or -[L]-diagnostic moiety, wherein R₄ may be optionally substituted;
[L] is absent or a linking group that is capable of carrying a plurality of diagnostic moieties, which may be the same or different;
R₇ is (C₁-C₅) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted, or
R₇ and R₈ together with the nitrogen atom to which they are attached, form a 4- to 7-membered heterocyclyl comprising 1 to 3 heteroatoms selected from O, N, and S;
R₈ is (C₁-C₅) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted; and
A is absent or a therapeutic moiety;
provided that the compound of formula (I or II) contains at least one -[L]-diagnostic moiety or therapeutic moiety,
and when A is a therapeutic moiety, R₄ is a cleavable linking group;
and when the compound contains at least one -[L]-diagnostic moiety and A is absent, R₁ and/or R₄ is a leaving group.

2. The compound of claim 1, wherein R₁ is hydrogen, an inductive electron group, a leaving group, or an optionally substituted -[L]-diagnostic moiety.

3. (canceled)

4. The compound of claim 2, wherein R₁ is an inductive electron group, wherein the inductive electron group is OR₅, SR₅, NR₅R₅, or a cyclic or acyclic amide, wherein each R₅ is independently hydrogen, (C₁-C₆) alkyl, (C₃-C₁₀) carbocyclyl, or 4- to 7-membered heterocyclyl, wherein said alkyl, carbocyclyl, or heterocyclyl is optionally substituted, or
R₁ is a leaving group, wherein the leaving group is iodo, bromo, chloro, OR₉, SR₉, —OC(O)R₉, —OC(O)OR₉, —OC(O)NR₉R₉, —OC(S)R₉, —OC(S)OR₉, —OC(S)NR₉R₉, —OS(O)₂R₉, —OS(O)₂OR₉, —OP(O)OR₉OR₉, —OP(O)R₉R₉, —SC(O)R₉, —SC(O)SR₉, or —SC(S)SR₉, wherein each R₉ is independently hydrogen, (C₁-C₆) alkyl, (C₃-C₁₀) carbocyclyl, or 4- to 7-membered heterocyclyl, wherein said alkyl, carbocyclyl, or heterocyclyl is optionally substituted, or
R₁ is an optionally substituted -[L]-diagnostic moiety, wherein the diagnostic moiety is a fluorescent dye, a chromogenic agent, a positron emission tomography (PET) tracer, or a magnetic resonance imaging (MRI) contrast agent, wherein [L] is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different, or
[L] is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different.

5.-9. (canceled)

10. The compound of claim 4, wherein the alkylene chain is a C₁-C₁₂ alkylene chain, or the polyethylene glycol chain has 1 to 10 —(CH₂CH₂—O)— units.

11. (canceled)

12. (canceled)

13. The compound of claim 1, wherein R₂ is hydrogen, an inductive electron withdrawing group, a π-electron withdrawing group, or an optionally substituted -[L]-diagnostic moiety.

14. (canceled)

15. The compound of claim 13, wherein R₂ is an inductive electron withdrawing group, wherein the inductive electron withdrawing group is halogen, OR₆, SR₆, or NR₆R₆, wherein each R₆ is independently hydrogen, C₁-C₆ alkyl, C₆-C₁₂ aryl, 5- to 10-membered heteroaryl, carbonyl, sulfonyl, sulfinyl, or phosphoryl, or
R₂ is a π-electron withdrawing group, wherein the π-electron withdrawing group is —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, or —P(O)NR₆R₆NR₆R₆, wherein each R₆ is independently hydrogen, C₁-C₆ alkyl, C₆-C₁₂ aryl, or 5- to 10-membered heteroaryl, or
R₂ is an optionally substituted -[L]-diagnostic moiety, wherein the diagnostic moiety is a fluorescent dye, a chromogenic agent, a positron emission tomography (PET) tracer, or a magnetic resonance imaging (MRI) contrast agent.

16. The compound of claim 15, wherein the inductive electron withdrawing group is halogen, wherein the halogen is fluoro or chloro.

17.-21. (canceled)

22. The compound of claim 1, wherein R₃ is hydrogen, an inductive electron withdrawing group, a π-electron withdrawing group, or an optionally substituted -[L]-diagnostic moiety.

23. (canceled)

24. The compound of claim 22, wherein R₃ is an inductive electron withdrawing group, wherein the inductive electron withdrawing group is halogen, OR₆, SR₆, or NR₆R₆, wherein each R₆ is independently hydrogen, C₁-C₆ alkyl, C₆-C₁₂ aryl, 5- to 10-membered heteroaryl, carbonyl, sulfonyl, sulfinyl, or phosphoryl, or
R₃ is a π-electron withdrawing group, wherein the π-electron withdrawing group is —C(O)R₆, —C(O)NR₆R₆, —C(O)NR₆R₆, —C(O)OR₆, —S(O)R₆, —S(O)₂R₆, —S(O)OR₆, —S(O)NR₆R₆, —S(O)₂NR₆R₆, —OP(O)OR₆OR₆, or —P(O)NR₆R₆NR₆R₆, wherein each R₆ is independently hydrogen, C₁-C₆ alkyl, C₆-C₁₂ aryl, or 5- to 10-membered heteroaryl, or
R₃ is an optionally substituted -[L]-diagnostic moiety, wherein the diagnostic moiety is a fluorescent dye, a chromogenic agent, a positron emission tomography (PET) tracer, or a magnetic resonance imaging (MRI) contrast agent.

25. The compound of claim 24, wherein the inductive electron withdrawing group is halogen, wherein the halogen is fluoro or chloro.

26.-30. (canceled)

31. The compound of claim 1, wherein R₁ and R₃ are each a -[L]-diagnostic moiety which may be the same or different.

32. The compound of claim 1, wherein R₁, R₂ and R₃ are each a -[L]-diagnostic moiety which may be the same or different.

33. The compound of claim 1, wherein R₄ is a leaving group or a cleavable linking group.

34. The compound of claim 33, wherein R₄ is a leaving group, wherein R₄ is iodo, bromo, chloro, OR₉, SR₉, —OC(O)R₉, —OC(O)OR₉, —OC(O)NR₉R₉, —OC(S)R₉, —OC(S)OR₉, —OC(S)NR₉R₉, —OS(O)₂R₉, —OS(O)₂OR₉, —OP(O)OR₉OR₉, —OP(O)R₉R₉, —SC(O)R₉, —SC(O)SR₉, or —SC(S)SR₉, wherein each R₉ is independently hydrogen, (C₁-C₆) alkyl, (C₃-C₁₀) carbocyclyl, or 4- to 7-membered heterocyclyl, wherein said alkyl, carbocyclyl, or heterocyclyl is optionally substituted, or
R₄ is a cleavable linking group, wherein R₄ is an alkylene chain, that is interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C((O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different, or
R₄ is a polyethylene glycol chain, that is interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclene, 3- to 12-membered heterocyclene, 5- to 12-membered heteroarylene or any combination thereof, wherein each R′ is independently H or optionally substituted C₁-C₂₄ alkyl, wherein the interrupting and the one or both terminating groups may be the same or different.

35. (canceled)

36. (canceled)

37. The compound of claim 34, wherein the alkylene chain is a C₁-C₁₂ alkylene chain, or
wherein the polyethylene glycol chain has 1 to 10 —(CH₂CH₂—O)— units.

38. (canceled)

39. (canceled)

40. The compound of claim 1, wherein R₇ is Me, Et, ⁿBu, iPr, Cy, 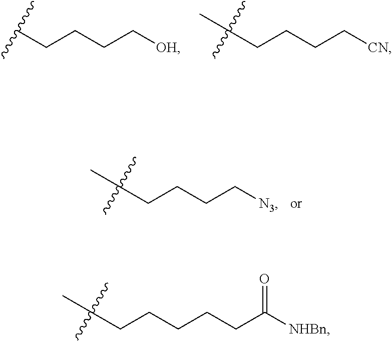
or
wherein R₇ and R₈ together with the nitrogen atom to which they are attached, form a 6-membered heterocyclyl comprising 2 heteroatoms selected from O and N, or
wherein R₈ is Me, Et, nBu, iPr, Cy,

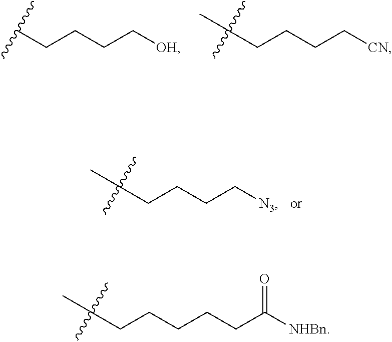

41. (canceled)

42. (canceled)

43. The compound of claim 1, wherein A is absent or a therapeutic moiety.

44. (canceled)

45. The compound of claim 43, wherein the therapeutic moiety is an anti-cancer agent, a hypoxia-inducible factor inhibitor, or an apoptotic agent.

46.-49. (canceled)

50. The compound of claim 1, which is: 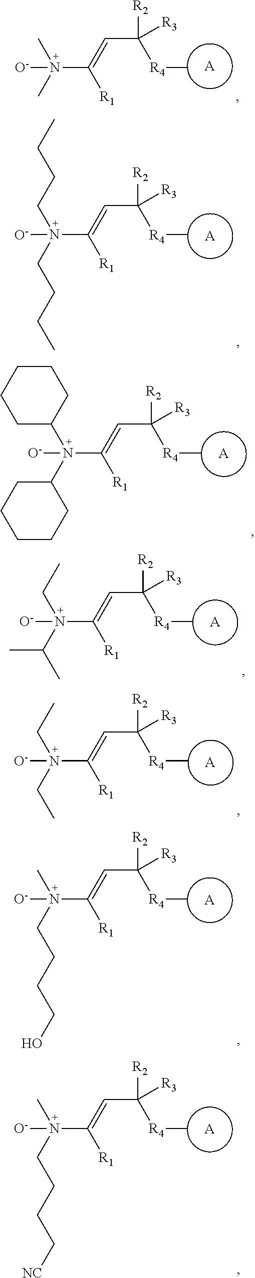 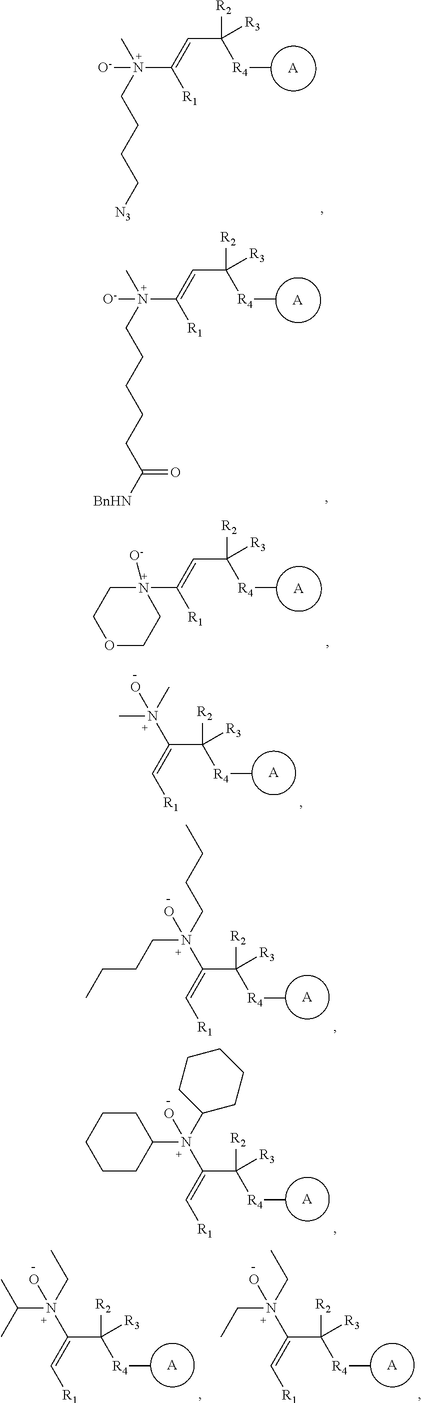 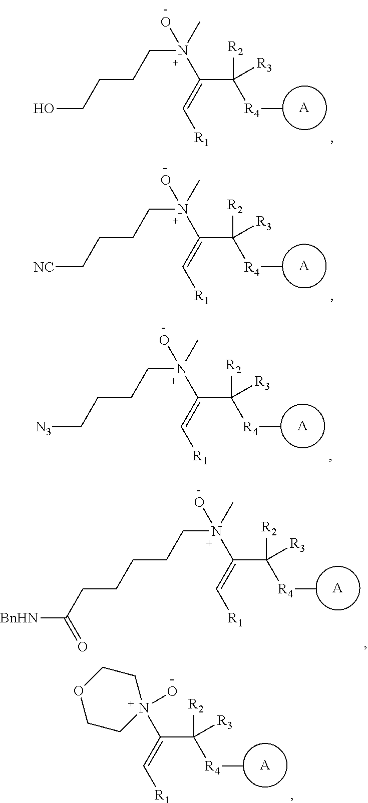
or a pharmaceutically acceptable salt or stereoisomer thereof, or 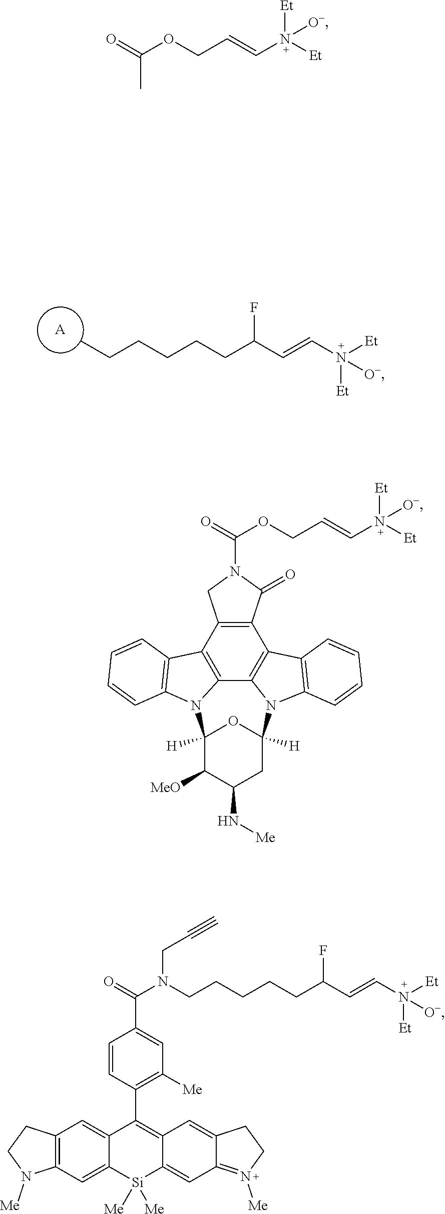
or a pharmaceutically acceptable salt or stereoisomer thereof.

51. (canceled)

52. A compound having a structure represented by formula III or IV: 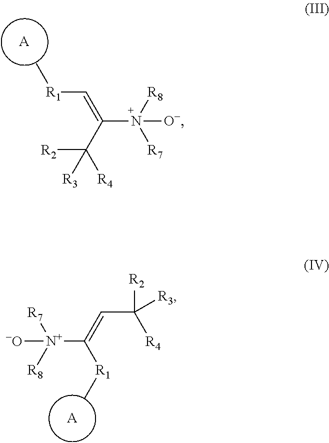
or a pharmaceutically acceptable salt or stereoisomer thereof,
wherein:
R₁ is hydrogen, CH₂, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a cleavable linking group, or -[L]-diagnostic moiety, wherein R₁ may be optionally substituted;
[L] is absent or a linking group that is capable of carrying a plurality of diagnostic moieties, which may be the same or different;
R₂ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a n-electron withdrawing group, a leaving group, or -[L]-diagnostic moiety, wherein R₂ may be optionally substituted;
[L] is absent or a linking group that is capable of carrying a plurality of diagnostic moieties, which may be the same or different;
R₃ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a n-electron withdrawing group, a leaving group, or -[L]-diagnostic moiety, wherein R₃ may be optionally substituted;
[L] is absent or a linking group that is capable of carrying a plurality of diagnostic moieties, which may be the same or different;
R₄ is hydrogen, C₁-C₆ alkyl, C₁-C₆ haloalkyl, C₁-C₆ alkoxy, C₁-C₆ haloalkoxy, OH, CN, NO₂, NH₂, (C₁-C₆ alkyl)NH, (C₁-C₆ alky)₂N, C₃-C₆ carbocyclyl, 5- to 6-membered heterocyclyl, an inductive electron withdrawing group, a n-electron withdrawing group, a leaving group, or -[L]-diagnostic moiety, wherein R₄ may be optionally substituted;
[L] is absent or a linking group that is capable of carrying a plurality of diagnostic moieties, which may be the same or different;
R₇ is (C₁-C₅) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted, or
R₇ and R₈ together with the nitrogen atom to which they are attached, form a 4- to 7-membered heterocyclyl comprising 1 to 3 heteroatoms selected from O, N, and S;
R₈ is (C₁-C₈) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted; and
A is absent, a leaving group or a therapeutic moiety,
provided that the compound of formula (III or IV) contains at least one -[L]-diagnostic moiety or therapeutic moiety,
and when A is a therapeutic moiety, R₁ is a cleavable linking group;
and when at least one of R₂, R₃, and R₄ is a -[L]-diagnostic moiety for a compound of formula (III), R₁ is CH₂ and A is a leaving group;
and when at least one of R₁, R₂, and R₃ is a -[L]-diagnostic moiety and A is absent for a compound of formula (IV), R₄ is a leaving group.

53-95. (canceled)

96. A pharmaceutical composition, comprising a therapeutically effective amount of the compound or pharmaceutically acceptable salt or stereoisomer of claim 1, and a pharmaceutically acceptable carrier.

97. A method of treating a disease or disorder characterized by, or associated with or exhibiting tissue hypoxia, comprising administering to a subject in need thereof a therapeutically effective amount of the compound or pharmaceutically acceptable salt or stereoisomer of claim 1, wherein A is a therapeutic moiety.

98.-105. (canceled)

106. A process of preparing a compound of formula I or II: 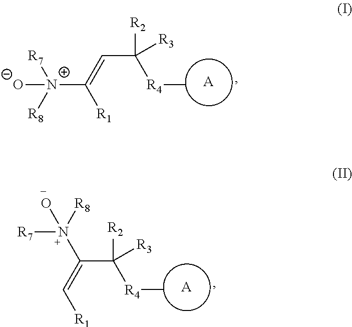
comprising reacting a compound of formula V: 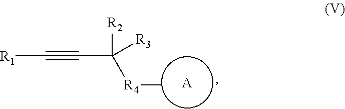
with a compound of formula VII:

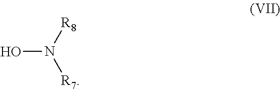

107. A process of preparing a compound of formula III or IV: 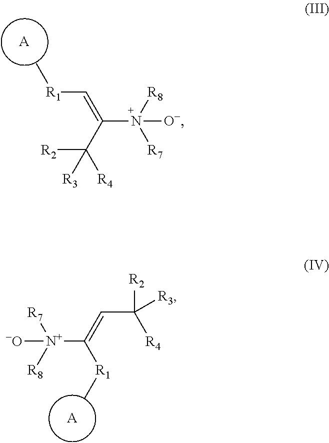
comprising reacting a compound of formula VI: 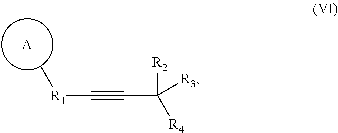
with a compound of formula VII:

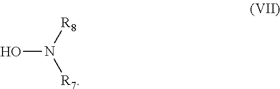

108.-123. (canceled)
